(19) United States
(12) Patent Application Publication
(10) Pub. No.: US 2025/0230492 A1
(43) Pub. Date: Jul. 17, 2025

(54) STOCHQUANT PROBABILISTIC DETECTION AND RELATED METHODS AND SYSTEMS

(71) Applicant: CALIFORNIA INSTITUTE OF TECHNOLOGY, Pasadena, CA (US)

(72) Inventors: Rustem F. ISMAGILOV, Altadena, CA (US); Matthew M. COOPER, Pasadena, CA (US)

(21) Appl. No.: 18/818,505

(22) Filed: Aug. 28, 2024

Related U.S. Application Data
(60) Provisional application No. 63/579,291, filed on Aug. 28, 2023.

(51) Int. Cl. C12Q 1/686; C12Q 1/6806; C12Q 1/6869

(52) U.S. Cl. CPC C12Q 1/686; C12Q 1/6806; C12Q 1/6869

(57) ABSTRACT

Methods and systems are described of a stochastic quantitative approach (StochQuant) that uses molecular counts obtained from a testing measurement, an absolute anchoring measurement of a reference molecule, and possibly additional physical parameters such as quantitatively measurable amounts of a sample, to identify a probability distribution, a confidence interval and/or a confidence level in outcome of a testing measurement of a target molecule, thus improving reliability and accuracy of quantitative detection of the target molecule performed by the testing measurement.

### CROSS-REFERENCE TO RELATED APPLICATION

The present application claims priority to U.S. Provisional Application No. 63/579,291 entitled “StochQuant Probabilistic Detection and Related Methods and Systems” filed Aug. 28, 2023, with docket number P2950-USP the content of which is incorporated herein by reference in their entirety.

### FIELD

The present disclosure relates to detection technology and in particular to stochastic quantification of molecules. More particularly the present disclosure relates to StochQuant probabilistic detection and related methods and system.

### BACKGROUND

Confidence is an inherent problem of any type of detection. It stems from the knowledge that a value obtained as a result of a detection process may not correctly represent a detected item, in view inaccuracies introduced by the detection technique used.

A confidence score is often used as a measure of the probability that a value provided in outcome of detection correctly correspond to a detected item.

In particular, with respect to detections, such as molecular detection, performed through sampling process and/or in sample or environments including target molecules present at low absolute and/or relative abundance, improving the confidence of qualitative and/or quantitative presence remains challenging in view of the inherent stochasticity of the detection system as understood by a skilled person.

### SUMMARY

The present disclosure describes methods and systems to perform molecular detection according to a quantitative stochastic approach (herein StochQuant approach or StochQuant), which provides probability distributions in place of single values for a parameter used in molecular detection.

In particular, in StochQuant detection methods and systems of the disclosure, a probability distribution of a target molecule abundance in an environment (herein StochQuant probability distribution) detected in outcome of a testing measurement, is obtained as a function of i) a molecular count of the target molecule detected in the environment or a sample thereof, ii) a molecular count of a reference molecule added to or detected in, the environment a sample or a subsample thereof, in combination with iii) an absolute anchoring value of the reference molecule; and in some embodiments also iii) a quantitively measured amount (e.g. volume) of a sample or a subsample of the environment.

In StochQuant detection methods and systems of the disclosure, the testing measurement comprises or consists of a measuring workflow in which a physical manipulation of the environment, a sample and/or a subsample thereof are performed to provide the molecular counts of the target molecule and of the reference molecule as well as the anchoring measurement required to provide StochQuant probability distribution.

In StochQuant detection methods and systems of the disclosure, the StochQuant probability distribution is obtained from the molecular counts detected during the measuring workflow of the testing measurement in the form of one or more testing parameters such as read counts from sequencing or fluorescence intensity in flow cytometry as well as additional testing parameters identifiable by a skilled person.

, The StochQuant probability distribution so obtained enables a quantitative and/or qualitative detection of the target molecule that takes into account the stochasticity inherent to the detection system due in particular to the need of performing physical manipulations of the environment, a sample and/or a subsample thereof such as sampling and/or additional manipulations inherent to the detection workflow of the testing measurement used for performing detection of the target molecule in the environment a sample and/or a subsample thereof.

The stochasticity inherent to the detection system characterizes in particular detection workflow performed in an environment, sample or subsample thereof comprising a known or expected small numbers of molecules from an environment, and/or obtained during the testing measurement, as understood by a skilled person upon reading of the disclosure.

Accordingly, in StochQuant detection methods and systems of the disclosure performing in an environment a sample and/or a subsample thereof, a testing measurement in which a detection workflow configured to detect molecular counts is modeled according with StochQuant methods and system herein described, provide in place of a single value of one or more testing parameters, a probability distribution of values indicative of the detected target molecule abundance in the environment, which will account for the probability that the target molecule is present or absent in the environment, as well as the probable count of target molecule in the environment.

As a consequence, the StochQuant detection methods and systems of the disclosure provide an improvement in detection technology because StochQuant testing measurements enable detection of a target molecule in an environment with an increased confidence with respect to corresponding testing measurement performed without StochQuant detection as understood by a skilled person upon reading of the present disclosure.

In particular according to a first aspect, a method and a systems are described to improve a testing measurement for detection of an abundance of a target molecule in a physical environment. In the method and system according to the first aspect the testing measurement comprises a measuring workflow for the molecular count of a target molecule and a reference molecule.

The method comprises: i) dividing the measuring workflow into one or more measuring segments arranged in a measuring workflow order, each of the one or more measuring segments comprising one or more physical manipulations impacting the molecular count of the target molecule and/or of the reference molecule.

The method further comprises: ii) calibrating the one or more measuring segments by building corresponding stochastic representations of each of the one or more measuring segments into a computer-based system, the stochastic representations taking as inputs physical parameters of the measuring workflow.

The method also comprises: iii) chaining the corresponding stochastic representations together into a model of the measuring workflow by connecting outputs of measuring segments into inputs of other measuring segments in the measuring workflow order, such that the model takes as model inputs the physical parameters including at least a target molecule molecular count, a reference molecule molecular count, and an absolute anchoring value of the reference molecule.

The method additionally comprises: iv) configuring the computer-based system to provide a probability distribution of an abundance of the target molecule based on the model of the measuring workflow when provided the model inputs.

The related system comprises reagents and/or equipment to perform a testing measurement and embodiments of methods described in the first aspect. Examples of system components include computing devices configured to carry out one or more embodiments of the methods, computer-readable non-transient mediums encoded with programs configured to carry out one or more embodiments of the methods, PCR kits, biotech library preparation kits, flow cells, microfluidic devices, genetic tags, etc.

According to a second aspect a method and system are described to build a computer-readable program that improves a measuring workflow of a testing measurement for detection of an abundance of a target molecule in a physical environment.

The method comprises: i) dividing the measuring workflow into one or more measuring segments arranged in a measuring workflow order, each of the one or more measuring segments comprising one or more physical manipulations of a molecular count of the target molecule and/or of a reference molecule in the environment, a sample and/or a subsample thereof.

The method further comprises: ii) calibrating the one or more measuring segments by building corresponding stochastic representations of each of the one or more measuring segments into a computer-readable program, the stochastic representations taking as inputs physical parameters of the measuring workflow.

The method also comprises: iii) chaining the corresponding stochastic representations together into a model of the measuring workflow by connecting outputs of measuring segments into inputs of other measuring segments in the measuring workflow order, such that the model takes as its inputs the physical parameters including at least a target molecule molecular count, a reference molecule molecular count, and an absolute anchoring value of the reference molecule.

The method additionally comprises: iv) configuring the computer-readable program to provide a probability distribution of an abundance of the target molecule based on the model of the measuring workflow when run on a computer system and given the inputs by a user of the computer-readable program.

The related system comprises reagents and/or equipment to perform a testing measurement and embodiments of methods described in the second aspect. Examples of system components include computing devices configured to carry out one or more embodiments of the methods, computer-readable non-transient mediums encoded with programs configured to carry out one or more embodiments of the methods, PCR kits, biotech library preparation kits, flow cells, microfluidic devices, genetic tags, etc.

According to a third aspect, a method and a system are described to probabilistically detect a target molecule in an environment through a measuring workflow of a testing measurement to measure abundance of the target molecule in the environment in combination with a reference molecule.

The method comprises: i) performing the measuring workflow on the environment, a sample and/or a subsample thereof, the measuring workflow comprising one or more physical manipulations of the target molecule and/or the reference molecule in the environment, the sample and/or the subsample thereof impacting a molecular count of the target molecule and/or of the reference molecule.

The method also comprises ii) providing a molecular count of the target molecule in the environment from performing the measuring workflow by detecting the molecular count of the target molecule in the environment, the sample and/or the subsample thereof.

The method further comprises iii) providing a molecular count of a reference molecule from performing the measuring workflow by adding a known amount of the reference molecule and/or by detecting the molecular count of the reference molecule in the environment, the sample and/or the subsample thereof.

The method additionally comprises iv) providing an absolute anchoring value of the reference molecule.

The method also comprises v) based on at least the absolute anchoring value of the reference molecule, the molecular count of the target molecule, and the molecular count of the reference molecule, forming a probability distribution of abundances of the target molecule in the environment based on a modeling of the measuring workflow, the modeling taking into account stochastic properties of the physical manipulations of the target molecule. and/or the reference molecule in the environment, the sample and/or the subsample thereof.

The related system comprises reagents and/or equipment to perform a testing measurement and embodiments of methods described in the third aspect. Examples of system components include computing devices configured to carry out one or more embodiments of the methods, computer-readable non-transient mediums encoded with programs configured to carry out one or more embodiments of the methods, PCR kits, biotech library preparation kits, flow cells, microfluidic devices, genetic tags, etc.

According a fourth aspect a method and a system to probabilistically detect a target molecule in an environment, are described. The method comprises:
performing a testing measurement comprising
   obtaining a molecular count of the target molecule in an environment or a sample thereof; and
   obtaining a molecular count of a reference molecule; and
providing an absolute anchoring value of the reference molecule in the sample; and
obtaining a probability distribution of the target molecule abundance in the sample as a function of
   the molecular count of the target molecule;
   the molecular count of the reference molecule; and
   the absolute anchoring value of the reference molecule;
      In the method to probabilistically detect a target molecule in an environment of the first aspect, the probability distribution of the target molecule abundance in the environment is indicative of the confidence of detection or non-detection or confidence of the quantitative value of the target molecule detected in the environment.

The related system comprises reagents and/or equipment to perform a testing measurement and embodiments of methods described in the fourth aspect. Examples of system components include computing devices configured to carry out one or more embodiments of the methods, computer-readable non-transient mediums encoded with programs configured to carry out one or more embodiments of the methods, PCR kits, biotech library preparation kits, flow cells, microfluidic devices, genetic tags, etc.

According to a fifth aspect a method and a system are described to probabilistically measure an abundance of a target molecule in an environment.

The method comprises: i) determining a) an absolute anchoring value of a reference molecule in the environment.

The method further comprises ii) performing a testing measurement comprising a measurement workflow, producing quantitative testing measurements, on the environment, a sample and/or a subsample thereof, to establish:
b) a corresponding molecular count of the target molecule in the environment; and
c) a corresponding molecular count of the reference molecule in the environment.

The method also comprises iii) inputting a), b) and c) into a computer-based system, the computer system being configured to generate a probability distribution of abundance of the target molecule in the sample based on the basis of a), b) and c) by a model of the quantitative testing measurements.

The method additionally comprises iv) based on the probability distribution, producing, through the computer-based system, one or more of:
confidence level of abundance values above and below a threshold abundance value of the target molecule input to the computer system;
confidence interval of abundance values based on an abundance value confidence level of the target molecule input to the computer system; and
abundance value confidence level based on a confidence interval of abundance values input to the computer system.

The related system comprises reagents and/or equipment to perform a testing measurement and embodiments of methods described in the fifth aspect. Examples of system components include computing devices configured to carry out one or more embodiments of the methods, computer-readable non-transient mediums encoded with programs configured to carry out one or more embodiments of the methods, PCR kits, biotech library preparation kits, flow cells, microfluidic devices, genetic tags, etc.

According to a sixth aspect a computer-based system is described comprising a processor, memory, input components, and output components.

The computer-based system is configured to: i) receive, process and store, through the input components, the processor and the memory, a) an absolute anchoring values of a reference molecule in an environment a sample and/or a subsample thereof, b) a molecular count of a target molecule in the environment as determined by a measuring workflow performed in the environment, the sample and/or a the subsample thereof, and c) a molecular count of the reference molecule in the environment as determined by the measuring workflow performed in the environment, the sample and/or a the subsample thereof.

The computer-based system is further configured to: ii) process, through the processor, a), b) and c) from i) into a model of the measuring workflow configured to obtain probabilistically distributed abundance values of the target molecule in the environment; and at least one of:
iiia) receive, through the input components, a threshold abundance value of the target molecule and process, through the processor, the threshold abundance value of the target molecule through the probabilistically distributed abundance values of the target molecule to obtain and output, through the output components, a confidence level of abundance values above and below the threshold abundance value of the target molecule; or
iiib) receive, through the input components, an abundance value confidence level of the target molecule and process, through the processor, the abundance value confidence level of the target molecule through the probabilistically distributed abundance values of the target molecule to obtain and output, through the output components, a confidence interval of abundance values of the target molecule; or
iiic) receive, through the input components, a confidence interval of abundance values of the target molecule and process, through the processor, the confidence interval of abundance values of the target molecule through the probabilistically distributed abundance values of the target molecule to obtain and output, through the output components, an abundance value confidence level of the target molecule.

The related method comprises the system running a program encoded to carry out one or more of the methods described herein, including from other aspects.

According to a seventh aspect a method is to probabilistically detect a target molecule in an environment, the method comprising:
separating a portion of the environment to obtain a sample of the environment the sample having a quantitatively measurable amount;
providing an absolute anchoring value of a reference molecule in the sample;
performing a testing measurement comprising
obtaining a molecular count of the target molecule in the sample; and
obtaining a molecular count of the reference molecule in the sample; and
obtaining a probability distribution of the target molecule abundance in the sample as a function of
the molecular count of the target molecule;
the molecular count of the reference molecule;
the absolute anchoring value of the reference molecule; and
a quantitively measured amount of the sample;
the probability distribution of the target molecule abundance in the sample indicative of the confidence of detection or non-detection or confidence of the quantitative value of the target molecule detected in the sample which is indicative of the probabilistic detection of the target molecule in the environment.

The related system comprises reagents and/or equipment to perform a testing measurement and embodiments of methods described in the seventh aspect. Examples of system components include computing devices configured to carry out one or more embodiments of the methods, computer-readable non-transient mediums encoded with programs configured to carry out one or more embodiments of the methods, PCR kits, biotech library preparation kits, flow cells, microfluidic devices, genetic tags, and additional system components identifiable by a skilled person.

In StochQuant detection methods and systems of the disclosure StochQuant probability distribution will thus provide an advantageous probabilistic detection (probability function) of the target molecule in the sample which is indicative and relates back to the probabilistic detection (quantitative or qualitative) of the target molecule in the environment from which the sample is obtained, as understood by a skilled person upon reading of the present disclosure.

StochQuant methods and systems provide an improvement to various fields of technology in which molecular detection is performed by method systems that determine molecular counts. In particular StochQuant methods and systems enable detection that account for the inherent stochasticity introduced by the manipulations required by a detection workflow, thus augmenting the accuracy, precision, confidence in, and reliability of the results of the detection, and solving a problem arising from the technology itself. Accordingly, StochQuant methods and systems also improve various technical fields, such as diagnostics, in-vitro diagnostics, cancer diagnostics, prenatal diagnostics, biotherapeutics, medical drug design and development, biotic treatment, bioanalysis, biotechnology, agricultural biotechnology, food testing, genetic testing, and immunology.

The StochQuant detection methods and systems herein described can be used in connection with various applications wherein accurate and/or reliable detection of a molecular count is desired, in particular in target environment including target molecule in low abundance. For example, the StochQuant detection methods and systems herein described allow in several embodiments herein described for qualitative and/or quantitative microbiome profiling and/or detection of target molecules in environments sch as tissues, organs, stool, biopsies and bodily fluids in human and veterinary medicine, or environmental sample analyses (e.g., soil and water) or sample thereof. Exemplary application of the StochQuant detection methods and systems herein described comprise, biotherapeutics, medical drug development, clinical application, diagnostic applications, in-vitro diagnostics, cancer diagnostics, prenatal diagnostics, drug development, biotic treatment, biotechnology, agricultural biotechnology, food testing, bioanalysis, genetic testing, immunology and additional applications identifiable by a skilled person.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1 shows a schematic representation of a StochQuant Workflow where an exemplary set of steps directed to Build the StochQuant Workflow and an exemplary set of steps directed to use the StochQuant Workflow are schematically identified.

FIG. 2 shows schematic representations of uses of probability distribution provided by a detection method StochQuantized by a workflow, such as the one exemplified in Example 00, based on the concept of the confidence interval (FIG. 2, Panel A). These include providing a confidence level for a given confidence interval of target abundance (FIG. 2, Panel B), or providing a confidence interval for a given confidence level (FIG. 2, Panel C).

FIGS. 3A-3E: show charts and schematics reporting the result of experiments demonstrating limitations of current approaches of 16S rRNA gene sequencing and analysis of low-to-moderate biomass samples that highlight the problem of confidence in making determinations based upon 16S rRNA gene sequencing measurements. In particular, FIG. 3A shows a schematic illustration of experimental design of the sequencing experiment of a defined microbial community prepared at a range of dilutions. In this example, each dilution is an environment. Symbols correspond to different taxa (target 16S rRNA gene molecules of a taxon), as given in FIG. 3B. FIG. 3B shows rates of differential abundance type I errors (incorrectly determining taxa to be differentially abundant when they are not) for each taxon among different dilutions. FIG. 3 Cshows an exemplary trial (described in Example 4) in which a taxon in dilution 4 (MD4) was found to be over 2.3× lower in mean relative abundance compared with MD1, and an example trial in which the same taxon in MD4 was found to be nearly 2.5× higher in mean relative abundance compared to MD1. FIG. 3D, shows the results of experiments in which four sequencing replicates of a no-template control (NTC), with the 10 most abundant taxa shown, highlighting the problem of confidence in the detection and quantitative detection of low numbers of target molecules in a NTC environment that will be used for the determination of the presence of targets in the dilution environments compared to the abundance of the targets in the NTC environment. FIG. 3E shows the result of exemplary experiments illustrating variability of PCA of relative abundances with four trials, highlighting the problem of confidence in the PCA results.

FIGS. 4A-4C show charts diagrams and schematics illustrating an exemplary stochastic representations of a measuring workflow (also referred to as a forward measurement model) of amplicon sequencing provided as a representative testing measurement. The illustration of FIGS. 4A-C provides a mathematical representation of the amplicon sequencing testing measurement and therefore a model of the measuring workflow, The stochastic representations of the measuring workflow of FIGS. 4A-C describes the intrinsic variability of amplicon sequencing data from low-to-moderate biomass samples as arising from two sequential stochastic sampling events. In particular, a the model of the exemplary measuring workflow of the amplicon sequence of FIGS. 4A-C allows identification of i) Segments of the measuring workflow (also referred to as steps of the detection workflow) which are characterized by stochasticity and can be represented as probabilistic stochastic mathematical functions, and ii) Physical parameters (measurable parameters of the testing measurement workflow that affect the molecular count of the target) which parameterize the probabilistic stochastic mathematical functions to account for the stochasticity. In particular, FIG. 4A shows a schematic of an exemplary model of the measuring workflow (forward measurement model) of the amplicon sequencing testing measurement described in FIGS. 4A-C. In particular the forward measuring model of FIG. 4A, describes how probable molecular counts of a target molecule is stochastically yielded from the combination of two stochastic processes that occur during an amplicon sequencing molecular detection workflow. FIGS. 4B-C describe simulations generated by the exemplary forward measurement model illustrating the stochastic sampling of input target/reference molecules during separation of a sample of the environment (also referred to as the loading of template DNA) into a library-preparation reaction. This illustration is of the first segment of the forward measuring model. In particular, FIG. 4B describes a schematic representation of results of experiments showing that when taxon absolute abundance (the number of target molecules in an environment) and total load (the number of reference molecules in an environment) are low (10³ 16S rRNA gene copies/mL of target and 5×10⁴ reference 16S rRNA gene copies/mL but relative abundance and the molecular count of the reference molecule (read depth) are sufficiently high (2% relative abundance and 100,000 total reads) detection and measurement noise are driven by the stochastic loading of molecules. The illustration of FIG. 4B shows that the stochasticity of the first segment of the forward measuring model accounts for the majority of the stochasticity/variability of the entire representation of the measurement workflow. FIG. 4C presents results of experiments showing that when taxon absolute abundance and total load are high (e.g. 10⁶ 16S rRNA gene copies/mL of target and 10¹⁰ total 16S rRNA gene copies/mL) but relative abundance and read depth are low (e.g. 0.01% relative abundance and 5,000 reads), detection and measurement noise are driven by the stochastic sampling of reads on the flow cell. Accordingly, the results present in FIG. 4C show that the stochasticity of the second segment of the forward measuring model accounts for the majority of the stochasticity/variability of the entire model as will be understood by a skilled person upon reading of the present disclosure.

FIGS. 5A-5F show charts and schematics reporting results of experiments showing that the exemplary model of the measuring workflow provided by the forward measurement model, accurately describes/represents detectability and measurement noise from the actual amplicon sequencing testing measurement. In particular, FIG. 5A shows a comparison of a StochQuant simulation of a sequencing experiment (simulated read counts transformed to relative abundance by dividing the molecular count of the target molecule by the molecular count of the reference molecule) assuming a taxon relative abundance of 1.9% relative abundance under identical conditions to the experimentally observed results reported in FIGS. 3A-3E) of Pseudomonas, a taxon that was present in the defined microbial community at 1.9% relative abundance. (FIGS. 5B-D report results of experiments directed to evaluate the accuracy of the exemplary representation of the measurement workflow provided by the forward measurement model by comparing the results yielded by the forward measurement model to the results yielded by the testing measurement. in the illustration of FIGS. 5B-D. In particular, in FIG. 5B shows comparison of a StochQuant simulation of a sequencing experiment provided as representative testing measurement assuming a constant absolute abundance of 500 16S rRNA gene copies/mL, compared to experimentally observed results from a contaminant taxon. (FIG. 5C shows Experimental result validating the performance of the StochQuant model of the molecular detection workflow of amplicon sequencing provided as a representative example of a testing measurement. The result measured the detection frequency of Salmonella (0.04% relative abundance) under each of the four dilution conditions (environments) (MD1-MD4) compared to StochQuant model simulations of the same experiment. FIG. 5D shows experimental result validating the performance of the StochQuant model of the molecular detection workflow of amplicon sequencing. The result measured the coefficient of variation (% CV) for each of the top 5 defined-community taxa under each of the four dilution conditions compared to StochQuant simulations of the same experiment. FIG. 5E shows an exemplary frequency of detection of taxa in an environment based on StochQuant detection of molecular counts of a 16S RNA as a biomarker of the taxa. In the illustration of FIG. 5E the frequencies of detection are simulated as a function of taxon absolute abundance, total bacterial load, template loading volume, and read depth. Plot gradients (on a 0.0-1.0 scale) indicate the probability of detection. Limits of detection (at least 95% probability of detection) are shown for relative abundance (diagonal line) and absolute abundance (horizontal line). FIG. 5F shows a generalized schematic of the relationship between detectability of a taxon at a given absolute abundance (taxon 16S rRNA gene copies/mL) as a function of total bacterial load (total 16S rRNA gene copies/mL), template loading volume, and read depth. “Sliders” indicate how the 4 detection zones are affected by changing read depths and template loading volumes.

FIG. 6A show a set of charts reporting results of experiments illustrating a comparison of the detectability of molecular counts in form of detected frequencies of targets yielded by StochQuant simulated detection frequencies, versus experimentally observed detection frequencies (from the amplicon sequencing testing measurement) for each mock community taxon under each dilution condition. The results reported in the illustration of FIG. 6A enable evaluating the accuracy of the exemplary StochQuant simulated detection frequencies as a model of the amplicon sequencing testing measurement providing the frequencies.

FIG. 6B-D: shows a set of charts reporting results of experiments illustrating a comparison of the measurement noise (% CV) yielded by a StochQuant simulated relative abundance CV versus experimentally observed relative abundance CV from the testing measurement of amplicon sequencing. In particular, the illustration of FIG. 6B shows a diagram reporting the comparison when only the second segment of the StochQuant simulated relative abundance CV (stochastic sampling of reads on the sequencing flow cell) is considered, FIG. 6C show a diagram reporting the comparison when only the first segment of the StochQuant simulated relative abundance CV (stochastic loading of target/reference template DNA molecules) is considered, and FIG. 6D show a diagram reporting the comparison when the entire measurement workflow representation of the StochQuant simulated relative abundance CV (both the stochastic loading of target/reference molecules and sampling of reads) is considered. The results reported in FIGS. 6B-D can be used to evaluate the Accuracy of each representation as will be understood by a skilled person upon reading of the present disclosure.

FIGS. 7A-G shows charts, diagrams and schematics reporting results of experiments showing that the StochQuant representation of a detection workflow directed to detect abundance of 16S RNA marker for target taxon in an environment enables inference of taxon abundance and measurement uncertainty by yielding a probability distribution of taxon (target) abundance from a molecular count of the target molecule obtained (a read count measurement), an absolute anchoring value (total load measurement), and other StochQuant parameters including a molecular count of the reference molecule obtained via the testing measurement (“read depth”), and a measurable amount of sample separated from an environment. The illustrations of FIGS. 7A-G show that StochQuant probability distributions of number of target molecules in an environment (or abundances computed form the number of target molecules in an environment) are obtained from a molecular count of the target (read count measurement), an absolute anchoring value of the reference molecule (total load measurement), and a measurable amount of sample separated from an environment (experimental parameters) and a molecular count of the reference molecule (experimental parameters). In particular, FIG. 7A, shows a schematic of an exemplary StochQuant representation of a detection workflow yielding a probability distribution of number of target molecules detected through amplicon sequencing as the testing measurement. In this example, the StochQuant Detection Workflow infers taxon abundance and measurement uncertainty by generating a probability distribution of a taxon abundance from an observed molecular counts in the form of read-count and read depths,in combination with additional psychical parameters provided by the total microbial load (absolute anchoring value of the reference), volumes used. FIG. 7B shows a chart reporting simulated data yielded by a StochQuant representation of a measuring workflow of the amplicon sequencing of 16S rRNA as markers of a taxon at 1% relative abundance in a sample with a total microbial load of 40,000 16S rRNA gene copies/mL, with 2 μL of template loaded into the library preparation reaction, sequenced with 100,000 reads. Probability distributions of molecular counts in the form of read counts from the simulation, colored by number of molecules loaded into the library preparation reaction are shown. For visualization purposes, only molecules less than or equal to 3 are shown.

FIG. 7C shows a chart reporting the StochQuant probability distributions of abundance generated from three read-count outcomes (No-detection, 1500, and 3000 reads) under the simulation conditions in FIG. 7B with the StochQuant representation of the amplicon sequencing detection workflow. FIG. 7D shows charts reporting StochQuant probability distribution of abundance generated from one sequencing replicate each from dilutions MD1, MD2, MD3, and MD4, all sequenced at similar read depths of 105,000-114,000. FIG. 7E shows charts reporting relative abundance values drawn from each of the four distributions shown in FIG. 7D compared with all experimentally observed replicate measurements. FIGS. 7F-G show report result of a demonstration of how the StochQuant detection workflow yields measurement uncertainties from a single read-count measurement for each dilution (MD1 and MD4). Each of FIGS. 7F-G, for two taxa in the defined community, Bacillus (light) and Pseudomonas (dark), plot: (i) the StochQuant probability distributions for each taxon's relative abundance, (ii) the relative abundances drawn from StochQuant probability distributions, and (iii) the relative abundances observed experimentally in all 20-22 replicates.

FIG. 8 shows charts reporting results of experiments showing exemplary probability distributions of (FIG. 8 Panel A) relative and (FIG. 8 Panel B) absolute of Bacillus in two sequencing replicates; one for which zero reads were detected (r01) and one for which nearly 1200 reads were detected (r22).

FIGS. 9A-9H: shows charts reporting results of experiments showing an exemplary comparison of standard detection vs StochQuant detection in the analysis of a defined microbial community. In particular FIGS. 9A-B show charts reporting absolute-abundance estimates and corresponding StochQuant probability distributions of (FIG. 9A) Spirosoma (a contaminant) and (FIG. 9B) Pseudomonas (a member of the defined community) using standard and StochQuant approaches in four NTC sequencing replicates and one sequencing replicate each from dilutions MD3 and MD4. FIG. 9C reports a chart reporting a number of genera remaining in the dataset after contamination filtering with the standard approach (based on absolute abundances) and with the StochQuant approach. FIG. 9D reports PCA of the StochQuant-derived center-log-ratio transformed relative abundances of trials from FIG. 3E. FIG. 9E show a comparison of total number of times each differential abundance approach (ALDEx2, DESeq2, Kruskal, and StochQuant) incorrectly determined a taxon to be differentially abundant between two groups (P-value <0.05). FIGS. 9F-H report examples for high (FIG. 9F), medium (FIG. 9G) and low (FIG. 9H) abundance taxa illustrating how increased variability at high dilutions (MD4) can lead to spurious “statistically significant” differences and how these spurious results are corrected by StochQuant by recognizing the broadening of probability distributions at high dilutions.

FIG. 10 shows charts reporting results of sampling from StochQuant probability distributions of target relative abundance, projected onto the principal components from FIG. 3E for each of the trials. The StochQuant PCA “clouds” for each sequencing replicate in each trial are shown.

FIGS. 11A-11H show charts and schematics illustrating an exemplary analysis performed with StochQuant which identifies compositional and taxon-level differences between locations along the gastrointestinal (GI) tract of Patient 12. FIG. 11A shows a schematic of a longitudinal sample collection along the GI tract. FIG. 11B shows a chart reporting PCA of relative abundances filtered by the standard absolute-abundance contamination identification approach. FIG. 11C shows a chart reporting PCA of relative abundances filtered by the StochQuant approach, with the StochQuant generated relative abundances projected onto the same principal component axes. FIG. 11D) shows a chart reporting top 15 feature loadings of PC1 and PC2 with standard approaches. Each taxon is colored by its most common habitat (human, environmental, or ambiguous). FIG. 11E) shows a chart reporting top 15 feature loadings of PC1 and PC2 with the StochQuant approach. FIG. 11F shows a chart reporting taxa that were identified to be differentially abundant between GI locations by any of the differential abundance methods, sorted by P StochQuant. P-values <0.001 were set to 0.001 for plotting purposes. FIGS. 11G-H) shows chart reporting (Left) Relative abundances from the original 16S rRNA gene sequencing data, (Middle) StochQuant probability distributions of abundance generated from the original sequencing data, and (Right) (n=2) additional sequencing replicates of each rectum biopsy. In each plot, relative abundances below the Limit of Detection (LoD) for each sample are colored in dark gray. FIG. 11G) shows a chart reporting results of analysis in which Dialister was called differentially abundant by all three standard methods (DESeq2, ALDEx2, Kruskal) and by StochQuant. In the exemplary analysis illustrated in FIG. 11G, StochQuant predicted that the observed differences in Dialister relative abundance was greater than the StochQuant predicted measurement noise (confirmed by sequencing replicates), and therefore Dialister was determined to be differentially abundant between the TI and R of Patient 12. (H) R. gnavus was called differentially abundant by standard approaches but not by StochQuant. the exemplary analysis illustrated in FIG. 11G, StochQuant correctly infers that the presence of R. gnavus in terminal ileum biopsies but absence of R. gnavus in rectum biopsies may have occurred due to the higher LoD in the rectum biopsies. StochQuant therefore correctly determines (as confirmed by sequencing replicates) that R. gnavus is not differentially abundant, as the taxon can be stochastically detected at similar abundances in the rectum as those found in the terminal ileum. LoD in FIG. 11G is not shown, as it is below the plotting limits of the y-axis of the plot.

FIG. 12 shows charts reporting results of experiments in which total bacterial loads, were measured via digital PCR with universal 16S rRNA gene primers in each of the biological replicate biopsies from the terminal ileum (TI), ascending colon (AC), descending colon (DC), and rectum (R) in (a) Patent 12 and (b) Patient 13. (c) Total bacterial loads of the no template control (NTC) processing blanks.

FIGS. 13A-13D shows charts reporting results of experiments showing that StochQuant improves PCA analysis of low-bacterial load biopsies in Patient 13. In particular FIG. 13A shows that PCA of relative abundances filtered by the standard absolute-abundance contamination identification approach. FIG. 13B show that PCA of relative abundances filtered by the StochQuant approach (including contamination removal), with the StochQuant generated relative abundances projected onto the same principal component axes. FIG. 13C shows top 15 feature loadings of PC1 and PC2 with standard approaches. Each taxon is colored by its most common habitat (human, environmental, or ambiguous). FIG. 13D shows top 15 feature loadings of PC1 and PC2 with the StochQuant approach. The separation among sampling sites observed in standard PCA is driven by contaminants, which play a larger role as total microbial load decreases along the GI tract of this patient, (FIG. 12 Panel B). leading to the spurious trend observed in FIG. 13A

FIGS. 14A-14D shows charts reporting relative abundances of the additional four taxa that were differentially abundant by all three standard differential abundance approaches (DESeq2, ALDEx2, Kruskal) and by StochQuant between biological replicates collected from the terminal ileum (TI) (n=3) and rectum (R) (n=3) from Patient 12 (see FIG. 14A-D). (Left) Relative abundances from the original 16S rRNA gene sequencing data, (Middle) StochQuant probability distributions of abundance generated from the original sequencing data, and (Right) (n=2) additional sequencing replicates of each rectum biopsy, with relative abundances below the Limit of Detection (LoD) for each sample colored in dark gray. LoDs in (FIG. 14B-C) are not shown, as the LoDs were below the plotting limits of the y-axis of the plots.

FIGS. 15A-E shows charts reporting results of experiments showing that StochQuant improves interpretation of differential abundance analysis of low-bacterial load biopsies in Patient 13. In particular FIG. 15A shows results for taxa that were identified to be differentially abundant between GI locations by any of the differential abundance methods, sorted by PStochQuant. P-values <0.001 were set to 0.001 for plotting purposes. 46 taxa differentially abundant from DESeq2, 33 from Kruskal, 21 from ALDEx2, and 2 from StochQuant (with contamination filtering). FIG. 15B shows subtle differences in Bacteroides (one of just two taxa determined to be differentially abundant by all standard approaches and StochQuant) relative abundance were observed in initial sequencing data. StochQuant accurately predicts the small measurement noise among TI biopsies and larger measurement noise among R biopsies, and correctly infers that the difference in abundance between TI and R in Patient 13 is greater than the measurement uncertainty within each biopsy. FIG. 15C shows results for Anaerococcus which is initially undetected in TI biopsies and determined to be differentially abundant by standard approaches, but StochQuant correctly predicts that Anaerococcus can be stochastically detected in terminal ileum biopsies, which is confirmed by re-sequencing the biopsies. FIG. 15D shows statistically significant differences in abundance of Streptococcus are initially observed by all standard approaches. Even though a 100-fold difference (1% relative abundance in the TI vs less than 0.01% in the R) in relative abundance is observed, StochQuant correctly infers that these differences at low total microbial loads may have been stochastically observed. Upon re-sequencing, Streptococcus is indeed stochastically detected at similar relative abundances (1%) the rectum as in the terminal ileum. FIG. 15E similarly shows that, Senegalimassilia is detected in all (n=3) TI biopsies but completely undetected in all (n=3) R biopsies of Patient 13, and is thus differentially abundant by all tested standard approaches. StochQuant correctly infers that Senegalimassilia may not be differentially abundant with statistical significance. Furthermore, StochQuant accurately predicts that Senegalimassilia is above LoD in the TI, and thus should be consistently detected upon resequencing, while Senegalimassilia is below LoD in the R, and thus can be stochastically detected upon resequencing of rectum biopsies.

FIGS. 16A-16D chart reporting results of exemplary experiments illustrating how StochQuant probability distributions of taxon abundance and StochQuant predictions of individual sample LoDs improves interpretation and conclusions from differential abundance analyses from human samples with different total microbial loads and read depths. In particular, FIGS. 16A-D show (Left) Relative abundances from the original 16S rRNA gene sequencing data, (Middle) StochQuant probability distributions of abundance generated from the original sequencing data, and (Right) (n=2) additional sequencing replicates of each rectum biopsy, with relative abundances below the Limit of Detection (LoD) for each sample colored in dark gray. LoDs in FIG. 16A-B are not shown, as the LoDs were below the plotting limits of the y-axis of the plots. More particularly FIG. 16A Panel A) shows that StochQuant probability distributions of abundance enables sensitive detection of subtle changes in taxon abundance, such as the increase in relative abundance of Lachnospiraceae between the TI and R of Patient 12 that conservative standard methods such as ALDEx2 miss. FIG. 16 Bshows that when measurement uncertainty predicted by StochQuant is larger than the observed difference in the relative abundance of Negativibacillus, StochQuant accurately determines (as confirmed by sequencing replicates) that the taxon is not differentially abundant, which is missed by non-parametric approaches such as Kruskal-Wallis.

FIG. 16C shows that StochQuant accurately determines that Aggregatibacter is not differentially abundant, and that the observed differences in abundance and lack of detection of Aggregatibacter in the rectum biopsies are due to the higher LoD of the rectum biopsies. FIG. 16D shows that StochQuant accurately determines that the observed differential abundance of Campylobacter between TI and R biopsies is irreproducible. Even though Campylobacter was initially detected in the 3 rectum biopsies, StochQuant predicted that Campylobacter was below the LoD of each biopsy, which was confirmed by the stochastic detection of Campylobacter in the re-sequenced biopsies.

FIG. 17 shows a schematic illustration of an exemplary detection workflow of a testing measurement comprising sampling of an environment as a physical manipulation directed to perform the absolute anchoring measurement to obtain the absolute anchoring value (Sample 1) and as a physical manipulation part of the detection workflow (Sample 2).

FIG. 18 shows a schematic illustration of an exemplary detection workflow of a testing measurement comprising sampling of an environment as a physical manipulation part of the workflow, and addition of a reference molecule (“Spike in”) into the environment to obtain the absolute anchoring value.

FIG. 19 shows a schematic illustration of an exemplary detection workflow of a testing measurement comprising sampling of an environment as a physical manipulation part of the workflow, and addition of a reference molecule (“Spike in”) into the sample to obtain the absolute anchoring value.

FIG. 20 shows a schematic illustration of an exemplary detection workflow of a testing measurement comprising sampling and subsampling of an environment as a physical manipulations which are part of the workflow.

FIG. 21 shows a schematic, illustration of an exemplary detection workflow of a testing measurement performed in an environment which is a solution of nucleic acids in a tube. In the exemplary workflow illustrated in FIG. 21 the contents of the tube can be a sample from somewhere else, but the environment is the tube, since that is the container for which the user is quantitatively detecting the target. Then, the same manipulations are performed as in Example 19.

FIG. 22 shows probability distributions of target relative abundance yielded by StochQuant in connection to Example 7.

FIG. 23. This is an example of using a “spike-in” of a reference molecule in an environment to obtain an absolute anchoring value of the reference molecule in an environment. This is also an example of an Assessment of a Measurement Representation Workflow. Here, the detectability of the 16S rRNA gene of a particular taxon (Escherichia), is compared between the Measurement Workflow Representation and the amplicon sequencing measurement workflow.

FIG. 24. This is an example of using qPCR with a standard curve to measure the PCR amplification efficiency of a target molecule (Listeria 16S rRNA gene). The target was amplified using the same primers and reagents that were used for the PCR amplification manipulation of the target/reference molecules as part of an exemplary amplicon sequencing measurement workflow.

FIG. 25: A comparison between (i) the probable distributions of target abundances in each environment yielded by the StochQuant measurement representation workflow described in Example 35 and (ii) the observed molecular count of the target yielded by the shotgun sequencing testing measurement for each environment (dashed vertical line in each plot). The target in this example is provided by the “ELFBKOLN_00270” geneid in the gene annotation provided as part of the product specification sheet of she defined microbial community (Zymo Cat #D6311).

FIG. 26: This shows a comparison between the probability distribution of target abundance (in this example, the probability distribution of the concentration (target molecules per microliter) of the gene target from FIG. 26. This example shows that for this gene target, each of the probability distributions of target abundance yielded by using the StochQuant workflow accurately determined that the “ground truth” concentration of the target from the defined community (dashed horizontal line) was within the bounds of the inferred one or more probability distributions.

FIGS. 27A-27G: This is part of Example 37 that shows stripplots of (FIG. 27A) the total reads sequenced from each environment of each participant in the example, (FIG. 27B) the normalized read counts per million of the gene target, (FIG. 27C) the non-normalized read counts of the gene target, and the longitudinal timeseries of the (FIG. 27D) total reads, (FIG. 27E) target read counts, (FIG. 27F) normalized target counts per million reads, and (FIG. 27G) StochQuant probability distribution of target molecules per microliter. In FIG. 27G, the horizontal dashed line is the inferred lower limit of detection.

FIG. 28: An example of the comparing the detectability of four ERCC RNA targets in (n=1030) cells in connection to the assessment of the accuracy of the measurement workflow for an exemplary measurement workflow of single cell RNA sequencing as part of Example 38.

FIG. 29: An example of the comparing the detectability of four ERCC RNA targets in (n=1030) cells in connection to the assessment of the accuracy of the measurement workflow for an exemplary measurement workflow of single cell RNA sequencing as part of Example 39.

FIG. 30. This is part an example showing that a Neural Network trained on the measurement workflow representation and physical parameters of the measurement workflow can yield probability distributions of target abundances (in this example target copies/μL or target concentration) that yield similar mean abundances (left) and measurement uncertainties (right) expressed as a % CV of the target concentrations of the probability distributions.

Additional, exemplary embodiments, features, objects, and advantages of the present disclosure will be apparent to a skilled person from the detailed description, the examples section and the claims and the instant disclosure in its entirety.

### DETAILED DESCRIPTION

The present disclosure describes methods and systems to perform detection of a target molecule in an environment according to a quantitative stochastic approach.

The term “environment” as used herein indicates a sum total of all the elements in a defined space of interest and subject to investigation. An environment can be a biological environment if it includes at least one biological elements, elements of an environment comprise molecule of any source and in particular biological molecule whether originated by living organisms or synthetically produced and/or engineered. Accordingly, environments can include different defined spaces of interest, such as their tissues, organs, and/or biofluids of an individual or aquatic or terrestrial environments. An environment in the sense of the disclosure can be subject to sampling. For example, for a blood test it could be the person, or the blood tube, or the plasma obtained from the blood, or the nucleic acids extracted from the plasma.

The term “molecule” as used herein indicates any group of two or more atoms held together by chemical bonds, subject to detection in the form of a molecular count. Molecules in the sense of the disclosure can comprise biological molecules (produced by cells and living organisms) and/or artificial molecules (artificially manufactured in a laboratory), the latter sometimes mimicking a biological molecule, as understood by a skilled person.

Accordingly, exemplary molecules in the sense of the disclosure comprise naturally occurring or synthetic nucleic acids as well as other substances attaching a nucleic acid or a nucleic acid mimic, e.g., as part of a molecular complex or as a barcode or a tag [1]. The term “nucleic acid” or “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Exemplary functional groups that can be comprised in an analog include methyl groups and hydroxyl groups and additional groups identifiable by a skilled person. Exemplary monomers of a polynucleotide comprise deoxyribonucleotide, ribonucleotides, LNA nucleotides and PNA nucleotides as understood by a skilled person.

The term “nucleic acid” or “polynucleotide” thus includes nucleic acids of any length, and in particular DNA, RNA, analogs thereof, such as LNA and PNA, and fragments thereof, each of which can be isolated from natural sources, recombinantly produced, or artificially synthesized. Polynucleotides can typically be provided in single-stranded form or double-stranded form (herein also duplex form, or duplex). A “single-stranded polynucleotide” refers to an individual string of monomers linked together through an alternating sugar phosphate backbone. The 5′-end of a single strand polynucleotide designates the terminal residue of the single strand polynucleotide that has the fifth carbon in the sugar-ring of the deoxyribose or ribose at its terminus (5′ terminus). The 3′-end of a single strand polynucleotide designates the residue terminating at the hydroxyl group of the third carbon in the sugar-ring of the nucleotide or nucleoside at its terminus (3′ terminus). A “double-stranded polynucleotide” or “duplex polynucleotide” refers to two single-stranded polynucleotides bound to each other through complementarily binding. The duplex typically has a helical structure, such as a double-stranded DNA (dsDNA) molecule or a double stranded RNA, which is maintained largely by non-covalent bonding of base pairs between the strands and by base stacking interactions. The term “5′-3′ terminal base pair” with reference to a duplex polynucleotide refers to the base pair positioned at an end of the duplex polynucleotide that is formed by the ‘5 end of one single strand of the two single strands forming the duplex polynucleotide base-paired with the 3′ end of the single strand forming the duplex polynucleotide complementary to the one single strand.

Additional molecules in the sense of the disclosure comprise naturally occurring or synthetic proteins. The term “protein” as used herein indicates a polypeptide with a particular secondary and tertiary structure that can interact with another molecule and in particular, with other biomolecules including other proteins, DNA, RNA, lipids, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. In particular, in a protein, the polypeptide provides the primary structure of the protein, wherein the term “primary structure” of a protein refers to the sequence of amino acids in the polypeptide chain covalently linked to form the polypeptide polymer. A protein “sequence”indicates the order of the amino acids that form the primary structure. Covalent bonds between amino acids within the primary structure can include peptide bonds or disulfide bonds, and additional bonds identifiable by a skilled person. Polypeptides in the sense of the present disclosure are usually composed of a linear chain of alpha-amino acid residues covalently linked by peptide bond or a synthetic covalent linkage. The two ends of the linear polypeptide chain encompassing the terminal residues and the adjacent segment are referred to as the carboxyl terminus (C-terminus) and the amino terminus (N-terminus) based on the nature of the free group on each extremity. Unless otherwise indicated, counting of residues in a polypeptide is performed from the N-terminal end (NH₂-group), which is the end where the amino group is not involved in a peptide bond to the C-terminal end (—COOH group) which is the end where a COOH group is not involved in a peptide bond. Proteins and polypeptides can be identified by x-ray crystallography, direct sequencing, immuno precipitation, and a variety of other methods as understood by a person skilled in the art. Proteins can be provided in vitro or in vivo by several methods identifiable by a skilled person. In some instances where the proteins are synthetic proteins in at least a portion of the polymer two or more amino acid monomers and/or analogs thereof are joined through chemically mediated condensation of an organic acid (—COOH) and an amine (—NH₂) to form an amide bond or a “peptide” bond. As used herein the term “amino acid”, “amino acid monomer”, or “amino acid residue” refers to organic compounds composed of amine and carboxylic acid functional groups, along with a side-chain specific to each amino acid. In particular, alpha- or a-amino acid refers to organic compounds composed of amine (—NH₂) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to forma polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D an L optical isomers.

Molecules in the sense of the disclosure includes aptamers which are short sequences of artificial nucleic acids, or peptides that bind a specific target substance, or family of target substance, exhibiting a range of affinities (K_{D} in the pM to μM range), with variable levels of off-target binding and are sometimes classified as chemical antibodies. [2] [3]

Molecules in the sense of the disclosure can also comprise any additional molecules that can be directly detected e.g., through use of a label of additional visualizing techniques such as microscopy. Direct single-molecule detection can be performed via methods such as the detection of RNA molecules via smFISH (as described e.g., in “Imaging individual mRNA molecules using multiple singly labeled probes” ref [4]) and “Third-generation in situ hybridization chain reaction: multiplexed, quantitative, sensitive, versatile, robust” ref. [5]).

Molecules in the sense of the disclosure can be distinguished in different types based on their capability to provide a unique molecular count following detection. Accordingly, a “type of molecule” in the sense of the present disclosure is a molecule that can provide a unique molecular count following detection. Examples comprise nucleic acid comprising different sequences of a same gene, nucleic acid from different genes, proteins labeled with different barcodes and additional types identifiable by a skilled person.

Molecules in the sense of the disclosure can also comprise molecules that can be conjugated to a nucleic acid, the nucleic acid which can be quantitatively detected via a testing measurement such as next generation sequencing. Examples of these types of molecules comprise synthetic or naturally occurring polymers, fatty acids, phospholipids, triglycerides, carbohydrates, nanoparticles, or macromolecules.

The term “target” as used herein indicates any referenced item which is selected as an item of interest. Therefore, a “target molecule” in the sense of the disclosure refers to molecule selected as molecule type of interest within the detection method: it can be formed by one type of molecule, or it can be form by a population of different types of molecules which are of interest and subject to investigation.

The term “detection” or “measurement” in the sense of the disclosure indicates the determination of the existence, presence or fact of a target in a limited portion of space, including but not limited to a sample, a reaction mixture, a molecular complex and a substrate.

A detection in the sense of the disclosure can be quantitative or qualitative. A detection is “qualitative” when it refers, relates to, or involves identification of a quality or kind of the target or signal in terms of relative abundance to another target or signal, which is not quantified, such as presence or absence. A detection is “quantitative” when it refers, relates to, or involves the measurement of quantity or amount of the target or signal (also referred as quantitation), which comprises any analysis designed to determine the amounts or proportions of the target or signal.

Accordingly, a quantitative detection or measurement in the sense of the disclosure indicates a detecting referring, relating to, or involving the measurement of quantity or amount of the target or signal (also referred as quantitation), which comprises to any analysis designed to determine the amounts or proportions of the target or signal. In quantitative detection in the sense of the disclosure the detection can be directed to detect an amount expressed as discrete value confined by integers, based number of molecule or elaboration thereof.

For example, quantitative detection of a nucleic acid can be provided using a fluorescence or spectrophotometric based method (e.g., Nanodrop or Qubit) which is considered to be proportional to the levels of the nucleic acid to be quantified as understood by a skilled person. Examples, as described e.g., in ref. [6] US Appl. Publ. 20210079447 (incorporated by reference in its entirety herein), absolute quantification of a nucleic acid can be provided by cell counting based methods such as flow cytometry, optical density, plating which is also considered to be proportional to the desired 16S nucleic acid levels. Absolute quantification of a nucleic acid can be provided by sequencing spike-in (adding a 16S sequence not in the sample at a known level, usually determined by dPCR/qPCR and then use the relative abundance after sequencing and the known abundance level that was inputted as the anchor) as will be understood by a skilled person. Absolute quantification of a nucleic acid can also be provided by detection of unique molecular identifiers (UMIs) via sequencing.

A: quantitative measurement of a total number of a referenced item provided in the form of total counts or of probability distribution of the total counts, is herein indicated also as an “absolute detection” or “absolute measurement” as understood by a skilled person upon reading of the disclosure.

In particular, in embodiments of the disclosure, the quantitative measurement in the sense of the disclosure can take the form of a molecular count. The term “molecular count” as used herein indicates a measurement indicative of the copy number of a molecule (e.g., number of read count for target nucleic acid, number of target gene as detected by digital PCR). Molecular count is a parameter related to (and often can be proportional to) absolute measurements. Molecular counts can be detected by a user (or software) who can count the number of molecules identified as the target based on physical characteristic(s) of the target as will be understood by a skilled person.

StochQuant methods and systems of the disclosure can be used in connection with one or more testing measurements directed to obtain a molecular count the target molecule in the environment in connection with detection of a reference molecule.

The term “reference” as used herein indicates an item that is selected as an item of comparison with respect to a target item. Accordingly, the term “reference molecule” as used herein indicates a molecule measured for comparison purposes in connection with the measurements, of a target molecule. As a consequence, a “reference molecule” in the sense of a disclosure is a molecule that i) can be detected, providing a molecular count, with a testing measurement providing a molecular count for the target in the sample and ii) can be measured with an absolute anchoring measurement and/or can be added in a known number of molecules.

In particular, the testing measurement of StochQuant methods and systems comprises at least one manipulation of the target molecules and/or the reference molecules which is known or expected to affect the number of the target molecules counted in the environment in view of the required manipulation of the target and/or or reference molecules and thus the molecular count which is detected in outcome of the testing measurement, thus impacting the accuracy and reliability of the measurement.

Accordingly, StochQuant methods and systems are preferably used in connection with testing measurement directed to detect target molecular known or expected to be present in the environment at a low abundance or moderate abundance since the related molecular count will be more impacted by the stochasticity introduced by the detection process, as will be understood by a skilled person.

In StochQuant methods and systems of the present disclosure, the wording “low abundance” of a target molecule in an environment, indicates a non-zero target molecule abundance that is expected to lead to irreproducible detection by a given testing measurement. Accordingly, low abundance indicates embodiments in which the target molecule is known or expected to give rise to non-zero detected molecular counts less than a certain precent of the time if the testing measurement were repeated, as understood by a skilled person. In other words, low abundance can be identified based on the ability (or lack thereof) to consistently detect a target molecule via a testing measurement. For example, less than 99% of the time, 97.5, 95% of the time can be chosen. An example of a low abundance target can be one for which the probability of detecting the target molecule at a given abundance via the testing measurement is less than 99% of the measurements, less than 97.5% of the measurements or less than 95%, as will be understood by a skilled person.

In StochQuant methods and systems of the present disclosure, the wording “moderate abundance” of a target molecule in an environment indicates a non-zero target molecule abundance that is expected to be consistently detected by a given testing measurement, but for which measurement uncertainty from the testing measurement is above a certain value, expected to impact the downstream analyses, conclusions, or decisions based on the testing measurement. For example, values of 50% uncertainty, 2× or 3× uncertainty can be used, as understood by a skilled person. An example of a moderate abundance target can be one for which the probability of quantifying the target molecule within 2× of the expected value of the testing measurement is less than 95%.

In embodiments herein described low abundance and moderate abundance can refer to a molecule known or expected to be present in an environment at low absolute and/or low relative abundance and that is detected with a testing measurement as will be understood by a skilled person upon reading of the present disclosure.

A “testing measurement” in the sense of the disclosure indicates quantitative detection performed through detection of a feature of a tested molecule which provides a molecular count. In particular, in StochQuant methods and systems herein described, a molecular count can be obtained by detection of structural features of a molecule to be counted, such as sequence of polynucleotide (typically DNA and RNA) or polypeptides (typically proteins or peptides) spatial conformation of the molecule resulting in specific binding of antibodies, and generation of specific mass spectrum which can be used to perform the count. Mass photometry can be used to count biomolecules and investigate their binding affinities, as described in ref. [7].

In particular, mass spectrometry can be used to detect a molecular count in connection with measured sequence of a polynucleotide or a polypeptide, and/or to a detected molecular mass of the molecular primarily by measuring the mass-to-charge ratio of ionized molecules. Accordingly, a measurement by mass spectrometry can be used in connection to specific structural features that can include molecular mass, isotropic composition, fragmentation patterns of the molecule, functional groups of the molecule, degree of unsaturation of a molecule, charge state of the molecule as will be understood by a skilled person.

Additional structural feature that can be detected to provide a molecular count comprise can be amino acid composition and amino acid structure of the molecular target based on an antibody-epitope interactions of the measurement performed for example by digital ELISA.

Further structural features that can detected to provide a molecular count, include presence of a tag which can advantageously performed for molecules that are not normally detected by sequencing. In some of those embodiments, the tag is provided by a nucleic acid sequence added in connection with a structural feature to be detected.

Additional structural features that can be used to perform quantitative detection with a testing measurement of the disclosure are identifiable by a skilled person.

In embodiments of StochQuant methods and systems of the disclosure, a testing measurement is directed to provide a molecular counts of detected molecules through detection of one or more structural features of the molecule provided by many detection method comprising a workflow directed to detect a molecular count.

Exemplary detection methods that can be used to perform one or more testing measurements in the sense of the disclosure comprise sequencing methods to detect a nucleic acid target, such as amplicon sequencing (16S rRNA gene sequencing described in the exemplary applications reported in Examples 3 to 15 and Examples 21 to 43 as well as in Appendix B of U.S. Provisional Application No. 63/579,291 incorporated by reference in its entirety), ITS gene sequencing, 18S rRNA gene sequencing, COI gene sequencing, ITS2 gene sequencing, RBP1 gene sequencing, RBP2 gene sequencing,V(D) J region sequencing, mitochondrial gene sequencing, functional gene sequencing). Sequencing methods may generate cDNA from either template DNA or template RNA (following reverse-transcription). Further examples of sequencing methods comprise bulk RNA sequencing (RNA-seq) to detect RNA target molecules, single cell RNA-seq to detect RNA target molecules or cell target molecules, metagenomic sequencing to detect DNA target molecules, metatranscriptomic sequencing to detect RNA target molecules, spatial transcriptomics to detect RNA target molecules, Chromatin Immunoprecipitation Sequencing (ChIP-seq) to detect DNA complex targets or DNA-protein complex targets, exome sequencing to detect exome (nucleic acid) target molecules, whole genome sequencing to detect nucleic acid target molecules, target capture gene panels, small RNA sequencing (microRNA-seq), methyl DNA sequencing, single-cell DNA-Seq, or Mate-Pair Sequencing. Examples of sequencing can be performed with short read or long read sequencing technologies. Additional methods to detect molecules such as target protein molecules include single molecule protein counting assays such as digital immunoassays such as SIMOA (as described e.g., in ref. [8], single molecule fluorescence in situ hybridization (smFISH), hybridization chain reaction (HCR) FISH, next generation sequencing (NGS) adapted for protein quantification.

Further examples of sequencing methods which can provide a testing measurement in a StochQuant methods and systems herein described comprise bulk RNA sequencing (RNA-seq), single cell RNA-seq, metagenomic sequencing, metatranscriptomic sequencing, spatial transcriptomics, Chromatin Immunoprecipitation Sequencing (ChIP-seq). These exemplary sequencing methods can be performed with short read or long read sequencing technologies as will be understood by a skilled person.

Additional methods that can be used to obtain molecular counts and can provide a testing measurement in a StochQuant methods and systems herein described comprise single molecule protein counting assays such as digital immunoassays such as SIMOA, single molecule fluorescence in situ hybridization (smFISH), hybridization chain reaction (HCR) FISH, next generation sequencing (NGS) adapted for protein quantification.

Additional methods that can be used to obtain molecular counts and can provide a testing measurement in a StochQuant methods and systems herein described comprise mass spectrometry directed to detect molecular counts for example from sequence a polypeptide or polynucleotide, or from the molecular mass of the molecular typically detected in form of mass-to-charge ratio of ionized molecules as will be understood by a skilled person.

Further methods that can be used to obtain molecular counts and can provide a testing measurement in a StochQuant methods and systems herein described comprises digital ELISA directed to detect molecular counts through detection of the amino acid composition and amino acid structure of the molecular target based on the antibody-epitope interactions of the measurement as will be understood by a skilled person.

Additional methods that can be used to obtain molecular counts and can provide a testing measurement in a StochQuant methods and systems herein described comprise detection of tagged molecular, e.g. by sequencing of a polynucleotidic tag, as will be understood by a skilled person.

Accordingly, a testing measurement in the sense of the disclosure can be performed according to any detection method configured to detect molecular counts of a target molecule as will be understood by a skilled person.

The molecular counts obtained in outcome of different measurements can take the form of one or more testing parameters which characterizes the testing measurement. For example, in testing measurement comprising RNA sequencing, the molecular count of a detected RNA can be indicated in the form or read counts. Additional example molecular counts can include: molecular counts of a target that are based on the exact match of physical characteristics of the target (e.g., the exact nucleic acid sequence), for example, the initial output of NGS is generally files that contain the physical characteristics of each sequenced “read” from the testing measurement—this could be a count of the number of reads that contain a sequencing that perfectly matches the sequence of the target of interest. Molecular counts also include molecular counts of a target identified by software or algorithms that identify key characteristics of the target to determine the number of detected target molecules—for example, a sequencing alignment software as will be understood by a skilled person.

Accordingly, molecular counts that can be obtained with testing measurement in the sense of the disclosure comprise, for example molecular counts obtained by sequencing nucleic acid target molecules, nucleic acid tags associated with target molecules, and/or amplicons generated from nucleic acid target molecules, and/or nucleic acid tags associated with one or more target molecules, as will be understood by a skilled person. Examples of sequencing methods include: amplicon sequencing (16S rRNA gene sequencing (as described in the exemplary applications reported in Examples 3 to 15 and Appendix B of U.S. Provisional Application No. 63/579,291 incorporated by reference in its entirety), ITS gene sequencing, 18S rRNA gene sequencing, COI gene sequencing, ITS2 gene sequencing, RBP1 gene sequencing, RBP2 gene sequencing,V(D) J region sequencing, mitochondrial gene sequencing, functional gene sequencing). Amplicons that can be generated by sequencing methods and then sequenced, comprise cDNA from either template DNA or template RNA (following reverse-transcription).

Other examples of molecular counting include quantifying protein-protein interactions by molecular counting with mass photometry [7] and single molecule multiplexed protein counting via modified DNA carriers with nanopore sequencing [9].

In StochQuant methods and systems, the testing measurement comprises or consist of a workflow (herein indicated as measuring workflow, detection workflow or measurement workflow) that yields a measurement of a molecular count of a molecule of interest (e.g., target molecule or reference molecule) from a target molecule in an environment. The testing measurement is formed by a set of activities which i) are required to perform the testing and ii) comprise manipulations that affect the number of detected target molecules and/or reference molecules.

The term “manipulation” as used herein in connection with a molecule, indicated modification of the physical, biological and/or chemical status of a molecule resulting from activities which form part of a testing measurement and are performed to enable detection of the molecule. Manipulations of a molecule in the sense of the disclosure is typically associated with a manipulation of the environment, sample and/or subsample thereof, where the molecule is known or expected to be present, the manipulation comprising or consisting of a modification of the physical, biological and/or chemical status of said environment, sample and/or subsample thereof.

Exemplary manipulations of a molecule in the sense of the disclosure comprise, sampling, fractionation, ligation of a barcode or an adapter, extraction such as liquid-phase extraction, fragmentation, cDNA synthesis amplification such as amplification by PCR or other amplification techniques. Additional exemplary manipulation comprise centrifugation, filtration, heat treatment, lyophilization, ultrasonication, mechanical shearing, electroporation, enzymatic digestion, cell lysis, hybridization, transfection, editing (e.g. by CRISP/Cas9), chemical crosslinking, chemical de-crosslinking, chemical denaturation, heat denaturation, precipitation, methylation/demethylation, chemical labeling, redox reactions, solid-phase extraction, chromatography, immunoprecipitation, encapsulation into droplets, microfluidic manipulations, in situ hybridization. Further exemplary manipulations in the sense of the disclosure include manipulations involved in the measurement/detection of the target/reference molecule such as fluorescent dye incorporation, nucleotide labeling, fluorophore quenching, real-time fluorescence detection, detecting emitted light from a fluorescent product, photometric detection, spectrophotometric detection. Another example is target enrichment, such as using capture probes that preferably bind to the target and/or reference molecules. Additional manipulations are identifiable by a skilled person.

In StochQuant methods and systems, the set of activities comprised in the measuring workflow of a testing measurement further comprises iii) detection of one or more physical parameters (herein also StochQuant parameters, StochQuant physical parameters or physical parameters) which are used to model the workflow and comprise at least: a) a molecular count of one or more target molecules, b) a molecule count of one or more reference molecules, and c) an absolute anchoring measurement providing a corresponding detected value.

The term “absolute anchoring measurement” in the sense of the disclosure indicates a quantitative measurement of the total number of a reference molecule the total number of the reference molecules is also indicated as the absolute anchoring value. The anchoring value can be provided in the form of a total number of molecular counts, or a probability distribution of a total number of molecular counts.

In StochQuant detection methods and systems of the disclosure absolute anchoring measurement and molecular counts of the reference molecule obtained during a testing procedure provide a standard for comparison against the molecular counts of the target molecule during the testing measurement as understood by a skilled person upon reading of the present disclosure.

In StochQuant methods and systems, the StochQuant parameters are used to provide stochastic representations of the activities of the workflow including manipulations which impact the count of detected targeted molecule and/or reference molecule. These stochastic representation form a model of the measuring workflow herein also indicated as measurement workflow representation as will be understood by a skilled person upon reading of the present disclosure.

In StochQuant methods and systems, a measurement workflow representation can thus be defined as a mathematical representation of the manipulations of the testing measurement that yields a distribution of probable molecular counts of the target that approximates the number and/or variability in the number of molecules counted resulting from the testing measurement. The measurement workflow representation can be used in a StochQuant detection workflow to obtain the probability distribution of the target molecule abundance in the environment based on the physical parameters.

In StochQuant methods and systems, a measurement workflow representation can be performed in connection with any testing measurement which result in a molecular count of a target molecule, and which affect the number of target molecules counted in an environment in view of the required manipulation of the molecules of importance (target or reference molecules) as will be understood by a skilled person upon reading of the present disclosure.

In StochQuant methods and systems of the disclosure a measurement workflow representation can include one or more measurement workflow representation segment (referred to as measuring segment or a “segment” for short).

Accordingly, in StochQuant methods and systems herein described, a testing measurement representation segment is a segment identified within the testing measurement workflow directed to detect a molecular count comprises at least one set of activities that is known or expected to impact the molecular count. The set of activities/manipulations that is selected to form segment of a measurement workflow representation depend on the abundance of the molecule, the specific activities that form part of the detection workflow, and the desired accuracy of the measurement workflow representation as will be understood by a skilled person upon reading of the present disclosure.

Exemplary segments include separation of a sample from an environment, flow cell binding (which is an example of a sampling step), amplification manipulations (e.g., PCR), isolation of target (e.g., nucleic acid extraction), and reverse transcription (RT). Other segments would be understood by one skilled in the art. In preferred embodiments, StochQuant detection methods and systems comprise a detection workflow comprising one or more of: (Segment 1) Separation of a sample from an environment and (Segment 2) a Measurement Segment.

For example, in the measurement workflow representation of amplicon sequencing provided as a proof of principle to investigate taxon abundance in a microbial community, two segments can be identified that comprise the measurement workflow representation (see e.g. Example 5). In this example, these segments are stochastic representations of Segments of the testing measurement that affect the molecular count of the target/reference molecules. It can be understood that segments of a measurement workflow representation can occur in sequence, such that the output number of molecules of a Segment are the input number of molecules into the subsequent segment. It can also be understood that the final segment of a measurement workflow representation yields a molecular count of the target molecule (or target molecules, in a workflow that includes more than one target molecule type).

In StochQuant methods and systems of the disclosure, a measurement workflow representation segment can be identified by identifying a manipulation or series of manipulations of a testing measurement workflow that: (i) can impact the molecular count of the target/reference molecule obtained via the testing measurement, (ii) can be measured via a segmental calibration that can yield a representation of the segment that can yield output numbers of target/reference molecules that approximate the output numbers of target/reference molecules of the manipulation(s) of the testing measurement, and (iii) for which the segment representation can be parameterized by the number of input target/reference molecules and/or the physical parameter of the manipulation(s) of the testing measurement that can impact the molecular count of the target/reference.

Accordingly, a user can identify the manipulations of a testing measurement workflow based on obtaining the procedures of the testing measurement workflow. These manipulations are commonly referred to as “steps of a protocol” that describe the sequential manipulations of a molecule of interest to yield a molecular count of the molecule of interest.

In StochQuant methods and systems given the manipulations of a testing measurement workflow, a user can identify the manipulation or series of manipulations for which a segmental calibration is to be performed.

In StochQuant methods and systems, at least one of the segment of a testing measurement workflow comprises a manipulation affecting of at least one of StochQuant parameter selected from the molecular count of one or more target molecules, the molecule counts of one or more reference molecule, an absolute anchoring measurement of the detection workflow providing a corresponding detected value. In StochQuant methods and systems, one or more segments of the workflow can comprise additional StochQuant parameters which are associated with and characterize the step of the protocol performed in the segment and affect the molecular count of one or more target molecules and/or one or more reference molecules. For examples, in a segment comprising a performing sampling and a polymerase chain reaction (PCR) a quantitatively measured amount of the sample, and the PCR amplification rate provides an additional StochQuant parameter for the representation of the segment as will be understood by a skilled person upon reading of the present disclosure.

In StochQuant methods and systems, at least one of the segment of a testing measurement workflow can be evaluated and the impact of the manipulations on molecular counts modeled through a segmental calibration. A “segmental calibration” can be defined as a calibration procedure that generates or acquires the data that characterizes the properties of the manipulation and that impact the molecular count to provide the physical parameters of the manipulation that will be used to parameterize the segment representation. Accordingly, data generated or acquired during segmental calibration comprise values for at least one or more StochQuant parameters as will be understood by a skilled person.

In StochQuant methods and systems, the data generated or acquired by the segmentation calibration are used to understand the physical properties of the manipulation such that the understanding can provide the physical parameters of the manipulation and the mathematical representation of the manipulation. It can be understood that generating and/or acquiring calibration data across a wider range of number of target molecules, and increasing the number of different numbers of target molecules used for the calibration, and performing more repeated measurements to obtain the calibration data can result in improved segmental calibration.

In some embodiments of the StochQuant methods and systems, performing a segmental calibration for a particular manipulation can be challenging as will be understood by a skilled person in view of technological limitations that can make it challenging to accurately characterize the properties of the manipulation that impact the molecular count. In some embodiments of the StochQuant methods and systems, performing a segmental calibration can be performed in view of the time and/or cost constraints which would limit the number of segments considered by a skilled person when performing identification of segment of a measuring workflow, which can be used for StochQuant segmental calibration.

Accordingly, in some embodiments, of the StochQuant methods and systems a segment of a measuring workflow can comprise more than one manipulation combined into a series of manipulations in a single segment of the workflow to be used for a single segmental calibration in accordance with the disclosure. For example, in those embodiments of StochQuant methods and systems, for a series of manipulations, Manipulation 1 and Manipulation 2, a segmental calibration can be performed by using a known number of molecules of interest in Manipulation 1, then subsequently performing Manipulation 2, and then obtaining calibration data that characterizes the properties of the series of Manipulation 1 and Manipulation 2. A non-limiting example is the isolation of nucleic acids from a biological specimen. In this example, the isolation of nucleic acids involves a series of manipulations. Measuring the number of molecules affected by each manipulation would be challenging, so it is common practice to measure the “extraction efficiency” or “extraction variability” that describes the number of molecules yielded by the series of manipulations that are grouped collectively to describe the manipulations of the workflow required to isolate the nucleic acids. In this case, extraction efficiency and extraction yield are physical parameters of the series of manipulations that characterize the properties of the manipulation that impact the molecular count. As such, these physical parameters characterize the fraction of molecules and the stochasticity of molecules that are yielded by the series of the manipulations as will be understood by a skilled person.

In some embodiments, identification of a segment of a measuring workflow fore related segmental calibration can be performed for a “proxy” manipulation which share the same physical biological and/or chemical properties of the manipulation comprised within the measuring workflow of the testing measurement which impact the molecular count of target and/or reference molecule detected by the testing measurement. A skilled person can understand that if a manipulation (Manipulation 1) shares the same properties of the manipulation that impact the molecular count as another manipulation (Manipulation 2), then the segmental calibration for Manipulation 1 can be used for Manipulation 2.

An exemplary proxy manipulation is provided by separating a sample from an environment. One may perform a segmentation calibration for target molecule A (e.g., a DNA molecule) (Manipulation 1). Based on the results of the segmentation calibration for molecule A and physical features of molecule A, one may use this segmentation calibration for molecule A as a proxy for the segmentation calibration for the manipulation of target moleculeB(e.g., another DNA molecule) (Manipulation 2), another exemplary proxy manipulation is provided Binding of a DNA molecule to a flow cell. One may perform a segmentation calibration for molecules of interest with a MiSeq v2 Kit Flow Cell (Manipulation 1), and one may use this segmentation calibration for a manipulation with a MiSeq v3 Kit Flow Cell (Manipulation 2). Additional proxy manipulation can be identified by a skilled person upon reading of the present disclosure.

In StochQuant methods and systems, the mathematical representation and physical parameters selected by the user can be guided by the desired accuracy of the measurement workflow representation Accordingly, skilled person will understand that in StochQuant methods and systems herein described, selection of a StochQuant Detection Accuracy can be obtained as by balancing the gain in accuracy via a Segment of the measurement workflow representation that approximate output numbers of molecules of the manipulations of a testing measurement with the cost of detection (the cost of performing the segmental calibrations, the increased complexity of the StochQuant detection, and increased computational requirements).

In some embodiment of StochQuant methods and systems, the data generation of a segmental calibration is obtained by the user.

In some embodiments of StochQuant methods and systems, the data generation of a segmental calibration has been previously performed by the user or by others (e.g., the data from the calibration is available in the literature) and as such a user can acquire the data. (see e.g. Examples 29, 35, 37)

In some embodiments of the StochQuant methods and systems, a segmental calibration is performed by retrieving data generated by measurements previously performed by the user or by others. In some embodiments of the StochQuant methods and systems, a the physical parameters of a segment to be used in a StochQuant segmental calibration are already known. (see e.g. Example 29, Example 33, Example 38)

In StochQuant methods and systems of the disclosure, segmental calibration preferably performed also in combination of assessing accuracy of the calibrate segment results in a mathematical representation of the stochasticity introduced by manipulations of the workflow segments. Exemplary common mathematical representations of the measurements of the segmental calibration can include a Poisson distribution, binomial distribution, Bernoulli distribution, normal distribution, exponential distribution, hypergeometric distribution, negative binomial distribution, and/or negative hypergeometric distribution.

It can also be understood that the mathematical representation and physical parameters selected by the user can be guided by the desired accuracy of the measurement workflow representation.

In some embodiments of the StochQuant methods and systems, the method comprises determining accuracy of a segment of a measurement workflow representation. Below are two examples:

Option 1 (verify segments in order to string them together):
1) For a measurement workflow representation, start with the final segment that yields the molecular count.
1a) Input known amounts of target/reference into an environment.
1b) Perform the manipulation(s) of the segment repeatedly on replicate environments containing the target/reference. Because this is the final segment, the manipulation(s) will yield a molecular count of the target/reference.
1c) The repeated manipulations yield a distribution of outcomes of the manipulation (in this case distributions of molecular counts of the target/reference).
1d) Compare the distribution of molecular counts of the target/reference from the manipulation(s) of the segment to the distribution of molecular counts yielded by the Segment Representation. Can compare using the same techniques/procedures described for the assessment of the Accuracy of the entire workflow representation.
2) Next, assess the accuracy of the preceding segment (the second to last segment).
2a) Repeat the procedure above, except perform the manipulations of the final two segments.
2b) Assess the Accuracy of representation of the two segments together.
3) Next, assess the accuracy of the preceding segment (the third to last segment) . . . repeat until you are at the start of the workflow (the first manipulation of the target in the environment).

Option 2 (verify a segment independently of all other segments):
1) Input known amounts of a molecule of interest (the molecule of interest can either be the target/reference or a molecule that shares the key physical features of the target/reference) into an environment.
2) Perform the manipulation repeatedly on replicate environments containing the molecule of interest.
3) Perform a measurement indicative of the number or state of the molecule of interest in each of the replicate environments to obtain a distribution of outcomes of the manipulation.
4) Compare the distribution of outcomes to the distribution of outcomes predicted by the segment representation (can use any of the assessment techniques/procedures described for the assessment of an entire representation workflow).

In StochQuant methods and systems of the disclosure, mathematical representations provided in outcome of a segmental calibration are chained together to provide a mathematical representation of a measuring workflow of the testing measurement as will be understood by a skilled person upon reading of the present disclosure.

In particular, in StochQuant methods and systems of the disclosure, a molecular count of a target molecule and a molecular count of a reference molecule detected during the testing measurement and typically modeled through a segmental calibration of one or more segments of a workflow of the testing measurement, are used together with an absolute anchoring value of the reference molecule; to obtain a probability distribution of the abundance of the target molecule in the environment The probability distribution provides a StochQuant detection in outcome of the testing measurement.

Accordingly, in StochQuant methods, the probability distribution of the abundance of the target molecule in an environment is obtained as a function of i) the molecular count of the target molecule; ii) the molecular count of the reference molecule; and iii) the absolute anchoring value of the reference molecule. The molecular count of the target molecule and the molecular count of the reference molecule are obtained in outcome of the testing measurement. The molecular count of the target molecule, the molecular count of the reference molecule, and the absolute anchoring measurement of the reference molecule are collectively referred to as the Physical parameters or StochQuant Parameters.

The term “probability distribution” as used herein indicates a mathematical expression (data, list, function, etc.) that describes the probability of different possible values for a given quantity of interest as understood to a skilled person.

A probability distribution can take many different forms as understood by a skilled person. For example, a probability distribution can be provided in non-parametric form as one or more target abundances, each with a probability of being the true target abundance. A probability distribution can be further provided in the form of shape parameters for a known discrete probability distribution. An example is containing the information of the probability distribution in the form of the rate parameters n and p of a negative binomial distribution. A probability distribution can be provided in the form of a list of target abundances where the representation of each target abundance (e.g., how many times the target abundance “2” occurs) is correlated with its probability. If abundance “2” is the most likely, it will appear more times than any other abundance.

In StochQuant methods and systems herein described, obtaining a probability distribution of the target molecule abundance in the environment as a function of the molecular count of the target molecule; the molecular count of the reference molecule; the absolute anchoring value of the reference molecule; and possibly additional StochQuant Parameter such as a quantitively measured amount of the sample and possibly others, as will be understood by a skilled person upon reading of the disclosure.

StochQuant methods and systems herein described the specific measurement workflow representation is used to obtain the probability distribution reporting the probable molecular counts of target molecule obtained via the testing measurement. The probable molecular count is thus based on the physical parameters modeled with segmental calibration and/or with a model of the entire workflow of the testing measurement selected to correspond to the molecular count and variability in the molecular count of the target resulting from the actual testing measurement performed.

[Accordingly, in StochQuant methods and systems herein described the number and the variation in the molecular count of the target molecule resulting from the specific activities of the testing measurement can be obtained by performing multiple testing measurements running the entire measuring workflow or multiple calibration of one or more segments of the measuring workflow as will be understood by a skilled person In some embodiment the number and the variation in the molecular count of the target molecule resulting from the specific activities of the testing measurement can be obtained by combining one or more measurement with data and/or representation of one or more segments previously obtained by the user or others as will be understood by a skilled person The StochQuant parameters so obtained can be used to obtain a mathematical representation of the segments and/or of the testing measurement.

In some embodiments of StochQuant methods and systems herein described the selection of a mathematical representation of a manipulation or series of manipulations is in the form of a known discrete probability distribution and the physical parameters which are representative of the number and variability in the number of molecules of interest yielded by the manipulation of a testing measurement as part of a StochQuant workflow (See Example 2).

In some of those embodiments the mathematical representation of the manipulation or series of manipulations can be identified with the aid of artificial intelligence (AI) approach such as machine learning approaches such as supervised learning, unsupervised learning, semi-supervised learning, reinforcement learning, deep learning through deep neural networks, neural networks, transfer learning, generative models, ensemble learning, and dimensionality reduction techniques. For example, the relevant parameter can be input into a trained neural network, trained to produce an expected distribution of outputs for the segment or series of segments (See Example 48).

In some embodiments of StochQuant methods and systems herein described the measurement workflow representation has been pre-identified and therefore the user can perform the StochQuant detection by inputting the detected values of StochQuant physical parameters in the pre-determined measurement workflow representation (See Example 2).

In some embodiments, the measurement workflow representation can be pre-identified and loaded in a devices (e.g. a microfluidic device) with an algorithm which inputs the detected values for the StochQuant parameters in the model and displays the probability distribution, confidence level, and/or a determination based upon the probability distribution or confidence level related to the target molecule abundance.

In some embodiments, the measurement workflow representation can comprise more than one probability distribution which corresponds, and are representative of, the changes in molecular count due to the manipulation of the biological environment required by the detection activities of one or more segments.

In particular, a measurement workflow representation can be prepared to account additional various factors due to the detection activities such as intra-operator variability (that can arise due to several factors including a user's mistake), inter-operator variability (that can arise due to differing levels of consistency/variability between different users performing the same workflow), or variability of equipment performance.

In some embodiments, the probabilistic abundance of a reference molecule is used to determine the probabilistic abundance (absolute or relative) of a target molecule. This is beneficial because, if the target molecule is in low or moderate absolute and/or relative abundance, one or more sampling step can provide a highly variable number of target molecules. This variable number of molecules can give rise to a variable ratio of target to non-target molecules. Therefore, StochQuant takes this into account by treating the loading processes(es) stochastically. This can be accomplished, for example, by taking virtual random samples and simulating the molecular counts at different quantities. A measurement is taken where the simulated read count matches the observed read count for each quantitative value, thereby building a probability distribution over multiple values, each probability score representing the confidence that the target molecule matches that given abundance value.

In StochQuant methods and systems of the disclosure an Inference Procedure is performed with the measurement workflow representation to yield a probability distribution of target abundances in an environment.

In some embodiments of StochQuant methods and systems, the inference is an algorithm that uses the physical parameters of the measurement workflow representation and the measurement workflow representation to identify probable target abundances in an environment that yield molecular count of the target that are approximately equal to the molecular count of the target yielded by the testing measurement. An example is Example 6, Example 35, Example 37, Example 38.

In some embodiments, the Inference Procedure is implemented in the form of Bayesian Inference method. Examples of Bayesian Inference methods can include Markov Chain Monte Carlo (that uses common algorithms such as Metropolis-Hastings, Gibbs Sampling, Hamiltonian Monte Carlo, or No-U-Turn Sample), Variational Inference that uses common techniques such as Mean-Field Variational Inference, Stochastic Variational Inference, or Black-Box Variational Inference, Laplace Approximation, Expectation Propagation, Sequential Monte Carlo (SMC)/Particle Filters, Approximate Bayesian Computation, Integrated Nested Laplace Approximation, Bayesian Model Averaging, Empirical Bayes methods, Bayesian Nonparametrics methods such as Dirihclet Process mixtures. These approaches and other approaches like these approaches can be implemented via a software package. Examples of a software package that can implement a Bayesian Inference method can include Stan, PyMC/PyMC3, JAGS, BUGS, TensorFlow Probability, Emcee, Greta, LibBi, Edward/Edward2, BayesPy, Infer.NET, Turing.jl, SVI in Pyro, R-INLA, TMB, Pyro, SMCTC, SMC, ABC-SysBio, PyABC, EasyABC, abc, DABC, BMA, Bayes VarSel, BMS, EBglmnet, limma, ashr, vmbp, DPpackage, BNP, LibDAI, pgmpy, GraphLab Create.

In another embodiment other forms of inference can perform the same inference task of taking the measurement workflow representation and the StochQuant physical parameters (molecular count of the reference molecule obtained via the testing measurement, molecular count of the target molecule obtained via the testing measurement, the absolute anchoring value of the reference molecule, and quantifiable measured amounts) and produces a probability distribution of target molecule abundance. For example, one can take StochQuant inputs and outputs, and train a neural network to perform the regression task of predicting the probability distributions (see Example 48)

Accordingly, StochQuant is a combined experimental and computational approach as would be understood by a skilled person, that improves the quality of detection and in particular, sequencing analysis, of target molecule with particular reference to low-to-moderate abundance targets, which are difficult to analyze with standard methods.

In preferred embodiments, StochQuant detection methods and systems comprise a detection workflow configured to measure from one or more of the following environments: a sample obtained from a human such as blood, biopsy, swab (vaginal, rectal, urethral, oral, nasal), urine, stool, respiratory specimen material derived from the sample obtained from a human, such as purified, cleaned-up, isolated, etc. (e.g., nucleic acids); cells and organisms (Plants, seeds, fungi, bacteria, animals, mammalian cells) for genetic identification of an organism or for detecting a contaminating cell or organism (such as for genetic testing of seeds/plants in agriculture or yeasts/fungi/bacteria/mammalian cells in biomanufacturing); sample/material as above, but from a non-human animal instead of a human (e.g. an animal that underwent a treatment for drug discovery, or an animal for agriculture like a cow or a pig); food (e.g., testing for pathogens, sterility, genetic composition); DNA-encoded/DNA-tagged library of target molecules; wastewater, built environment, sterility filtration collection; and pooled samples of any of the preceding. In preferred embodiments, StochQuant detection methods and systems comprise a workflow configured to measure one or more target molecules related to: prenatal, cancer, infectious diseases, STIs, and BV.

In preferred embodiments, StochQuant detection methods and systems comprise a detection workflow utilizing one or more of the following reference molecules: A synthetic nucleic acid that contains a unique sequence that can easily be differentiated from target sequence and other sequences in the environment; a synthetic nucleic acid that contains similar physical properties to the target molecule(s) such that the manipulations of the workflow have a similar effect on the target and the reference. For example, a reference of similar length and GC composition to the target; plurality of 16S rRNA gene molecules (e.g., those obtained from 16S with universal primers); a molecule that is expected to be in the environment of interest, such as a gene marker of a commensal organism expected to be in the environment; and a molecule that is expected to be in the environment of interest, such as a non-mutated human sequence expected to be in the environment. In preferred embodiments, StochQuant detection methods and systems comprise a detection workflow comprising one or more of the following testing measurements: amplicon sequencing; multiplex amplicon sequencing; shotgun metagenomic sequencing; bulk RNA sequencing; and single cell RNA sequencing.

In preferred embodiments, StochQuant detection methods and systems comprise a detection workflow utilizing absolute anchoring values determined by one or more of: spike-in of a target into an environment for the absolute anchoring value and/or measurement of the efficiency and/or variability of a segment or workflow; digital PCR measurement to yield the absolute anchoring value of the reference; and qPCR with a standard curve.

In preferred embodiments, StochQuant detection methods and systems comprise manipulations comprising one or more of: separation of a sample from an environment, flow cell binding (which is an example of a sampling step), amplification manipulations (e.g., PCR), isolation of target (e.g., nucleic acid extraction), reverse transcription (RT), and target enrichment (e.g., via capture probes).

In some embodiments, StochQuant can be used in methods and a systems to improve a testing measurement for detection of an abundance of a target molecule in a physical environment. In those embodiments to the first aspect the testing measurement comprises a measuring workflow for the molecular count of a target molecule and a reference molecule to be improved by providing a molecular detection that account for stochasticity impacting the detection itself introduced by the measuring workflow.

In those embodiments the method comprises: dividing the measuring workflow into one or more measuring segments arranged in a measuring workflow order, each of the one or more measuring segments comprising one or more physical manipulations impacting the molecular count of the target molecule and/or of the reference molecule.

The method further comprises: ii) calibrating the one or more measuring segments by building corresponding stochastic representations of each of the one or more measuring segments into a computer-based system, the stochastic representations taking as inputs physical parameters of the measuring workflow.

The method also comprises: iii) chaining the corresponding stochastic representations together into a model of the measuring workflow by connecting outputs of measuring segments into inputs of other measuring segments in the measuring workflow order, such that the model takes as model inputs the physical parameters including at least a target molecule molecular count, a reference molecule molecular count, and an absolute anchoring value of the reference molecule.

The method additionally comprises: iv) configuring the computer-based system to provide a probability distribution of an abundance of the target molecule based on the model of the measuring workflow when provided the model inputs.

In some embodiments at least one of the one or more physical manipulation comprises sampling the environment or a sample or a subsample thereof from a previous measuring segment.

In some embodiments, at least one of the one or more measuring segments includes amplicon sequencing.

In some embodiments, at least one stochastic representation of the one or more measuring segments comprises calculating a distribution of data for output for said at least one stochastic representation.

In some embodiments, the distribution is one of: a Poisson distribution, binomial distribution, discrete random uniform distribution, or a negative binomial distribution.

In some embodiments, the method includes configuring the computer-based system to also provide a confidence level of an abundance of the target molecule based on the model of the measuring workflow when further provided with a threshold abundance value.

In some embodiments, the computer-based system provides the confidence level by determining a total amount of probability above the threshold abundance value within the probability distribution.

In some embodiments, the computer-based system is also configured to provide a confidence level of an abundance of the target molecule by calculating a total amount of probability within a confidence interval within the probability distribution.

In some embodiments, the confidence interval is a pre-set value.

In some embodiments, the computer-based system is also configured to provide a confidence interval of an abundance of the target molecule matching a given confidence level by calculating a total amount of probability matching the given confidence level within the confidence interval within the probability distribution.

In some embodiments, the given confidence level is input by the user of the computer-based system.

In some embodiments StochQuant can be used in methods and a systems to build a computer-readable program that improves a measuring workflow of a testing measurement for detection of an abundance of a target molecule in a physical environment. The improvement of the measuring workflow is performed by StochQuant by enabling a probabilistic detection which account for and inform the user of the stochasticity impacting the detected molecular count and resulting from the activities of the detection workflow.

The method comprises: i) dividing the measuring workflow into one or more measuring segments arranged in a measuring workflow order, each of the one or more measuring segments comprising one or more physical manipulations of a molecular count of the target molecule and/or of a reference molecule in the environment, a sample and/or a subsample thereof.

The method further comprises: ii) calibrating the one or more measuring segments by building corresponding stochastic representations of each of the one or more measuring segments into a computer-readable program, the stochastic representations taking as inputs physical parameters of the measuring workflow.

The method also comprises: iii) chaining the corresponding stochastic representations together into a model of the measuring workflow by connecting outputs of measuring segments into inputs of other measuring segments in the measuring workflow order, such that the model takes as its inputs the physical parameters including at least a target molecule molecular count, a reference molecule molecular count, and an absolute anchoring value of the reference molecule.

The method additionally comprises: iv) configuring the computer-readable program to provide a probability distribution of an abundance of the target molecule based on the model of the measuring workflow when run on a computer system and given the inputs by a user of the computer-readable program.

In some embodiments, at least one of the one or more measuring segments is a step of taking samples from the environment or from a result from a previous measuring segment.

In some embodiments, at least one of the one or more measuring segments includes amplicon sequencing.

In some embodiments, at least one stochastic representation of the one or more measuring segments comprises calculating a distribution of data for output for said at least one stochastic representation.

In some embodiments, the distribution is one of: a Poisson distribution or a negative binomial distribution.

In some embodiments, the computer-readable program is further configured to provide a confidence level of an abundance of the target molecule based on the model of the measuring workflow when further provided with a threshold abundance value.

In some embodiments, the computer-readable program provides the confidence level by determining a total amount of probability above the threshold abundance value within the probability distribution.

In some embodiments, the computer-readable program is further configured to provide a confidence level of an abundance of the target molecule by calculating a total amount of probability within a confidence interval within the probability distribution.

In some embodiments, the confidence interval is a pre-set value.

In some embodiments, the computer-readable program is further configured to provide a confidence interval of an abundance of the target molecule matching a given confidence level by calculating a total amount of probability matching the given confidence level within the confidence interval within the probability distribution.

In some embodiments, the given confidence level is input by the user of the computer-readable program.

In some embodiments StochQuant can be used in methods and systems to probabilistically detect a target molecule in an environment by performing measuring workflow of a testing measurement to measure abundance of the target molecule in the environment in combination with a reference molecule. In those embodiments StochQuant enables detection of the abundance of the target molecule providing probability distributions which inform the user of the impact of stochasticity introduced by the detection workflow on the detected abundance thus improving the related testing measurement.

The method comprises: i) performing the measuring workflow on the environment, a sample and/or a subsample thereof, the measuring workflow comprising one or more physical manipulations of the target molecule and/or the reference molecule in the environment, the sample and/or the subsample thereof impacting a molecular count of the target molecule and/or of the reference molecule.

The method also comprises ii) providing a molecular count of the target molecule in the environment from performing the measuring workflow by detecting the molecular count of the target molecule in the environment, the sample and/or the subsample thereof.

The method further comprises iii) providing a molecular count of a reference molecule from performing the measuring workflow by adding a known amount of the reference molecule and/or by detecting the molecular count of the reference molecule in the environment, the sample and/or the subsample thereof.

The method additionally comprises iv) providing an absolute anchoring value of the reference molecule.

The method also comprises v) based on at least the absolute anchoring value of the reference molecule, the molecular count of the target molecule, and the molecular count of the reference molecule, forming a probability distribution of abundances of the target molecule in the environment based on a modeling of the measuring workflow, the modeling taking into account stochastic properties of the physical manipulations of the target molecule. and/or the reference molecule in the environment, the sample and/or the subsample thereof.

In some embodiments, the absolute anchoring value of the reference molecule is obtained by performing in a sample of the environment an absolute anchoring measurement of the reference molecule.

In some embodiments, the absolute anchoring value of the reference molecule is a known value because the reference molecule would be added for the measuring workflow in a known amount.

In some embodiments, the reference molecule is not present in the environment but is added to the measuring workflow at some point.

In some embodiments, the absolute anchoring value is an adjusted value of an absolute anchoring measurement of the reference molecule.

In some embodiments, the measuring workflow includes amplicon sequencing.

In some embodiments, the amplicon sequencing includes one or more of: 16S rRNA gene sequencing, ITS gene sequencing, 18S rRNA gene sequencing, COI gene sequencing, ITS2 gene sequencing, RBP1 gene sequencing, RBP2 gene sequencing,V(D) J region sequencing, mitochondrial gene sequencing, functional gene sequencing.

In some embodiments, the reference molecule is a mRNA of a gene.

In some embodiments, the reference molecule is selected from: Glyceraldehyde-3-phosphate dehydrogenase (GAPDH), Phosphoglycerate kinase 1 (PGK1), Peptidylpropyl isomerase A (PPIA), ribosomal protein L13a (RPL13A), ribosomal protein large P0 (RPLP0), Beta-2-microglobulin (B2M), YWHAZ, SDHA, TFRC, GUSB, HMBS, HPRT1, TBP; bacterial housekeeping genes such as 16S, tus, rpoD, glyA, dnaB, gyrA, pykA/F, pfkA/B, mdoG, arcA; fungal housekeeping genes such as DUF221, ubcB, ADA, fis1, Cu-ATPase, psm1, spo7, spt3, DUF500, sac7, AP-2 beta, npl1, Beta-tubulin, Arabinofuranosidase-B2, Xylanase C.

In some embodiments, the reference molecule is a plurality of types of molecules simultaneously detected during the testing measurement to provide a same count.

In some embodiments, the reference molecule is multiple 16S genes which all amplify from the same primer.

In some embodiments, the plurality of molecule types that are simultaneously detected during the testing measurement are selected from multiple genes, portions of genes, regions, or portions of regions which all amplify from the same primer Lipopolysaccharides (LPS), Peptidoglycan, Teichoic acids, and specific DNA or RNA targets.

In some embodiments, the reference molecule is a plurality of types of molecules each separately detected during the testing measurement to provide separate unique counts that are used to determine at least the molecular count of the reference molecule.

In some embodiments, the forming a probability distribution of abundances of the target molecule is further based on multiple molecular counts of the reference molecule.

In some embodiments, the plurality of types of molecules are selected from multiple RNA expression reference molecules.

In some embodiments, the method also includes determining a probability that an actual abundance of the target molecule in the environment is above (or below) a threshold abundance by calculating a total area of the probability distribution higher than (or lower than) the threshold abundance. Calculating the area of the probability distribution can be done by calculating the area under the curve, by integration, by Monte Carlo integration, and other analytical, numerical, algebraic, and discrete methods identifiable by a skilled person.

In some embodiments, the method also includes determining a probability that an actual abundance of the target molecule in the environment is above (or below) or equal to a threshold abundance by calculating a total area of the probability distribution higher than (or lower than) or equal to the threshold abundance.

In some embodiments, the method also includes determining a confidence level by calculating the area of the probability distribution within a given confidence interval.

In some embodiments, the method also includes determining a confidence interval by calculating what interval within the probability distribution provides a given confidence level.

In some embodiments, the interval is centered around a given abundance value.

In some embodiments the StochQuant methods and systems, comprise determining accuracy of the measurement workflow.to assess if a measurement workflow representation yields a sufficiently accurate approximation of the testing measurement:

In some embodiments the StochQuant methods and systems the accuracy of the measurement workflow representation can be measured/assessed by comparing (a) the molecular counts of the target molecule yielded by the measurement workflow representation to (b) the molecular counts yielded by a testing measurement for which the number of target molecules in an environment is known. In some embodiments, a user can perform multiple (replicate) testing measurements to obtain a distribution of molecular counts of a target yielded by the testing measurement. Then, the user can use the measurement workflow representation (with the known number of molecules in an environment and the physical parameters obtained for the corresponding testing measurement) to yield a distribution of target molecular counts yielded by the measurement workflow representation. Then the distribution of molecular counts of the target yielded by the testing measurement and the measurement workflow representation can be compared to yield a measure of accuracy.

Exemplary procedure to perform an assessments of accuracy comprise comparing the detectability of a target via the testing measurement e.g. by comparing the number of times a target is detected to the number of times the measurement workflow representation predicts the target should be detected (see Example B6). In those embodiments, the comparison in detectability between the testing measurement and the measurement workflow representation is a measure of accuracy. In those embodiments, the Testing Representation is considered “accurate enough” if the actual detectability from the testing measurement fell within the range of detectability predicted by the testing representation.

Exemplary procedure to perform an assessments of accuracy comprise comparing the measurement noise of the testing measurement of the target, e.g. by comprising the measurement noise (in the form of a CV calculation) of a target relative abundance (target molecular count divided by reference molecular count) yielded by the testing measurement compared to the CV yielded by them measurement workflow representation. (see Example 5). Alternatively, the comparison can be performed using a test statistic-test such as the Kolmogorov-Smirnov (KS) Test to compare the distributions of molecular counts.

In some embodiments the StochQuant methods and systems for a given measure of accuracy, a user can identify an accuracy threshold. An “accuracy threshold” can be defined as a minimum value, maximum value, interval of values of a measurement of accuracy, or similar indication of accuracy. For example, in the exemplary procedure of comparing the measurement noise between the testing measurement and the measurement representation, one can set an “accuracy threshold” of 3×, meaning that the measurement noise yielded by the representation must be within 3× of the measurement noise yielded by the testing measurement.

Exemplary accuracy thresholds can include a percentage (e.g. 5%) which can be used in embodiments in which the accuracy is assessed by comparing the detectability of a target via the testing measurement.

Exemplary accuracy thresholds can also comprise a signal to noise ratio which can be used in embodiments in which the accuracy is assessed by comparing the measurement noise of the testing measurement of the target.

Exemplary accuracy thresholds can further comprise p-level value which can be used in embodiments in which a test statistic is used to assess accuracy such as the KS-Test to compare the distributions of molecular counts. In those embodiments, if the obtained p-value is below the significance level (e.g., 0.05), then the null hypothesis is rejected and one can determine that the distribution of molecular counts yielded from the measurement workflow representation differs from the distribution of molecular counts yielded from the testing measurement. In this example, if a p-value greater than 0.05 is obtained, then the measurement workflow representation is within the accuracy threshold and can be used in a StochQuant detection workflow. In embodiments of the StochQuant methods and systems, a user can perform this procedure repeatedly and for different numbers of target molecules in an environment to improve the accuracy assessment. It can be understood that increasing the number of different numbers of target molecules in an environment and performing more repeated measurements can result in improved assessment of accuracy.

In embodiments of the StochQuant methods and systems the desired accuracy of the measurement workflow representation can be defined as a measurement of how closely the measurement workflow representation can approximate the distribution of probable molecular counts of the target obtained via a testing measurement to the actual distribution of probable molecular counts of the target obtained via the testing measurement. (see Example 6).

In some embodiments the StochQuant methods and systems if a measurement workflow representation is not accurate in accordance with a desired accuracy indicated e.g. as a pre-set confidence level. In such cases, a user can improve the measurement workflow by means of any one of or combination of (i) acquiring more segmentation calibration data, (ii) further splitting the manipulations of a segment into additional segments, and/or (iii) using an alternative (but potentially more complicated and/or more computationally intensive) mathematical representation of the segment.

In many embodiments the StochQuant methods and systems StochQuant thus takes advantage of 1) an absolute anchoring measurement), 2) in combination with other known experimental parameters (physical parameters or StochQuant parameters) and in particular detection of molecular counts of the target molecule and of the reference molecule as well as quantified amount of the sample, to apply a measurement workflow representation (that in some cases utilizes Poisson statistics) to derive a probabilistic relationship between actual target molecule abundance in an environment and molecular counts obtained via a testing measurement. StochQuant was demonstrated on amplicon sequencing (16S rRNA gene sequencing) and in connection with determined of taxon abundance as explained in the exemplary experiments of Appendix A and Appendix B of U.S. Provisional Application No. 63/579,291 incorporated by reference in its entirety.

In embodiments of the disclosure probability distribution of abundance of a target molecule in an environment determined by StochQuant detection methods allows the user to identify confidence intervals of target molecule abundances, the interval giving a confidence level, which can be calculated based on the probability distribution of target molecule abundances.

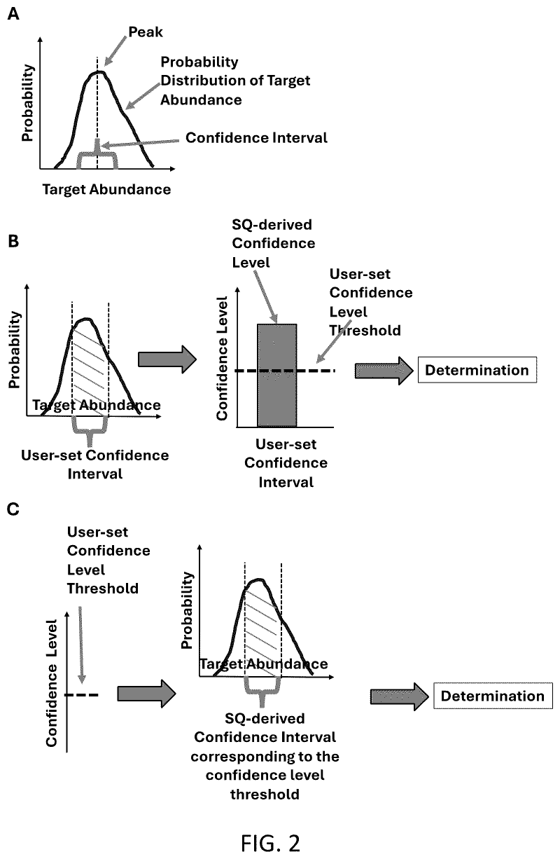

The wording_“confidence interval” indicates the interval (e.g., a range of abundances of target molecule in an environment from some minimum abundance value of the confidence interval to a maximum abundance value of the confidence interval. In some embodiments, the methods and systems use a provided abundance threshold to determine a confidence level above and/or a confidence level below that threshold. In some embodiments, the methods and systems use a provided confidence interval to determine a confidence level for that interval. In some embodiments, the methods and systems use a provided confidence level to determine a confidence interval that has that level. (see FIG. 2). The units of the confidence interval (number of molecules, number of molecules per unit of volume, ratio of target molecules to another target molecule or reference molecule) should match the units of the probability distribution of target abundance in the environment. For example, if the probability distribution of target molecules in an environment is in molecules per microliter, then the confidence interval is provided in molecules per microliter. Examples of a “confidence interval” can include: from 500 target molecules to 1000 target molecules in an environment, from 50 target molecules/μL to 1000 target copies/mL in an environment, from 5 Target A molecules per Target B molecule to 10 Target A molecules per Target B molecule.

In some embodiments, the methods and systems use a provided abundance threshold to determine a confidence level above and/or a confidence level below that threshold. In some embodiments, the methods and systems use a provided confidence interval to determine a confidence level for that interval. In some embodiments, the methods and systems use a provided confidence level to determine a confidence interval that has that level. (see FIG. 2)

The wording “confidence level” indicates probability that the target molecule abundance is within the range of the confidence interval. In practice, the confidence level can be obtained from a probability distribution of target abundances in an environment by integrating over the probability distribution from the lower-bound of the confidence interval to the upper-bound of the confidence interval. In practice, this can be described as “the area under the curve” of the probability distribution, or sum of probabilities within a given confidence interval (see Examples 13-15 and Examples 40-47.

Mathematically, this can be represented:

$$Prob ( {CI}_{LowerBound} \leq X \leq {CI}_{UpperBound} = {\int }_{{CI}_{LowerBound}}^{{CI}_{UpperBound}} f ( x ) dx$$

where f(x) is the probability distribution.

In some embodiments, the Confidence Interval is pre-determined (e.g. +/−some set value around the measurement with maximum probability, or between two set values) and the confidence level is calculated by integrating over the probability distribution from the lower-bound of the confidence interval to the upper-bound of the confidence interval. In other words, in some embodiments, a probability distribution of target abundance and confidence interval are obtained to yield a confidence level. For example, the confidence interval can be set to 5×10\hat{ }5 to 1.5×10\hat{ }6 molecules, and when the confidence level is calculated for a given probability distribution of target abundance in an environment, the confidence level that the number of target molecules is within that range of values is 23.4%. (see Examples 40-47).

In some embodiments, the confidence level is pre-determined (e.g. 50%) and the Confidence Interval is calculated as the interval above and/or below a selected target abundance value that provides that confidence level. In other words, in some embodiments, a probability distribution of target abundance, a selected target abundance, and a confidence level are obtained to yield a Confidence Interval. For example, for a given probability distribution of target abundance, selected target abundance of 1,000 copies/mL, and confidence level of 50% (that the selected target abundance is greater than or equal to 1,000 copies/mL), the resulting calculation can yield a Confidence Interval of 1,000 copies/mL to 6,000 copies/mL For example, the interval to be demined is the range of 75% probable target molecule abundances centered around whatever the maximum probable count is, and the resulting curve can show that the interval of +/−6×10 \hat{ }4 around 1.5×10\hat{ }5 molecules gives the range of values that have a 75% probability to include the correct count.

In some embodiments, a confidence level threshold is predetermined, and the confidence levels for the two options (above and below) are calculated based on confidence interval bounded by the confidence level threshold.

The wording “confidence level threshold” indicates a pre-set minimum or maximum confidence level that can be used to make a binary decision (above vs. below the threshold). For example, if a minimum confidence level threshold of 95% is needed to determine that a target is present within a confidence interval, and a confidence level of 99% is obtained, then it is determined that the target is present within the confidence interval. (see Example 14).

For example, in some embodiments of StochQuant methods and systems a confidence level threshold of 25% is provided, with confidence levels above the confidence level threshold yielding a “positive” test result determination, and confidence level below the confidence level threshold yielding “negative” test result determination. Provided a probability distribution of target abundance and a confidence interval, a confidence level can be obtained. If the obtained confidence level is below the confidence level threshold (e.g., a confidence level of 10% for a confidence level threshold of 25%), a “negative” test result determination is yielded. If the obtained confidence level is above the confidence level threshold (e.g., a confidence level of 90% for a confidence level threshold of 25%), a “positive” test result determination is yielded.

Embodiments of StochQuant detection methods and system can comprise obtaining a confidence level from a confidence interval probability distribution of target abundance, thus improving accuracy of detection.

Accordingly, in StochQuant methods and systems herein described, in embodiments where the probability distribution of target molecules in an environment is so narrow to be approximated to a deterministic value, the StochQuantization of the related detection allows to derive a confidence interval which correspondence to a confidence level.

Consequently, each and every detection involving a molecular count in which a reference count can be obtained can be StochQuantized including single step detection and completely deterministic detections. In particular in detection workflow comprising single step detection approximated to deterministic detection, the StochQuantization will add an understanding of the confidence level of the resulting count that will otherwise be absent. This confidence level can also account for background noise and other factors such as user's mistakes if the probability distribution is chosen that account for those mistakes.

In some embodiments, StochQuant can be used to provide a method and a system to probabilistically detect a target molecule in an environment, accounting for the stochastic impact affecting the target molecule the detection due to the stochasticity introduced by the detection process. The method comprises:
performing a testing measurement comprising
   obtaining a molecular count of the target molecule in an environment thereof; and
   obtaining a molecular count of a reference molecule; and
providing an absolute anchoring value of the reference molecule; and
obtaining a probability distribution of the target molecule abundance in the environment as a function of
   the molecular count of the target molecule;
   the molecular count of the reference molecule; and
   the absolute anchoring value of the reference molecule;
      In the method to probabilistically detect a target molecule in an environment of the first aspect, the probability distribution of the target molecule abundance in the environment is indicative of the confidence of detection or non-detection or confidence of the quantitative value of the target molecule detected in the environment.

In some embodiments, the absolute anchoring value of the reference molecule is a value obtained by a previous measurement.

In some embodiments, the absolute anchoring value of the reference molecule is obtained by performing in the environment an absolute anchoring measurement of the reference molecule.

In some embodiments, the reference molecule is added to the environment and the absolute anchoring value of the reference molecule is a known absolute count or distribution of absolute counts of the reference molecule added to the environment.

In some embodiments, the absolute anchoring value is a single detected count.

In some embodiments, the absolute anchoring value is a plurality of detected counts.

In some embodiments, the plurality of detected counts is comprised in a distribution.

In some embodiments, the absolute anchoring value is a number which is proportional to the count and is adjusted to obtain the true count.

In some embodiments, the testing measurement is performed by 16S rRNA gene sequencing, ITS gene sequencing, 18S rRNA gene sequencing, COI gene sequencing, ITS2 gene sequencing, RBP1 gene sequencing, RBP2 gene sequencing,V(D) J region sequencing, mitochondrial gene sequencing, functional gene sequencing, bulk RNA sequencing (RNA-seq), single cell RNA-seq, metagenomic sequencing, metatranscriptomic sequencing, spatial transcriptomics, Chromatin Immunoprecipitation Sequencing (ChIP-seq SIMOA, single molecule fluorescence in situ hybridization (smFISH), hybridization chain reaction (HCR) FISH, and next generation sequencing (NGS) adapted for protein quantification.

In some embodiments, the reference molecule is a single type of molecule is one or more of the mRNA of a gene Glyceraldehyde-3-phosphate dehydrogenase (GAPDH), Phosphoglycerate kinase 1 (PGK1), Peptidylpropyl isomerase A (PPIA), ribosomal protein L13a (RPL13A), ribosomal protein large P0 (RPLP0), Beta-2-microglobulin (B2M), YWHAZ, SDHA, TFRC, GUSB, HMBS, HPRT1, TBP; 16S, tus, rpoD, glyA, dnaB, gyrA, pykA/F, pfkA/B, mdoG, arcA; DUF221, ubcB, ADA, fis1, Cu-ATPase, psm1, spo7, spt3, DUF500, sac7, AP-2 beta, npl1, Beta-tubulin, Arabinofuranosidase-B2, and Xylanase C.

In some embodiments, the reference molecule is a plurality of types of molecules simultaneously detected during the testing measurement to provide a same count such as multiple 16S genes which all amplify from the same primer.

In some embodiments, the reference molecule formed by a plurality of molecule types that are simultaneously detected during the testing measurement comprise multiple genes, portions of genes, regions, or portions of regions which all amplify from the same primer such as ITS, ITS2, 18S, COI, ITS2,V (D) J region.

In some embodiments, the reference molecule formed by a plurality of molecule types that are simultaneously detected during the testing measurement comprise types of multiple molecules all which give rise to a fluorescent signal, provided the same probe or fluorophore, such as Lipopolysaccharides (LPS), Peptidoglycan, Teichoic acids, specific DNA or RNA targets.

In some embodiments, the reference molecule is a plurality of types of molecules each separately detected during the testing measurement to provide separate unique counts.

In some embodiments, the testing measurement comprises bulk RNA-seq or shotgun metagenomic sequencing.

In some embodiments, the reference molecule comprises one or more of: a fungal cell-type specific reference molecule formed by multiple DNA molecule types; a bacterial cell-type specific reference molecule formed by multiple DNA molecule types; and a reference molecule formed by a reference DNA molecule and a reference RNA molecule.

In some embodiments, the probability distribution is obtained in non-parametric form as one or more molecular counts, each with a probability of being the true molecular count.

In some embodiments, the probability distribution is obtained in the form of shape parameters for a known discrete probability distribution.

In some embodiments, the probability distribution is obtained in the form of a list of target abundances where the representation of each target abundance is correlated with its probability.

In some embodiments, the target molecule is known or expected to be comprised in the environment and/or the sample at a low absolute abundance.

In some embodiments, the target molecule is known or expected to be comprised in the environment and/or the sample at a low relative abundance.

In some embodiments, the target molecule is comprised in a microorganism included in a microbial community, such as a microbiome.

In some embodiments, the probabilistic detection is performed in connection with detection of abundance of a microorganism and/or related taxa.

In some embodiments, the obtaining a probability distribution is performed on a computer with a processor and a memory.

In some embodiments, the computer is a network of computers.

In some embodiments, StochQuant can be used in a method and a system to probabilistically measure an abundance of a target molecule in an environment accounting for the stochasticity impacting the detected abundance which is introduced by the measurement process.

The method comprises: i) determining a) an absolute anchoring value of a reference molecule in the environment.

The method further comprises ii) performing a testing measurement comprising a measurement workflow, producing quantitative testing measurements, on the environment, a sample and/or a subsample thereof, to establish:
b) a corresponding molecular count of the target molecule in the environment; and
c) a corresponding molecular count of the reference molecule in the environment.

The method also comprises iii) inputting a), b) and c) into a computer-based system, the computer system being configured to generate a probability distribution of abundance of the target molecule in the sample based on the basis of a), b) and c) by a model of the quantitative testing measurements.

The method additionally comprises iv) based on the probability distribution, producing, through the computer-based system, one or more of:
confidence level of abundance values above and below a threshold abundance value of the target molecule input to the computer system;
confidence interval of abundance values based on an abundance value confidence level of the target molecule input to the computer system; and
abundance value confidence level based on a confidence interval of abundance values input to the computer system.

In some embodiments, the absolute anchoring value of the reference molecule is obtained by performing in a sample of the environment an absolute anchoring measurement of the reference molecule.

In some embodiments, the absolute anchoring value of the reference molecule is a known value because the reference molecule would be added for the measuring workflow in a known amount.

D4 In some embodiments, the reference molecule is not present in the environment but is added to the measuring workflow at some point.

In some embodiments, the absolute anchoring value is an adjusted value of an absolute anchoring measurement of the reference molecule.

In some embodiments, the measuring workflow includes amplicon sequencing.

In some embodiments, the amplicon sequencing includes one or more of: 16S rRNA gene sequencing, ITS gene sequencing, 18S rRNA gene sequencing, COI gene sequencing, ITS2 gene sequencing, RBP1 gene sequencing, RBP2 gene sequencing, V(D) J region sequencing, mitochondrial gene sequencing, functional gene sequencing.

In some embodiments, the reference molecule is a mRNA of a gene.

In some embodiments, the reference molecule is selected from: Glyceraldehyde-3-phosphate dehydrogenase (GAPDH), Phosphoglycerate kinase 1 (PGK1), Peptidylpropyl isomerase A (PPIA), ribosomal protein L13a (RPL13A), ribosomal protein large P0 (RPLP0), Beta-2-microglobulin (B2M), YWHAZ, SDHA, TFRC, GUSB, HMBS, HPRT1, TBP; bacterial housekeeping genes such as 16S, tus, rpoD, glyA, dnaB, gyrA, pykA/F, pfkA/B, mdoG, arcA; fungal housekeeping genes such as DUF221, ubcB, ADA, fis1, Cu-ATPase, psm1, spo7, spt3, DUF500, sac7, AP-2 beta, npl1, Beta-tubulin, Arabinofuranosidase-B2, Xylanase C.

In some embodiments, the reference molecule is a plurality of types of molecules simultaneously detected during the testing measurement to provide a same count.

In some embodiments, the reference molecule is multiple 16S genes which all amplify from the same primer.

In some embodiments, the plurality of molecule types that are simultaneously detected during the testing measurement are selected from multiple genes, portions of genes, regions, or portions of regions which all amplify from the same primer Lipopolysaccharides (LPS), Peptidoglycan, Teichoic acids, and specific DNA or RNA targets.

In some embodiments, the reference molecule is a plurality of types of molecules each separately detected during the testing measurement to provide separate unique counts that are used to determine at least the molecular count of the reference molecule.

In some embodiments, the forming a probability distribution of abundances of the target molecule is further based on multiple molecular counts of the reference molecule.

In some embodiments, the plurality of types of molecules are selected from multiple RNA expression reference molecules.

In some embodiments, the method also includes determining a probability that an actual abundance of the target molecule in the environment is above (or below) a threshold abundance by calculating a total area of the probability distribution higher than (or lower than) the threshold abundance.

In some embodiments, the method also includes determining a probability that an actual abundance of the target molecule in the environment is above (or below) or equal to a threshold abundance by calculating a total area of the probability distribution higher than (or lower than) or equal to the threshold abundance.

In some embodiments, the method also includes determining a confidence level by calculating the area of the probability distribution within a given confidence interval.

In some embodiments, the method also includes determining a confidence interval by calculating what interval within the probability distribution provides a given confidence level.

In some embodiments, the interval is centered around a given abundance value.

In some embodiments, StochQuant can be used in connection with a computer-based system comprising a processor, memory, input components, and output components and configured to perform StochQuant detection methods and systems of the disclosure.

In those embodiments, the computer-based system is configured to: i) receive, process and store, through the input components, the processor and the memory, a) an absolute anchoring values of a reference molecule in an environment a sample and/or a subsample thereof, b) a molecular count of a target molecule in the environment as determined by a measuring workflow performed in the environment, the sample and/or a the subsample thereof, and c) a molecular count of the reference molecule in the environment as determined by the measuring workflow performed in the environment, the sample and/or a the subsample thereof;

The computer-based system is further configured to: ii) process, through the processor, a), b) and c) from i) into a model of the measuring workflow configured to obtain probabilistically distributed abundance values of the target molecule in the environment; and at least one of:
iiia) receive, through the input components, a threshold abundance value of the target molecule and process, through the processor, the threshold abundance value of the target molecule through the probabilistically distributed abundance values of the target molecule to obtain and output, through the output components, a confidence level of abundance values above and below the threshold abundance value of the target molecule; or
iiib) receive, through the input components, an abundance value confidence level of the target molecule and process, through the processor, the abundance value confidence level of the target molecule through the probabilistically distributed abundance values of the target molecule to obtain and output, through the output components, a confidence interval of abundance values of the target molecule; or
iiic) receive, through the input components, a confidence interval of abundance values of the target molecule and process, through the processor, the confidence interval of abundance values of the target molecule through the probabilistically distributed abundance values of the target molecule to obtain and output, through the output components, an abundance value confidence level of the target molecule.

In some embodiments, the absolute anchoring value of the reference molecule is obtained by performing in a sample of the environment an absolute anchoring measurement of the reference molecule.

In some embodiments, the absolute anchoring value of the reference molecule is a known value because the reference molecule would be added for the measuring workflow in a known amount.

In some embodiments, the reference molecule is not present in the environment but is added to the measuring workflow at some point.

In some embodiments, the absolute anchoring value is an adjusted value of an absolute anchoring measurement of the reference molecule.

In some embodiments, the measuring workflow includes amplicon sequencing.

In some embodiments, the amplicon sequencing includes one or more of: 16S rRNA gene sequencing, ITS gene sequencing, 18S rRNA gene sequencing, COI gene sequencing, ITS2 gene sequencing, RBP1 gene sequencing, RBP2 gene sequencing, V(D) J region sequencing, mitochondrial gene sequencing, functional gene sequencing.

In some embodiments, the reference molecule is an mRNA of a gene.

In some embodiments, the reference molecule is selected from: Glyceraldehyde-3-phosphate dehydrogenase (GAPDH), Phosphoglycerate kinase 1 (PGK1), Peptidylpropyl isomerase A (PPIA), ribosomal protein L13a (RPL13A), ribosomal protein large P0 (RPLP0), Beta-2-microglobulin (B2M), YWHAZ, SDHA, TFRC, GUSB, HMBS, HPRT1, TBP; bacterial housekeeping genes such as 16S, tus, rpoD, glyA, dnaB, gyrA, pykA/F, pfkA/B, mdoG, arcA; fungal housekeeping genes such as DUF221, ubcB, ADA, fis1, Cu-ATPase, psm1, spo7, spt3, DUF500, sac7, AP-2 beta, npl1, Beta-tubulin, Arabinofuranosidase-B2, Xylanase C.

In some embodiments, the reference molecule is a plurality of types of molecules simultaneously detected during the testing measurement to provide a same count.

In some embodiments, the reference molecule is multiple 16S genes which all amplify from the same primer.

In some embodiments, the plurality of molecule types that are simultaneously detected during the testing measurement are selected from multiple genes, portions of genes, regions, or portions of regions which all amplify from the same primer Lipopolysaccharides (LPS), Peptidoglycan, Teichoic acids, and specific DNA or RNA targets.

In some embodiments, the reference molecule is a plurality of types of molecules each separately detected during the testing measurement to provide separate unique counts that are used to determine at least the molecular count of the reference molecule.

In some embodiments, the forming a probability distribution of abundances of the target molecule is further based on multiple molecular counts of the reference molecule.

In some embodiments, the plurality of types of molecules are selected from multiple RNA expression reference molecules.

In some embodiments, the computer-based system is further configured to determine a probability that an actual abundance of the target molecule in the environment is above (or below) a threshold abundance by calculating a total area of the probability distribution higher than (or lower than) the threshold abundance.

In some embodiments, the computer-based system is further configured to determine a probability that an actual abundance of the target molecule in the environment is above (or below) or equal to a threshold abundance by calculating a total area of the probability distribution higher than (or lower than) or equal to the threshold abundance.

In some embodiments, the computer-based system is further configured to determine a confidence level by calculating the area of the probability distribution within a given confidence interval.

In some embodiments, the computer-based system is further configured to determine a confidence interval by calculating what interval within the probability distribution provides a given confidence level.

In some embodiments, the interval is centered around a given abundance value.

A skilled person will understand that the StochQuant methods and systems exemplified in Examples 1 to 48 as well as in Appendix A and Appendix B of U.S. Provisional Application No. 63/579,291 incorporated by reference in its entirety in connection with testing measurements performed by amplicon sequencing provides a proof of principle and a representative example of the StochQuant methods and systems performed with other testing measurement, samples, target molecules, reference molecule and anchoring measurements in the sense of the disclosure.

In particular, a skilled person will understand from the examples of Appendix A and Appendix B of U.S. Provisional Application No. 63/579,291 incorporated by reference in its entirety in view of the remaining parts of the disclosure, that StochQuant includes two key capabilities: (1) StochQuant provides a probability distribution of probable target abundance in an environment (from aa molecular count of target molecule obtained via the testing measurement, a molecular count of reference molecule obtained via the testing measurement, an absolute anchoring value of the reference molecule, and in some cases other physical StochQuant parameters such as quantitatively measurable amount(s)) and mathematically explains why detecting low-to-moderate-abundance targets will intrinsically result in unreliable and irreproducible detection and quantification. (2) StochQuant provides a probability distribution of target abundance (relative or absolute) from a molecular count of the target molecule (from the testing measurement), an absolute anchoring value of the reference molecule, and quantitatively measurable amount(s) and other StochQuant physical parameters) StochQuant probability distributions of target abundance mathematically explain and integrate in the results of the testing measurement the stochasticity inherent to molecular detection in a sample.

This is an improvement in detection technology which is particularly valuable in connection with detection of low-to-moderate-abundance targets which intrinsically result in unreliable and irreproducible detection and quantification due to the heightened impact of the stochasticity introduced by the detection workflow on the related molecular count as will be understood by a skilled person.

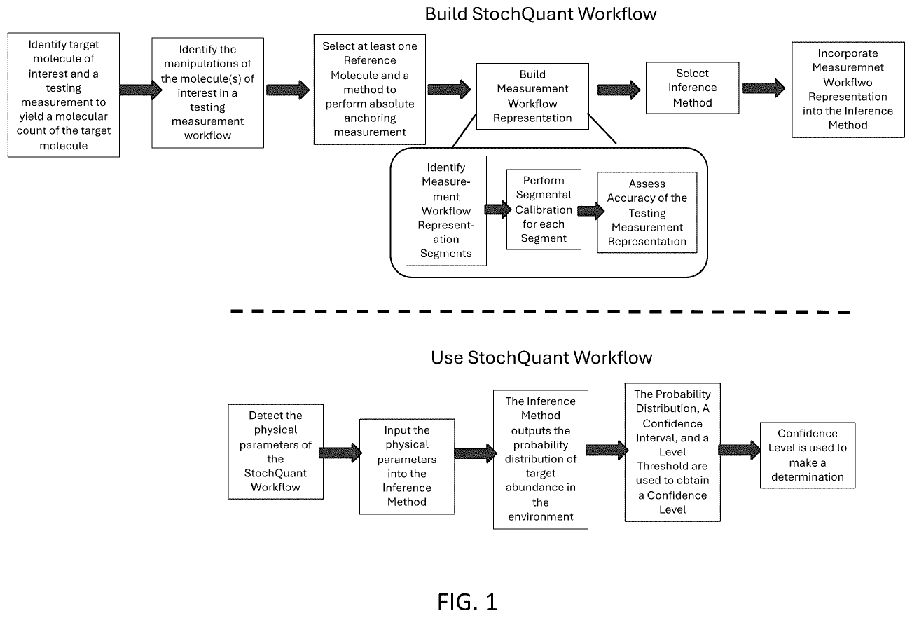

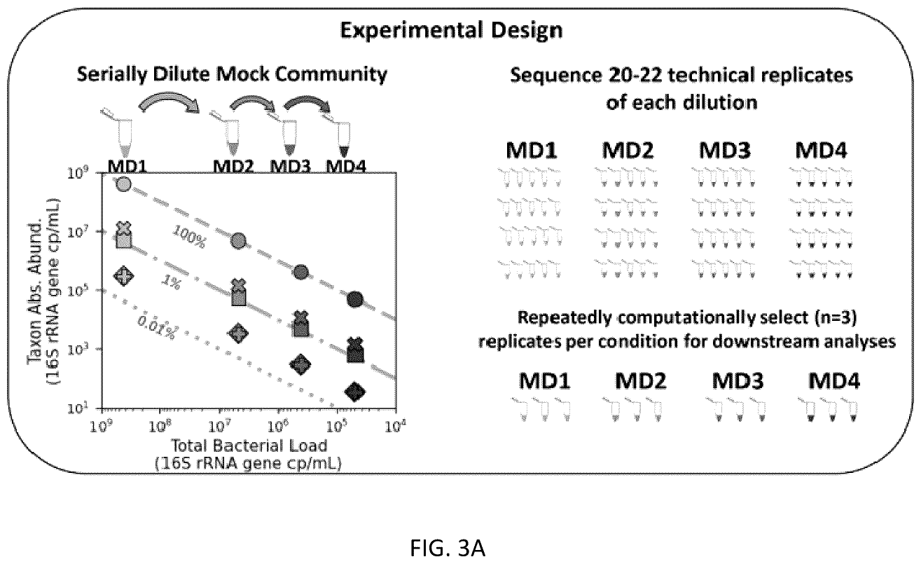

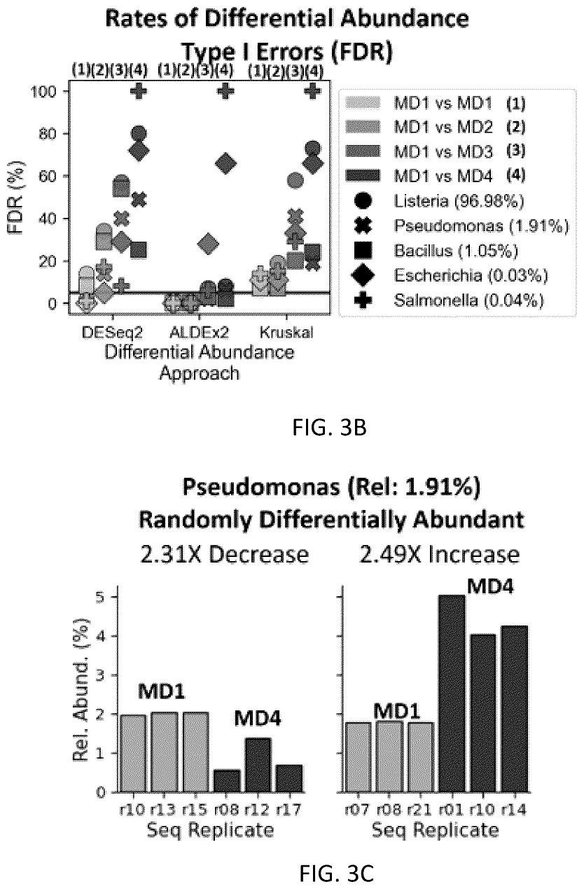

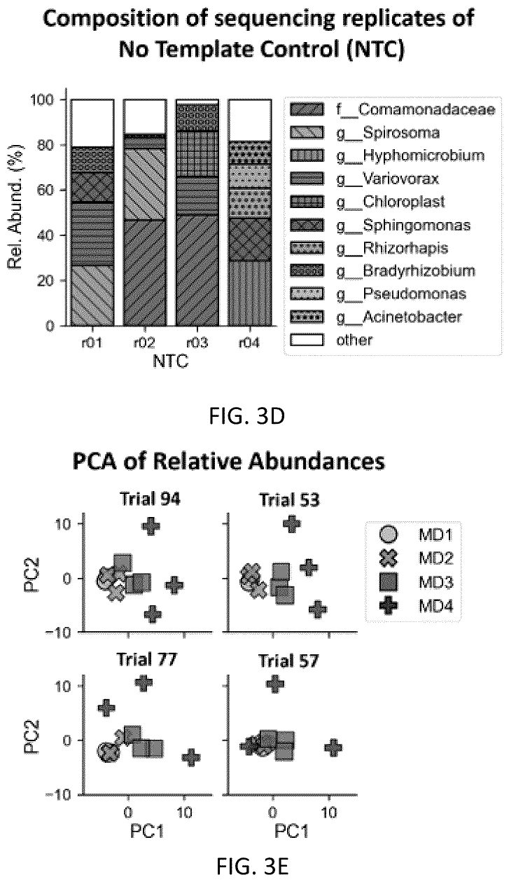

In particular, in embodiments of StochQuant methods and systems, by relying on absolute quantification, StochQuant mathematically explains how molecular count data is generated from small numbers of target molecules, including the possible range of reads generated from a single molecule and integrates such explanation in the detection process thus improving confidence of the detection. StochQuant also informs experimental design because it describes the conditions under which detecting (e.g., sequencing) low-to-moderate abundance target molecule microbes intrinsically results in reliable or unreliable detection and quantification. For example, StochQuant simulations of sequencing accurately predict the detectability and measurement noise of taxa across a wide range of absolute and relative abundances as shown in the exemplary methods and systems of exemplified in Examples 1 to 48 and in Appendix A and Appendix B of U.S. Provisional Application No. 63/579,291 incorporated by reference in its entirety (see FIGS. 3, Supplementary FIGS. 1, 2, and main text in the “Testing of StochQuant with low-to-moderate load human gut biopsies” section).

In some embodiments the probability distribution of the target molecule abundance in the sample indicative of the confidence of detection or non-detection or confidence of the quantitative value of the target molecule detected in the sample which is indicative of the probabilistic detection of the target molecule in the environment.

The term “probabilistic detection” as used herein refers to the use of a set of one or more data points each with determined probability of occurrence to determine the quantitative likelihood of one or more possible counts of an item or the likelihood of a qualitative occurrence of the item.

A probabilistic detection can be an absolute or relative measurement-and in some cases be directed to qualitative detection (presence/absence detection) or quantitative detection.

In embodiments of the present disclosure probabilistic detection is obtained by generating a StochQuant probability distribution as understood by a skilled person upon reading of the present disclosure.

In some embodiments, wherein the testing measurement is performed sample is for the purpose of detecting abundance of the target molecule in the environment from which the sample has been taken an additional StochQuant parameter is be included in the determination of the probability distribution, a quantitively measured amount of the sample.

The term “sample” as used herein indicates a limited quantity of something that is indicative of a larger quantity of that something and is used in testing examination or study. Accordingly, a sample of an environment is a portion of the environment subject to testing. Accordingly, samples of a biological environment comprise for example cultures, tissues, commercial recombinant proteins, synthetic compounds or portions thereof. In particular, biological sample can comprise one or more cells of any biological lineage including microbial and in particular prokaryotic cells, as being representative of the total population of similar cells in the sampled individual. Exemplary biological samples comprise the following: whole venous and arterial blood, blood plasma, blood serum, dried blood spots, cerebrospinal fluid, lumbar punctures, nasal secretions, sinus washings, tears, corneal scrapings, saliva, sputum or expectorate, bronchoscopy secretions, transtracheal aspirate, endotracheal aspirations, bronchoalveolar lavage, vomit, endoscopic biopsies, colonoscopic biopsies, bile, vaginal fluids and secretions, endometrial fluids and secretions, urethral fluids and secretions, mucosal secretions, synovial fluid, ascitic fluid, peritoneal washes, tympanic membrane aspirate, urine, clean-catch midstream urine, catheterized urine, suprapubic aspirate, kidney stones, prostatic secretions, feces, mucus, pus, wound draining, skin scrapings, skin snips and skin biopsies, hair, nail clippings, cheek tissue, bone marrow biopsy, solid organ biopsies, surgical specimens, solid organ tissue, cadavers, or tumor cells, among others identifiable by a skilled person. Biological samples can be obtained using sterile techniques or non-sterile techniques, as appropriate for the sample type, as identifiable by persons skilled in the art. Some biological samples can be obtained by contacting a swab with a surface on a human body and removing some material from said surface, examples include throat swab, nasal swab, nasopharyngeal swab, oropharyngeal swab, cheek or buccal swab, urethral swab, vaginal swab, cervical swab, genital swab, anal swab, rectal swab, conjunctival swab, skin swab, and any wound swab. Depending on the type of biological sample and the intended analysis, biological samples can be used freshly for sample preparation and analysis, or can be fixed using fixative.

In some embodiments, samples can also comprise a plurality of samples in the form of DNA-encoded libraries provided following conjugation of target molecule within an environment or a sample with DNA tags. Exemplary DNA-encoded libraries comprise library provided by attaching a DNA barcode (e.g., a unique sequence of nucleic acids that can be read out via a sequencing technology) to target molecule such as nucleic acids, amino acids, synthetic particles, drugs, natural or synthetic compounds, or theranostic particles. DNA encoded libraries can be used for several applications. Exemplary applications of DNA libraries include drug discovery, testing efficacy of anti-cancer drugs and other therapeutics, studying ligand-receptor binding affinity, testing efficacy of immune checkpoint blockade against cancer by DNA barcoding, detection of micro-organisms, detection of allergens, detection of viruses, identification and detection of cells, multiplex detection, and others (as described e.g., by ref. [10]). Other examples include high resolution mapping of chromatin-associated proteins and chromatin modifications across the genome (CHIP-seq), determination of genome structure (DNase-seq and HI-C), protein translation dynamics (ribosome profile, phage display, yeah-2-hybrid screening, protein evolution, high-throughput biochemistry, materials science, DNA labeling of carbohydrates, DNA labeling of nanoparticles, and others (as described e.g., by ref. [11]).

Exemplary samples according to the instant disclosure samples comprise tear fluid, saliva, nasal, oral, tonsillar, and pharyngeal swabs, sputum, bronchoalveolar lavage (BAL), gastric, small-intestine, and large-intestine contents and aspirates, feces, bile, pancreatic juice, urine, vaginal samples, semen, skin swabs, tissue and tumor biopsy, blood, lymph, cerebrospinal fluid, amniotic fluid, mammary gland secretions/breast milk. Examples of environmental and industrial samples: soil and other media for (agricultural) plant growth, water, sediment, oil well samples, bioreactors (e.g., complex/mixed probiotics). Samples can also include clean room swabs, hospital surfaces, and mucosal brush biopsies as understood by a skilled person.

In particular, in some embodiments, StochQuant methods and systems of the disclosure comprise a method to probabilistically detect a target molecule in an environment, the method comprising: separating a portion of the environment to obtain a sample of the environment the sample having a quantitatively measurable amount; and providing an absolute anchoring value of a reference molecule in the sample. The StochQuant methods and systems further comprise performing a testing measurement comprising obtaining a molecular count of the target molecule in the sample; and—obtaining a molecular count of the reference molecule in the sample.

The StochQuant methods and systems herein described, also comprise obtaining a probability distribution of the target molecule abundance in the sample as a function of the molecular count of the target molecule; the molecular count of the reference molecule; the absolute anchoring value of the reference molecule; and a quantitively measured amount of the sample.

A quantitatively measurable amount of sample is an amount quantitatively measurable such as the volume of the sample the mass of the sample the weight of the sample and others. In some embodiments, a quantitatively measurable amount can be a value of or indicative of amount of sample material that can be expressed in numbers (volume, or mass, weight, or additional parameters identifiable by a skilled person).

A quantitatively measurable amount of the sample is factored in the determination of the target molecule abundance in the sample in view of the proportionality distribution between the absolute anchoring measurement and the molecular count of the absolute anchor as understood by a skilled person.

In some embodiments, of StochQuant methods and systems wherein the reference molecule is spiked into an environment and the testing measurement is performed in the environment a quantitatively measured amount of the sample is optional as understood by a skilled person. Those embodiments are particularly directed to environment where the amount of target molecule is included at low and moderate relative or absolute abundance.

StochQuant method and systems of the disclosure, can include sampling as manipulation which is part of the detection workflow of a testing measurement and is comprised in one or more segments of the workflow, which can be modeled by StochQuant parameters further including a quantitively measured amount of the sample as will be understood by a skilled person upon reading of the present disclosure.

In some embodiments, StochQuant is used in connection with a method is to probabilistically detect a target molecule in an environment, the method comprising:
separating a portion of the environment to obtain a sample of the environment the sample having a quantitatively measurable amount;
providing an absolute anchoring value of a reference molecule in the sample;
performing a testing measurement comprising
obtaining a molecular count of the target molecule in the sample; and
obtaining a molecular count of the reference molecule in the sample; and
obtaining a probability distribution of the target molecule abundance in the sample as a function of the molecular count of the target molecule;
the molecular count of the reference molecule;
the absolute anchoring value of the reference molecule; and a quantitively measured amount of the sample;
the probability distribution of the target molecule abundance in the sample indicative of the confidence of detection or non-detection or confidence of the quantitative value of the target molecule detected in the sample which is indicative of the probabilistic detection of the target molecule in the environment.

In some embodiments, the sample obtained from the separating is obtained by serially and/or in parallel sampling of the environment.

In some embodiments, the sample is a plurality of samples and the absolute anchoring measurement, the molecular count of the target molecule, and the probability distribution are obtained in one or more same or different samples of the plurality of samples.

In some embodiments, the absolute anchoring value of the reference molecule is a value obtained by a previous measurement.

In some embodiments, the absolute anchoring value of the reference molecule is obtained by performing in the sample an absolute anchoring measurement of the reference molecule.

In some embodiments, the reference molecule is added to the sample and the absolute anchoring value of the reference molecule is a known absolute count or distribution of absolute counts of the reference molecule added to the sample.

In some embodiments, the absolute anchoring value is a single detected count.

In some embodiments, the absolute anchoring value is a plurality of counts.

In some embodiments, the plurality of counts is comprised in a distribution.

In some embodiments, the absolute anchoring value is a number which is proportional to the count, and is adjusted to obtain the true count.

In some embodiments, the anchoring measurement and testing measurement are performed in a same sample.

In some embodiments, the anchoring measurement and testing measurement are performed in separate samples from a same environment.

In some embodiments, the anchoring measurement is performed in a sample and testing measurement is performed in a sub-sample of the sample.

In some embodiments, obtaining a molecular count of the target molecule and obtaining a molecular count of the reference molecule are performed in a same sample or in subsamples of a same sample.

In some embodiments, the testing measurement is performed by amplicon sequencing (16S rRNA gene sequencing, ITS gene sequencing, 18S rRNA gene sequencing, COI gene sequencing, ITS2 gene sequencing, RBP1 gene sequencing, RBP2 gene sequencing, V(D) J region sequencing, mitochondrial gene sequencing, functional gene sequencing).

In some embodiments, the reference molecule is a single type of molecule, such as the mRNA of a gene.

In some embodiments, the reference molecule is selected from Glyceraldehyde-3-phosphate dehydrogenase (GAPDH), Phosphoglycerate kinase 1 (PGK1), Peptidylpropyl isomerase A (PPIA), ribosomal protein L13a (RPL13A), ribosomal protein large P0 (RPLP0), Beta-2-microglobulin (B2M), YWHAZ, SDHA, TFRC, GUSB, HMBS, HPRT1, TBP; bacterial housekeeping genes such as 16S, tus, rpoD, glyA, dnaB, gyrA, pykA/F, pfkA/B, mdoG, arcA; fungal housekeeping genes such as DUF221, ubcB, ADA, fis1, Cu-ATPase, psm1, spo7, spt3, DUF500, sac7, AP-2 beta, npl1, Beta-tubulin, Arabinofuranosidase-B2, Xylanase C.

In some embodiments, the reference molecule is a plurality of types of molecules simultaneously detected during the testing measurement to provide a same count such as multiple 16S genes which all amplify from the same primer.

In some embodiments, the plurality of molecule types that are simultaneously detected during the testing measurement are selected from multiple genes, portions of genes, regions, or portions of regions which all amplify from the same primer Lipopolysaccharides (LPS), Peptidoglycan, Teichoic acids, and specific DNA or RNA targets.

In some embodiments, the reference molecule is a plurality of types of molecules each separately detected during the testing measurement to provide separate unique counts.

In some embodiments, of the plurality of types of molecules each separately detected during the testing measurement to provide separate unique counts are selected from multiple RNA expression reference molecules.

In some embodiments, obtaining a molecular count of the target molecule and obtaining a molecular count of the reference molecule are performed in a same sample.

In some embodiments, obtaining a molecular count of the target molecule and obtaining a molecular count of the reference molecule are performed in subsamples of a same sample.

In some embodiments, the probability distribution is obtained in non-parametric form as one or more molecular counts, each with a probability of being the true molecular count.

In some embodiments, the probability distribution is obtained in the form of shape parameters for a known discrete probability distribution.

In some embodiments, the probability distribution is obtained in the form of a list of target abundances where the representation of each target abundance is correlated with its probability.

In some embodiments, the target molecule is known or expected to be comprised in the environment and/or the sample at a low absolute abundance.

In some embodiments, the target molecule is known or expected to be comprised in the environment and/or the sample at a low relative abundance.

In some embodiments, the target molecule is comprised in a microorganism included in a microbial community, such as a microbiome.

In some embodiments, the probabilistic detection is performed in connection with detection of abundance of a microorganism and/or related taxa.

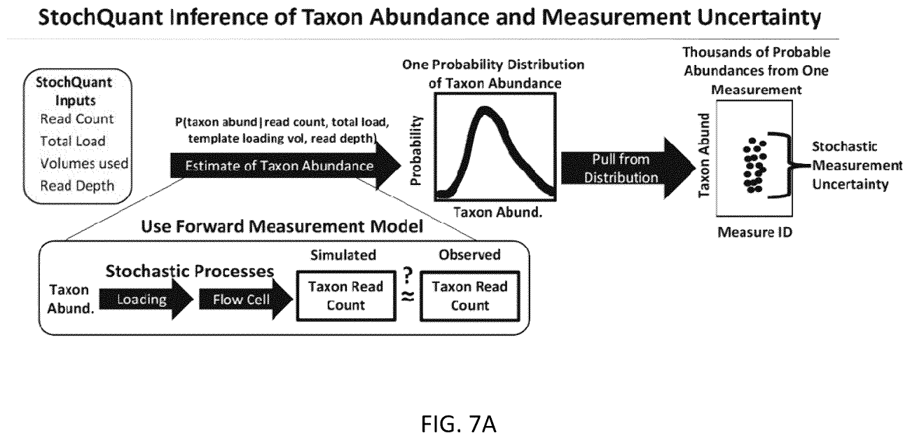

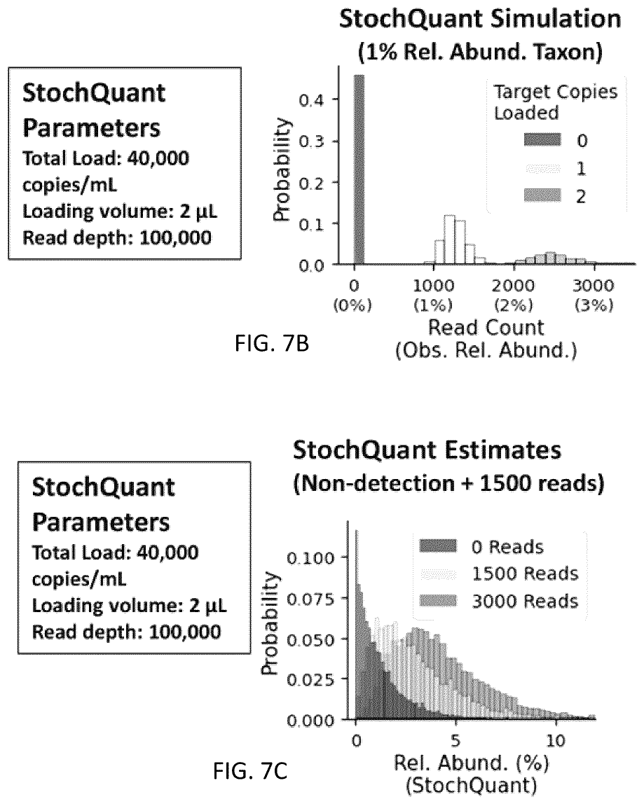

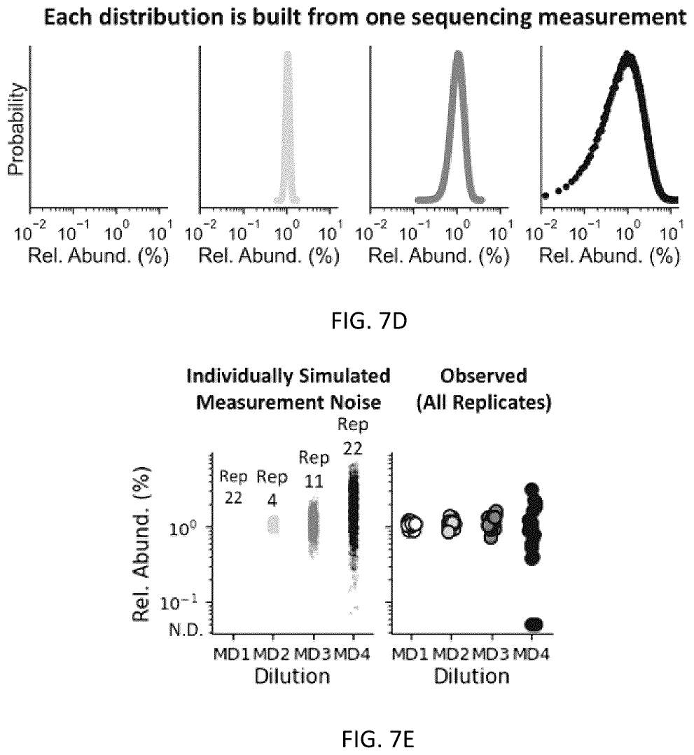

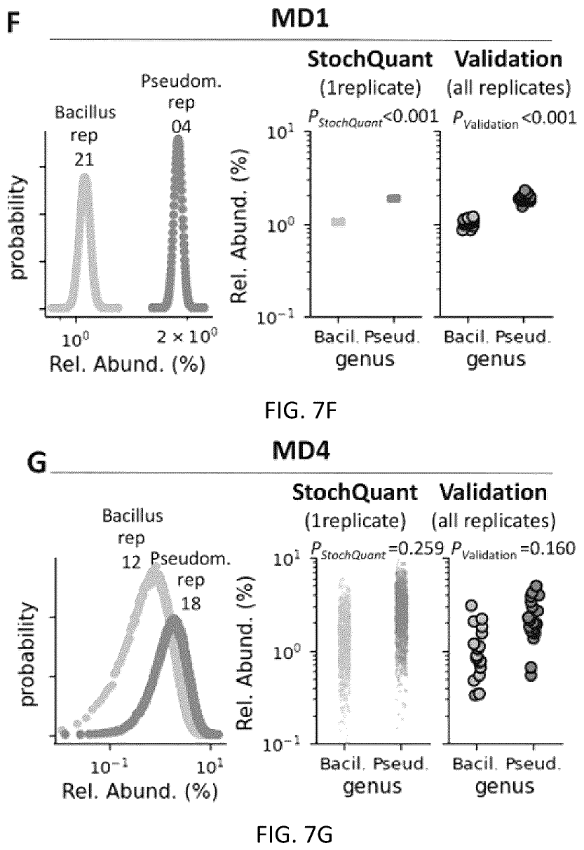

In some embodiments, in StochQuant methods and systems of the disclosure the sample obtained from the separating is obtained by serially and/or in parallel sampling of the environment (see Examples 16 to 20, Appendix A of U.S. provisional No. 63/579,291, and Examples 3 to 15 and Appendix B of U.S. provisional No. 63/579,291 in particular FIG. 1A, 1B, 6G, 6H, Supplementary FIGS. 7, 8, 10).

In some embodiments, in StochQuant methods and systems of the disclosure the sample is a plurality of samples and the absolute anchoring measurement, the molecular count of the target molecule, and the probability distribution are obtained in one or more same or different samples of the plurality of samples (see Examples 1 to 47 as well as Appendix A and Appendix B U.S. Provisional Application No. 63/579,291 incorporated by reference in its entirety).

In some embodiments, in StochQuant methods and systems of the disclosure the absolute anchoring value of the reference molecule is a value obtained by a previous measurement (see Appendix A and Appendix B U.S. Provisional Application No. 63/579,291 incorporated by reference in its entirety).

In some embodiments, in StochQuant methods and systems of the disclosure the absolute anchoring value of the reference molecule is obtained by performing in the sample an absolute anchoring measurement of the reference molecule.

In some embodiments, in StochQuant methods and systems of the disclosure the reference molecule is added to the sample and therefore the absolute anchoring value of the reference molecule is a known absolute count, or distribution of absolute counts, of the reference molecule added to the sample. Since the reference molecule is added (“spiked-in”) by the tester, the amount (or distribution of possible amounts) is known by the tester, and therefore it has an “absolute count”.

In some embodiments the absolute anchoring measurement performed according to methods and systems herein described results in a single detected count in other embodiments results in a plurality of detected counts (e.g., comprised in a distribution) as understood by a skilled person.

In some embodiments an absolute anchoring measurement can be performed by adding a predetermined amount of reference molecule to the samples understood by a skilled person upon reading of the present disclosure.

In some embodiments, the absolute anchoring measurement results in a number which is proportional to the count and is adjusted to obtain the true count. For example, in embodiments where anchoring measurement is performed by reverse transcription usually only half of the RNA molecules are reversed transcribed in cDNA, therefore in those embodiments, the true count is twice the observed count through adjustments identifiable by a skilled person.

In some embodiments, in StochQuant methods and systems of the disclosure, the testing measurement is performed by: Sequencing methods such as amplicon sequencing (16S rRNA gene sequencing, ITS gene sequencing, 18S rRNA gene sequencing, COI gene sequencing, ITS2 gene sequencing, RBP1 gene sequencing, RBP2 gene sequencing, V(D) J region sequencing, mitochondrial gene sequencing, functional gene sequencing). Sequencing methods may generate cDNA from either template DNA or template RNA (following reverse-transcription). Further examples of sequencing methods comprise bulk RNA sequencing (RNA-seq), single cell RNA-seq, metagenomic sequencing, metatranscriptomic sequencing, spatial transcriptomics, Chromatin Immunoprecipitation Sequencing (ChIP-seq), exome sequencing, whole genome sequencing, target capture gene panels, small RNA sequencing (microRNA-seq), methyl DNA sequencing, single-cell DNA-Seq, or Mate-Pair Sequencing. Examples of sequencing can be performed with short read or long read sequencing technologies. Additional methods include single molecule protein counting assays such as digital immunoassays such as SIMOA (as described e.g., in ref. [8]), single molecule fluorescence in situ hybridization (smFISH), hybridization chain reaction (HCR) FISH, next generation sequencing (NGS) adapted for protein quantification.

In some embodiments, in StochQuant methods and systems of the disclosure, a reference molecule detected by the testing measurement is a single type of molecule (e.g., the mRNA of a reference gene). (see e.g. ref. www.genomics-online.com/resources/16/5049/housekeeping-genes/). Examples include mammalian housekeeping genes such as Glyceraldehyde-3-phosphate dehydrogenase (GAPDH), Phosphoglycerate kinase 1 (PGK1), Peptidylpropyl isomerase A (PPIA), ribosomal protein L13a (RPL13A), ribosomal protein large P0 (RPLP0), Beta-2-microglobulin (B2M), YWHAZ, SDHA, TFRC, GUSB, HMBS, HPRT1, TBP; bacterial housekeeping genes such as 16S, tus, rpoD, glyA, dnaB, gyrA, pykA/F, pfkA/B, mdoG, arcA; fungal housekeeping genes such as DUF221, ubcB, ADA, fis1, Cu-ATPase, psm1, spo7, spt3, DUF500, sac7, AP-2 beta, npl1, Beta-tubulin, Arabinofuranosidase-B2, Xylanase C (as described e.g., in refs . . . and [14]).

In some embodiments, StochQuant can be used to quantitatively detect a target molecule that is a nucleic acid, conjugated to a nucleic acid, or a nucleic acid target that is a proxy for another molecule type. Examples of how StochQuant methods and systems of the disclosure may be performed with a sequencing testing measurement are described herein:

In some embodiments of StochQuant detection methods and systems of the disclosure Multiple RNA expression reference molecules can be measured by a testing measurement such as bulk RNA-seq. A set of external RNA controls can be added to the sample. An example of a set of external RNA controls is the ThermoFisher Scientific ERCC RNA Spike-In Mix (ThermoFisher Scientific Cat. No. 4456740). A set of internal RNA reference molecules from the sample may be measured, a cell-type-specific reference molecule formed by multiple mRNA expression molecules. Examples of multiple DNA reference molecules. Multiple DNA expression reference molecules can be measured by a testing measurement such as shotgun metagenomic sequencing. A set of external DNA controls may be added to the sample. A set of internal DNA reference molecules from the sample may be measured: a fungal cell-type specific reference molecule formed by multiple DNA molecule types such as the ITS2 region and RPB2 gene; a bacterial cell-type specific reference molecule formed by multiple DNA molecule types such as the 16S gene and an antibiotic-resistance gene; a reference molecule formed by a reference DNA molecule and a reference RNA molecule (such as 16S DNA and 16S RNA).

In some embodiments of StochQuant detection methods and systems of the disclosure, a testing measurement can be performed by amplicon sequencing (16S rRNA gene sequencing, ITS gene sequencing, 18S rRNA gene sequencing, COI gene sequencing, ITS2 gene sequencing, RBP1 gene sequencing, RBP2 gene sequencing, V(D) J region sequencing, mitochondrial gene sequencing, functional gene sequencing). Other non-limiting amplicons that may be sequenced Sequencing methods can generate cDNA from either template DNA or template RNA (following reverse-transcription). Further examples of sequencing methods: bulk RNA sequencing (RNA-seq), single cell RNA-seq, metagenomic sequencing, metatranscriptomic sequencing, spatial transcriptomics, Chromatin Immunoprecipitation Sequencing (ChIP-seq) SIMOA, single molecule fluorescence in situ hybridization (smFISH), hybridization chain reaction (HCR) FISH, and next generation sequencing (NGS) adapted for protein quantification.

In some embodiments, additional experimental procedures, detection methods and approach for the related StochQuantization can be performed according to methods known or identifiable by a skilled person upon reading of the present disclosure.

In some embodiments, additional physical parameters can be used in the measurement representation in connection to manipulations or series of manipulations of the measurement workflow. Examples of physical parameters of a manipulation or series of manipulations can include: the efficiency and/or variability of a manipulation such as the capture or enrichment of molecule of interest (e.g., via capture probes), the yield of a nucleic acid via nucleic acid extraction/isolation, the efficiency of a reverse transcription manipulation, the efficiency of an amplification manipulation (e.g. PCR efficiency), the variability of an operator, of operators, or of instrumentation, the size and variability of fragments of a molecule yielded by fragmentation, the rate or efficiency of ligation of a molecule to another molecule, the rate, efficiency, or variability of physical and or chemical modifications to a molecule, the rate of degradation of a molecule, temperature that impacts the manipulation, time that impacts the manipulation, the number of times the manipulation is performed, and the duration for which a manipulation is performed.

In some embodiments, a sample or samples of an environment can be collected, and a sample or samples can be flash frozen, stored in a preservation buffer, or immediately processed. In some embodiments, the efficiency or variability of this step can be measured and incorporated into the quantitative detection of the target molecule described herein.

In some embodiments, target molecule nucleic acids can be isolated, extracted, and/or concentrated. In some embodiments, the efficiency or variability of this step can be measured and incorporated into the quantitative detection of the target molecule described herein.

In some embodiments, exogenous nucleic acids (commonly referred to as a “spike-in”) can be used as a reference molecule and may be added to a sample. In some embodiments, a spike-in or a plurality of spike-ins can be added into a sample at various stages of a workflow such as in an unprocessed sample, a preserved sample before nucleic acid extraction or isolation, a sample after nucleic acid extraction, a sample before library preparation, or a sample after library preparation.

In some embodiments, one or more absolute anchoring measurements of a reference molecule can be used as part of the segmentation calibration to measure the efficiency or variability of a manipulation or series of manipulations. Examples of a manipulation, manipulations, or series of manipulations that can be measured may include efficiency and variability of sample degradation over time, cell lysis, tagmentation, fixation, extraction, amplification (such as PCR) (see Example 30), reverse-transcription (see Example 38), ligation, and/or fragmentation. In some embodiments, the efficiency or variability of a manipulation, multiple manipulations, or combination of manipulations can be measured and incorporated into the quantitative detection of the target molecule described herein. In some embodiments, distributions of a reference molecule can be obtained and used, such as a distribution of fragment sizes. In some embodiments, fragment size or distribution of fragment size can be used to account for efficiency and yield of fragment binding to a sequencing flow-cell (as described, e.g., in ref. [15]. In some embodiments, the mechanism of fragmentation such as fragmentation with a Covaris sonicator, and/or the settings for which fragmentation occurs (such as a Duty cycle of 20%, Intensity of 55, Cycles per burst of 200, Time of 60 sec) can be used. In some embodiments, the efficiency and variability of a nucleic acid clean-up step can be incorporated. In some embodiments, the efficiency of A-tailing can be incorporated. In some embodiments, a step or combination of steps of the sequencing processes such as sequencing by synthesis (SBS) can be incorporated beyond Poisson sampling processes.

Library preparation of target nucleic acid molecules can also be performed. Examples of commercial library kits and methods are provided herein e.g. in the Examples section and other portions of the preset disclosure, as well as in Appendix B of U.S. Provisional Application No. 63/579,291 incorporated by reference in its entirety (see e.g. Methods Section, subsection: 16S rRNA gene Sequencing Library Preparation). In some embodiments, the efficiency or variability of this step may be measured and incorporated into the quantitative detection of the target nucleic acid molecule described herein. An example can include accounting for PCR efficiency, GC content, and/or amplicon length.

A testing measurement or testing measurements can be performed to detect the target nucleic acid molecule, such as with an Illumina MiSeq instrument or other instruments (appropriate sequencing measurements are described herein). Examples are provided herein e.g. in the Examples section and other portions of the preset disclosure, as well as in Appendix B of U.S. Provisional Application No. 63/579,291 incorporated by reference in its entirety, which describes exemplary using an Illumina MiSeq instrument to perform the testing measurement to detect 16S rRNA gene fragment target molecules. In some embodiments, the efficiency or variability of this step can be measured and incorporated into the quantitative detection of the target molecule described herein.

In some embodiments of StochQuant methods and systems herein described Data Processing Computations can be performed according to methods known or identifiable by a skilled person upon reading of the present disclosure.

In some embodiments, a basecaller (examples provided herein) can be used to determine the nucleic acid sequence of a barcoded nucleic acid fragment, wherein the target molecule is the nucleic acid sequence or the barcoded nucleic acid fragment.

In some embodiments, target nucleic acid molecule sequences can be stored in various file formats (see. e.g. Examples described herein).

In some embodiments, a sequence alignment tool, de novo assembly tool, post alignment processing tool, or combination of tools can be used to further process and/or filter the sequenced reads, indicative of the target nucleic acid molecule.

In some embodiments, a database (examples described herein) can be used for sequence alignment of the target nucleic acid molecule.

In some embodiments, other sequencing processing tools (examples described herein) can be used for further quality control filtering and processing of the molecular count of the target molecule via the testing measurement.

In some embodiments, other software tools to aid in the visualization, interpretation, and processing of sequences can be utilized as described e.g. the Examples section and other portions of the preset disclosure as well as in Appendix B U.S. Provisional Application No. 63/579,291 incorporated by reference in its entirety Methods Section, Subsection: 16S rRNA gene amplicon data processing).

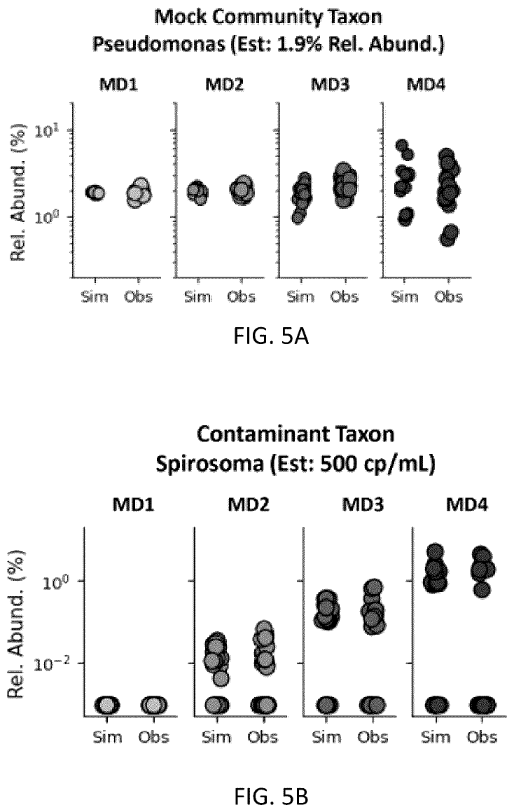

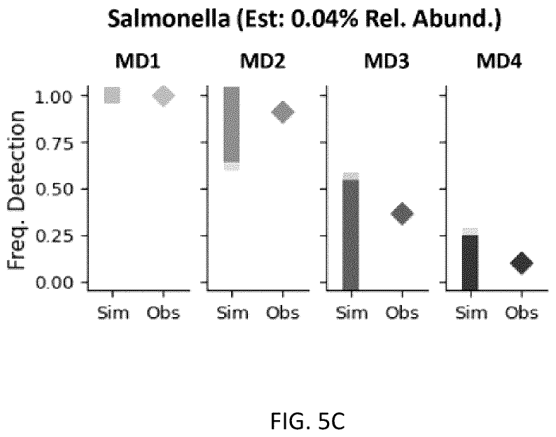

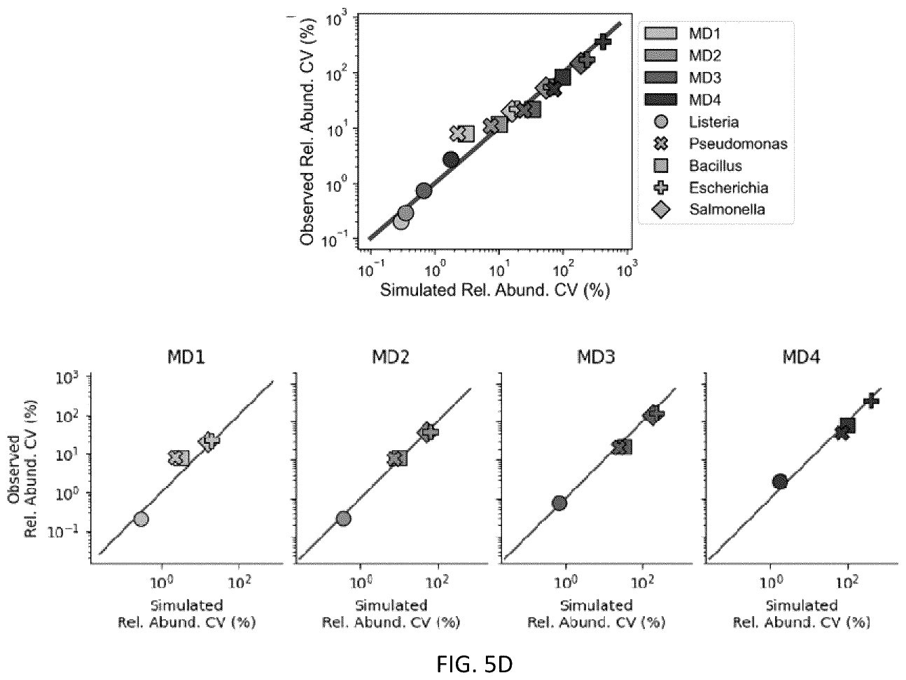

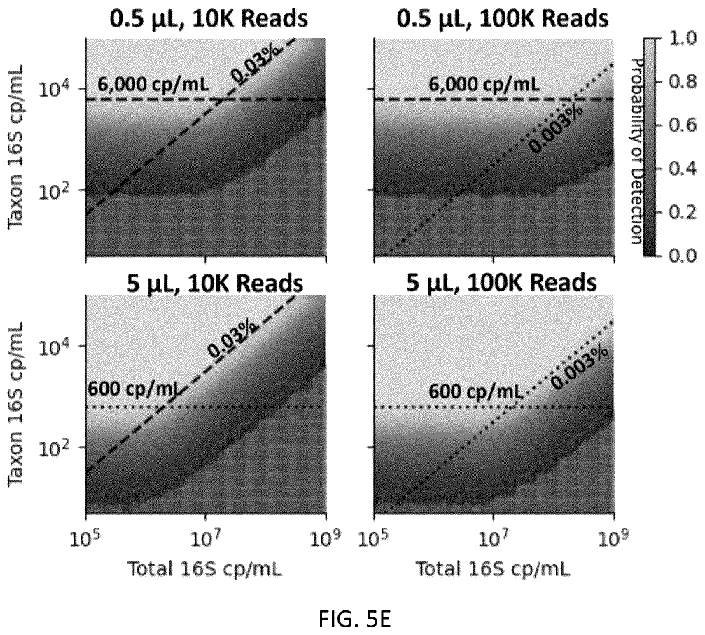

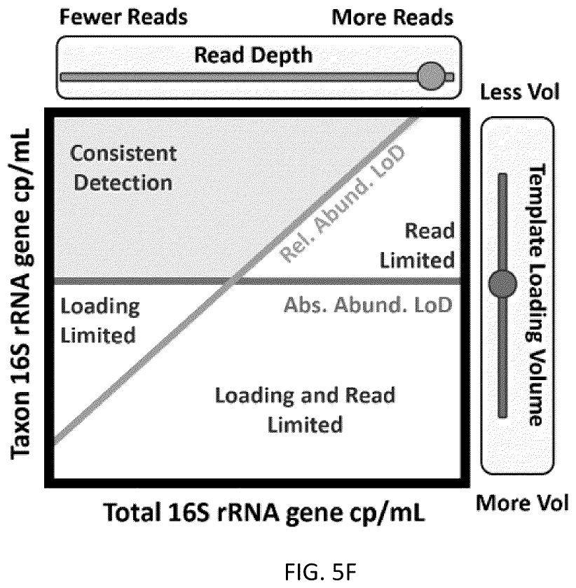

In some embodiments, differential abundance analysis software can be utilized on the molecular counts of the target nucleic acid molecule or on values obtained based upon the molecular counts of the target nucleic acid molecule. Examples are provided in the Examples section and other portions of the preset disclosure as well as in Appendix B U.S. Provisional Application No. 63/579,291 incorporated by reference in its entirety, (see FIGS. 1B, 5E-H, 6F-H, Supplementary FIG. 7, 8, 10 of Appendix B).

In some embodiments, an absolute anchoring measurement value of the reference molecule can be obtained from quantification algorithms or software. Examples can include using a commercial quantification software such as the BioRad QuantaSoft Software to obtain an absolute anchoring measurement of a nucleic acid reference molecule from a digital PCR measurement or performing directly performing a computation or computations directly on a digital PCR measurement as exemplified in the present discussion and throughout Appendix B of U.S. Provisional Application No. 63/579,291 incorporated by reference in its entirety and in Appendix B Methods Subsection Total bacterial load quantification with digital PCR of U.S. Provisional Application No. 63/579,291 incorporated by reference in its entirety. Examples of performing a computation directly can include computing the formula for calculating concentration of the nucleic acid reference molecule based on droplet counts from digital PCR (as described e.g., in the BioRad Droplet Digital PCR Applications Guide as published at the filing date of the present disclosure) through the use of functions in a computer programming language, implemented on a computer, the use of functions and operations within a spreadsheet software platform such as Microsoft Excel or Google Sheets, or a calculator.

In some embodiments, sequences or counts of sequences of the target nucleic acid molecule can be further processed and filtered according to methods known or identifiable by a skilled person upon reading of the present disclosure.

In some embodiments, a plurality of samples can be used to quantitatively detect a target molecule in an environment. An example can include measuring a reference molecule in an unprocessed sample, and a reference molecule in a processed sample, and using the differences in quantitative detection of the reference molecule to determine the efficiency of the processing step, to quantitatively detect a target molecule in an environment.

In some embodiments, replicate samples or replicate measurements of a sample can be used to improve quantitative detection of a target molecule in an environment. An example can include obtaining library-preparation replicates of a sample.

In some embodiments, such as an example described in Examples 5, 35, 37-39 as well as in Appendix B of U.S. Provisional Application No. 63/579,291 incorporated by reference in its entirety, a forward measurement model can be created and/or used for quantitative detection of a target molecule. An example of a forward measurement model is described in Examples 5, 35, 37-39 as well as in Appendix B of U.S. Provisional Application No. 63/579,291 incorporated by reference in its entirety (see Appendix B FIGS. 2, 3).Examples of using the forward measurement model for quantitative detection of a target molecule can be found throughout the present disclosure and throughout Appendix B of U.S. Provisional Application No. 63/579,291 incorporated by reference in its entirety for example in Appendix B FIGS. 2-6.

In some embodiments, a machine learning approach (as described herein) can be created and/or used for quantitative detection of a target molecule.

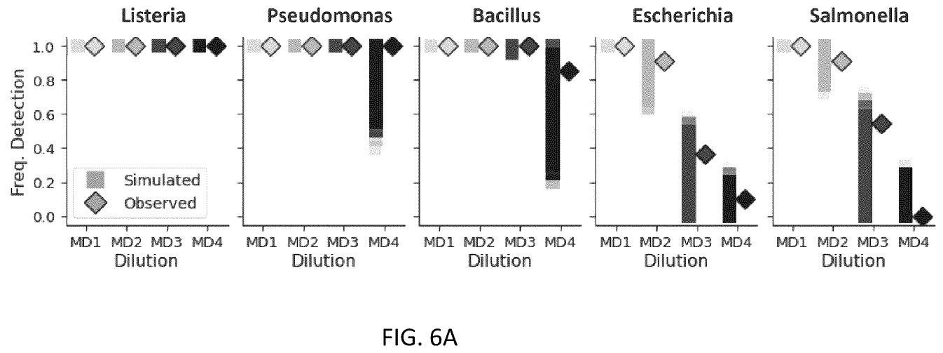

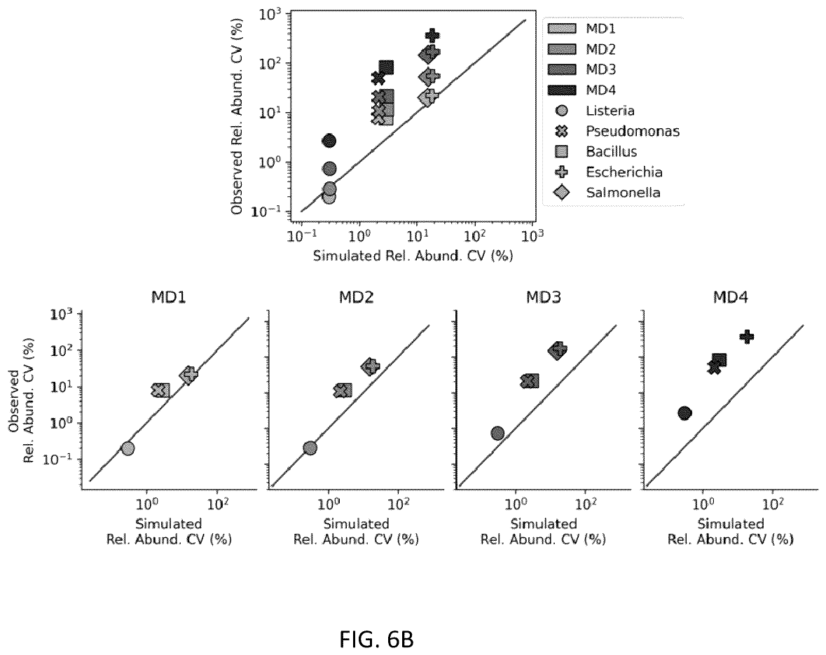

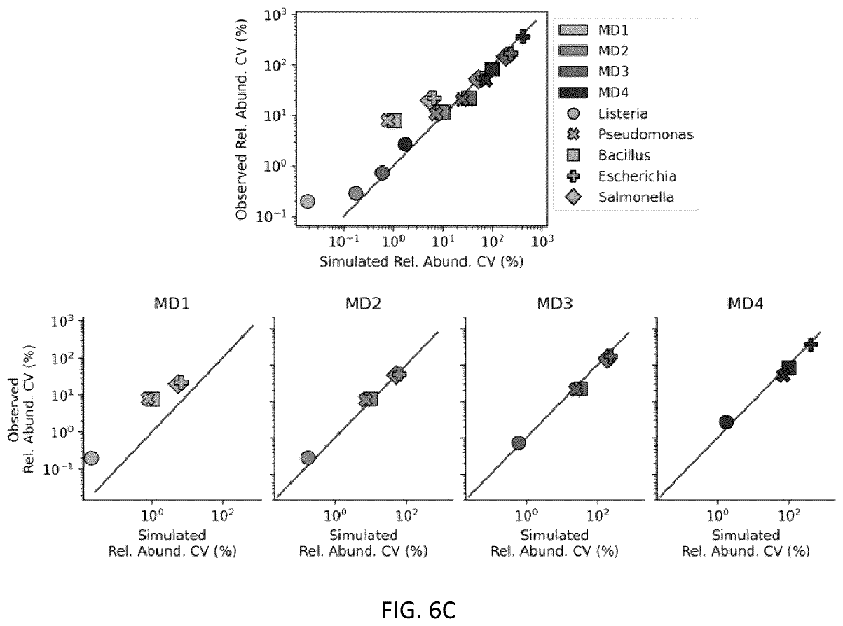

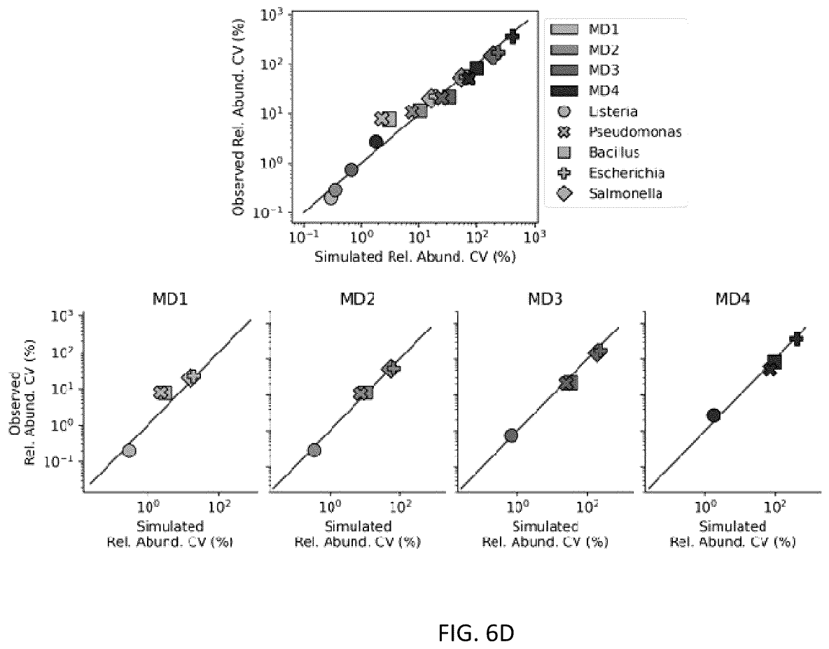

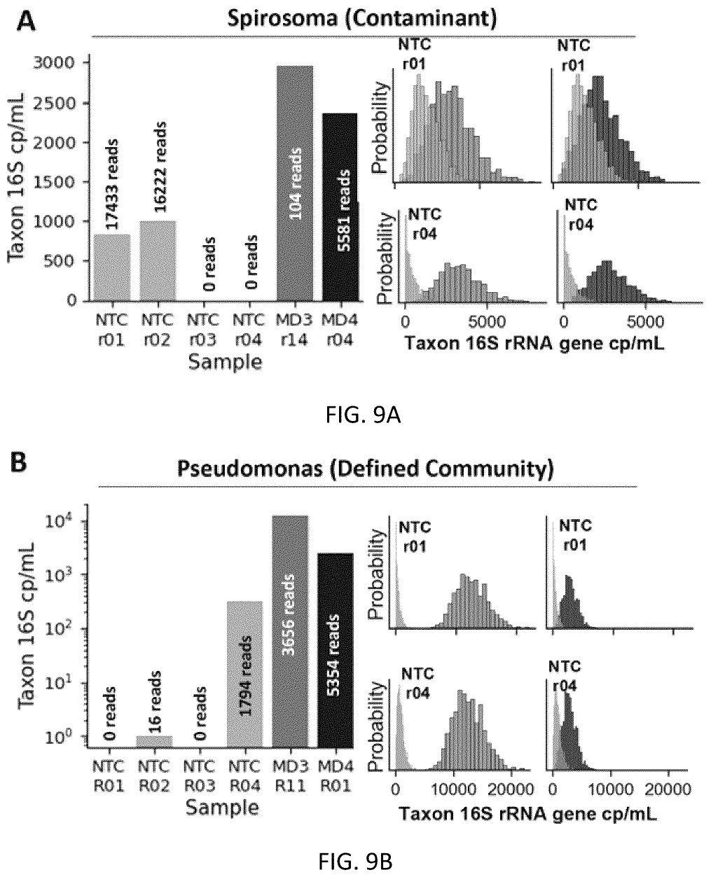

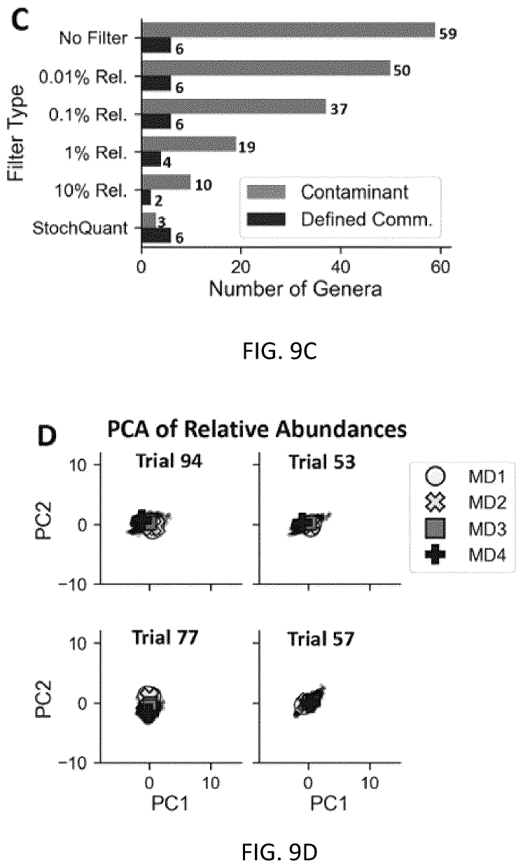

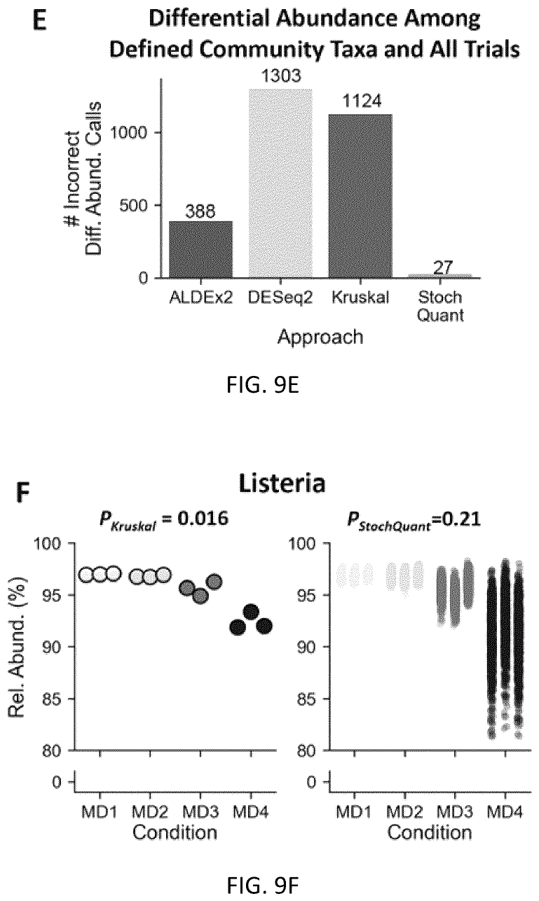

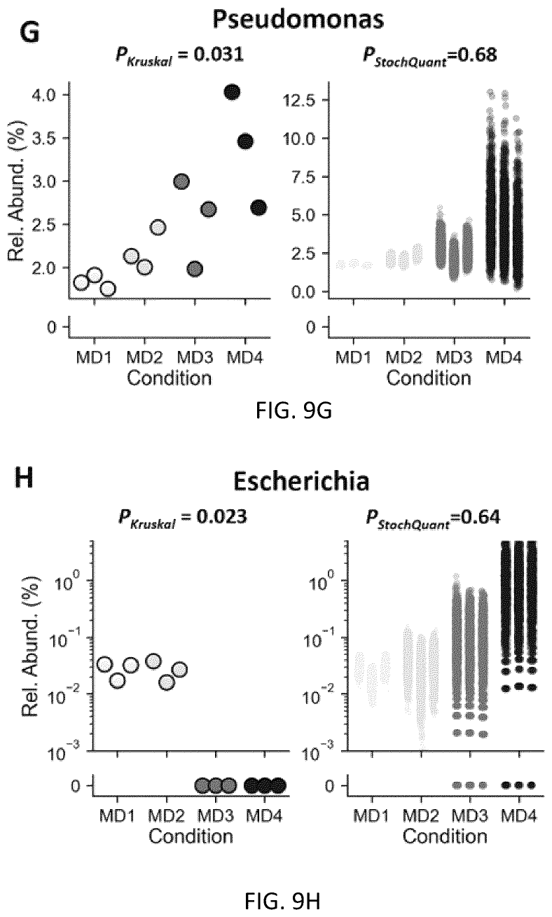

In some embodiments, quantitative detection of a target molecule can be used to further filter and process the sequencing data (as described in Example 13 as well as in Appendix B of U.S. Provisional Application No. 63/579,291 incorporated by reference in its entirety FIGS. 5, 6, Supplementary FIG. 9).

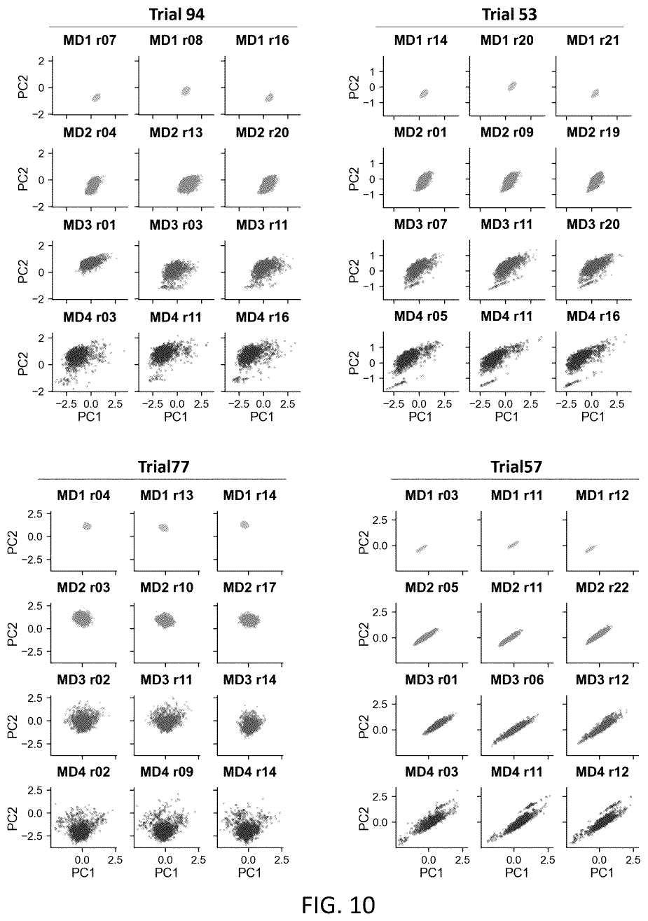

Examples can include filtering read counts that are estimated to be less that a single target molecule with a certain level of confidence, filtering measurements with quantitative detection below a given threshold (such as a target abundance that can be reproducibly detected with confidence with at least 99% probability), or filtering measurements with quantitative detection below the quantitative detection value obtained from a measurement in a processing blank or control measurement, (see examples described in Example 13 as well as in Appendix B of U.S. Provisional Application No. 63/579,291 incorporated by reference in its entirety (see FIGS. 5, 6, Supplementary FIGS. 9, 10 of the Appendix B).

In some embodiments the quantitative detection of a target molecule can be transformed or scaled (as described in the exemplary applications reported in Example 36 as well as in Appendix B of U.S. Provisional Application No. 63/579,291 incorporated by reference in its entirety in the context of relative abundances, absolute abundance, log 10 transformed absolute or relative abundances, center-log transformed (CLR) relative abundances, and pseudo-log transformed relative and absolute abundances).

Examples can include: transforming the number of target molecules in an environment to concentration of target molecules in an environment. In some embodiments, the transformation of number of target molecules in an environment to concentration of target molecules in an environment can occur by dividing the number of molecules by a quantitative amount. In some embodiments, the quantitative amount is instead a probability distribution of a quantitative amount. If the quantitative amount is provided as a probability distribution of a quantitative amount, the transformation can occur by iteratively sampling from the probability distribution of target molecules and iteratively sampling from the probability distribution of quantitative amounts, and dividing a computationally sampled target molecule by a computationally sampled quantitative amount; transforming the number of target molecules in an environment to a relative abundance of target molecules in an environment. In some embodiments, the relative abundance of a target molecule can be in relation to the total number of molecules of interest, such as a target 16S molecule relative to total 16S molecules. In some embodiments, a relative abundance can be a target molecule relative to another target molecule, such as the ratio between two markers such as the bacterial genes porB and rpmB; In some embodiments a target abundance can be further scaled, transformed, or normalized via a log transformation, MinMaxScaling, the addition of a pseudocount, or other linear transformations.

In embodiments of StochQuant methods and systems analysis computations can be performed according to methods known or identifiable by a skilled person upon reading of the present disclosure.

In some embodiments, probability distributions can be provided to perform an analysis task. Examples of analysis tasks can include:
performing differential abundance analysis with differential abundance tools and software as exemplified herein throughout the disclosure and in Appendix B of U.S. Provisional Application No. 63/579,291 incorporated by reference in its entirety (see FIGS. 1, 5, 6, Supplementary FIGS. 7, 8, 10 of the Appendix B);
performing dimensionality reduction as exemplified herein throughout the disclosure and in Appendix B of U.S. Provisional Application No. 63/579,291 incorporated by reference in its entirety (see FIGS. 1, 5, 6, Supplementary FIG. 9 of the Appendix B); and
performing other types of analyses that involve dynamic programming, artificial neural networks, hidden Markov models, support vector machine, clustering, Bayesian networks, regression analysis, sequence mining, alignment-free sequence analysis, Fourier transforms, least-squares spectral analysis, alignment visualization (described herein), phylogenetic tree visualization software (described herein), protein structure prediction (described herein), RNA structure prediction software (described herein).

In embodiments of StochQuant detection methods and systems decision-making based on quantitative detection. can be performed according to methods known or identifiable by a skilled person upon reading of the present disclosure.

In some embodiments, a decision or course of action can occur as a result of data filtering, data processing, or data analysis from quantitative detection of a target molecule or plurality of target molecules (described herein). Examples can include selection of a therapy, identification of a compound, diagnosis of a disease, determination for additional tests, observations, or diagnostic tools, re-collection, re-processing, or re-measurement of a sample, decision that a result is otherwise invalid or indeterminant. Examples can include selection of a cancer treatment based upon the quantitative detection, diagnosis of a genetic disease based on a pathogenic variant in the CFTR gene, detection of a genetic disease during prenatal screening, quantitative detection of a specific microbe or group of microbes in a sample of a vaginal microbiome environment to diagnose a disease such as aerobic vaginitis, bacterial vaginosis, cytolytic vaginosis, recurrent UTI, or yeast infection, quantitative detection of a biomarker or plurality of biomarkers for the diagnosis of sepsis which can impact course of treatment, or for quantitative detection of a microbe or plurality of microbes for the diagnosis of a microbial related disease, decision of a personalized treatment, decision of a general treatment, or development of a therapeutic based on quantitative detection of a microbe or microbes, or quantitative detection of another biomarker (described in further detail elsewhere in the document).

In some embodiments, a decision or course of action can occur as a result of the confidence of the quantitative detection, or confidence of an analysis based upon the quantitative detection of a target molecule or plurality of target molecules. The confidence of quantitative detection can be used as an additional piece of metric to reach a decision or decide on a course of action, such that a minimum threshold of confidence is needed to make a decision.

In some embodiments, in StochQuant methods and systems of the disclosure, a reference molecule can be formed by a plurality of molecule types that are simultaneously detected during the testing measurement to provide a same count (like multiple 16S genes which all amplify from the same primer). Examples of a reference molecule formed by a plurality of molecule types that are simultaneously detected during the testing measurement are described herein: Examples of multiple genes, portions of genes, regions, or portions of regions which all amplify from the same primer such as ITS, ITS2, 18S, COI, ITS2, V(D) J region. Examples of other types of multiple molecules all which give rise to a fluorescent signal, provided the same probe or fluorophore: Lipopolysaccharides (LPS), Peptidoglycan, Teichoic acids, specific DNA or RNA targets.

In some embodiments, in StochQuant methods and systems of the disclosure, a reference molecule can be formed by a plurality of molecule types each separately detected during the testing measurement to provide separate unique counts, as understood by a skilled person. Examples of multiple RNA expression reference molecules comprise Multiple RNA expression reference molecules can be measured by a testing measurement such as bulk RNA-seq. A set of external RNA controls can be added to the sample. An example of a set of external RNA controls is the ThermoFisher Scientific ERCC RNA Spike-In Mix (ThermoFisher Scientific Cat. No. 4456740). A set of internal RNA reference molecules from the sample can be measured, a cell-type-specific reference molecule formed by multiple mRNA expression molecules. Examples of multiple DNA reference molecules comprise multiple DNA expression reference molecules measured by a testing measurement such as shotgun metagenomic sequencing in which a set of external DNA controls can be added to the sample. A set of internal DNA reference molecules from the sample can be measured: a fungal cell-type specific reference molecule formed by multiple DNA molecule types such as the ITS2 region and RPB2 gene; a bacterial cell-type specific reference molecule formed by multiple DNA molecule types such as the 16S gene and an antibiotic-resistance gene; a reference molecule formed by a reference DNA molecule and a reference RNA molecule (such as 16S DNA and 16S RNA).

In some embodiments, in StochQuant methods and systems of the disclosure, obtaining a molecular count of the target molecule and obtaining a molecular count of the reference molecule are performed in a same sample or in subsamples of a same sample.

An example of how StochQuant methods and systems of the disclosure can be performed comprise shotgun metagenomic sequencing: In some embodiments, a forward measurement model may take as inputs the number of target barcoded nucleic acid fragments (target molecule) (within the size range of the sequencing technology being used), total number of barcoded fragments within the size range of the sequencing technology being used (reference molecule). The value of total number of barcoded fragments may be obtained via an absolute anchoring measurement, total number of sequenced reads (molecular count of the reference molecule). This value may be obtained from the testing measurement, a quantitively measured amount (e.g. volume) of the sample. A measurement workflow representation (referred to elsewhere as a forward measurement model) can produce a molecular count or multiple probable molecular counts of the target molecule from the testing measurement. An inference step may take the input physical parameters (total number of barcoded fragments, total number of sequenced reads, a quantitatively measured amount, and a molecular count of the testing measurement) to provide a probability distribution of a target molecule. In some embodiments, a probability distribution of fragment abundance, gene abundance, or taxon abundance is provided. In some embodiments, additional parameters, such as efficiency and variability of sample degradation over time, cell lysis, extraction, amplification (such as PCR), ligation, or fragmentation may be incorporated into the forward measurement model or into the inference procedure. In some embodiments, a distribution or distributions of coverage along a target is used to provide a probability distribution of target abundance.

A further example of how StochQuant methods and systems of the disclosure can be performed comprise StochQuant for bulk RNA-seq. In particular, in some embodiments, StochQuant may be used with a bulk RNA-seq testing measurement, following the general principles as described herein (see e.g. Example 37). In some embodiments, StochQuant for bulk RNA-seq may involve the quantitative detection of a fragment, gene, cell, or category of cells. In some embodiments, StochQuant for bulk-RNA-seq may also incorporate additional physical parameters to account for reverse-transcription efficiency.

An example of how StochQuant methods and systems of the disclosure can be performed comprise StochQuant for single-cell RNA-seq. In particular, in some embodiments, in StochQuant methods and systems of the disclosure, StochQuant may be used with a single-cell RNA-seq testing measurement. The single-cell RNA-seq testing measurement may be performed following a workflow (such as a workflow described in DOI: 10.1186/s13073-017-0467-4). In some embodiments, StochQuant for bulk RNA-seq may involve the quantitative detection of a fragment, gene, cell, or category of cells. In some embodiments, StochQuant for single-cell RNA-seq may also incorporate parameters to account for efficiency and variability of steps in a workflow (see e.g. Examples 38, 39) Examples include cell sorting or collection, lysis, mRNA capture, reverse transcription, amplification, pooling, or barcode hoping.

Examples of anchoring measurements in connection with various testing measurements comprise an absolute anchoring measurement of a nucleic acid target can be obtained. Examples of absolute anchoring measurement can include digital PCR, other digital technologies based on isothermal amplification techniques such as rolling circle amplification (RCA), nucleic-acid sequence-based amplification (NASBA), loop-mediated amplification (LAMP), helicase-dependent amplification (HAD), recombinase polymerase amplification (RPA), strand-displacement amplification (SDA), multiple displacement amplification (MDA), and exponential amplification reaction (EXPAR), or other digital isothermal chemistries (e.g., as described in ref. [16]) or other isothermal amplification techniques (e.g., as described in Zhao, Y., et al., Isothermal Amplification of Nucleic Acids. Chem Rev, 2015. 115 (22): p. 12491-545) for digital or absolute quantification. Other examples also include digital immunoassays such as SIMOA, single molecule fluorescence in situ hybridization (smFISH), hybridization chain reaction (HCR) FISH, flow-cytometry, optical density, plating, real-time PCR.

In some embodiments of the StochQuant methods and systems of the disclosure, the reference molecule is a nucleic acid and the anchoring value is obtained by adding a known quantity of reference molecule to a sample.

In some embodiments, in StochQuant methods and systems of the disclosure, a probability distribution can be provided in non-parametric form as one or more target molecule abundances, each with a probability of being the true target molecule abundance. A non-limiting simple example of three probable target abundances such as [(value1=130 target molecules, probabilityl =0.2), (value2=133 target molecules, probability2=0.6), (value3=139 target molecules, probability3=0.2)]. In this example, the probabilities of the target abundances do not need to follow a known discrete probability distribution, such as the Poisson distribution.

In some embodiments, in StochQuant methods and systems of the disclosure, the probability distribution can be provided in the form of shape parameters for a known discrete probability distribution or parameters that can be used to determine the shape parameters for a known discrete probability distribution. An example is containing the information of the probability distribution in the form of the rate parameters n and p of a Negative binomial distribution (see Example 36). In some embodiments, the expected value (mean) target molecule abundance may be 100 molecules with an uncertainty (variance) of 200, and the probability distribution of target abundance may follow a negative binomial distribution. In this example, the probability distribution can be provided by the shape parameters n=100 and p=0.5.

In some embodiments, in StochQuant methods and systems of the disclosure, the probability distribution can be provided in the form of a list of target molecule abundances where the representation of each target molecule abundance (e.g., how many times the target molecule abundance “2” occurs) is correlated with its probability. In this example, if target molecule abundance 2 is the most likely, it will appear more times than any other target molecule abundance. In an exemplary embodiments, instead of describing the probability distribution of three probable target abundances such as [(value1=132 target molecules, probabilityl=0.2), (value2=133 target molecules, probability2=0.6), (value3=134 target molecules, probability3=0.2)], the probability distribution can be provided in the form of a list of target abundances such as [132, 132, 133, 133, 133, 133, 133, 133, 134, 134], where the representation of each target abundance is representative of the probability of the target abundance. (See Example 2).

In some embodiments, in StochQuant methods and systems of the disclosure, a probability distribution is provided by a machine learning approach (See Example 48). A machine learning approach may improve the computational efficiency of StochQuant by using StochQuant inputs and outputs to train a machine learning approach to predict the StochQuant outputs, thereby replacing any computationally inefficient steps involved in providing a probability distribution. A machine learning approach can be trained to take StochQuant input parameters (including but not limited to an absolute anchoring measurement, reference molecular count, target molecular count, and quantitatively measured amount(s)), to predict a probability distribution. An example is described herein: a simulated dataset can be created by randomly sampling from a parameter-space to create combinations of a target molecular count, reference molecular count, absolute anchoring measurement, and quantitatively measured amount(s). In this example, a target molecular count may vary from zero to the total molecular counts in the testing measurement. The total number of molecular counts in the testing measurement may vary among experimentally observed values. In the case of 16S amplicon sequencing, this value may range from 1,000 total read counts to 200,000 total read counts. For other applications, such as shotgun metagenomic sequencing, this value may range to hundreds of millions of total reads. In the case of 16S amplicon sequencing, if the total number of 16S molecules measured by digital PCR is the absolute anchoring measurement, this value may range from 1 copy to 1011 copies. In some embodiments, the absolute anchoring measurement may be expressed as a concentration (copies per quantitatively measurable amount). For each set of input parameters, probability distributions can be generated by an embodiment of StochQuant. In some embodiments, these probability distributions can be provided by negative binomial shape parameters. A machine learning approach, such as a neural network, can be trained on the input parameters to predict the negative binomial shape parameters, where the training data is the simulated dataset that spans the parameter-space of values for which inference will be performed.

In some embodiments, in StochQuant methods and systems of the disclosure detection of a target molecule is performed in connection with detection of abundance of a microorganism and/or related taxa (See Example 2).

The term “microbial” “microbe” or “microorganism”, as used herein indicates a microscopic organism selected from viruses and living organisms which can exist in a single-celled form or in a colony of cells form. Accordingly, microorganisms in the sense of the disclosure, viruses and an extremely diverse unicellular organisms, including prokaryotes and in particular bacteria, but also including fungi (yeast and molds), and protozoal parasites as understood by a skilled person.

The term “virus” and “viruses” as used herein indicates a submicroscopic microbe capable of replicating only inside the living cells of an organism. A complete virus particle, known as a virion, consists of nucleic acid surrounded by a protective coat of protein called a capsid. These are formed from protein subunits called capsomeres. Viruses can have a lipid “envelope”derived from the host cell membrane. Viruses can have a lipid “envelope” derived from the host cell membrane. The capsid is made from proteins encoded by the viral genome and its shape serves as the basis for morphological distinction.

Exemplary non-enveloped viruses comprise DNA viruses such as Adenoviruses, Parvoviruses Polyomaviruses and Anelloviruse and RNA viruses such as Caliciviruses, Picornaviruses, Reoviruses, Astroviruses, Hepeviridae and additional viruses identifiable by a skilled person. Viruses in the sense of the disclosure also comprise enveloped viruses which further include the membrane bilayer of the envelope possibly presenting one or more proteins. Exemplary enveloped viruses comprise DNA viruses such as Herpesviruses, Poxviruses, Hepadnaviruses, Asfarviridae and RNA viruses such as Flaviviruses Alphaviruses, Togaviruses Coronaviruses, Hepatitis D, Orthomyxoviruses, Paramyxoviruses, Rhabdovirus · Bunyaviruses, Filoviruses as well as Retroviruses and additional viruses identifiable by a skilled person. [20].

Viruses in the sense of the disclosure can also be categorized in view of the related viral NA according to the Baltimore classification as double-stranded viruses (dsDNA viruses), single-stranded DNA viruses (ssDNA), double-stranded RNA viruses (dsRNA viruses), positive-strand

RNA viruses (+ssRNA viruses), negative-strand RNA viruses (-ssRNA viruses), single-stranded RNA-reverse transcriptase viruses (ssRNA-RT viruses), and double-stranded DNA-reverse-transcriptase viruses (dsDNA-RT viruses).

The term “prokaryote” is used herein interchangeably with the terms “prokaryotic cell”and refers to a microbial species which contains no nucleus or other membrane-bound organelles in the cell. Exemplary prokaryotic cells include bacteria and archaea.

The term “bacteria” or “bacterial cell”, as used herein indicates a large domain of prokaryotic microorganisms. Typically, a few micrometers in length (from 0.5 to 6 um), bacterial cell can have a diameter from 1 to 10 μm or be as large as 750 um as understood by a skilled person. Bacteria have a number of shapes, ranging from spheres to rods and spirals, and are present in most habitats on Earth, such as terrestrial habitats like deserts, tundra, Arctic and Antarctic deserts, forests, savannah, chaparral, shrublands, grasslands, mountains, plains, caves, islands, and the soil, detritus, and sediments present in said terrestrial habitats; freshwater habitats such as streams, springs, rivers, lakes, ponds, ephemeral pools, marshes, salt marshes, bogs, peat bogs, underground rivers and lakes, geothermal hot springs, sub-glacial lakes, and wetlands; marine habitats such as ocean water, marine detritus and sediments, flotsam and insoluble particles, geothermal vents and reefs; man-made habitats such as sites of human habitation, human dwellings, man-made buildings and parts of human-made structures, plumbing systems, sewage systems, water towers, cooling towers, cooling systems, air-conditioning systems, water systems, farms, agricultural fields, ranchlands, livestock feedlots, hospitals, outpatient clinics, health-care facilities, operating rooms, hospital equipment, long-term care facilities, nursing homes, hospice care, clinical laboratories, research laboratories, waste, landfills, radioactive waste; and the deep portions of Earth's crust, as well as in symbiotic and parasitic relationships with plants, animals, fungi, algae, humans, livestock, and other macroscopic life forms. Bacteria in the sense of the disclosure refers to several prokaryotic microbial species which comprise Gram-negative bacteria, Gram-positive bacteria, Proteobacteria, Cyanobacteria, Spirochetes and related species, Planctomyces, Bacteroides, Flavobacteria, Chlamydia, Green sulfur bacteria, Green non-sulfur bacteria including anaerobic phototrophs, Radioresistant micrococci and related species, Thermotoga and Thermosipho thermophiles as would be understood by a skilled person. Taxonomic names of bacteria that have been accepted as valid by the International Committee of Systematic Bacteriology are published in the “Approved Lists of Bacterial Names” as well as in issues of the International Journal of Systematic and Evolutionary Microbiology. More specifically, the wording “Gram positive bacteria” refers to cocci, nonsporulating rods and sporulating rods that stain positive on Gram stain, such as, for example, Actinomyces, Bacillus, Clostridium, Corynebacterium, Cutibacterium (previously Propionibacterium), Erysipelothrix, Lactobacillus, Listeria, Mycobacterium, Nocardia, Staphylococcus, Streptococcus, Enterococcus, Peptostreptococcus, and Streptomyces. Bacteria in the sense of the disclosure refers also to the species within the genera Clostridium, Sarcina, Lachnospira, Peptostreptococcus, Peptoniphilus, Helcococcus, Eubacterium, Peptococcus, Acidaminococcus, Veillonella, Mycoplasma, Ureaplasma, Erysipelothrix, Holdemania, Bacillus, Amphibacillus, Exiguobacterium, Gracilibacillus, Halobacillus, Saccharococcus, Salibacillus, Virgibacillus, Planococcus, Kurthia, Caryophanon, Listeria, Brochothrix, Staphylococcus, Gemella, Macrococcus, Salinococcus, Sporolactobacillus, Marinococcus, Paenibacillus, Aneurinibacillus, Brevibacillus, Alicyclobacillus, Lactobacillus, Pediococus, Aerococcus, Abiotrophia, Dolosicoccus, Eremococcus, Facklamia, Globicatella, Ignavigranum, Carnobacterium, Alloiococcus, Dolosigranulum, Enterococcus, Melissococcus, Tetragenococcus, Vagococcus, Leuconostoc, Oenococcus, Weissella, Streptococcus, Lactococcus, Actinomyces, Arachnia, Actinobaculum, Arcanobacterium, Mobiluncus, Micrococcus, Arthrobacter, Kocuria, Nesterenkonia, Rothia, Stomatococcus, Brevibacterium, Cellulomonas, Oerskovia, Dermabacter, Brachybacterium, Dermatophilus, Dermacoccus, Kytococcus, Sanguibacter, Jonesia, Microbacteirum, Agrococcus, Agromyces, Aureobacterium, Cryobacterium, Corynebacterium, Dietzia, Gordonia, Skermania, Mycobacterium, Nocardia, Rhodococcus, Tsukamurella, Micromonospora, Propioniferax, Nocardioides, Streptomyces, Nocardiopsis, Thermomonospora, Actinomadura, Bifidobacterium, Gardnerella, Turicella, Chlamydia, Chlamydophila, Borrelia, Treponema, Serpulina, Leptospira, Bacteroides, Porphyromonas, Prevotella, Flavobacterium, Elizabethkingia, Bergeyella, Capnocytophaga, Chryseobacterium, Weeksella, Myroides, Tannerella, Sphingobacterium, Flexibacter, Fusobacterium, Streptobacillus, Wolbachia, Bradyrhizobium, Tropheryma, Megasphera, Anaeroglobus, Escherichia-Shigella, Klebsiella, muribaculum, alloprevotella, paraprevotella, oscillibacter, candidatus arthromitus, aeromonas, romboutsia, campylobacter, salmonella, faecalibacterium, roseburia, blautia, oribacterium, ruminococcus.

The term “Archaea” or “Archaea cell” as used herein refers to prokaryotic microbial species of the division Mendosicutes, such as Crenarchaeota and Euryarchaeota, which comprises methanogens (prokaryotes that produce methane); extreme halophiles (prokaryotes that live at very high concentrations of salt (NaCl); extreme (hyper) thermophiles (prokaryotes that live in extremely hot environments), Methanobrevibacter, and methanosphaera. Archaea are single-celled organisms that lack a nucleus (prokaryotes), may have morphology including but not limited to coccus, bacillus, square, and triangular. Archaea lack a peptidoglycan cell wall and Md range from 0.1 μm to 100 μm. Archaea in the disclosure refer to archaea within the genera: Halostagnicola (pleiomorphic, 1.0-3.0 μm length, non-motile), Caldisphaera (coccus, 0.8-1.1 μm diameter, non-motile), Cenarchaeum (rod-shaped, 0.5-0.9 μm diameter), Caldococcus (coccus, 0.7-2.1 μm size), Ignisphaera (coccus, 1-1.5 μm diameter), Acidilobus (coccus, 1-2 μm diameter, non-motile), Acidococcus, Aeropyrum (coccus, 0.8-1.2 μm diameter), Desulfurococcus (coccus, 0.5-15 μm diameter), Ignicoccus (coccus, 1-3 μm diameter, motile), Staphylothermus (coccus, 0.8-1.3 μm diameter), Stetteria (coccus, 0.5-1.5 μm diameter), Sulfophobococcus (coccus), Thermodiscus (coccus, 0.2-3 μm diameter), Thermosphaera (coccus, 0.5-1.5 μm diameter), Geogemma (coccus, ˜1 μm diameter), Hyperthermus (coccus, ˜1.5 μm diameter), Pyrodictium (coccus, 0.3-2.5 μm diameter), Pyrolobus (coccus, 0.7-2.5 μm diameter), Nitrosopumilus (candidatus) (rod-shaped, 0.15-0.27 μm diameter and 0.49-2.00 μm length, some motile), Acidianus (spindle-shaped, 900 × 24 nm), Metallosphaera (coccus, ˜1 μm diameter), Stygiolobus (cocci, 0.5-2 μm diameter, carries Stygiolobus rod-shaped virus), Sulfolobus (cocci, 0.5-2 μm diameter, carries virus), Sulfurisphaera (cocci, 1.2-1.5 μm diameter), Thermofilum (rod-shaped, 0.17-0.35 μm diameter and 4-100 μm length), Caldivirga (rod-shaped, 0.4-0.7 μm diameter and 4-100 μm length), Pyrobaculum (rod-shaped, 0.4-0.5 μm diameter and 4-100 μm length), Thermocladium (rod-shaped, 4-100 μm length), Thermoproteus (rod-shaped, 0.4-0.5 μm diameter and 4-100 μm length), Vulcanisaeta (rod-shaped, 0.4-0.6 μm diameter and 4-100 μm length), Aciduliprofundum (pleiomorphic coccus, 0.6-1.0 μm diameter), Archaeoglobus (triangular, 0.4-1.2 μm wide), Ferroglobus (coccoid), Geoglobus (coccoid), Haladaptatus (coccus, 1.0-1.2 μm diameter, motile), Halalkalicoccus (pleiomorphic, ˜5 μm), Haloalcalophilium (pleiomorphic, ˜5 μm), Haloarcula (pleiomorphic, 1.0-2 μm diameter 2.0-3.0 μm length), Halobacterium (rod-shaped, 2-5 μm length), Halobaculum (rod-shaped, 0.4 μm diameter and 0.6 μm length), Halobiforma (pleomorphic, 0.5-2 μm diameter), Halococcus (cocci, 0.6-1.5 μm diameter), Haloferax (pleiomorphic, 1.1-2.0 μm), Halogeometricum (pleomorphic), Halomicrobium (rod-shaped, 1.80-2.25 μm diameter and 2.25-2.80 μm length, non-motile), Halopiger (rod-shaped, ˜3.75 μm diameter and ˜0.75 μm length), Haloplanus (rod-shaped, ˜1.5 μm length), Haloquadra (square, 40×40 μm), Halorhabdus (pleiomorphic, 3-5 μm), Halorubrum (pleiomorphic, 44×55 nm), Halosarcina (pleiomorphic, 0.8-2 μm diameter), Halosimplex, Haloterrigena (coccoid, 1.5 μm-2.0 μm diameter), Halovivax (rod-shaped, 0.4-0.5 μm diameter and 4-5 μm length), Natrialba, Natrinema (pleomorphic, 0.5-2.0×1.5-11.0 μm), Natronobacterium (rod-shaped), Natronococcus (coccoid, 1-2 μm diameter), Natronolimnobius (rod-shaped), Natronomonas (pleomorphic), Natronorubrum (pleomorphic, 0.8-3.6 μm), Methanoregula (candidatus) (rod-shaped, 0.2-0.8 μm in diameter or coccoid, 0.2-0.3 μm diameter and 0.8-3.0 μm length), Methanocalculus (coccoid, ˜1 μm diameter), Methanobacterium) (rod-shaped, 2.5-5 μm in diameter), Methanobrevibacter (rod-shaped, 0.34 to 1.6 μm), Methanosphaera (coccoid), Methanothermobacter (rod-shaped, 7 μm length), Methanothermus (rod-shaped, 2-5 μm length), Methanocaldococcus (coccoid, 0.1-100 μm length), Methanotorris (coccoid, 0.1-100 μm length), Methanococcus (coccoid, 0.9-1.3 μm diameter), Methanothermococcus (coccoid), Methanocorpusculum (cocci, <2 μm diameter), Methanoculleus (cocci, 0.5 to 2.0 μm diameter), Methanofollis (cocci, 0.8-1.8 μm diameter), Methanogenium (cocci, 1.2-2.5 μm diameter), Methanolacinia (rod-shaped, 0.6 μm diameter and 1.5-2.5 μm length), Methanomicrobium (rod-shaped, 0.6-0.7 diameter 1.5-2.5 length), Methanoplanus (cocci,1-3.5 μm diameter), Methanospirillum (rod-shaped,2-5 μm length), Methanosaeta (rod-shaped,2.5-6 μm length), Methanimicrococcus (cocci, 0.8 μm diameter), Methnococcoides (cocci, 0-1.8 μm diameter), Methanohalobium (cocci, 1.0-1.2 μm), Methanohalophilus (rod-shaped), Methanolobus (cocci, 1.0-1.25 μm diameter), Methanomethylovorans (cocci), Methanosalsum (rod-shaped), Methanosarcina (rod-shaped,2.3=0.2 μm), Methanopyrus (rod-shaped, 2-14 μm length and 0.5 μm diameter), Palaeococcus, Pyrococcus (cocci,0.8-2 μm diameter), Thermococcus (cocci,0.6-2 μm diameter), Ferroplasma (pleomorphic or cocci, 0.66±0.18×0.57±0.20 μm), Picrophilus (pleomorphic), Thermoplasma (cocci, ˜1 μm diameter), and Nanoarchaeum (cocci, 0.4 μm diameter).

The term “fungi” or “fungal cells” as described herein, indicates eukaryotes such as yeasts and molds that exist in single unicellular forms (yeast) or multicellular forms (molds such as hyphae and mycelium) which are characterized by a cell wall that contains of glucans, glycoproteins, and chitin. By weight, fungal cell walls typically contain up to 60% glycans, up to 30% glycoproteins, and up to 20% chitin. Fungi can typically range from about 0.5 to 50 μm and in particular 0.5 to 20 μm 5-50 μm in size. Fungi in the disclosure refer to fungi within the genera: Aaosphaeria, Acaromyces, Agaricus, Alternaria, Amorphotheca, Annulohypoxylon, Antrodia, Apiotrichum, Aplosporella, Arthroderma, Ascochyta, Ascoidea, Aspergillus, Aureobasidium, Babjeviella, Bacidia, Batrachochytrium, Baudoinia, Beauveria, Bipolaris, Blastomyces, Boeremia, Botrytis, Brettanomyces, Brettanomyces, Candida, Cantharellus, Capronia, Ceraceosorus, Cercospora, Chaetomium, Chaetomium, Cladophialophora, Clavispora, Coccidioides, Colletotrichum, Coniophora, Coniosporium, Coprinopsis, Cordyceps, Cryptococcus, Cucurbitaria, Cutaneotrichosporon, Cyberlindnera, Cyphellophora, Dacryopinax, Daldinia, Debaryomyces, Diaporthe, Dichomitus, Didymella, Diplodia, Dissoconium, Diutina, Dothidotthia, Drechmeria, Drepanopeziza, Emericellopsis, Endocarpon, Epithele, Eremomyces, Eremothecium, Exophiala, Fibroporia, Filobasidium, Fomitiporia, Fomitopsis, Fonsecaea, Fulvia, Fusarium, Gaeumannomyces, Geosmithia, Glarea, Gloeophyllum, Grosmannia, Guyanagaster, Heterobasidion, Hirsutella, Histoplasma, Hyaloscypha, Hyphopichia, Ilyonectria, Jaminaea, Kalmanozyma, Kazachstania, Kluyveromyces, Kockovaella, Komagataella, Kuraishia, Kwoniella, Laccaria, Lachancea, Lachnellula, Laetiporus, Lasiodiplodia, Lentinula, Leptosphaeria, Letharia, Linderina, Lindgomyces, Lobosporangium, Lodderomyces, Macroventuria, Malassezia, Marasmius, Meira, Melampsora, Metarhizium, Metschnikowia, Meyerozyma, Microdochium, Microsporum, Mitosporidium, Mixia, Moesziomyces, Mollisia, Morchella, Mycena, Mytilinidion, Nannizzia, Naumovozyma, Nematocida, Neohortaea, Neurospora, Ogataea, Orbilia, Paecilomyces, Paracoccidioides, Paraphaeosphaeria, Parastagonospora, Penicilliopsis, Penicillium, Pestalotiopsis, Phaeoacremonium, Phanerochaete, Phialophora, Phycomyces, Pichia, Pleurotus, Pneumocystis, Pochonia, Podospora, Postia, Protomyces, Pseudocercospora, Pseudogymnoascus, Pseudomassariella, Pseudomicrostroma, Pseudovirgaria, Pseudozyma, Pseudozyma, Psilocybe, Puccinia, Punctularia, Purpureocillium, Pyrenophora, Pyricularia, Ramularia, Rasamsonia, Rhinocladiella, Rhizoctonia, Rhizophagus, Rhizopus, Rhodotorula, Saccharomyces, Saitoella, Saprochaete, Scedosporium, Scheffersomyces, Schizophyllum, Schizosaccharomyces, Sclerotinia, Serpula, Sodiomyces, Sordaria, Sparassis, Spathaspora, Sphaerulina, Spizellomyces, Sporisorium, Sporothrix, Stereum, Sugiyamaella, Suhomyces, Suillus, Synchytrium, Talaromyces, Tetrapisispora, Thermothelomyces, Thyridium, Tilletiaria, Tilletiopsis, Torulaspora, Trametes, Trematosphaeria, Tremella, Trichoderma, Trichophyton, Truncatella, Tuber, Uncinocarpus, Ustilaginoidea, Ustilago, Vanderwaltozyma, Venustampulla, Verruconis, Verticillium, Wallemia, Westerdykella, Wickerhamiella, Wickerhamomyces, Xylaria, Xylona, Yamadazyma, Yarrowia, Zasmidium, Zygosaccharomyces, Zygotorulaspora, Zymoseptoria.

The term “taxonomy” or “taxon” refers to a group of one or more microbial organisms that are classified into a group based on their common characteristics. Taxonomic hierarchy refers to a sequence of categories arranging various organisms into successive levels of the biological classification either in a decreasing or increasing order from domain to species or vice versa. Taxonomic rank is the relative level of a group of organisms (a taxon) in a taxonomic hierarchy. Examples of taxonomic ranks include strain, species, genus, family, order, class, phylum, kingdom, domain and others as understood by a person skilled in the art. Species is the basic taxonomic group in microbial taxonomy. Groups of species are then collected into genus. Groups of genera are collected into family, families into order, orders into class, classes into phylum, phyla into kingdom, and kingdoms into domain.

As a person skilled in the art will understand, each taxonomic level has increasing sequence similarity between individual members of the same taxonomic level from domain down to sub-species. As described herein, sequences that differ by single nucleotide may be quantitatively detected and subsequently analyzed.

In some embodiments, the target molecule is known or expected to be comprised in a microbe part of a microbial community. The term “microbial community” as used herein refers to a group of microorganisms sharing an environment which can comprise one or more microbes or individual genera or species of microbes. A microbial community in the sense of the disclosure can thus include two or more microorganisms two or more strains, two or more species. two or more genera, two or more families, or any mixtures of microorganisms in the sense of the disclosure with additional life form such as viruses, comprised in the shared environment. The interaction between the two or more community members may take different forms and can be in particular commensal, symbiotic and pathogenic as understood by a skilled person. An exemplary microbial community is the ‘microbiome” of an individual which is an aggregate of all microbiota (all microorganisms found in and on all multicellular organisms) residing on or within tissues and biofluids of the individual.

Microbial communities can be comprised within an individual as understood by a skilled person. The term “individual” or “host” as used herein indicates any multicellular organism that can comprise microorganisms, thus providing a biological environment for microbes and in in particular an environment for microbial communities, in any of their tissues, organs, and/or biofluids. Exemplary individual in the sense of the disclosure includes plants, algae, animals, fungi, and in particular, vertebrates, mammals more particularly humans. Exemplary biological samples from an individual comprise the following: whole venous and arterial blood, capillary blood, blood plasma, blood serum, dried blood spots, cerebrospinal fluid, interstitial fluid, sweat, lumbar punctures, nasal secretions, sinus washings, tears, corneal scrapings, saliva, sputum or expectorate, bronchoscopy secretions, transtracheal al aspirate, endotracheal aspirations, bronchoalveolar lavage, vomit, endoscopic biopsies, colonoscopic biopsies, subcutaneous and mesenteric adipose tissue biopsies, bile, vaginal fluids and secretions, endometrial fluids and secretions, urethral fluids and secretions, mucosal secretions, synovial fluid, ascitic fluid, peritoneal washes, tympanic membrane aspirate, urine, clean-catch midstream urine, catheterized urine, suprapubic aspirate, kidney stones, prostatic secretions, feces, mucus, pus, wound draining, skin scrapings, skin snips and skin biopsies, hair, nail clippings, cheek tissue, bone marrow biopsy, solid organ biopsies, surgical specimens, solid organ tissue, cadavers, breast milk, or tumor cells, among others identifiable by a skilled person. Biological samples can be obtained using sterile techniques or non-sterile techniques, as appropriate for the sample type, as identifiable by persons skilled in the art. Depending on the type of biological sample and the intended analysis, biological samples can be used freshly for sample preparation and analysis or can be fixed using fixative.

In some embodiments, in StochQuant methods and systems of the disclosure, StochQuant eliminates the need for special treatment of molecular counts of zero because it integrates them, together with other quantitative experimental information, in the StochQuant probability distributions as understood by a skilled person upon reading of the present disclosure.

In some embodiments, in StochQuant methods and systems of the disclosure, StochQuant probability distributions are in turn used to estimate taxon abundances and measure uncertainties.

In some embodiments, in StochQuant methods and systems of the disclosure, the StochQuant distributions of abundance are also used to perform comparative analyses. Some examples of analysis include: identification and computational filtering of contaminant reads and sequencing artifacts, differential abundance analysis, longitudinal analysis, and dimensionality reduction techniques (such as principal component analysis). Sampling from distributions of abundance can also be used to improve data visualization by presenting “clouds” of probable values rather than single values.

In some embodiments, in StochQuant methods and systems of the disclosure, StochQuant can be used in connection with 16S amplicon sequencing.

In some embodiments, in StochQuant methods and systems of the disclosure, StochQuant can be expanded beyond 16S amplicon sequencing to other types of amplicon sequencing.

The term “16S rRNA” indicates the 16S ribosomal ribonucleic acid of component of the ribosome 30S subunit of a prokaryote, or a DNA encoding therefor (herein 16S rRNA gene). A 16S rRNA of a prokaryote can be identified by its a sedimentation coefficient which, an index reflecting the downward velocity of the macromolecule in the centrifugal field. 16S rRNA performs various functions in a prokaryote such as providing scaffolding for the immobilization of ribosomal proteins, binds the shine Dalgarno sequence of mRNAs, interacts with 23S to help integrate two ribosome units (50S+30S). Accordingly, the 16S ribosomal RNA is a necessary for the synthesis of all prokaryotic proteins and is therefore comprised in all prokaryotes as understood by a skilled person.

The 16S rRNA is highly prevalent and highly conserved (overall) across a broad diversity of prokaryotes/in view of its role in the physiology of prokaryotes, 16S ribosomal RNA is the most conserved among prokaryotes. Accordingly, 16S rRNA is a key parameter in molecular classification and phylogenetic analysis of prokaryote possibly applied to the identification of clinical bacteria, sequence analysis and related therapeutic and/or diagnostic application. In particular classification and grouping of prokaryotes can be performed based on a sequence similarity in the 16S rRNA varying among prokaryotes based on their taxonomical ranks. Accordingly, 16S rRNA in the sense of the disclosure comprises conserved regions and variable regions. The conserved regions being conserved among prokaryotes with different degree of conservation among different taxa based on their taxonomic rank. The variable regions are instead specific for specific taxa with different degree of specificity among different taxa based on their taxonomic rank, as understood by a skilled person.

Accordingly, 16S rRNA can be used as a target molecule in StochQuant methods directed to detect abundance of microbes in a sample as understood by a skilled person.

Accordingly, a molecule that shares the features of 16S rRNA can be used as a target molecule in StochQuant methods directed to detect abundance of microbes in a sample as understood by a skilled person. Additional molecule can be used as a biomarker for a target microbes or target biological as will be understood by a skilled person.

The term a “biomarker” or “marker” is a measurable molecule which is specific for a referenced item and that provides information about the presence or activity of the reference items. The reference item can be a identity of an organisms or microorganism, a condition a physical or biological status of an endorsements. Accordingly, a biomarker is measurable molecule that is specific to a referenced item, such as a biological condition, disease, or process, and provides information about the presence or activity of that referenced item. Biomarkers can be used to detect and monitor various states of health or disease, offering insights into normal biological processes, pathogenic processes, or responses to therapeutic interventions. They are crucial in fields like medicine, environmental science, and biotechnology for their ability to provide specific and quantifiable data about complex biological systems. In particular a biomarker as used herein can be used to be specifically indicative microbial identity, assessing microbial biomass, and linking microbial presence to specific ecological or pathogenic processes.

The computational aspects of the methods described herein can be performed in systems as understood by a skilled person. Examples include a computer or network of computers (e.g., cloud) having one or more processors and memory accessible by those processors, a device comprising hardware or firmware designed to implement the method, a non-transitory computer-readable media that contains code to implement the method when read by a computer.

Examples of next generation sequencing technologies that one may use to perform a testing measurement for nucleic acid molecules include but are not limited to sequencing technologies by Illumina (see the web page genohub.com/ngs-instrument-guide/at the filing date of the present disclosure) such as the GAIIx, HiScanSQ, HiSeq 3000/4000, HiSeq High-Output v3, HiSeq High-Output v4, HiSeq Rapid Run, HiSeq X, MiSeq, MiSeq v2, MiSeq v2 micro, MiSeq v2 nano, MiSeq v3, MiniSeq High-Output, MiniSeq Mid-Output, MiniSeq Rapid, NextSeq 1000/2000, NextSeq 500, NovaSeq, NovaSeq X, NovaSeq SP, NovaSeq X Plus, or iSeq 100; Thermo Fisher Scientific such as the Ion Torrent PGM 314, 316, 318 chips, Proton I chip, S5/S5 XL chip, BGI; Agilent Technologies; Qiagen, Macrogen; Pacific Biosciences of California (Pacbio) such as PacBio RS, RS II, Revio. Sequel, Sequl II; Genewiz; 10× Genomics; Oxford Nanopore Technologies such as Flongle, GridION, MinION, PromethION 2, PromethION; Roche454 Gs FLX PTP, GS Junior 1 PTP; Element BioSciences such as AVITI; Complete Genomics such as DNBSEQ-E25, G400 FAST, G400 FCL, G400 FCS, G50 FCL, G50 FCS, G99, DNBSEQ-T7; Singular Genomics G4-F3.

Examples of applications and commercially available kits of next-generation sequencing library preparation which may be needed to perform the testing measurement of the target nucleic acid molecule (see the website genohub.com/ngs-library-preparation-kit-guide/at the filing date of the present disclosure) include:
a. exome sequencing such as Agilent HaloPlex, Agilent SureSelect, Agilent SureSelect QXT, IDT xGen, Illumina Nextera Rapid Capture, Illumina TruSeq, MYcroarray Mybaits, Roche Nimblegen SeqCap;
b. DNA-Seq (whole genome) sequencing such as Beckman SPRIworks Fragment Library, Beckman SPRIworks HT, Bioo Scientific NEXTflex DNA, Bioo Scientific NEXTflex PCR-Free, Bioo Scientific NEXTflex Rapid DNA, Illumina Nextera DNA, Illumina Nextera DNA Flex, Illumina Nextera XT, Illumina TruSeq DNA, Illumina TruSeq DNA PCR-Free, IntegrenX PrepX ILM DNA, Kapa DNA Library, Life Tech Ion Plus Fragment, NEB NEBNext DNA, NEB NEBNext Ultra DNA, NuGEN Encore Rapid Library, PacBio DNA Template Prep;
c. Target capture gene panels such as Agilent ClearSeq, Agilent SureSelect Capture, Archer FusionPlex, Bioo Scientific NEXTflex, Illumina TruSight, Nimblegen SeqCap, Qiagen GeneRead;
d. CHIP sequencing such as Bioo Scientific NEXTflex CHIP, Diagenode iDEAL CHIP, Epigentek EpiNext CHIP, Illumina TruSeq CHIP;
e. RNA sequencing (mRNA or cDNA sequencing) such as Directional RNA (polyA-selected) library prep, Directional RNA (rRNA-depleted) Illumina library prep, RNA (polyA-selected) Illumina library prep, RNA (rRNA depleted) Illumina library prep, Bioo Scientific NEXTflex Rapid Directional qRNA, Bioo Scientific NEXTflex Rapid Directional RNA, Bioo Scientific NEXTflex Rapid qRNA, Clonetech SMARTer, Epicentre ScriptSeq, Gnomegen RNA Profiling Kit, Illumina TruSeq Stranded, NEB NEBNext Ultra Directional RNA, NEB Next Ultra II Directional RNA;
f. small RNA sequencing (microRNA-seq) such as Bioo Scientific NEXTflex Small RNA Seq v2, Epicentre ScriptMiner, Illumina TruSeq Small RNA, NEB NEBNext Small RNA, Seqmatic TailorMix miRNA Kit;
g. metagenomics sequencing such as Beckman SPRIworks FragmentL, BiooScientific NEXTflex PCR, Illumina Nextera XT, Illumina TruSeq DNA, Illumina TruSeq DNA PCR-Free, Illumina TruSeq Nano DNA, Kapa DNA library, Life Tech Ion Plus Fragment, Life Tech Ion Xcpress Plus, MGIEasy PCR-Free DNA, MGIEasy Universal DNA, PacBio DNA Template Prep;
h. 16S amplicon sequencing such as Bioo Scientific Nextflex 18S ITS Amplicon, Bioo Scientific NEXTFlex 16S V4 Amplicon-Seq Kit, Bioo Scientific NEXTflex 16S V1-V3 Amplicon-Seq Kit Illumina MiSeq Reagent Kits;
i. Mate-Pair Sequencing such as Illumina Nextera Mate Pair;
j. Methyl DNA sequencing such as Bioo Scientific NEXTflex Methyl-Seq, Roche Nimblegen SeqCap Epi Kit, Illumina TruSeq Methyl Capture EPIC Library Prep Kit;
k. Single Cell DNA-Seq such as Qiagen REPLI-g Single Cell, Rubicon Genomics PicoPlex;
l. Single cell RNA-seq such as Clontech SMARTer Ultra Low Input, Takara SMART-Seq Ultra Low Input RNA Kit, Nextera XT DNA Library Preparation Kit. In some embodiments, a next generation sequencing technology may be combined with a method or methods to process an output or outputs from a sequencing technology.

Examples of file types that can store sequences of target nucleic acid molecules (Ref: www.formbio.com/blog/your-essential-guide-different-file-formats-bioinformatics) include FASTQ, FASTA, SRA, BAM, CRAM, SFF, SAM, BED, GTF/GFF, VCF, Wiggle, BigWig, BigBed, BCF, tar.gz, PDB, PED, MAP, CSV, JSON,

Examples of methods to process an output (which can be considered as part of the testing measurement) from sequencing technologies may include but are not limited to the following, as described in e.g., Rute Pereira, Jorge Oliveria, Mario Sousa “Bioinformatics and Computational Tools for Next-Generation Sequencing Analysis in Clinical Genetics” J Clin Med. 2020, DOI: 10.3390/jcm9010132:
i. basecallers such as BlindCal, AYB, Freelbis, TotalReCaller, Srfim, BayesCall, Ibis, Rolexa, Softy, OnlineCall, BM-BC, ParticleCall, TotalReCaller, NaiveBayesCall, Srfim, Ibis, Alta-Cyclic; read filtering and trimming tools such as NGS QC toolkit, QC-Chain, FastQC, Btrim, leeHom, AdapterRemoval, Trimmonatic, TorrentSuite;
ii. sequence alignment tools such as Burrows-Wheeler Aligners (BWAs), Bowtie, Torrent Mapping Alignment Program (TMAP), Bowtie 2, Novoalign, SHRIMP, SOAPv2, mapping tools using Smith-Waterman or Needleman-Wunsch algorithms), kallisto, eXpress;
iii. de novo assembly tools that rely on Overlap-Layout-Consensus (OLC), de Brujin graph (DBG, K-mer graph, or greedy graph algorithms that can use either OLC or DBG;
iv. post alignment processing tools such as TMAP software, Illumina tools, SAMtools, Genoma Analysis Toolkit (GATK), Picard, IndelRealigner from GATK, BaseRecalibrator from GATK, Freebayes, Torrent Variant Caller (TVC);
v. structural variant calling tools such as BreakDancer, PEMer, Pindel, SLOPE;
vi. tertiary analysis tools such as variant annotation using tools such as SIFT, PolyPhen-2, CADD, Condel, ANNOVAR, variant effect predictor (VEP), snpEff, SeattleSeq, Galaxy, GKNO;
vii. databases such as Ensemble, REfSeq, and UCSC, 1000 genome project, Exome Aggregation Consortium (ExAC), and the Genome Aggregation Database (gnomAD); markergene reference databases such as a Greengenes 16S rRNA database, a Silva 16S or 18S rRNA database such as the Silva 18 SSURef NR99 full length database or the Silva 138 SSURef NR99 515F/806R region database, or others as described e.g., in DOI: 10.1371/journal.pcbi.1009581, DOI: 10.1101/2020.10.05.326504, or 10.1093/nar/gks1219 all incorporated by reference in their entirety, fungal reference databases such as UNITE for fungal ITS, or SEPP reference databases.
viii. Taxonomy classifiers such as Naïve Bayes classifiers (as described e.g., in DOI: 10.1101/2020.10.05.326504, DOI: 10.1186/s40168-018-0470-z, DOI: 10.1101/2022.12.19.520774, or DOI: 10.1186/s40168-018-0470-z each incorporated by reference in its entirety)
ix. and other tools such as Phevor, VarSeq/VSClinical (Golden Helix), Ingenuity Variant Analysis (Qiagen), Alamut, and VarElect.
x. Other software tools may include Qiime2 (as described e.g., in DOI: 10.1038/s41587-019-0209-9 incorporated by reference in its entirety).

Examples of sequence alignment software (which can be used as part of the data processing step(s) of the testing measurement) include: (see the website en. wikipedia.org/wiki/List_of_sequence_alignment_software at the filing date of the present disclosure)
i. database search only sequence alignment software such as BLAST, HPC-BLAST, CS-BLAST, CUDASW++, DIAMOND, FASTA, GGSEARCH, GLSEARCH, Genome Magician, Genoogle, HMMER, HH-suite, IDF, Internal, KLAST, LAMBDA, MMseqs2, USEARCH, OSWALD, parasail, PSI-BLAST, PSI-Search, R&R, ScalaBLAST, Sequilab, SAM, SSEARCH, SWAPHI, SWAPHI-LS, SWIMM, SWIMM2.0, SWIPE;
ii. pairwise alignment tools such as ACANA, AlignMe, ALLALIGN, Bioconductor, BioPerl dpAlign, BLASTZ, LASTZ, CUDAlign, DOTLET, FEAST, Genome Compiler, G-PAS, GapMis, Genome Magician, GGSEARCH, GLSEARCH, Jaligner, K*Sync, LALIGN, NW-align, mAlign, matcher, MCALIGN2, MegAlign Pro (Lasergene Molecular Biology), MUMmer, needle, Ngila, NW, parasail, Path, PatternHunter, ProbA, PyMOL, REPuter, SABERTOOTH, Satsuma, SEQALN, SIM, GAP, NAP, LAP, SIM, SPA: Super pairwise alignment, SSEARCH, Sequences Studo, SWIFOLD, SWIFT suit, stretcher, tranalign, UGENE, wordmatch, YASS;
iii. multiple sequence alignment tools such as ABA, ALE, ALLALIGN, AMAP, anon, Bali-Phy, Base-By-Base.

Examples of differential abundance analysis software (which can use molecular counts or data based upon the molecular counts of the testing measurement) include (see e.g. the website microbiome.github.io/OMA/differential-abundance.html at the filing date of the present disclosure) ALDEX2, corncob, DACOMP, eBAY, GMPR, Rarefy, TSS, MaAsLin2, megatenomeSeq, LDA, RAIDA, ANCOM-BC, Omnibus, DESeq2, edgeR (as described e.g., in DOI: 10.1186/s40168-022-01320-0 incorporated by reference in its entirety). Other examples may include lefser, limma, LinDA, ZicoSeq, LDM, ZINQ, fastANCOM, t-test, and Wilcoxon test, Kruskal-Wallis test, Mann-Whitney U test.

Examples of dimensionality reduction techniques (which can use molecular counts or data based upon the molecular counts of the testing measurement) include (see. the website towardsdatascience.com/11-dimensionality-reduction-techniques-you-should-know-in-2021-dcb9500d388b) at the filing date of the present disclosure).
i. linear methods such as principal component analysis (PCA), factor analysis (FA), linear discriminant analysis (LDA), truncated singular value decomposition (SVD);
ii. non-linear methods such as kernel PCA, t-distributed stochastic neighbor embedding (t-SNE), multidimensional scaling (MDS), isometric mapping (Isomap);
iii. and other methods such as Backward Elimination, Forward Selection, and Random forests.

Examples of alignment visualization software include (en. wikipedia.org/wiki/List_of_alignment_visualization_software):
i. Alan, Ale, AliView, alv, arb, Base-By-Base, BioEdit, BioNumerics, bioSyntax, BoxShade, CINEMA, CLC viewer, CIAlign, ClustalX viewer, Cylindrical Alignment App, Cylindrical BLAST Viewer, DECIPHER, Discovery Studio, DnaSP, DNASTAR, emacs, FLAK, Genedoc, Geneious, Integrated Genome Browser (IGB), Interactive Tree of Life (iTOL), IVISTMSA, JalView, Jevtrace, JSAV, Lucid Align, Maestro, MEGA, Molecular Operating Environment (MOE), MSAReveal, Multiseq, MView, PFAAT, Ralee, S2S RNA editor, Seaview, Seqotron, Sequilab, SeqPup, Sequlator, SnipViz, Strap, Tablet, UGENE, VISSA, DNApy, Alignment Annotator.

Examples of phylogenetics software can include (see en. wikipedia.org/wiki/List_of_phylogenetics_software):
i. AMDIXTOOLS, AncesTree, AliGROOVE, ape, Armadillo Workflow Platform, Bali-Phy, BATWING, BayesPhylogenies, BayesTraits, BEAST, BioNumerics, Boseque, BUCKy, Canopy, CITUP, ClustalW, Dendroscope, EXACT, EZEditor, fastDNAml, FastTree2, fitmodel, Geneious, Genozip, HyPhy, IQPPNI, IQ-TREE, jModelTest2, JolyTree, LisBeth, MEGA, MegAlign Pro, Mesquite, MetaPIGA2, MicrobeTrace, Modelgenerator, MOLPHY, MorphoBank, MrBayes, Network, Nona, PAML, ParaPhylo, PartitionFinder, PASTIS, PAUP, Phybase, PHYLIP, PhyloQuart, QuickTree, SimPlot++, SplitsTree, Treefinder, T-REX, UGENE, VeryFastTree, Xrate

Examples of instruments that can be used to obtain absolute abundance measurements of a reference molecule.
a. Digital PCR instruments can be used to measure a reference nucleic acid; examples include instruments such as the Qiagen QiAcuity Digital PCR System, ThermoFisher Scientific QuantStudio Absolute Q Digital PCR System, BioRad QX600 Droplet Digital PCR System, Biorad QX600 Biorad AutoDG Droplet Digital PCR System, BioRad QX ONE Droplet Digital PCR System Biorad QX200 AutoDG Droplet Digital PCR System, BioRad QX200 Droplet Digital PCR (ddPCR) System, JN Medsys Clarity Digital PCR system, Stilla Technologies Digital PCR System, Sysmex Corporation BEAMing Digital PCR Technology, Standard BioTools Inc Digital PCR System, or Precigenome LLC Digital PCR System.
b. Flow cytometry instruments can be used to measure a reference cell; examples include instruments such as the Agilent Technologies NovoCyte Quanteon Flow Cytometer, Miltenyi Biotec MACSQuant Analyzer 16 Flow Cytometer, BD Biosciences BD FACSLyric Flow Cytometer Integrated with the BD FACSDuet Sample Preparation System, Revvity Cellometer Spectrum, Sartorius iQue 3 Advanced Flow Cytometry Platform, Agilent Technologies NovoCyte Benchtop Flow Cytometer, Bio-Rad ZE5 Cell Analyzer, BD Biosciences BD Accuri C6 Plus Flow Cytometer, Thermo Fisher Scientific Attune NxT Flow Cytometer.
c. Digital protein quantification platforms can be used to measure a reference protein molecule; examples include instruments such as Quanterix HD-X Automated Immunoassay Analyzer, Quanterix SP-X Imaging and Analysis System, or SR-X Biomarker detection system.
d. Real-Time PCR instruments can be used to measure a reference nucleic acid; examples include instruments such as Bio-Rad CFX Opus Real-Time PCR System, Agilent Technologies AriaMx Real-time PCR System, Bio-Rad CFX96 Touch Deep Well Real Time PCR Detection System, Roche LightCycler96 Instrument, Roche LightCycler96 Instrument, Qiagen QIAquant 384 5plex Real-time PCR System, QIAquant 96 2plex Real-time PCR System, or Thermo Fisher Scientific QuantStudio 3 Real-Time PCR System.

The StochQuant methods and systems herein described can be used in connection with various applications wherein detection of a target molecule is desired. For example, methods and systems herein described and related composition can be used in application to detect and/or analyze biomarker molecules e.g., for diagnostic, therapeutic and/or investigative purposes, samples. In particular, StochQuant is useful for detection in a number of practical applications, including microbiome analysis, infectious disease diagnostics, cancer diagnostics, prenatal diagnostics and additional detections identifiable by a skilled person. Additional exemplary applications include detection of target molecule in testing measurements performed in several fields including basic biology research, applied biology, bio-engineering, medical research, medical diagnostics, therapeutics, and in additional fields identifiable by a skilled person upon reading of the present disclosure.

In embodiments of methods and systems herein described, StochQuant approach is particularly useful in any field where one or more procedures comprise a molecular detection. Methods to perform molecular require manipulations of an environment, sample and/or sub-sample thereof which introduce stochasticity which can impact the molecular count of molecule of interest.

Accordingly, StochQuant detection improves: molecular detection of one or more target molecules e.g., by allowing a more accurate and effective detection, identification and quantification of target molecules than traditional methods and by allowing more accurate and reliable comparisons of molecular levels or concentrations (e.g. differential abundance), including for the same molecule across samples and for different molecules within or across samples.

For example StochQuant-derived probability distribution of molecular counts of one or more biomarkers in an environment allows a skilled person to obtain with a single testing measurement a more accurate and reliable information concerning the actual number of biomarker molecule in an environment (and therefore, for example when microbial biomarkers are analyzed, the actual number of microorganisms of the taxa in the environment or the extent of disease progression). This, the use of the probability distribution reduces the errors (which are inevitably associated with a single counts detection of the molecules when not accounting for stochasticity) in the set of experiments or actions the skilled person will perform downstream of and based on the identification, detection, quantitative detection, and differential abundance analysis of biomarker molecules.

StochQuant-derived confidence interval of molecular counts of one or more biomarkers corresponding to a specified (e.g. by the user) confidence level threshold allows a skilled person to, for example 1) identify a range of molecular counts for the biomarker with a user-set degree of certainty or probability that the true molecular counts of a biomarker is comprised within the inferred range of molecular counts and/or 2) identify one or more ranges within which the true molecular count of the one or more biomarkers is expected to lie, each range associated with a corresponding degree of certainty or probability indicated by the confidence level. Thus, StochQuant-derived confidence interval of molecular counts allows better informing the skilled person concerning 1) the counts for a downstream use which requires a set degree of probability (e.g. expensive set of testing measurement, or testing that cannot be repeated which are based on the identified counts and may be required by a company to be performed with counts having at least a certain % confidence level) and/or 2) the likely range of molecular counts of the target biomarker with a set degree of probability and therefore a) informing the skilled person about the precision with which the quantitative detection of the biomarker has been performed, b) informing the skilled person about the usability of this biomarker measurement for interpretation, decision making, and downstream analyses.

A StochQuant-derived confidence level associated with a specified (e.g. by the user) confidence interval of molecular counts of one or more biomarkers allows a skilled person to identify the confidence level that the molecular counts of one or more biomarkers are within the user-specified range of molecular counts, thus 1) better informing about which range of molecular counts can the skilled person can select a downstream action based on the skilled person's intent and/or 2) better informing a skilled person's decision which depends on the likelihood of the biomarker being in the particular range (for example, the range associated with “health” or “disease”), such as a decision to administer a therapy or another intervention. Note that the skilled person may choose to specify the confidence interval of molecular counts of a biomarker in the form of a threshold (such as “above threshold X” or “below threshold X”, where it should be understood that the term “above X” maybe be also used to mean “inclusive of X and above X” and the term or “below X” maybe used to mean “inclusive of X and below X”). Then the corresponding StochQuant-derived confidence level corresponding to such interval can be used to better inform on the probability that the actual molecular counts of the biomarker are in the ranges above or below the threshold. Such StochQuant-derived confidence level would better inform a skilled person's actions associated with this confidence level, for example the skilled person's decision to administer intervention (e.g. therapy) if a biomarker is likely above or below the “normal” value threshold. Taking an action may require a certain confidence level such as for example, about 80%, 90% 95%, 98%, 99%, 99.5%, 99.9% confidence level. A certain desired confidence level for taking an action may be set by the skilled person and/or may be by an external body such as a regulatory agency such as US FDA.

Accuracy and reliability of the measurement is important in many fields for decision making in a number of technologically important areas including medicine, agriculture, farming, biotechnology, and environmental monitoring with particular reference to detection of the abundance of molecules of interest.

Therefore, anyone of the outcomes of the StochQuant detection improves the detection process itself in providing the user with information concerning the stochasticity introduced by the necessary manipulation of the molecules that are detected. Accordingly in improving the detection process, StochQuant improves many fields of technology where having a more accurate and reliable information concerning a detected molecular count of the target molecule is important for an effective understanding and manipulation of biological and chemical systems.

For example, StochQuant detection improves any technical fields where microbial measurements, microbial diagnostics, and microbiome studies are e.g., by allowing a more accurate and effective detection, quantitative detection, and differential abundance analysis of microbial taxa and microbial biomarker molecules than traditional methods. For example, StochQuant-derived probability distribution of molecular counts of one or more microbial biomarkers used to identify the taxa or, for example, their functions (e.g. 16S RNA gene or gene product and including other biomarkers specified herein) allows a skilled person to obtain with a single testing measurement a more accurate and reliable information concerning the actual number of biomarker molecule in an environment (and therefore, for example, the actual number of microorganisms of the taxa in the environment). StochQuant-derived confidence interval of molecular counts of one or more biomarkers corresponding to a specified (e.g. by the user) confidence level threshold and StochQuant-derived confidence level associated with a specified (e.g. by the user) confidence interval of molecular counts of one or more biomarkers allows a skilled person to enable significant improvements in commercial applications of microbial measurements. These include controlled change in microbiome to obtain a technical purpose (e.g., creating a microbiome with controlled absolute abundances of specific taxa); identification of therapeutic approaches; measurements of effects of drugs on the microbiome; measuring the effect of microbiome on metabolism of or on effectiveness of drugs, vaccines, and dietary interventions; analyzing microbes in tumors and tumor microenvironments to improve development and delivery of cancer vaccines and therapeutic treatments, including immunotherapeutics, small molecules, and antibody-drug conjugates; analyzing tumor neoantigens and microbial antigens to develop improved immunotherapies and identify patients more likely to respond to them. Commercial applications of accurate measurements of microbial targets as provided by StochQuant include drug development, drug delivery, and diagnostics, as also described in the present disclosure.

Examples of the value of improved measurements of microbial targets as provided by StochQuant include areas being commercially pursued by a number of companies, including [22]) Axial Biotherapeutics (developing biotherapeutics based on microbiome characterization), BiomeSense (tracking microbiome profiles during clinical trial), ResBiotic (development of anti-inflammatory probiotic to reduce neutrophilic inflammation to restore human lung microbiome), Finch Therapeutics (developing microbiome therapeutics), Viome (providing human microbiome nutritional information through RNA-seq), Second Genome (identifying novel proteins and peptides within microbiome for precision therapies), Sun Genomics (creating custom probiotics based on gut DNA to treat dysbiosis), Microgenesis (developing non-invasive test to detect imbalance of vaginal and intestinal microbiome), AnimalBiome (developing microbiome diagnostics and therapeutics for pets), BrickBuiltTherapeutics (developing treatments for oral health), Rebiotix (delivering microbes into a sick patient's intestinal tract), Oralta (producing probiotic supplement for bad breath), Evelo Biosciences (developing orally derived medicines to act on cells in the small intestines and provide therapeutic effects), Siolta Therapeutics (developing therapeutics using human microbiome to treat inflammatory disease), Nexilico (using computational technologies to understand microbiome-related drug metabolism), Seres Therapeutics (developing therapeutics to treat dysbiosis in the colonic microbiome and prevent Clostridium difficile infection), Scioto Biosciences (delivering live therapeutic bacteria to the gut), Azitra (developing novel microbiome-based therapies to treat skin conditions and diseases like ichthyosis vulgaris, eczema, inflammatory skin), Vedanta Biosciences (developing novel therapies designed from a consortium of human commensal bacteria using information from human interventional studies).

Furthermore, StochQuant improves any technical fields performing differential abundance analysis of one or more target molecules in one or more environments by utilizing StochQuant-derived probability distribution of molecular counts of one or more target molecules in one or more environments to provide a more reliable and accurate differential abundance analysis. Such differential abundance analysis of target molecules is needed in a number of practical areas including microbial analysis, transcriptomic analysis, genetic analysis, in vitro diagnostics, and drug development.

Furthermore, StochQuant (including by providing probability distribution of target abundance in an environment, confidence Interval of abundance values derived from a specified Confidence Level; and confidence Level for a specified Confidence interval of abundance values) improves the technical field of genomics. by improving many aspects of genomic analysis, including the following ones. Copy number variation (CNV) analysis includes accurately comparing copy number (molecular count) of different genes within an environment and comparing copy number of a gene among environments. CNV analysis in practice can be used, for example, to identify genomic regions that have been duplicated or deleted, and reveal copy number variations associated with diseases. Rare Variant Detection, which improves the detection of rare genetic variants or mutations present at low frequencies in an environment. It requires accurate and confident detection (and optionally quantification) of rare genetic variants. This type of detection has applications in cancer genomics and non-invasive prenatal testing. Single-Cell Genomics requires quantitatively detecting and analyzing DNA molecules and their sequences from individual cells, and allows, for example, identification of rare cells, which is important in cancer detection and analysis, and in identifying rare clones which is also important in biotechnology (e.g. to identify cells producing the desired biotechnological product such as an antibody).

Furthermore, any one of the StochQuant outcomes improves any technical field in which gene expression analysis is performed. Gene expression analysis includes quantitatively detecting RNA molecules, including quantitatively detecting RNA molecules from individual cells (including single cell RNA analysis and single cell RNA sequencing). It allows examining gene expression and gene expression heterogeneity within cell populations and identifying cells with unique expression profiles, which is beneficial in many technological areas including medicine and biotechnology. These technological areas include cancer (e.g. characterizing tumor heterogeneity and identify rare cell populations and monitoring tumor progression); immunology (including developing and monitoring treatment of autoimmune diseases, identifying patients who are likely to respond to certain treatments, and monitoring immune response to infectious agents); drug discovery and development (e.g. identifying cell-specific drug responses and potential side effects and characterizing cellular heterogeneity in drug resistance, making prognostic predictions based on intra-tumor cellular diversity); precision medicine (including identifying patient-specific cellular markers for targeted therapies and monitoring treatment responses at the cellular level). These applications also include analyzing crop responses to environmental stresses.

Furthermore, improved molecular detection provided by StochQuant improves any technical field involving bioproduction and biotechnology by, for example, cell line development for bioproduction, quality control in cell-based therapies, and optimization of cellular engineering processes. StochQuant furthermore improves validation and quantitative detection of synthesized molecules. Examples include nucleic acid and protein libraries commercially produced such as those produced by Twist Bioscience (see www.twistbioscience.com/products/libraries/spread-out-low-diversity-libraries) such as clonal genes, gene fragments, oligo pools, NGS panels such as custom panels, long read panels, exome panels, human comprehensive exome, human core exome, human methylome panel, human refseq panel, mitochondrial panel, mouse exome panel, respiratory virus research panel, comprehensive viral research panel; variant libraries such as CAR libraries, TCR libraries, combinatorial variant libraries, spread out low-diversity libraries, site-saturation libraries, synthetic controls such as cfDNA pan-cancer reference standards and infectious disease controls such as respiratory virus controls, SARS-COV-2 controls, or monkeypox virus controls; or for antibody discovery, antibody optimization, antibody sequencing, antibody screening, or antibody characterization.

Furthermore, improved molecular detection provided by StochQuant improves the technical field of drug discovery and drug development, including analysis of DNA-encoded libraries and screening experiments involving DNA-encoded libraries. Also, including gene expression analysis, including genes that are differentially expressed in disease states compared to healthy states, genes differentially responsive to drugs and drug candidates, genes associated with drug efficacy or drug toxicity, identifying off-target drug action, and including identifying and quantitatively detecting novel transcripts and splice variants that may be involved in pathological processes.

Furthermore, improved molecular detection provided by StochQuant improves the technical field of diagnostics, including in vitro diagnostics and including molecular diagnostics in humans and in other animals, including veterinary medicine and in agricultural biotechnology. StochQuant's improvements include the reduction of false positives or false negatives in the diagnosis of a disease from the testing measurement, which is technologically important because an inaccurate determination of presence or absence of a target biomarker can lead to having false positive or false negative responses in outcome of the diagnostic test. Having a probability distribution of target abundance in an environment leads to a more reliable determination on whether the molecular count result is positive or negative or whether the result is within a certain reference range of values or whether the result is above or below a certain threshold. Also, the probability distribution allows the user to assign a confidence level to detected values which allows better decision making on further course of action (whether to repeat the test or whether to proceed based on the determination), leading to the improved detection of a disease and monitoring of health. This capability of StochQuant is technologically important because an inaccurate determination of presence, abundance, or change in abundance of a target biomarker (e.g., in comparison to a previous measurement) can lead to having a false negative response in outcome of a diagnostic test leading to delayed diagnosis and treatment of a disease. Similarly, improved molecular detection provided by StochQuant improves the technical field of the monitoring of disease treatment response via analogous approaches to the ones used for diagnostics and including improvement in the quantitatively detecting of the levels of nucleic acids used in gene therapy, including therapy delivered via viral vectors or lipid nanoparticles.

Furthermore, improved molecular detection provided by StochQuant improves the technical field of agricultural biotechnology including analysis and monitoring of technologically important plants, birds, mammals, fish, and invertebrates such as shrimp. These improved capabilities include monitoring and diagnosing of diseases of farmed animals, including methods and approaches analogous to the human in vitro diagnostics described herein, including environmental monitoring for pathogens affecting farmed animals. These improved capabilities also include environmental monitoring for pathogens affecting organisms of interest to agricultural biotechnology. These capabilities improved by StochQuant further include genetic analysis of crops and food products, including identifying the presence, absence, or quantity of a genetically modified organism in an agricultural and or food product. Common genetically modified crops include soybeans, corn, cotton, canola, sugar beets, alfalfa, papaya, squash, potatoes, apples, eggplant, and rice. These capabilities improved by StochQuant further include detection of desired or undesired organisms within a food product, for example detection of meat adulterated with additional organisms, including: Pork in beef and lamb products, Chicken in beef and lamb products, Duck in beef and lamb products, Horse meat in beef products; Goat meat in lamb products, Lower-cost meats like chicken or turkey in more expensive meat products. Furthermore, the capabilities improved by StochQuant include Species Identification to verify the identity of seafood products and detect species substitution. In these examples, StochQuant can be used, for example, to have confidence that a certain adulterant is not present above a certain threshold.

Furthermore, improved molecular detection provided by StochQuant improves the technical field of veterinary medicine, including in vitro diagnostic improvements analogous to those described for human in vitro diagnostic described herein.

Furthermore, improved molecular detection provided by StochQuant improves the technical field of environmental monitoring, including monitoring of air, water, and waste streams, including performing such monitoring in the context of public health, including one to monitor pathogens, pathogen variants and strains, and genetic features associated with antimicrobial resistance. Wastewater and waste stream analysis is described, for example, in (ref [23]) and utilizes, for example, amplicon sequencing, shotgun metagenomics, and hybrid capture enrichment.

Additional commercial applications for which StochQuant can improve the detection of a target molecule or target molecules comprise the following

Cancer detection: StochQuant improves cell-free DNA detection and methylation pattern detection such as the Grail Gelleri test (as described e.g., in (ref. incorporated by reference in its entirety) tissue-based companion diagnostics for solid tumors such as the FoundationOne CDx or Liquid CDx such as (as described e.g., by www.foundationmedicine.com/test/foundationone-cdx as of Aug. 22, 2023): small-non cell lung cancer with the following biomarkers: EGFR exon 19 deletions and EGFT exon 21 L858R alterations, EGFR exon 20 T790M alterations, ALK rearrangements, BRAF V600E, MET single nucleotide variants (SNVs) and indels that lead to MET exon 14 skipping, ROS1 fusions; melanoma with the following biomarkers: BRAF V600E, BRAF V600K, BRAF V600 mutation-positive; breast cancer with the following biomarkers: ERBB2 (HER2) amplification, PIK3CA, C420R, E542K, E545A, E545D [1635G>T only], E545G, E545K, Q546E, Q546R, H1047L, H1047R, and H1047Y alterations; colorectal cancer with the following biomarkers: KRAS wild-type (absence of mutations in codons 12 and 13), KRAS wild-type (absence of mutations in exons 2, 3, and 4) and NRAS wild type (absence of mutations in exons 2, 3, and 4), ovarian cancer with the following biomarkers: BRCA1/2 alterations, cholangiocarcinoma with the following biomarkers: FGFR2 fusions and select rearrangements; prostate cancer with the following biomarkers: Homologous Recombination Repair (HRR) gene (BRCA1, BRCA2, ATM, BARD1, BRIP1, CDK12, CHEK1, CHEK2, FANCL, PALB2, RAD51B, RAD51C, RAD51D, RAD54L) alterations; solid tumors with the following biomarkers: MSI-High, TMB>10 mutations per megabase, NTRK1/2/3 fusions.

StochQuant improves circulating tumor DNA (ctDNA) detection such as detection performed by Natera Signatera. Detection of genomic targets such as detection performed by Natera Altera Comprehensive Genomic Profiling, which involves somatic profiling that includes RNA sequencing (call fusions with established clinical reference, detect novel fusions), introns, promoters; reporting TMB, MSI, and genes related to HRD (ref: [25]). Pathogenic variants in the CFTR gene (as described e.g., by DOI: 10.1038/s41436-020-0822-5 incorporated by reference in its entirety). An example of a test to screen for variants in the CFTR gene is the LabCorp Cystic Fibrosis (CF) Full0-gene Carrier screen (Test 482632).

Monitoring the minimal residual disease and/or measurable residual diseases (MRD) after treatment via detection and quantification of molecules associated with the presence of the disease [26-29]. It is useful in a number of diseases, including cancer, including Hematological Malignancies/blood cancers: Acute Lymphoblastic Leukemia (ALL): NGS-based MRD detection has shown strong prognostic value in pediatric and adult ALL. Acute Myeloid Leukemia (AML): MRD status is associated with survival outcomes in AML patients. Chronic Myeloid Leukemia (CML): MRD monitoring helps guide treatment decisions and predict relapse risk. MRD monitoring is also applicable in solid tumors: Circulating Tumor DNA (ctDNA): Analysis of ctDNA in blood samples is emerging as a promising approach for MRD detection in various solid tumors.

Prenatal diagnostics: StochQuant improves exome sequencing for prenatal structural anomalies (as described e.g., in ref. incorporated by reference in its entirety). Genetic prenatal screening such as the Natera Panorama test (as described e.g., in refs. [31, 32] each incorporated by reference in its entirety). Other examples of prenatal genetic screening commercial applications include Myriad genetics Prequel Prenatal Screen, Illumina NIPT, Luna Genetics Luna Prenatal Test, Invitae NIPS.

Vaginal microbiome diagnostics and tests: Examples of applications of quantitative detection of targets related to the vaginal microbiome may include to characterize the vaginal microbiome for identification of aerobic vaginitis, bacterial vaginosis, cytolytic vaginosis, recurrent UTIs, good health, yest infections, or Mycoplasma/Ureaplasma. Non-limiting commercial examples may include Coriell Life Sciences, Evvy Vaginal Health Test, the Juno Vaginal Microbiome Test, BiomeFX Vaginal Microbiome Test Kit.

Sepsis diagnostics: Examples may include detection of microbial cell free DNA in blood as performed by the Karius Test (described in ref. incorporated by reference in its entirety), through host transcriptomics as performed by the Inflammatix tests, or through a combination of approaches (as described e.g., in ref. incorporated by reference in its entirety).

Infectious disease diagnostics: including detection and quantification of pathogens and of biomarkers of and mutations associated with antimicrobial resistance or susceptibility, antibiotic resistance or susceptibility, drug resistance or susceptibility. Viral load testing, including viral load testing for HIV, Cytomegalovirus (CMV); Hepatitis B virus (HBV); Hepatitis C virus (HCV). Viral load testing is crucial for: Diagnosing viral infections, Monitoring disease progression, Guiding treatment decisions, assessing response to antiviral therapy, Detecting treatment failure or viral resistance. Furthermore, viral load testing may be used to assess respiratory infections, including SARS-COV-2 infections, influenza infections, and RSV infections.

Quantitative detection of specific viral sequences, including sequence determination of the target viruses such as HIV, HCV, HBV, HSV, including quantitative detection of viral groups, types, subtypes, strains, and including quantitative detection of viral mutations, including mutations associated with drug resistance and/or vaccine resistance and including mutations indicating pandemic potential and host-jumping.

Detection and sequence analysis of organisms associated with sexually transmitted infections, including Chlamydia: Chlamydia trachomatis; Gonorrhea: Neisseria gonorrhoeae; Syphilis: Treponema pallidum; Trichomoniasis: Trichomonas vaginalis; Mycoplasma genitalium: Mycoplasma genitalium; including detection of mutations associated with antimicrobial resistance, antibiotic resistance.

Detection and sequence analysis of pathogenic fungi, including those listed on the World Health Organization (WHO) Fungal Priority Pathogens List [35-37], including Cryptococcus neoformans; Candida auris; Aspergillus fumigatus; Candida albicans; Nakaseomyces glabrata (Candida glabrata); Histoplasma species; Eumycetoma causative agents; Mucorales; Fusarium species; Candida tropicalis; Candida parapsilosis; Scedosporium species; Lomentospora prolificans; Coccidioides species; Pichia kudriavzeveii (Candida krusei); Cryptococcus gattii; Talaromyces marneffei; Pneumocystis jirovecii; Paracoccidioides species.

Reference molecules: Multiple RNA expression reference molecules can be measured by a testing measurement such as bulk RNA-seq A set of external RNA controls may be added to the sample. An example of a set of external RNA controls is the ThermoFisher Scientific ERCC RNA Spike-In Mix (ThermoFisher Scientific Cat. No. 4456740). A set of internal RNA reference molecules from the sample may be measured, a cell-type-specific reference molecule formed by multiple mRNA expression molecules. Examples of multiple DNA reference molecules. Multiple DNA expression reference molecules may be measured by a testing measurement such as shotgun metagenomic sequencing. A set of external DNA controls may be added to the sample. A set of internal DNA reference molecules from the sample may be measured: a fungal cell-type specific reference molecule formed by multiple DNA molecule types such as the ITS2 region and RPB2 gene; a bacterial cell-type specific reference molecule formed by multiple DNA molecule types such as the 16S gene and an antibiotic-resistance gene; a reference molecule formed by a reference DNA molecule and a reference RNA molecule (such as 16S DNA and 16S RNA).

Examples of systems that can carry out the StochQuant methods include: portable or desktop computing devices (tablets, laptops, smartphones, etc.) configured to carry out one or more embodiments of the methods by software, hardware, and/or firmware on the device configured to carry out the computational steps of the methods, including a user interface to take in inputs and display and store outputs; computer-readable non-transient mediums (disks, USB drives, memory chips, etc.) encoded with programs configured to carry out one or more embodiments of the methods when run on a computing device.

The details of one or more embodiments of the disclosure are set forth in the following examples.

### EXAMPLES

The StochQuant methods and systems of the disclosure are further illustrated in the following examples, which are provided by way of illustration and are not intended to be limiting.

In particular, in the following example of the StochQuant methods and systems of the disclosure are described in connection with exemplary testing measurements, target molecule, reference molecule, absolute anchoring measurements, physical parameters probability distributions and environment. A skilled person will understand how to adapt the guidance indicated in the examples to additional testing measurements, target molecule, reference molecule, absolute anchoring measurements, physical parameters probability distributions and environments in view of the remaining portions of the disclosure.

The following Materials and Methods were used in connection with the experiments of Examples 3 to 13.

Defined Microbial Community Dilutions. The ZymoBIOMICS™ Microbial Community DNA Standard II (Log Distribution) (Cat #D6311) was serially diluted in nuclease-free water (Sigma Cat #W4502-1L) by 25×, 2500×, 25,000×, and 250,000×. We refer to each of these defined Microbial Dilutions as MD1, MD2, MD3, and MD4 respectively. In the context of Examples 3 to 6 and 8 to 15, each of the dilutions MD1, MD2, MD3, and MD4 are the environments of interest (e.g., MD1 is an environment). Total microbial loads (absolute anchoring value of the reference molecule) in each dilution were obtained using digital PCR with universal 16S primers using the digital PCR pipeline described previously [38].

Human (tissue samples/specimens/biopsies). Remnant nucleic acids from human tissue samples were obtained from a previous study [39]. Each solution of remnant nucleic acids is considered an environment in Example 7. All activities related to enrollment of participants, collection of samples, and sample analysis were approved by the University of Chicago IRB and performed under IRB protocols #15573A and #13-1080. De-identified samples were received at Caltech and analyzed under Caltech IRB protocol #21-1083. Adults scheduled for routine colon cancer screenings via colonoscopy at the University of Chicago Medicine (UCM) were screened for diagnosis and eligibility criteria for enrollment in the study on a weekly basis. Exclusion criteria included: participants with chronic infectious diseases such as human immunodeficiency virus (HIV) or hepatitis C(HCV); active, untreated Clostridium difficile infection; active infection with severe acute respiratory syndrome coronavirus 2 (SARS-COV-2); intravenous or illicit drug use such as cocaine, heroin, non-prescription methamphetamines; active use of blood thinners; severe comorbid diseases; participants on active cancer treatment; and participants who were pregnant. Approaching prospective participants was at the discretion of their treating physician and was not done in cases that would put participants at any increased risk, regardless of reason. Participants were approached the day of their procedure and informed, written consent was obtained before any samples were acquired.

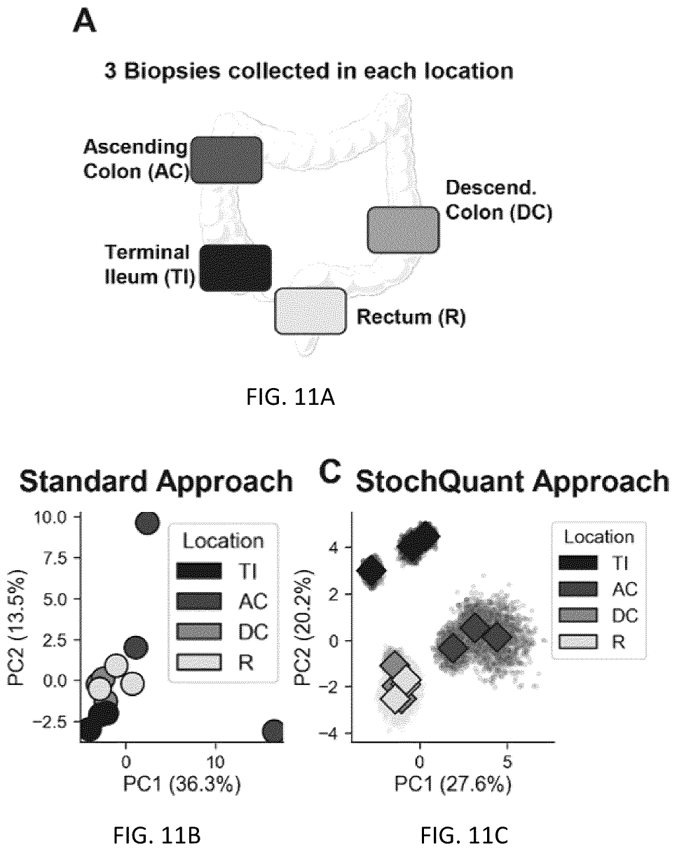

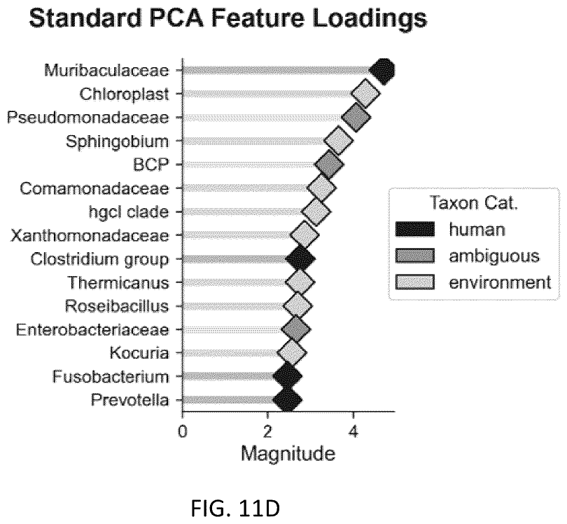

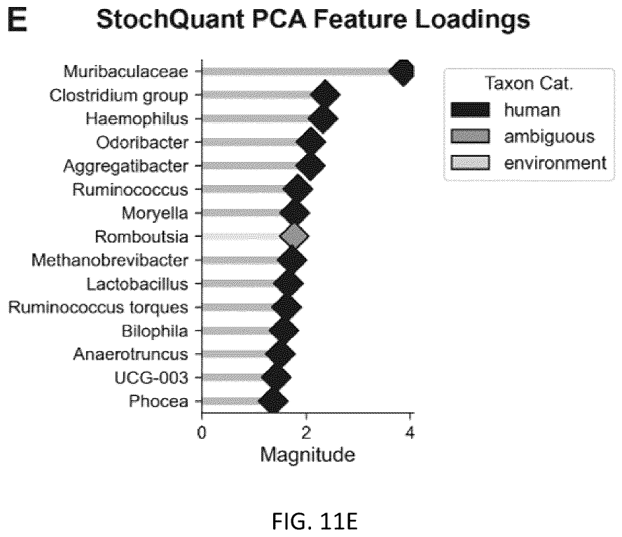

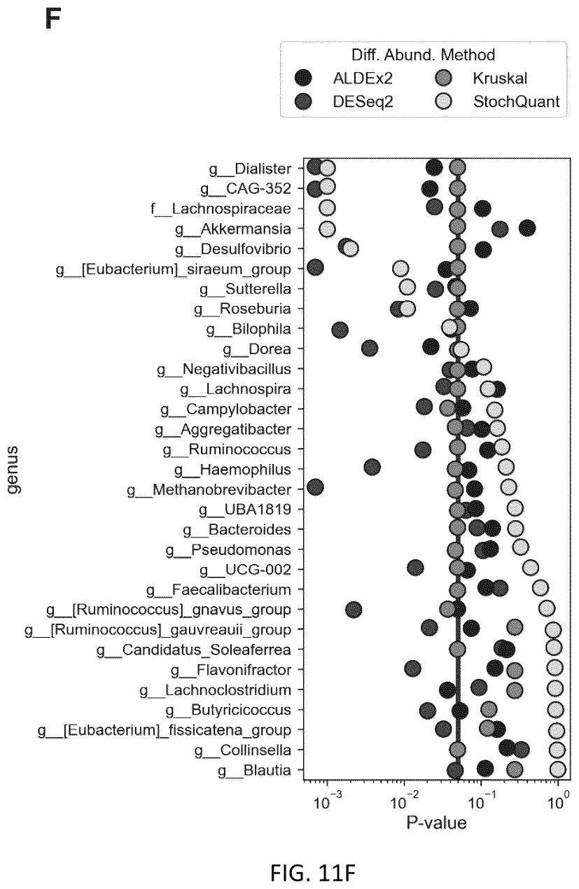

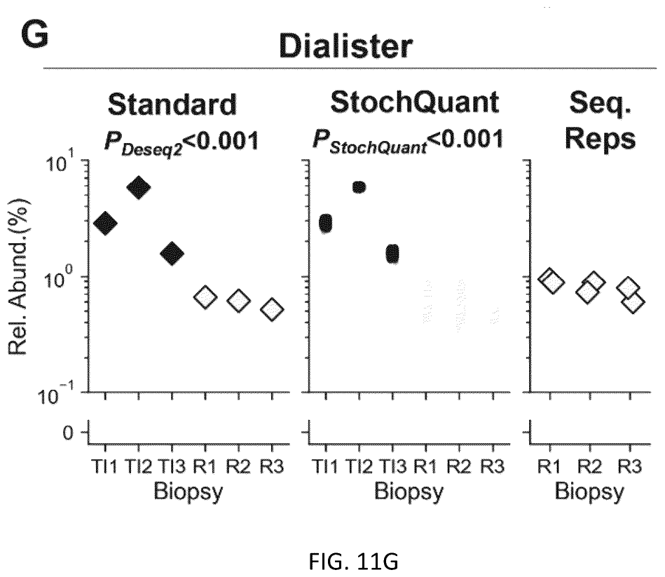

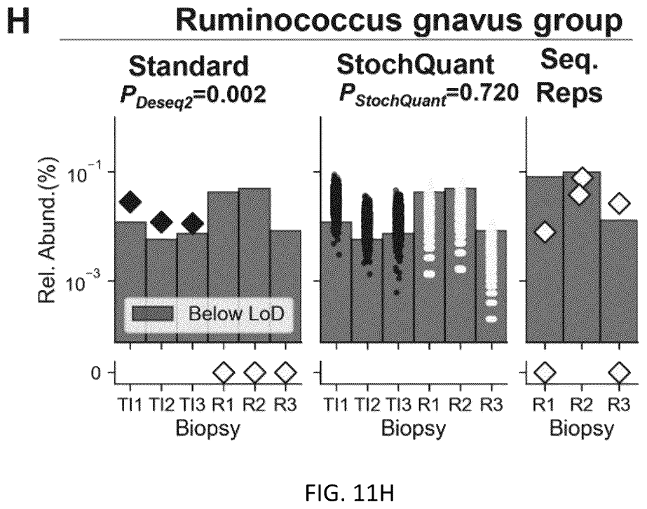

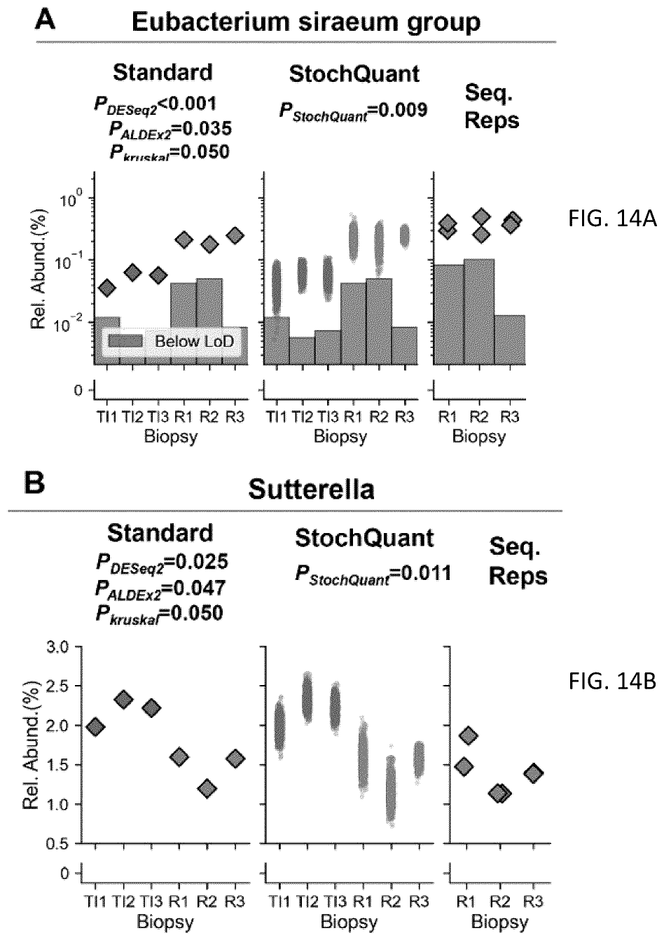

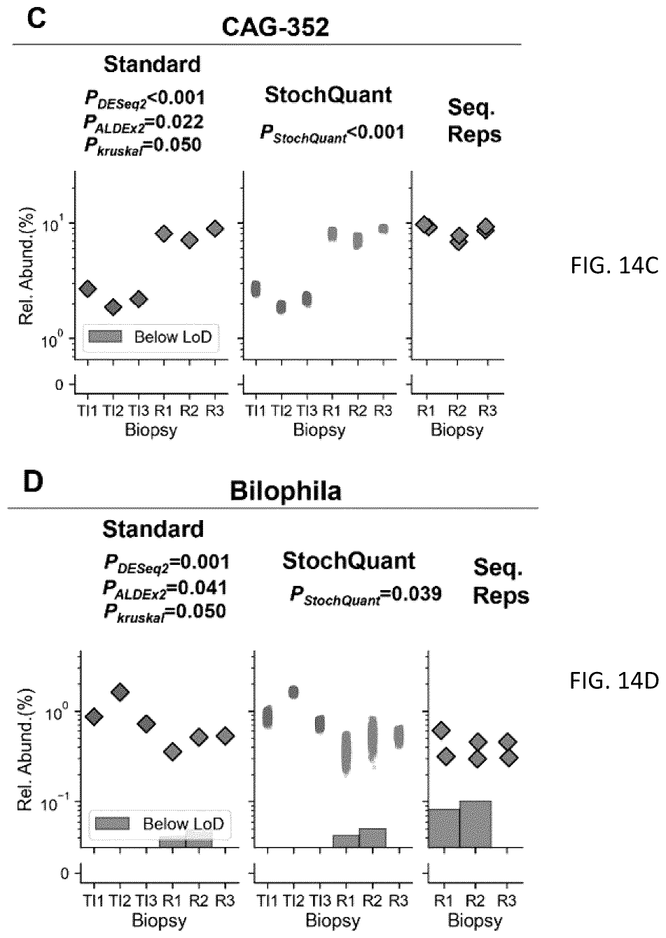

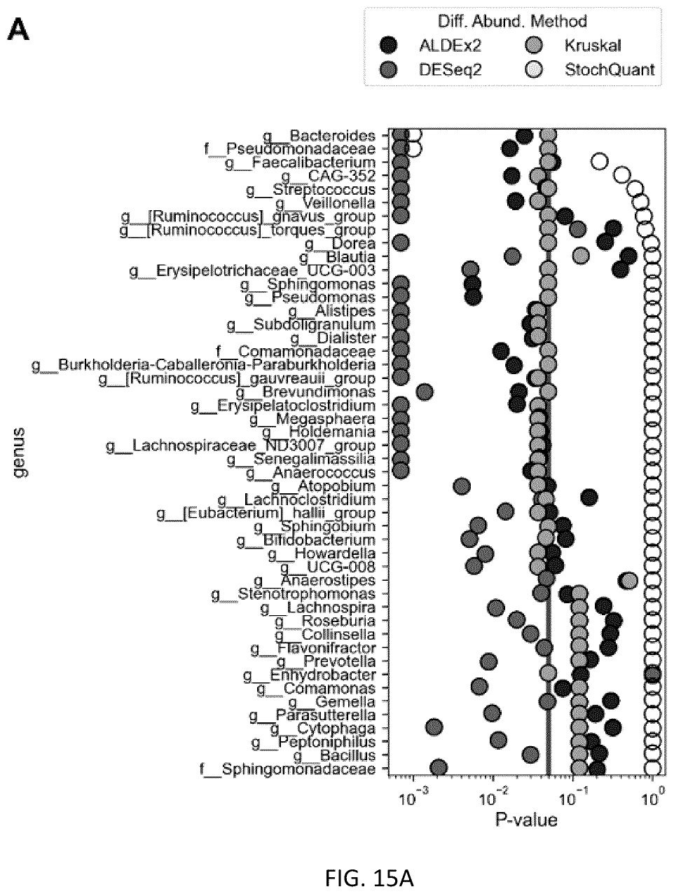

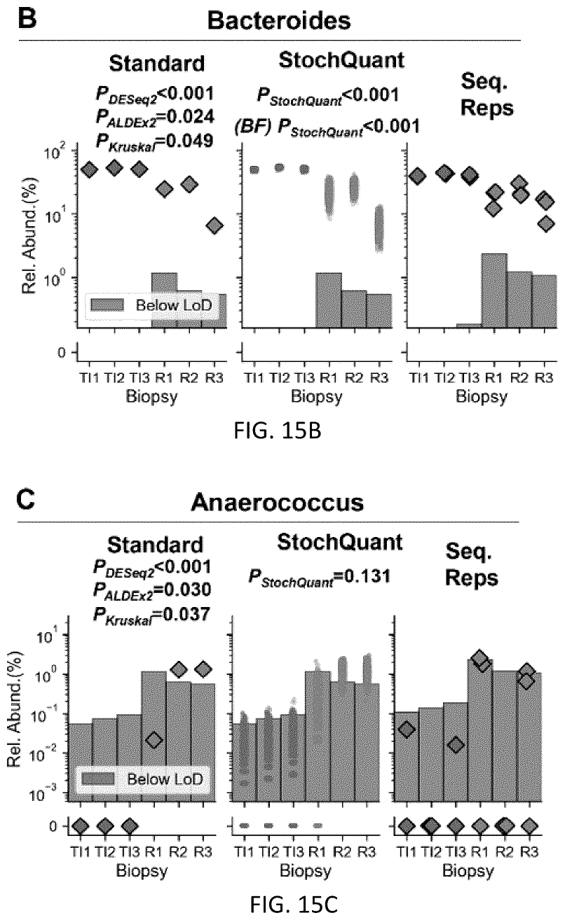

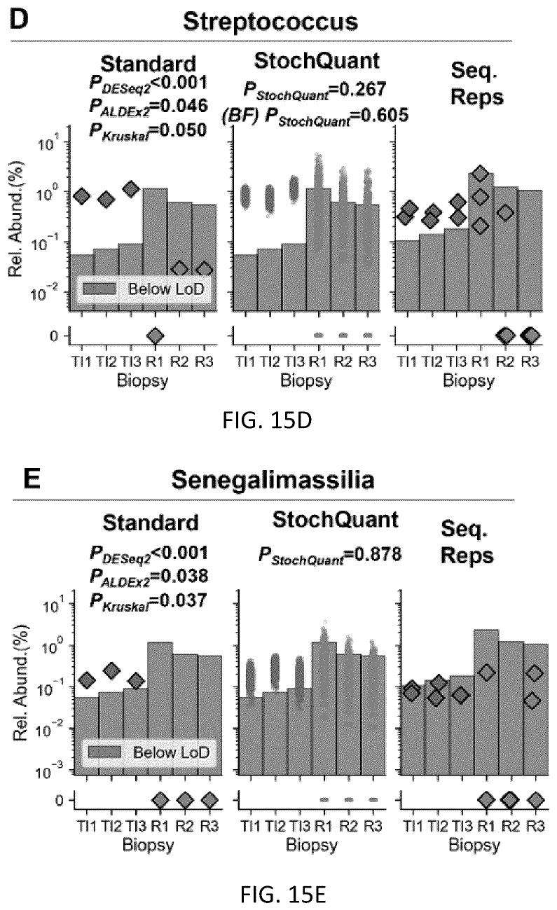

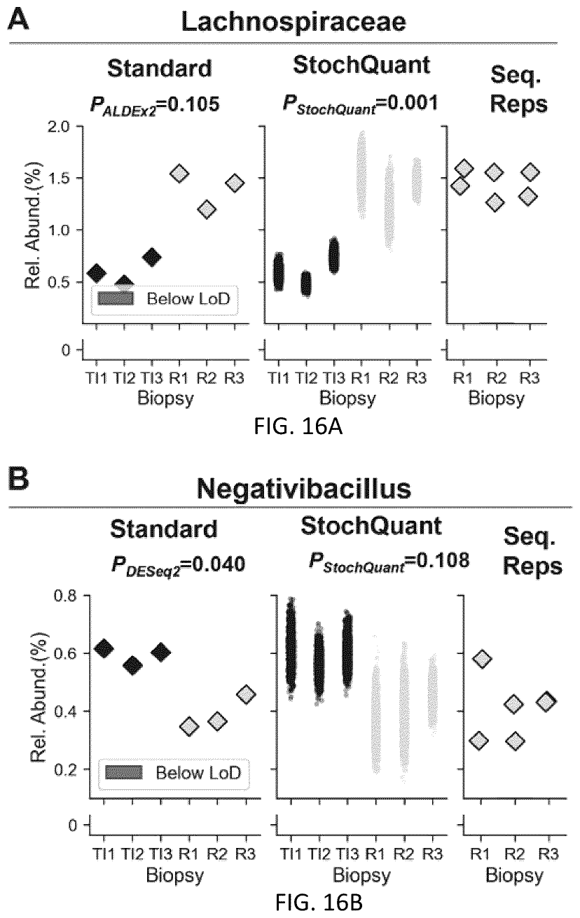

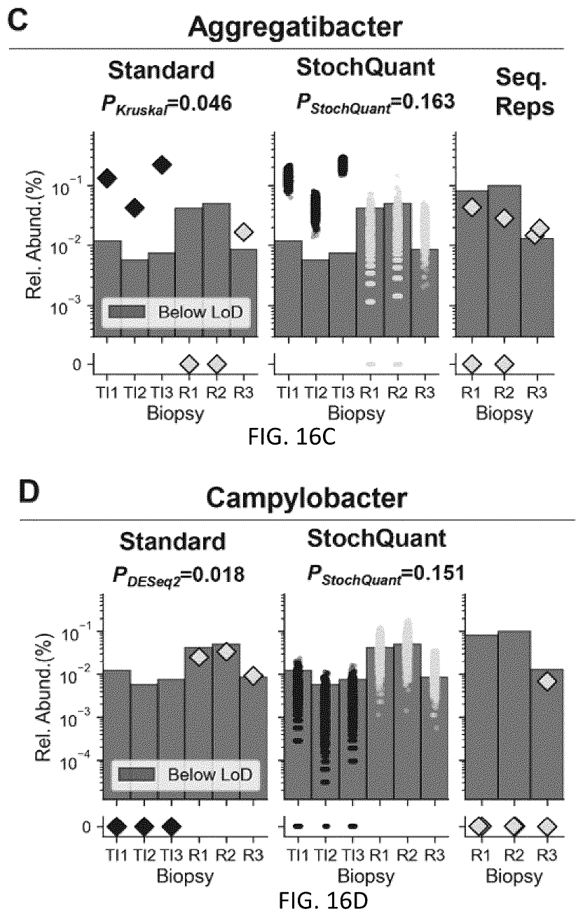

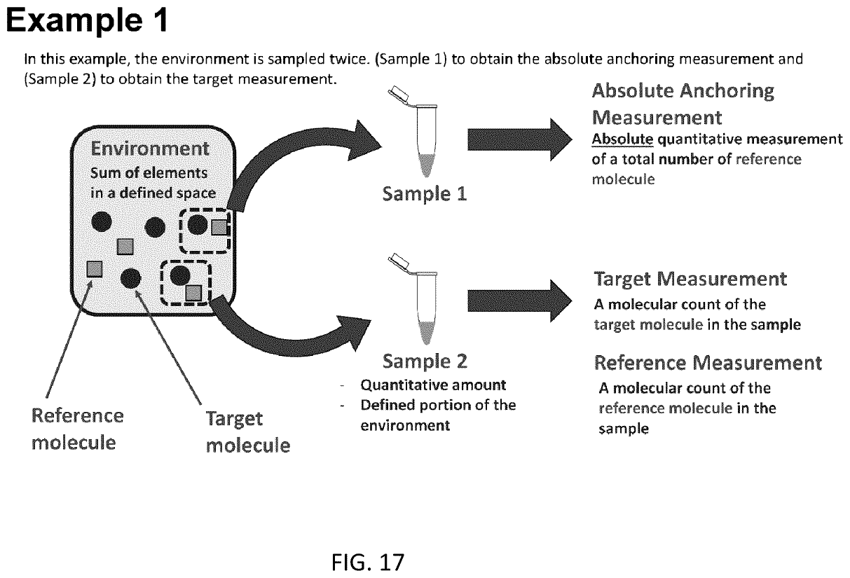

16S rRNA gene Sequencing Library Preparation (Testing Measurement yielding a molecular count of target molecules and molecular count of reference molecules). The sequencing library for the defined microbial community was prepared as previously described [39], with the exception that 2 μL of template were used as inputs into the library preparation reaction, and library preparation reactions were performed in singlicate. Accordingly2 μL of sample were separated from a dilution (environment). With the exception of the sequencing re-runs (described in FIGS. 11A-H, FIGS. 14A-D, FIGS. 16A-D, FIGS. 15A-E), sequencing data and total load measurements (absolute anchoring value of the reference molecule) from the human biopsy samples were previously obtained [39]. The sequencing library for the re-sequenced biopsy samples was prepared as previously described [39], with the exception that 5 μL of template were used as inputs into the library preparation reaction. In particular, according to the cited reference, Extracted DNA was amplified and sequenced using barcoded universal primers and protocol modified to reduce amplification of host DNA. The variable 4 (V4) region of the 16S rRNA gene was amplified in triplicate with the following PCR reaction components: 1×5Prime Hotstart mastermix, 1× Evagreen, 500 nM forward and reverse primers. Input template concentration varied. Amplification was monitored in a CFX96 RT-PCR machine (Bio-Rad) and samples were removed once fluorescence measurements reached ˜10,000 RFU (late exponential phase). Cycling conditions were as follows: 94° C. for 3 min, up to 40 cycles of 94° C. for 45 s, 54° C. for 60 s, and 72° C. for 90 s. Triplicate reactions that amplified were pooled together and quantified with Kapa library quantification kit (Kapa Biosystems, KK4824, Wilmington, MA, USA) before equimolar sample mixing. Libraries were concentrated and cleaned using AMPureXP beads (Beckman Coulter, Brea, CA, USA). The final library was quantified using a High Sensitivity D1000 Tapestation Chip. Sequencing was performed by Fulgent Genetics (Temple City, CA, USA) using the Illumina MiSeq platform and 2×300 bp reagent kit for paired-end sequencing.”

16S rRNA gene amplicon data processing. Raw sequencing data was processed as previously described [39] with the exception that an updated version of QIIME2 (v 2023.2) was used. All datasets were collapsed to the Phylum, Class, Order, Family, and Genus levels, and downstream analyses were primarily performed at the Genus level. In particular, data was processed to yield molecular counts of targets and reference, such that sequences that were identified to be similar to each other at the genus-level were considered to be the same target sequence. In this connection according to the cited Ref. “Processing of all sequencing data was performed using QIIME 2 2019.1 [41]. Raw sequence data were demultiplexed and quality filtered using the q2-demux plugin followed by denoising with DADA2 [42]. Chimeric read count estimates were estimated using DADA2. Taxonomy was assigned to amplicon sequence variants (ASVs) using the q2-feature-classifier classify-sklearn naïve Bayes taxonomy classifier against the Silva 132 99% OTUs references from the 515F/806R region. All datasets were collapsed to the genus level before downstream analyses.”

Total bacterial load quantification with digital PCR._Total bacterial loads (absolute anchoring value of the number of reference molecules) were quantified using digital PCR, as previously described [38].

Data analysis visualization. Data were analyzed with Python scripts using Pandas and Numpy. Plots were generated in Python using Matplotlib and Seaborn.

All StochQuant specific methods were implemented via Python functions. Unless specified otherwise, all sampling from distributions was performed using the Numpy random number generator, with the specified functions and parameters described below.

### Example 1: StochQuant Approach for Detections Requiring Counting Molecule and Stochastic Inference

Many determination concerning features of environments of interests, are performed through detection of a molecule. For example, microbes with low-to-moderate biomass play key roles in ecosystems, agricultural biotechnology, and human health and microbes can be detected through detection of molecular markers such as 16S RNA. However, characterization of microbes in such samples with current methods is often irreproducible because current approaches struggle to reliably and reproducibly, detect and quantify microbes, differentiate them from background contamination, and determine which microbes are differentially abundant among samples.

Current detection methods present key challenges in detecting molecular counts in particular with respect to molecules present in low abundance, because they do not factor in, changes in molecular counts introduced by the manipulation of the molecules of interest in an environment (e.g. sampling, extractions, library constructions, sequencing and others) required by the detection method.

These key challenges can be addressed through a StochQuant approach which allows identification of i) one or segments of a detection workflow which forms part of a testing measurement in which the activities required to perform the detection result in a change of the molecular count of target molecules thus introducing stochasticity, and ii) physical parameters of the testing measurement workflow that affect the molecular count of the target) which parameterize the probabilistic mathematical representations to account for the stochasticity which can be detected and used to select probability distributions that are representative of the changes in molecular counts introduced by the testing measurement and thus account for the stochasticity. The physical parameters (StochQuant parameters) are used to provide stochastic representation of the segment and of the workflow in accordance with the StochQuant approach of the disclosure, which in turn provide a probability distribution which informs the user of impact on the molecular count introduced by the detection process as will be understood by a skilled person

The probability distribution of target abundance in an environment identified by StochQuant allows a user to identify confidence intervals of target molecule abundances, the interval giving a confidence level, which can be calculated based on the probability distribution of target molecule abundances affected by stochasticity

Providing detected values with confidence level of detection allows one of skill to obtain a more reliable detection of the corresponding feature of the environment. The confidence level obtained through StochQuant improves any determination performed based on the detected values of the physical parameters. For example, StochQuant detection of 16S rRNA markers of microorganisms allows a more reliable and reproducible quantification of the microorganisms in an environment, which in turn can be used to diagnose a condition and/or to decide action to modify the environment in line with clinical, medical and/or experimental design as will be understood by a skilled person.

Experiments performed through the development of StochQuant demonstrate that experimentally tracking absolute (rather than relative) numbers of molecules throughout the entire detection pipeline is necessary and sufficient to overcome these limitations of methods involving detection of a number of target molecules such as current amplicon sequencing methods used here to provide a proof of principle.

The following Example 2 provides an exemplary illustration of a StochQuant workflow and related us in connection with confidence intervals and confidence levels.

Examples 3 to 15 provide a proof of principle for the workflow of Example 2 using amplicon sequencing as detection method.

Examples 3 to 15 show that StochQuant amplicon sequencing combines a testing measurement of molecular count of target molecules such as the (16S RNA of a taxon) and reference molecules (16 S RNA of all taxa) performed by a sequencing measurement with an absolute anchoring measurement of the total number of target molecules in a sample. Then, these testing measurements and anchoring measurement (the Physical parameters) are used with a StochQuant Representation of the testing measurement to yield probability distributions of the absolute abundance of the target molecule in the environment of each microbial taxon thus allowing a determination of the absolute abundance of each taxon.

In particular, Examples 3 to 15 demonstrate in a defined microbial community and human biopsies that accounting for stochastic sampling of absolute numbers of molecules dramatically improves downstream analyses including contamination filtering, principal component analysis, and differential abundance analysis. While Examples 3 to 15 validated and showed a proof-of-concept example of StochQuant with microbial 16S rRNA gene amplicon sequencing, the StochQuant detection workflow, which uses this combination of absolute quantification measurement of a reference molecule, a testing measurement to yield a molecular count of a target molecule and a molecular count of a reference molecule, and obtaining a measurement workflow representation (stochastic modeling of the testing measurement) t can improve reliability of other sequencing pipelines as well as of other detection methods that involve the detection or analysis of numbers of molecules, in particular when the number is small and stochasticity is usually higher, such as shotgun metagenomic sequencing and RNA sequencing.

Additional examples in this connection are provided by Examples 21 to 48 where additional testing measurement are exemplified together with exemplary process for the related StochQuantization.

Example 16 to 20 show exemplary embodiments, where testing measurements and/or anchoring measurement for a biological environment are detected in samples or sub-samples of the environments.

### Example 2: StochQuant Workflow and Related Use in Connection with Confidence Intervals and Confidence Levels

StochQuant approach can be applied to any base detection method which result in detection of molecular counts according to the schematic illustration of FIG. 1.

In the exemplary illustration of FIG. 1 the StochQuant workflow is divided in two set of activities: “Build StochQuant Workflow” and “Use StochQuant Workflow” which can be performed by a same user or different users identified in the following examples as user 1 (Build StochQuant Workflow) and user 2 (Use StochQuant Workflow).

In the schematic of FIG. 1, the “Build the StochQuant Workflow” comprise the following steps: (1) Identify the target molecule of interest and a testing measurement that yields a molecular count of the target molecule. (2) Identify the manipulations of the molecule(s) of interest in a testing measurement workflow. (3) Select at least one reference molecule and selecting a method to perform an absolute anchoring measurement to obtain an absolute anchoring value of the reference molecule. (4) Build the Measurement Workflow Representation which involves (a) identifying measurement workflow representations, (b) performing segmental calibration for each segment, and (c) assessing the accuracy of the measurement workflow representation. It can be understood that the testing measurement representation and the one or more segments of the representation corresponding to the manipulations of the target molecule activities performed by the base detection method.

A testing measurement in the sense of the disclosure indicates a quantitative detection performed through detection of a feature of a tested molecule which provides a molecular count. In particular, molecular count can be performed by detection of structural features such as sequence of polynucleotide (typically DNA and RNA) or polypeptides (typically proteins or peptides) spatial conformation of the molecule resulting in specific binding of antibodies, and generation of specific mass spectrum which can be used to perform the count. The structural feature(s) that are detected can comprise features of the target/reference molecule in the environment and/or features of the target/reference molecule that are present due to manipulations. For example, in the 16S rRNA amplicon sequencing example, detection is performed through detection of a nucleic acid sequence that includes (i) part of the nucleic acid sequence of the target molecule in the environment and (ii) a nucleic acid sequence (a sequencing adapter sequence and a “barcode” sequence) that is added to the target due to a manipulation (during library preparation).

In particular, according to the StochQuant workflow it will be understood that a testing measurement is performed on target molecules and reference molecules within an environment to obtain a corresponding molecular count which is then used in combination with an absolute anchoring value to StochQuantize the base the detection method.

As will be understood by a skilled person in an exemplary Stoch Quant workflow

The target molecule is molecule of interest;
A reference molecule is molecule that can be detected, providing a molecular count, with the same testing measurement applied to the target molecule and that can be measured with an absolute anchoring measurement and/or can be added in a known number of molecules. The reference molecule can be a molecular of a same type of the molecule of interest;
An Environment such as biological environment can be a complex of physical, chemical, and biotic factors that act upon an organism and/or an ecological community and ultimately determine its form and survival such as soil, and living things;
An absolute anchoring value can be provided by a total number of a reference molecule in the biological environment;
A molecular count of the target molecule is molecular count obtained through quantitatively detected values of the feature of the target molecule measured through the testing measurement (such as a read count yielded from sequencing);
A Molecular count of the reference molecule is molecular count obtained through quantitatively detected values of the feature of the reference molecule measured through the testing measurement (such as a read count of total number of yielded from sequencing of a reference molecule).

As illustrated in the schematics of FIG. 1 in a StochQuant Workflow once the manipulation of the molecule of interest that occur during the testing measurement is identified, a measurement workflow representation can be identified which can approximate the number and variability in molecular counts yielded by the testing measurement for a given number of molecules in an environment.

More than one measurement workflow representation can be identified that approximates the number and the variability in molecular counts of the target molecule obtained via the testing measurement. Selection of an appropriate measurement workflow representation can depend on the user's choice. Exemplary factors that can impact the user's choice can include: the measurability of a manipulation or series of manipulations of the testing measurement, the representability of a manipulation or series of manipulations of the testing measurement, the desired accuracy of the measurement workflow representation, and the computational requirements (e.g., computational time and space) to yield the probability distributions of target abundance from the measurement workflow representation and the physical parameters. The measurement workflow representation can then be incorporated into an inference method, so that the inference method uses the measurement workflow representation and the physical parameters to yield a probability distribution of target abundance in an environment.

The selected inference procedure with the measurement workflow representation can then be used in the steps of the “Use the StochQuant Workflow” section of FIG. 1 by a user who will perform the base detection method and the absolute anchoring measurement while to detect the 1) the molecular count of the target molecule; ii) the molecular count of the reference molecule; and iii) the absolute anchoring value of the reference molecule (physical parameters) obtained in outcome of the testing measurement.

The user can then StochQuantize the base detection method by inputting the detected physical parameters into the inference method with the measurement workflow representation of the StochQuant workflow to obtain a probability distribution of target abundance in an environment

Steps described in the instant application and exemplified in this section can then be performed to identify a confidence level for a user-specified confidence interval or to identify a confidence interval for a user-specified confidence level, to be provided to the user alone or in combination with each other and/or with the probability distribution as will be understood by a skilled person.

As illustrated in the FIG. 2 Panels A and B, the confidence interval shown is set to +/−n around the peak value (the value of n is pre-determined, but the peak value will not be known until the resulting curve is known). The area under the curve from (peak-n) to (peak+n) is the confidence level that the true value is within that confidence interval.

As also illustrated in FIG. 2 Panels A and B, in addition or in the alternative a user can identify the set of values which are above or below a set threshold e.g. a threshold provided for diagnostic purposes

### Example 3 Rigorous and Reproducible Amplicon Sequencing of Bacteria with Low-to-Moderate Abundance can be Used to Provide a Proof of Principle for StochQuant

Analyses of samples with low microbial biomass are key to many areas of science, medicine, and biotechnology. Microbes-even at low absolute or relative abundances—can play a critical role in the health and disease of humans [38, 39, 45-56], marine ecosystems [57], and soil [58, 59], and can be informative diagnostic markers [60-64]. However, analysis of such samples is challenging. For example, as research has advanced into samples with lower microbial loads, evidence has emerged for the presence of microbes in human samples that were previously believed to be sterile, including the placenta [47, 65-67], blood [61], breastmilk [68, 69], fetal lung [70], and cancerous tumors [45, 71, 72]. But some of these studies have been challenging to reproduce [45, 66, 71-77]. However, some of these studies have been challenging to reproduce [66, 73-80]. sparking debates over whether such sites are truly inhabited by microbes or whether at least some of these results are artifacts (such as contamination) of applying current experimental and computational microbiome analysis pipelines [66, 73-77] to these low microbial biomass samples.

To advance these and other areas of microbiome research [38, 45-54, 57-64] there is an unmet need to (1) reliably and reproducibly identify and quantify microbes in samples with low-to-moderate microbial biomass, and then (2) differentiate these microbes from background contaminants and determine whether a particular microbe or group of microbes are differentially abundant between two or more conditions (e.g., control vs treated, healthy vs disease, location A vs location B).

Here, reference is made to low-abundance taxa as taxa that are detected with less than 95% probability, and refer to moderate-abundance taxa as taxa for which quantification gives rise to more than ˜2×-3× variability (measurement noise).

One cause for the lack of reproducibility in microbiome studies involving low-to-moderate microbial biomass is the inability to reliably remove contaminant DNA and sequencing artifacts from downstream analyses using existing bioinformatics approaches. Contaminant microbial DNA introduced during sample handling and artifacts generated during PCR and sequencing are known to lead to false conclusions and decrease the statistical power in downstream analyses [73, 79, 81]. Excellent experimental and computational approaches have been developed to remove contamination [48, 78, 81-83], including computationally filtering using prevalence thresholds (e.g., minimum number of samples in which a taxon is be detected), total read count thresholds (e.g., minimum number of reads across all samples of a given taxon), and relative abundance thresholds [82, 83]. [82, 83]. [82, 83]. However, none of the current approaches effectively remove sequencing artifacts and contaminants from low biomass samples while robustly preserving biological features [84]. [84]. [84]. Instead, in many cases, key biological features are inadvertently removed from datasets, while contaminants and artifacts are kept [78].

Accordingly, there is an issue of confidence in the determination of whether a 16S rRNA gene target molecule is present in an environment in an amount that is greater than the background contamination levels. Depending on the context, a user can choose to require more confidence that a target is greater than the background contamination levels to make the determination, and in other contexts, a user may choose to require less confidence that a target is greater than the background contamination levels to make the determination.

A second cause for the lack of reproducibility is the intrinsic inability to robustly detect and quantify microbes present at low-to-moderate relative or absolute abundances, which in turn challenges the ability to perform differential-abundance analyses. Several state-of-the-art software packages such as DESeq2 and ALDEx2 have been developed to take into account the discrete, compositional [85], and sparse [86] nature of sequencing data. These packages utilize several normalization methods, statistical tests, Bayesian approaches, and mathematical modeling to accommodate the often zero-inflated and over-dispersed nature of sequencing data. Yet, in practice, even state-of-the-art methods often perform poorly and variably [87-89]. [90] and ALDEx2 [87-89] have been developed to take into account the discrete, compositional [85], and sparse [86, 90] nature of sequencing data. These packages utilize several normalization methods, statistical tests, Bayesian approaches, and mathematical modeling to accommodate the often zero-inflated and over-dispersed nature of sequencing data. Yet, in practice, even state-of-the-art methods often perform poorly and variably [87-89]. Several benchmarking studies have shown that the choice of data pre-processing method and differential-abundance method substantially impacts the conclusions one draws from a given dataset, and that the performance of each method greatly varies among datasets [90]. Furthermore, in most cases, each method results in unacceptably high false discovery rates (FDR), in which a taxon is determined (with statistical significance) to be differentially abundant between two conditions, even though it in fact is not [87-89]. It remains unclear why some data pre-processing methods and differential-abundance methods perform well in some contexts, but not in others [87-89, 91, 92]. Accordingly, there is an issue of confidence in the determination of whether a different amount of target is present in an environment, a sample and/or a subsample thereof.

In this example, a proof of concept of the StochQuant method is provided in the context of 16S rRNA gene sequencing. In this example, a target molecule is selected and a corresponding reference molecule that are both detectable via the same testing measurement. An absolute anchoring value of the reference measurement is also obtained via digital PCR. Accordingly in the proof of concept of this examples StochQuant detection is performed with the following features
Testing measurement: 16S rRNA gene sequencing (amplicon sequencing)
Target molecule: 16S rRNA gene sequence of a particular microbe of interest (e.g., Bacillus).
Reference molecule: 16S rRNA gene sequences of all microbes
Environment: A solution of isolated nucleic acids also referred as the dilutions MD1, MD2, MD3, and MD4.
Absolute anchoring value: total number of 16S rRNA gene copies in a solution of nucleic acids. This solution is the environment from which (i) a sample is taken to perform the absolute anchoring measurement (which is digital PCR measurement of total number of 16S rRNA gene copies) and (ii) another sample is taken to perform amplicon sequencing (testing measurement).
Molecular count of the target: a read count of number of 16S reads from the target microbe of interest yielded from the testing measurement (amplicon sequencing)
Molecular count of the reference: a read count of the total number of 16S reads yielded from the testing measurement (amplicon sequencing). This value is also commonly referred as the “read depth” to refer to total number of sequenced 16S reads in a sample.

In this proof-of-concept example, a measurement workflow representation is identified of an amplicon sequencing testing measurement which provide a StochQuant method workflow (discussed in detail below). In doing so, segments of the measurement workflow representation are identified. Each segment uses probability distribution(s) and physical parameters to parameterize the distribution(s) to enable tracking of the probable numbers of output molecules for a given number of input molecules of a manipulation or series of manipulations of the target/reference molecule (discussed in more detail below). Then, through the validation of the proof-of-concept workflow, the central hypothesis was tested that the seemingly irreproducible amplicon sequencing results obtained from samples with low-to-moderate abundance microbes emerge from stochastic noise introduced through sequencing based quantification and detection, and this process can be mathematically described via a forward measurement model (exemplary measurement workflow representation). This example model uses experimentally observable parameters (or physical parameters) that affect the molecular count of the target/reference molecules to model amplicon sequencing as a series of stochastic Poisson processes. The model tracks absolute numbers of molecules moving through the sequencing pipelines, which is in contrast to the compositional (relative abundance) data commonly used in microbiome analyses.

Recent advances in quantitative sequencing [38] enable a user to perform an absolute anchoring measurement to yield an absolute anchoring value of the reference molecule (e.g., the total number of 16S rRNA gene molecules via a digital PCR measurement with universal 16S primers) to test this hypothesis and develop a proof-of-concept example of StochQuant, a combined experimental and computational approach that utilizes a Measurement Workflow Representation (forward measurement model) to derive a probabilistic relationship between the number of taxon 16S rRNA gene molecules in a sample and molecular counts (also referred to as read counts) of the target and reference molecules obtained from the amplicon sequencing testing measurement. Note, throughout this example, the term “taxon” is used for brevity to refer to target 16S rRNA gene molecules of a taxon according to common use.

The forward measurement model uses an absolute measure of total microbial load (e.g., an absolute anchoring value of the reference molecule), experimentally used sample volumes (in this example, these are the Physical parameters used to parameterize the probability distributions of each Segment), sequencing read depth (the molecular count of the reference molecule), and Poisson statistics (the type of probability distributions used to track the number of probable output molecules yielded in a Segment) to generate simulated read counts from known taxon abundances. Thus, this forward measurement model is a mathematical representation of the testing measurement workflow that enables tracking of the probable molecular counts of the target molecule yielded via the testing measurement for a given number of input target molecules. This forward measurement model can mathematically explain why sequencing low-to-moderate-abundance microbes will intrinsically result in seemingly unreliable and irreproducible detection and quantification.

Furthermore, this example of the StochQuant approach uses this forward measurement model to estimate probability distributions of taxon abundance (relative or absolute) from observed read count data and other quantitative data. These distributions are then leveraged to improve the analysis of sequencing data that rely on accurate detection, quantification, and estimation of measurement noise, including contamination filtering and differential abundance.

Accordingly, these probability distributions of the number of target molecules in an environment are used to obtain a measure of confidence in a detection or quantitative detection of a target molecule. Then, a determination is made based on the confidence of the detection or quantitative detection of a target or targets. It is demonstrated through a proof-of-concept example describing the development of StochQuant that, at least in the context of our study, experimentally and computationally tracking absolute (rather than relative) numbers of molecules throughout the entire sequencing pipeline was necessary and sufficient to overcome the limitations of current methods. It isfurther demonstrate in multiple environments (a defined microbial community and human biopsies) that accounting for stochastic sampling of absolute numbers of molecules dramatically improves downstream analyses-including contamination filtering, principal component analysis, and differential abundance analysis.

### Example 4 High Variability of Results is Intrinsic to Standard Amplicon Sequencing Approaches and Samples with Low-to-Moderate Bacterial Loads

To first illustrate why current state-of-the-art sequencing approaches perform inconsistently, a 16S rRNA gene sequencing experiment was performed with serial dilutions of a defined microbial community and processed and analyzed the data with several existing approaches (FIG. 3A). Briefly, the log-distributed defined community were serially diluted to total microbial loads of 4.2×10⁸, 4.8×10⁶, 4.2×10⁵, and 4.9×10⁴ DNA copies/mL of the 16S rRNA gene (“microbial dilutions” MD1, MD2, MD3, and MD4 respectively) and sequenced 20-22 technical replicates of each dilution 4 replicates of a no-template control were additionally sequenced (NTC; a processing blank that undergoes similar processing steps and uses the same reagents as the biological samples, except no biological sample is added).

Here, each of the dilutions (MD1, MD2, MD3, and MD4) and the NTC is an environment containing numbers of target molecules (16S rRNA gene molecules, where the 16S rRNA gene sequence of a particular taxon is a target molecule).

Then, to emulate a commonly used experimental design, 3 sequencing replicates were repeatedly computationally selected at random from each dilution and 1 sequencing replicate of the NTC, and these data were analyzed with existing methods. The results of the analysis are reported in FIG. 3B-E.

In particular, the analysis for FIG. 3B was performed as follows. For each comparison between dilution conditions (MD1 vs MD1, MD1 vs MD2, MD1 vs MD3, and MD1 vs MD4), 3 sequencing replicates from each condition were randomly selected without replacement with the numpy.random.choice function. For the MD1 vs MD1 condition, 6 sequencing replicates were randomly selected without replacement, and then the 6 replicates were split into two groups (of 3 sequencing replicates each) at random. Each time sequencing replicates were randomly selected, downstream differential abundance analysis (described below) was performed. This procedure was repeated 100 times.

Two examples are shown in FIG. 3C that demonstrate some of the largest observed differences in mean Pseudomonas relative abundance between 3 sequencing replicates of MD1 and MD4.

To generate FIG. 3D, taxa that were detected in any of the 4 NTC sequencing replicates were sorted by maximum observed relative abundance. The top 10 taxa were then plotted in each of the 4 NTC sequencing replicates, and the sum of all other taxa in each replicate were summed and labeled as “other”. Differential abundance analysis was performed with DESeq2, ALDEx2, and the Kruskal-Wallis statistical test. Briefly, Kruskal-Wallis was performed in a Python script using the scipy.stats package. The R package of DESeq2 was used with default settings. The R package of ALDEx2 was used with default settings.

The analysis for FIG. 3E was performed as follows. To generate a trial, 3 sequencing replicates from each dilution (MD1, MD2, MD3, and MD4) were randomly selected without replacement with the numpy.random.choice function. For each trial, principal component analysis (PCA) was performed on the pseudo center log transformed (CLR) relative abundances with the sklearn.decomposition package. This procedure was performed 100 times.

In the context each set of randomly selected samples are referred as a “trial”. Also in the experiments of the present example, each sequencing replicate is a sample of an environment because a portion of the environment is separated from the environment.

In the experiments of the present example these trials were used to compare the FDR (rate at which a taxon is incorrectly determined to be differentially abundant between two conditions) of three differential abundance approaches (DESeq2, ALDEx2, and Kruskal-Wallis) for each of the top 5 defined community taxa (0.03-97% relative abundance) (FIG. 3B). Relative abundance is a commonly used terminology in 16S rRNA gene sequencing to refer to either (a) the amount of target 16S rRNA gene relative to the amount of total 16S rRNA or (b) the molecular count of a target 16S rRNA relative to the molecular count of the total 16S rRNA gene molecules (also referred to as read depth). It is apparent that here, the total 16S rRNA gene molecules is the reference molecule. When total microbial load between both groups was high (MD1 vs MD1), the FDR remained low (0-14%) among the three methods for all defined community taxa. However, when one group contained low total microbial loads (MD1 vs MD4), the FDR increased to as high as 100% for some taxa (FIG. 3B). Because larger measurement noise occurs at low total loads (MD4), Pseudomonas (1.9% abundance) could yield a ˜2.3-fold decrease or ˜2.5-fold increase in mean relative abundance compared to MD1 in different trials (FIG. 3C). These results highlight the problem of confidence molecular detection with particular reference to detection of target molecules in low or moderate abundance as will be understood by a skilled person.

The experimentally observed community composition of an NTC varied considerably among sequencing replicates (FIG. 3D). In total, 55 contaminant genera were detected in this NTC sequencing experiment, yet only a small subset (6 to 15) were detected in any given NTC replicate. Therefore, the results of filtering and downstream analyses that use a single sequenced NTC to identify contaminants will vary considerably. These results highlight the problem of confidence molecular detection introduced by manipulations of the detection procedure as will be understood by a skilled person.

Finally, to illustrate this high degree of variability among low-to-moderate microbial load samples, PCA was performed on the center-log-ratio (CLR) transformed relative abundance data from (n=100) trials, and found that the clustering (or lack thereof) of samples in PC space by dilution varied substantially among trials (FIG. 3E). These results highlight the problem of confidence in molecular detection of target molecules in low abundance as will be understood by a skilled person.

### Example 5 Variability of Amplicon Sequencing Results can be Predicted Via a Forward Measurement Model

In order to provide a model of the amplicon sequencing as a representative testing measurement, first, the steps and segments of the measurement workflow representation which provide a StochQuant method workflow. For each Segment, the probability distribution and physical parameters were identified to parameterize the distributions to enable tracking of the probable numbers of output molecules for a given number of input molecules of a segment.

Then, in order to test the hypothesis that the variability of amplicon sequencing results can be addressed by StochQuant, the accuracy of the measurement workflow representation was verified and validated through an exemplary forward measurement mode) of the proof-of-concept example of StochQuant reported in Example 5.

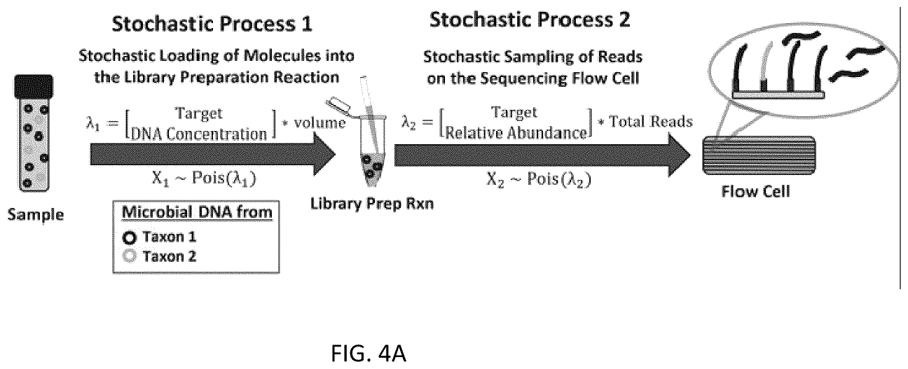

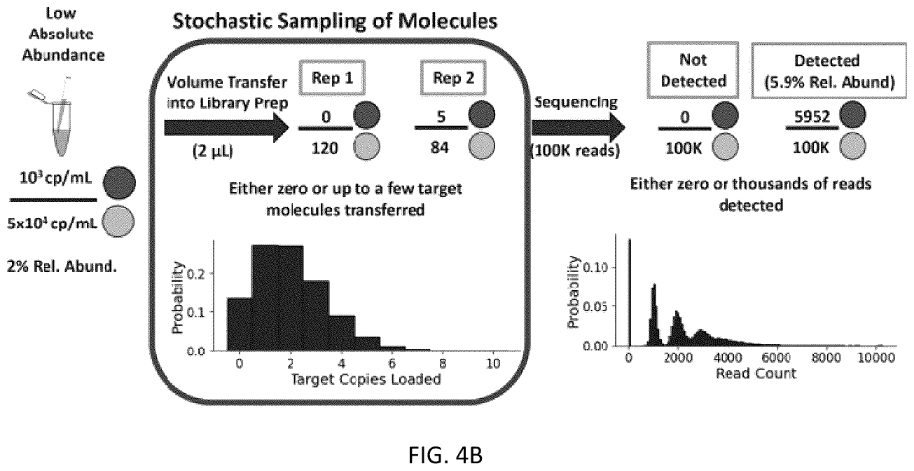

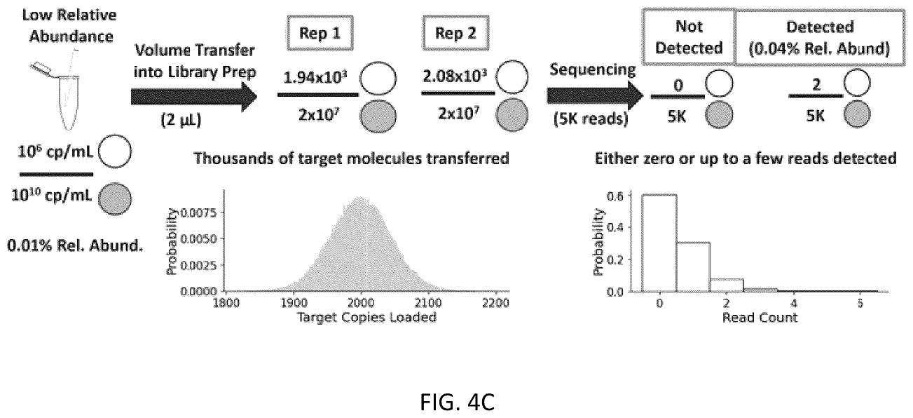

The results are reported in FIGS. 4A-C, which shows results of simulations performed using the StochQuant function simulate_loading_and_sequencing. Histograms were plotted using the Seaborn histogram function. For FIG. 4B, the following parameters were used: abs_abund=1, total_load=50, template_input_vol=2, read_depth=100,000, n_iters=10⁵. For FIG. 4C, the following parameters were used: abs_abund=10³, total_load=10⁷, template_input_vol=2, read_depth=5,000, n_iters=10⁵.

In particular, the forward model mathematically describes the process of generating read count data (probable molecular counts of a target) (FIG. 4A) as two successive stochastic sampling events, quantitatively described by Poisson sampling, that occur during amplicon sequencing.

In the illustration of FIGS. 4A-C, Stochastic Process 1 is the stochastic sampling of molecules during volume transfers (e.g., the loading of template DNA into the library-preparation reaction) that occurs when a sample is separated from an environment (FIG. 4B). In cases of low-to-moderate absolute abundances (or number of 16S rRNA gene molecules) of a taxon, the volume of transferred liquid (quantitatively measurable amount of the environment) determines the likelihood that at least one DNA molecule of the taxon is transferred (and therefore is potentially detectable), and the variability in number of molecules transferred (setting the minimum measurement noise). This stochasticity explains why two library-preparation reactions from the same sample using the same sample volume can contain substantially different compositions of molecules. Accordingly, this step is (i) a manipulation that can change the number of output molecules compared to the number of input molecules, (ii) is a stochastic step, and (iii) a Poisson probability distribution parameterized by the number of input molecules and a quantitatively measurable amount of sample separated from the environment can be used to track the number of probable output molecules.

In the illustration of FIGS. 4A-C, Stochastic Process 2 is the stochastic sampling of barcoded amplicons (or, more generally, library molecules) on the sequencing flow-cell. To make more intuitive connections to concepts like “read depth”, this process is referred as “sampling of reads” (FIG. 4C). In cases of high absolute but low relative taxon abundance, there is high variability in the number of taxon reads sequenced because only a small fraction of amplicons (determined by read depth) is sampled during sequencing.

It was then hypothesized that together, these two successive stochastic sampling events determine the fundamental limits of performance (which in this example, is defined detectability and measurement noise) from amplicon sequencing and explain several observations related to the issue of confidence from 16S amplicon sequencing of samples with low-to-moderate microbial load: (1) the stochastic detection and quantification of 16S rRNA gene; (2) the stochastic detection and quantification of reagent contaminants in processing blanks and samples; and (3) for a given total microbial load and read depth, there should be a minimum number of reads that are generated from a single molecule; read counts below this minimum threshold are likely artifacts from barcode hopping [93], [93], sequencing errors [42], [42], or taxonomic misclassification [94]. Furthermore, in some cases (stochastic loading) the difference between zero and thousands of reads can arise from just a few loaded molecules, and in other cases (stochastic sampling of reads) the difference between zero and a few reads can arise from thousands of loaded molecules.

Next, to test this hypothesis and provide validation of this proof-of-concept example of StochQuant, StochQuant simulations of the sequencing experiment from FIGS. 3A-E were compared with the experimentally observed results. In the context of, StochQuant simulations” is used to refer to computational simulations that use the StochQuant forward measurement model to yield a distribution probable molecular counts of a target. This validation step is performed to demonstrate that the forward measurement model has a level of performance or precision such that the model can yield distributions of probable molecular counts of a target that are similar to those obtained via experimental validation experiments.

These StochQuant simulations use the absolute concentration of quantified total microbial load (the number of reference molecules per unit of volume of the environment-here it is target molecules per microliter), read depth (molecular count of the reference molecule obtained via the testing measurement), and the volume of sample loaded into the library-preparation reaction (quantitatively measurable amount of sample separated from the environment) in combination with estimates of taxon absolute abundance (the number of target molecules in an environment) as inputs into the forward measurement model to generate a simulated read count (a probable molecular count of the target molecule) (FIG. 4A).

These simulations correctly predicted experimentally observed read counts and their variability across dilution conditions for taxa present in the sample at a given relative abundance (FIG. 5A) and for contaminant taxa introduced into the dilutions at a given absolute abundance, such as Spirosoma (FIG. 5B).

In particular, the simulation shown in FIG. 5A was performed as follows. For each sequencing replicate, an estimate of absolute abundance, total load, observed read depth, and template loading volume (2 μL) were used as inputs into the StochQuant simulate_readcounts function to generate one simulated read count. An estimate of absolute abundance was computed by multiplying the estimated relative abundance of Pseudomonas by the total load. The relative abundance of Pseudomonas was approximated by computing the mean relative abundance of Pseudomonas among sequencing replicates in the MD1 dilution condition. The same method described for FIG. 5A was used for FIG. 5B, except an absolute abundance of 500 copies/mL was approximated.

To evaluate how well StochQuant predicts the detectability and measurement noise of each defined-community taxon under each dilution condition (FIG. 5C, FIGS. 6A-D), the same sequencing experiment from FIG. 3A were simulated 100,000 times, and in each simulated sequencing experiment, the number of times that each taxon was detected were computed. By repeating this procedure, a 95% confidence interval was obtained for the frequency at which a taxon is estimated to be detected in the experiment. This estimate of detectability was compared to the experimentally observed detection frequency for each taxon in each dilution. Simulations were performed using the same procedure described above for FIG. 5A.

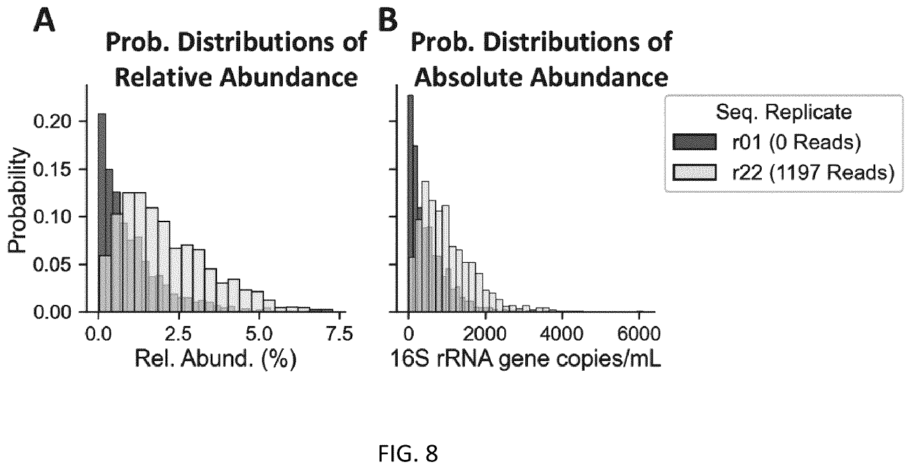

To compare observed measurement noise to StochQuant simulated measurement noise (FIG. 5D, FIGS. 6A-D), the coefficient of variation (% CV) of each taxon relative abundance was computed in each dilution in the experimental data, and in each of the (n=100,000) simulated experiments. The mean CV among simulated CVs for each taxon under each dilution condition was used for analysis. One taxon in one dilution (Salmonella in MD4) was excluded, as it was undetected in all sequencing replicates. Simulations for FIG. 8 Panel D, FIG. 6D were performed using the StochQuant simulate_readcounts function, using input parameters in the same fashion as described in the methods for FIG. 5A. Simulations for FIG. 6B were performed by using the Numpy random.poisson function with the input parameter lambda set to the product of the mean taxon relative abundance in MD1 and the read depth of the sequencing replicate). Simulations for FIG. 6C were performed by using the StochQuant function simulate_loading_and_sequencing, and multiplying the relative abundance of target copies loaded (target_copies_loaded/total_copies_loaded) by the read depth of the sequencing replicate.

The simulations for FIG. 5E were performed as follows: The Numpy.arange function was used to generate evenly spaced arrays (spacing=0.05) for taxon abundance and total bacterial load in log 10 space. Arrays from 3-10⁵ copies/μL and 10⁵-109 copies/μL were generated for taxon abundance and total bacterial load, respectively. The cartesian product of the two arrays was used to obtain possible combinations of taxon abundance and total bacterial load, and combinations for which the taxon abundance was less than or equal to the total load were retained. For a given taxon abundance, total bacterial load, read depth (10,000 or 100,000), template loading volume (0.5 or 5 μL), the StochQuant simulate_readcounts function was used to generate 1,000 simulated read counts. Then, for each set of parameters, probability of detection was calculated by dividing the number of nonzero read counts by the total number of simulated read counts (n=1,000). Each combination of parameters was plotted, colored by the probability of detection. Probabilities of detection less than 5% were set to an opacity alpha of 0.1. Relative abundance limit of detection was calculated by dividing 3 by the simulated read depth, and absolute abundance limit of detection was calculated by dividing 3 by the template input volume.

Furthermore, StochQuant simulations (n=100,000) were found to accurately predict the detectability of each taxon under all four dilution conditions. Thus, the measurement workflow representation accurately tracked the number of target molecules leading to the detection or stochastic non-detection of the target molecule via the amplicon sequencing testing measurement. Among the 5 defined-community taxa and 4 dilution conditions, 95% (19/20) of the frequencies of detection fell within the confidence interval of detection predicted by StochQuant (FIGS. 6A-D), including the change in detectability of a low-relative abundance taxon (0.04%) as total microbial load decreased from MD1 (100% detection) to MD4 (10% detection) (FIG. 5C). StochQuant simulations (n=100,000) also accurately predicted the measurement noise of each defined-community taxon under each dilution condition. The majority (17/19) of StochQuant % CVs were within 0.5 to 2-fold of the StochQuant simulations (FIG. 5D). Although simulations of only stochastic sampling of reads accurately predicted measurement noise in the high load samples (MD1), these simulations systematically underestimated measurement noise at lower load samples (MD2-MD4) (FIG. 6B). In cases of high total microbial loads (MD1), simulations of only stochastic loading of molecules underestimated measurement noise (Figure. 6C).

The StochQuant forward measurement model generalized the relationship between the four key input parameters (the number of input target molecules, an absolute anchoring value of the reference molecule, a molecular count of the reference molecule, and a quantitatively measurable amount of sample separated from the environment) and detectability and measurement noise (Figure. 5E-F). Accordingly, the results illustrated in FIG. 5E-F, indicate the StochQuant model could accurately track the number of molecules in the molecular detection workflow of amplicon, which yielded stochastic detection and distributions of molecular counts of the targets. In this case the stochastic detection was measured by “detectability” discussed above, and the distribution of molecular counts of the target was measured by % CV and referred to as “measurement noise”. The StochQuant forward measurement model was used to simulate read counts across 4 orders of magnitude in taxon concentration and total bacterial DNA load, and across template loading volumes and read depths used for amplicon sequencing experiments. In general, there are three key limiting regimes that hinder taxon detection in this molecular detection workflow, each with its own solution to improve detection (FIGS. 5E-F). (1) (Read Limited) When taxon relative abundance is low but absolute abundance is sufficiently high, at least one taxon molecule is loaded into the library preparation reaction. During library preparation, millions of barcoded progenies of the original taxon 16S molecule are generated.

However, at low relative abundance, these barcoded progenies may not be sufficiently sampled (other, higher relative-abundance amplicons will have a higher probability to bind to the sequencing flow-cell). Therefore, increasing read depth improves the likelihood that the molecule is detected. (2) (Loading Limited) When taxon absolute abundance is low, but relative abundance is sufficiently high (or read depth is sufficiently high), detection can fail because sometimes zero taxon molecules are stochastically loaded into the library preparation reaction. In this regime, loading more volume (but not increasing read depth) improves detection and quantification (3) (Both Read and Loading Limited) When both absolute and relative taxon abundance are low, sometimes zero taxon molecules are stochastically loaded, and even in cases where at least one molecule is loaded, the amplicons of this molecule may not be detected by sequencing. Simulations describe graphically (Figure. 5F) how changing read depth and template loading volumes can be adjusted to achieve consistent detection for a given combination of total load and taxon load. It is noted that in these simulations, increasing template loading volumes can be used interchangeably with concentrating the total DNA of the sample, and for clarity, only the former is used.

### Example 6 Development and Validation of StochQuant Inference of Taxon Abundance

Next, a capability of this proof-of-concept example of StochQuant was developed to generate a probability distribution of taxon abundance (absolute or relative) from the physical parameters of the testing measurement workflow (a single sequencing read-count measurement, total microbial load measurement, experimentally used volumes, and read depth) (FIG. 7A). Accordingly, the results show that the inference method uses the Measurement Workflow Representation and the physical parameters of the testing measurement workflow to yield a distribution of probable numbers of target molecules in an environment.

In this example, the distribution of probable number of target molecules in an environment can be scaled (i) by a quantitatively measurable amount of environment to yield a distribution of probable absolute abundances (also referred to as concentration of target or copies/mL or copies/μL of target) or (ii) by the total number of 16S rRNA gene molecules in an environment to yield a distribution of probable relative abundances. The StochQuant method builds these probability distributions for each taxon in each sample by performing a maximum likelihood inference procedure to compute the inverse probability of the forward measurement model (see Methods). Accordingly, StochQuant infers, for a given experimental setup and total microbial load, the probability that a given absolute or relative abundance would lead to the observed read count for that taxon.

Using these probability distributions, StochQuant naturally handles the issue of confidence that arises when one obtains a target molecular count of zero, or zero-counts (non-detection), which otherwise often require special treatments [90, 91, 95]. To illustrate this concept, a “loading-limited” simulation was shown (see FIG. 7B) that results in non-detection of a target over 40% of the time, and more than 1000 reads (molecular count of the target) in the remaining simulations (FIG. 7B).

In particular to generate the simulated data for FIG. 7B, read counts were simulated using the StochQuant simulate_loading_and_sequencing function with the following parameters: abs_abund=0.4, total_load=40, template_input_vol=2, read_depth=100,000, n_iters=10,000). A histogram of the simulated read counts was generated using the Seaborn histplot function. Read counts from zero to 3,500 are shown.

When StochQuant is used to estimate taxon abundance from three of the simulated read-counts (0, 1500 and 3000) which without the probability distributions of abundance would be interpreted as three different target abundances, it becomes visually clear that the three simulated read counts (simulated molecular counts of the target) could have arisen from the same taxon abundance (their probability distributions of abundance overlap substantially) (FIG. 7C).

In particular, probability distributions of taxon abundance in FIG. 7C were generated by using the StochQuant high_throughput_qsa_rev function with the read count parameter set to 0, 1500, and 3000, and the following other parameters: read_depth=100,000, elution_vol=100, seq_fold_dilution=1, seq_template_vol=2, seq_reps_used=1, total_load=40. Relative abundance values were sampled from each distribution using the StochQuant generate_data_from_distributions function. Histograms were generated using the Seaborn histplot function.

The improvement was assessed in performance of quantitative detection of targets when a target molecular count yielded by the testing measurement was zero (zero counts) with the defined-community sequencing experiment. Analysis with a standard approach (without the probability distributions of abundance yielded by StochQuant and without the confidence level yielded by StochQuant) can lead to the incorrect conclusion that a target is not present (false negative) in all 69 instances of non-detection. With analysis with the StochQuant workflow, it was found that in 97% (67/69) of measurements of non-detection (zero taxon read counts), StochQuant correctly identified non-detected taxa as being within the 95% confidence interval of the taxon's mean relative abundance in MD1, which was used as the “ground-truth” relative abundance.

Accordingly, in this example, an improvement in technology was validated obtained with a StochQuant workflow that arises from the use of a probability distribution of target abundance, a confidence interval, a confidence level threshold, a confidence level, and a determination based on the confidence level. In this example, the ground truth abundance values of each target in the environment were known, and so it was known that the non-detection of the target was due to stochasticity of the molecular detection workflow. Therefore, it was possible to validate the capability of StochQuant to yield a level of confidence indicative of probabilistic detection of the target, even when a molecular count of zero was obtained for the target via a testing measurement. In this case, a target was considered to be probabilistically detected if a confidence level between 2.5 and 97.5% was obtained for a confidence interval set at the “ground truth” abundance of the target. A minimum confidence level threshold was chosen of 2.5 because it was considered a taxon to have a reasonable probability of being in the environment if the confidence was at least 2.5%, and chosen to set a maximum confidence level threshold of 97.5% because a taxon would have a reasonably low probability of being in an environment and not being detected by the testing measurement.

The widths of StochQuant probability distributions of abundance (which were also referred to as measurement uncertainty in probable numbers of target molecules in an environment) increase with decreasing total microbial load (decreasing number of reference molecules), volume transfers (amount of sample separated from the environment), and read depth (molecular count of the reference molecule). Accordingly, when the number of target molecules decreases (and the number of reference molecules proportionally decreases to maintain the same amount of target relative to reference) and the quantitatively measurable amount of sample separated from an environment remains constant and the molecular count of the reference molecule (read depth in this example) remains constant, the variability in probable numbers of target molecules in an environment yielded by StochQuant increases. Consider probability distribution of relative abundance provided by StochQuant for Bacillus (1% relative abundance) (FIG. 7D).

In particular in FIG. 7D, one representative probability distribution of Bacillus relative abundance from a sequencing replicate from each of the four dilution conditions was plotted (MD1 r22, MD2 r04, MD3 r11, MD4 r22). Then, in FIG. 7E, (n=1000) estimates of taxon relative abundance were sampled from each representative distribution (as described in above within StochQuant Specific Methods). These sampled abundance values were compared to all observed taxon relative abundances.

Although each of the four dilution conditions have similar read counts and read depths, StochQuant appropriately identifies the increase in measurement uncertainty as the total microbial load (and therefore the number of taxon molecules loaded into the sample) decreases. When abundance values are sampled from each of the distributions from FIG. 7D, the simulated abundance values and their variability closely match those experimentally observed among all sequencing replicates (FIG. 7E).

Accordingly, this example, show a proof of principle of an improvement in technology (reduction in false positives) with the StochQuant workflow. The “Use StochQuant Workflow”can use probability distributions to determine whether a set of molecular counts of a target (the experimentally observed molecular counts from the testing measurements) could have arisen from the same taxon abundance (whether two or more taxa are differentially abundant). Accordingly, the distribution of number of probable target molecules obtained via StochQuant from one testing measurement can be compared to a distribution of number of probable target molecules obtained via StochQuant of a second testing measurement; the comparison of distributions can be used to obtain a measure of confidence that the target molecule is present at an abundance that is within the range of probable abundances of both distributions. Given that a taxon should be at the same relative abundance regardless of dilution or sequencing replicate in this example, it was tested how often StochQuant distributions of relative abundance from two sequencing replicates (both within and between dilution conditions) correctly did not reject the null hypothesis (that the two measurements arose from the same taxon abundance).

In this example, the improvement enabled by the of StochQuant workflow was validated in the detection technology through molecular count, to determine if the number of target molecules in one or more environments differs between two or more measurements obtained via a testing measurement. Accordingly, in an exemplary implementation if a user detects a target in Environment 1 and the user detect the target in Environment 2, the user wants to determine if the target is more/less abundant in Environment 2 compared to Environment 1. In reality, the user often get two different measurements, but a confidence level yielded by StochQuant can improve the user's ability to determine whether the target is actually more/less abundant in Environment 1 vs Environment 2. Accordingly, this experiments show that StochQuant enables a user to determine if the difference between the two measurements is larger than the stochasticity/uncertainty/noise as will be understood by a skilled person.

At low abundance, such as for Bacillus, even when a taxon is detected with over 1000 reads in one measurement but goes undetected in another measurement, StochQuant correctly does not reject the null hypothesis as shown by the data reported in FIG. 8.

In particular the data in FIG. 8 was generated as follows. One representative sequencing replicate among the MD4 sequencing replicates was chosen (MD4 r22), and one sequencing replicate of similar read depth for which Bacillus was undetected was chosen (MD4 r01). Histograms (generated with the Seaborn histplot function) of the (A) relative and (B) absolute StochQuant abundances (described in StochQuant Specific Methods) for each sequencing replicate are shown.

Accordingly, these results show that StochQuant, enables the correct determination that the target is NOT more abundant in an environment (even though the target was undetected in the other environment). Overall, with a significance threshold (or confidence threshold) of 0.05, StochQuant comparisons of distributions had a Type I error rate (incorrectly reject the null hypothesis/the determination based on the confidence threshold yielded an incorrect result) of 7% (1293/18275 comparisons). Nearly half (n=577) of these incorrect calls came from Listeria (97% relative abundance).

StochQuant can also identify small differences in relative abundance (1.0% versus 1.9%) when stochastic measurement noise is small, but correctly does not identify such differences as significant when stochastic noise is large. Accordingly, the probability distributions yielded by StochQuant and the confidence level obtained from the probability distributions reflect the inherent stochasticity of the molecular detection. To illustrate, comparisons are shown of StochQuant probability distributions of abundance from a representative sequencing replicate (see Methods) of Bacillus (1% relative abundance) and Pseudomonas (1.9% relative abundance) in MD1 and MD4 (FIGS. 7F-G).

In particular FIG. 7F, in the left panel one StochQuant probability distribution of taxon relative abundance is shown each for Bacillus (MD1 r21) and Pseudomonas (MD1 r04). To select each distribution, the observed relative abundances were sorted for each taxon among the MD1 sequencing replicates and selected the sequencing replicate with the median observed relative abundance. In the middle panel, (n=1000) estimates drawn from the Bacillus probability distribution from (MD1 r21) and Pseudomonas probability distribution from (MD1 r04) are shown. P StochQuant was computed as described above in StochQuant Specific Methods. In the far-right panel, all observed relative abundances of Bacillus and Pseudomonas from the MD1 sequencing replicates are shown. Pvalidation was computed using a similar approach to the computation for P StochQuant, as follows. First, the observed Bacillus and Pseudomonas relative abundances among MD1 sequencing replicates were randomly sampled (n=####) with replacement. Then, the frequency at which each randomly sampled Pseudomonas relative abundance was greater than each randomly sampled Bacillus relative abundance was recorded. This frequency was subtracted from 1 to get P_{Validation}. FIG. 7G was generated as described for FIG. 7F, except comparisons among MD4 sequencing replicates were analyzed and plotted.

When total microbial load was high (MD1), StochQuant predicts (and experimental validation confirms) that these two measurements arose from two different taxon abundances with statistical significance (P_{StochQuant}<0.001, P_{Validation} <0.001) (FIG. 7F). At low total microbial loads (MD4) StochQuant predicts (and experimental validation confirms) that differential abundance cannot be confidently determined (P_{StochQuant}=0.259, P_{Validation}=0.160) (FIG. 7G).

As another validation of this proof-of-concept example of StochQuant, it was next assessed whether StochQuant probability distributions of abundance could be used to improve the 16S rRNA gene amplicon sequencing technology through the improved identification and removal sof equencing artifacts and contaminants to make analyses and/or determinations based upon analyses more reliable and reproducible. The procedure with the probability distributions of taxon abundance yielded by StochQuant was performed as follows: For each taxon, the absolute abundance distribution in the NTC was compared against the distributions of the taxon in each sample. If the lower 1^{st} percentile of the distribution in any sample is higher than the upper 99^{th} percentile of the distribution in the NTC, the taxon remains in the analysis. If this does not occur in any sample, a taxon is considered a contaminant. Depending on the tolerance for including contaminants or excluding biological taxa, these thresholds may be changed. Accordingly, a determination was made for whether more molecules of a target (taxon) were present in a given dilution (e.g., MD2) compared to an NTC. To do so, a probability distribution of the number of target molecules in a dilution (e.g., MD2) was compared to a probability distribution of the number of target molecules in a NTC to obtain a measure of confidence.

To illustrate the utility and improvement in molecular detection technologies, consider Spirosoma (contaminant) and Pseudomonas (member of defined community), taxa that were stochastically detected in the NTC replicates. Spirosoma was measured in dilutions at as high as 4.7% relative abundance, was only detected in 2/4 NTC replicates, and (with standard absolute abundance estimation) was quantified at lower absolute abundances than in some dilutions (FIG. 9A), and would therefore not be removed as a contaminant by most standard approaches.

In particular FIG. 9A was generated as follows. Standard estimates of taxon absolute abundance were computed as previously described [38]. Spirosoma absolute abundance estimates are shown for each of the four NTC sequencing replicates, and one representative sequencing replicate each from MD3 (r14) and MD4 (404). Histograms of StochQuant estimates of Spirosoma absolute abundance in NTC r01 (top panel), NTC r04 (bottom panel) to MD3 r14 (purple) and MD4 r04 (blue) are shown.

In contrast, analysis with StochQuant probability distributions of absolute abundance revealed that the differences in experimentally observed read counts (given the other physical parameters) were within the intrinsic stochastic noise of the measurements, even when Spirosoma is undetected in the NTC. For Pseudomonas, only when the total microbial load was sufficiently high (MD3 but not MD4), the differences in abundance between the NTC and sample could be determined with statistical significance (P<0.0001) (FIG. 9B).

In particular, FIG. 9B was generated following the same procedures for comparisons of Pseudomonas absolute abundance as those described for for Spirosoma absolute abundance

Accordingly, these results show the probability distributions yielded by the StochQuant method can be used to obtain a measurement of confidence in the quantitative detection of a target in one or more environments. This measurement of confidence can be used to make a determination.

Accordingly, an exemplary improvement in molecular detection technologies provided by the StochQuant workflow was demonstrated. A contamination-identification procedure was developed based upon comparing StochQuant probability distributions of absolute taxon abundances, on the entire defined-community dataset, and compared StochQuant to standard approaches. Before filtering, 61 genera were detected across the 90 samples in the defined-community dataset, and standard relative abundance filtering failed to remove the majority of contaminants while preserving the defined-community taxa (FIG. 9C). In contrast, StochQuant filtering enabled robust identification and elimination of contaminant taxa (52 out of 55 contaminants removed), while retaining defined-community taxa (Figure. 9C). It can be understood that the improved identification and computational elimination of contaminant taxa enabled by StochQuant improves the detection of molecular counts as will be understood by a skilled person.

In particular, contamination filtering for FIG. 9C was performed as follows. Relative abundance filtering was performed by sub-setting the genus-level relative abundance data for taxa that are greater than or equal to the threshold for 0.001, 0.1, 1, and 10 percent relative abundances. For each relative abundance threshold, the number of taxa detected above the threshold that are classified as either part of the defined community or not (contaminant) were computed by using the Pandas.groupby.nunique ( ) function. StochQuant filtering was performed as described in StochQuant Specific Methods.

In these set of experiments, the determination is whether a taxon is present at a higher absolute abundance, more target molecules in a dilution) compared to an NTC.

StochQuant-derived taxon probability distributions can also improve PCA results. Standard PCA results varied considerably between trials (FIG. 3E). However, when the (center-log transformed) StochQuant-filtered probability distributions of taxon relative abundance was projected onto the principal components from each trial from FIG. 3E, it was found that regardless of the trial and the dilution condition, that all samples clustered together (FIG. 9D, FIG. 10).

In particular, PCA in FIG. 9D was performed for each of the sets of sequencing replicates from the four trials from FIG. 3E as follows. First, the processed sequencing data collapsed to the genus level was subset for the sequencing replicates that were used in the trial, and taxa that were considered above the limit of blank (described in StochQuant Specific Methods) were identified. Only taxa that were above the limit of the blank were used in downstream analysis for a given trial. Then, StochQuant estimates of absolute abundance for each sample were filtered for taxa that were above the limit of the blank, and transformed into relative abundances by dividing each absolute abundance by the sum of the retained absolute abundances for each StochQuant iteration. PCA was then performed by projecting the StochQuant relative abundance estimates onto the principal components computed in FIG. 3E. This procedure was performed as described in StochQuant Specific Methods. Because most of the data overlaps, the same data is also shown in separate panels (FIG. 10), where each “cloud” of PCA values for each sequencing replicate is shown in a separate panel.

Next, differential-abundance analysis with StochQuant for was performed each trial (two dilution conditions, each with 3 samples) (from FIGS. 3A-E) on five of the defined-community taxa, and compared performance against the three methods (DESeq2, ALDEx2, and Kruskal-Wallis) used in FIGS. 3A-E. It can be understood that in this example, a confidence level yielded by StochQuant is used to make a determination of differential abundance between two or more environments or two or more groups of environments. In this example, a confidence level of less than or equal to 0.05 is used to determine differential abundance. It was chosen to use less than or equal to 0.05 to more closely follow the confidence scales used for statistical differential abundance approaches. In the congruent approaches, the null hypothesis is that the target is not differentially abundant, and thus, a low confidence score can be interpreted as less confidence that a target is not differentially abundant (and is thus more likely that the target is differentially abundant).

In this proof-of-concept example of improvement in technology with the StochQuant method, differential-abundance analyses are performed by iteratively generating probability distributions of test statistics and P-values. In each iteration, for a given taxon in each sample, StochQuant repeatedly draws one estimate of abundance from each probability distribution of this taxon abundance. Then, a statistical test (e.g., Kruskal-Wallis) is performed on the sampled abundance estimates. This procedure is repeated many (n >1000) times to obtain a distribution of test statistics and P-values, which are in turn used to establish the likelihood that the observed differential abundance is greater than the measurement noise. Because taxon abundances and total microbial loads in this dataset span several orders of magnitude, the three standard methods yielded incorrect “statistically significant” results (P<0.05) in 388, 1124, and 1303 (out of 5000) comparisons. Accordingly, these methods incorrectly concluded that the same taxon is differentially abundant between replicate measurements of either the same dilution or across dilutions. In contrast, StochQuant only yielded such incorrect results in 27 out of 5000 comparisons (FIG. 9E) demonstrating a reduction in the number of false positives yielded by differential abundance analysis.

In particular, the analysis for FIG. 9E was performed as follows. The same differential abundance data from each trial for each of the three differential abundance methods (DESeq2, ALDEx2, Kruskal) used in FIG. 3B was used in FIG. 9E, with the addition of the StochQuant differential abundance analysis. This StochQuant differential abundance analysis was performed on the same trials as FIG. 3B, as described in Example 5. The total number of false positives (incorrectly determining a taxon to be differentially abundant, when it in fact is not) was computed by summing the false positives across all comparisons and all defined-community taxa for each differential abundance approach.

Differential-abundance analysis can also be performed on more than two conditions (e.g., all four dilution conditions) to determine if one condition contains a taxon that is differentially abundant compared to the other conditions. To perform differential abundance on each taxon across multiple conditions (including conditions where a taxon goes completely undetected), in this example, the (standard) Kruskal Wallis statistical test was used on each of the sets of samples from the (n=100) PCA trials from FIG. 1E, 5D. With the standard Kruskal-Wallis test, incorrect differential abundance was inferred (PKruskal<0.05) near the expected false discovery rate for Bacillus and Pseudomonas (FDR: 3%, 4%) but high for Listeria, Escherichia, and Salmonella (FDR: 51%, 26%, 29%). In contrast, StochQuant analyses correctly yielded zero differentially abundant taxa. The results of these analyses can be visualized with plots of the estimates drawn from each probability distribution. Here, it is illustrated with some of the most extreme examples from three taxa present at high, medium, and low relative abundances in the defined community (FIG. 9F-H), showing visually that even in these extreme cases (including cases of non-detection),

In particular, the plots for FIG. 9F-H were generated as follows. Example trials from a high (Listeria, 98%), medium (Pseudomonas, 1.9%), and low (Escherichia, 0.03%) relative abundance are shown, where taxa incorrectly are determined to be differentially abundant based on the Kruskal-Wallis statistical test (significance threshold <0.05), but are not differentially abundant based on StochQuant differential abundance analysis. Statistical tests were performed as previously described for FIGS. 3A-E and FIGS. 9A-H.

StochQuant distributions from each condition overlap (P_{StochQuant}>0.05), and differential abundance is not inferred. Accordingly, the results of the experiments shown in these examples, indicate the determination of differential abundance is improved by using a confidence level yielded by StochQuant.

### Example 7 Testing of StochQuant with Low-to-Moderate Load Human Gut Biopsies

To test and validate the performance of this proof-of-concept example of StochQuant beyond dilutions of a defined microbial community, 16S rRNA gene sequencing data were analyzed and re-sequenced a subset of specimens from longitudinally collected mucosal gut biopsies from 2 humans (FIG. 11A) from a previous study [39].

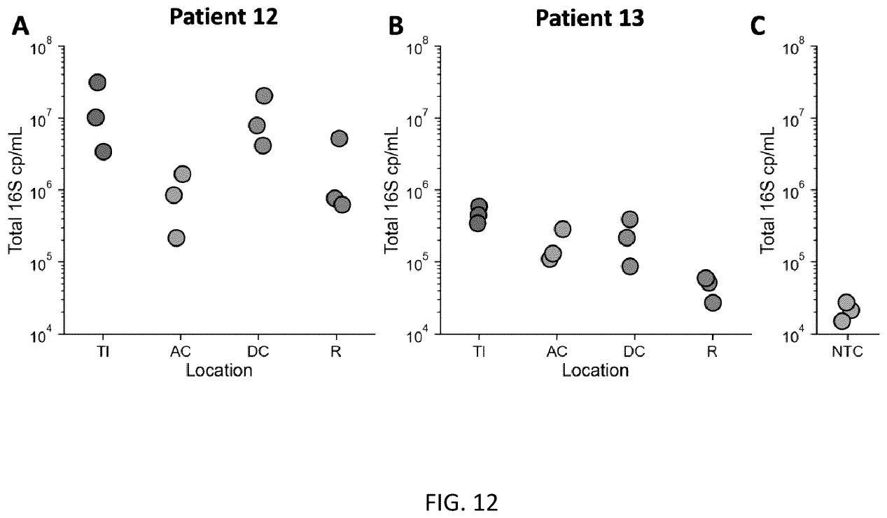

In this context, the solution of nucleic acids from each clinical specimen is an environment. The target molecule, reference molecule, and absolute anchoring values are the same as the previous dilution example. Briefly, 24 biopsies (3 biopsies from each GI location in each patient) were used that were previously collected and sequenced from the terminal ileum (TI), descending colon (DC), ascending colon (AC), and rectum (R) from 2 patients (Patient 12 or P-12, Patient 13 or P-12). These two patients were chosen because biopsies from one patient (P-12) contained moderate microbial loads (10⁵-10⁸ 16S rRNA gene copies/mL) and taxa at both low relative and absolute abundances, while biopsies from the other (P-13) contained low total microbial loads (10⁴-10⁶ 16S rRNA gene copies/mL) (FIG. 12).

First, it was tested whether reproducible detection of taxa via 16S rRNA gene sequencing in complex human clinical samples is predicted by StochQuant. StochQuant estimates of absolute abundance were used from a subset of (n=9) biopsies from P-12 and P-13 as inputs into the StochQuant forward measurement model, which predicted that 195 measurements would be consistently detected among sequencing replicates. We then re-sequenced 2-3 additional replicates of each of these biopsies and found that 192 of those 195 predicted measurements (98.4%) were detected among all sequencing replicates of a given biopsy ( )

Next, it was determined the proportion of sequencing measurements occurring below the Limit of Detection (LoD; taxon abundance at which there is at least a 95% probability of detection), and therefore how often standard methods could lead to irreproducible detection and analyses. An exemplary measurement: workflow representation (StochQuant forward measurement model) to compute the LoD of each sequenced biopsy (see Methods), and used the StochQuant probability distributions of abundance to determine the probability that a taxon was present above the LoD in each biopsy. It was found that even at reasonably high total microbial loads (P-12), taxa were only detected above LoD 61% (658/1074) of the time. At lower total microbial loads (P-13), taxa were only detected above LoD 42% (310/734) of the time. Furthermore, of the 210 detected taxa in P-12 biopsies and 190 detected taxa in P-13 biopsies, with StochQuant contamination filtering, only 75 and 23 taxa (respectively) were confidently detected above contaminant levels in the processing blanks.

Next, the analysis results of standard approaches were compared to the StochQuant approach.

In particular, PCA for the Standard Approach (FIG. 11B) was performed (as previously described in FIGS. 3A-E) on the unfiltered relative abundance data from the Patient 12 biopsies.

PCA for StochQuant Approach (FIG. 11C) was performed as described in the StochQuant Specific Methods on the StochQuant-filtered relative abundances from the same samples.

Magnitude of Feature Loadings in PC1 and PC2 space were computed by multiplying each eigenvector by the square-root of its corresponding eigenvalue. The 10 highest magnitude feature loadings each for the Standard Approach (FIG. 11D) and StochQuant Approach (FIG. 11E) are shown.

(FIG. 11F, FIG. 15A) Differential abundance analysis with Aldex2, DESeq2, and Kruskal-Wallis was performed on the 16S rRNA gene sequencing read count data from the terminal ileum (TI) and rectum (R) biopsies within each patient (Patient 12, Patient 13) described in FIGS. 3A-E. Differential abundance analysis with StochQuant was performed as described in StochQuant Specific Methods.

The strip plots used in FIG. 11G-H, and Figures14A-D, FIGS. 16A-D, and FIG. 15B-E were generated as follows. (Left) Standard relative abundance estimates (obtained by dividing the number of taxon reads by the total reads of the sample) for a given taxon from the original sequencing data (not including the re-sequenced replicates) were plotted for each of the (n=3) terminal ileum and (n=3) rectum biopsies in a given patient. Taxon-specific P-values from standard differential abundance analysis results were displayed on top of the plot. (Middle) Taxon relative abundances sampled from StochQuant probability distributions of taxon relative abundance from the original sequencing data were plotted for each of the terminal ileum and rectum biopsies in a given patient. Taxon-specific P-values from StochQuant differential abundance analysis results were displayed on top of the plot. (Right) Standard relative abundances of re-sequenced biopsies were plotted.

Accordingly, another example is described of how StochQuant improves molecular detection technology in the context of PCA analysis. In P-12, it was found that with standard approaches and standard absolute abundance filtering, PCA did not reveal clear clustering by GI location (FIG. 11B).

However, PCA with StochQuant revealed clear separation and clustering between the terminal ileum (TI) and ascending colon (AC), with the descending colon (DC) and rectum (R) clustering together (FIG. 11C).

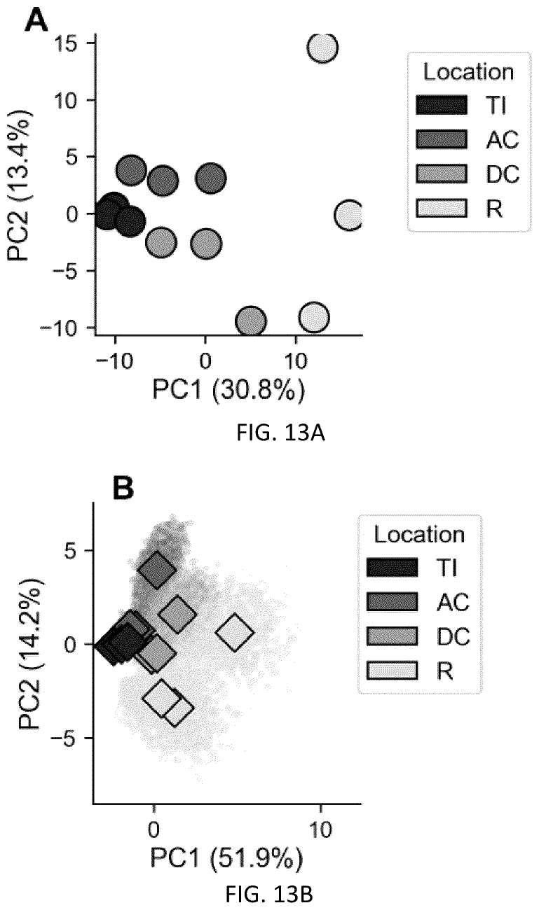

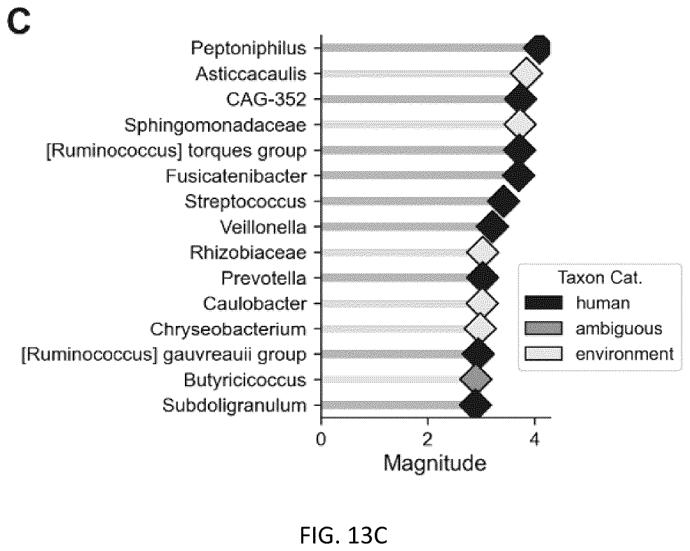

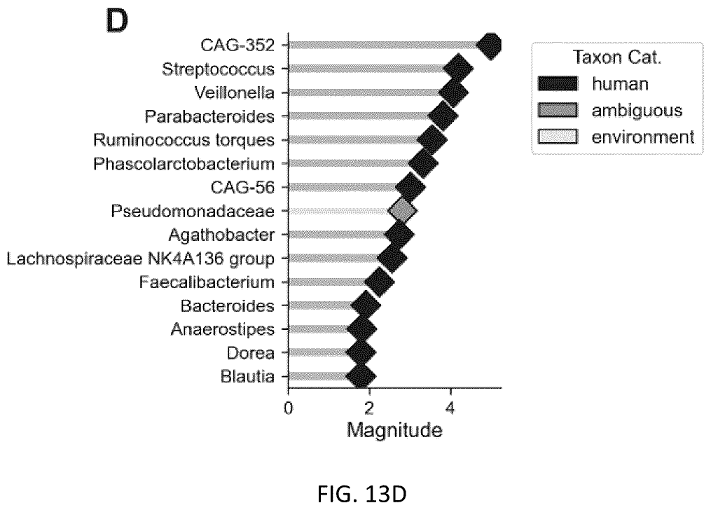

Furthermore, with the standard approach the variance observed in PC1 and PC2 was primarily driven by environmental taxa (typically implicated as contaminants in sequencing data) (FIG. 11D). In contrast, the top feature loadings from the StochQuant approach were primarily human gut microbes (FIG. 11E). These same analyses were repeated for P-13, whose samples had lower total microbial loads which further decreased along the GI tract. To account for the increasing effect of contaminants with decreasing total microbial load, contaminants were filtered out using StochQuant and re-calculated taxon relative abundances. PCA with these data indicated that differences among GI locations observed via PCA with standard approaches were primarily driven by contaminants (FIGS. 13A-D).

Finally, taxa were identified that were differentially abundant between terminal ileum (TI) and rectum (R) locations along the GI tract of each patient. The 3 standard approaches (Deseq2, Aldex2, Kruskal-Wallis) and StochQuant found 31 differentially abundant taxa (P<0.05) in total for P-12 (FIG. 11F) and 48 for P-13. In P-12, Kruskal-Wallis found the most differentially abundant taxa (n=25), followed by DESeq2 (n=22), StochQuant (n=9), and ALDEx2 (n=8). In the lower-load, contamination driven P-13, the order was DESeq2 (n=46), Kruskal-Wallis (n=33), ALDEx2 (n=21), and StochQuant (n=2). Only a few (n=5 for P-12) and (n=2 for P-13) taxa were called differentially abundant by all four approaches; all of such taxa were above LoDs predicted by StochQuant (FIG. 11G, FIGS. 14A-D, and FIG. 15B).

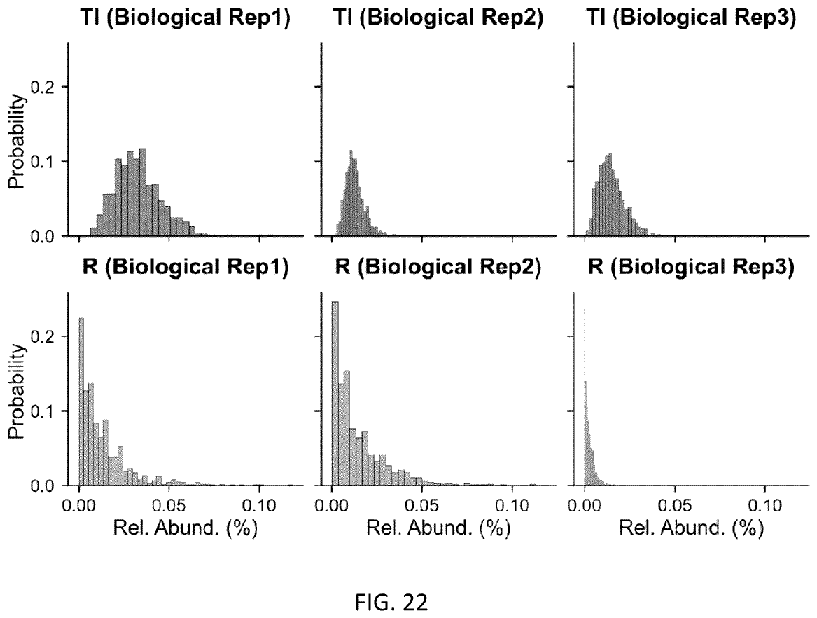

At moderate taxon abundances, accurate estimation of measurement uncertainty by StochQuant (as confirmed by sequencing replicates) was needed to correctly infer differential abundance (FIGS. 16A-B). At low taxon relative abundance, LoDs (predicted by StochQuant) and inference of probable taxon abundances were needed for analysis. StochQuant correctly predicted the lack of reproducible detection in re-sequenced replicates (FIG. 16D), and identified cases where differential abundance was incorrectly inferred when taxa were initially undetected in one GI location (FIG. 11H, FIG. 22, FIG. 16C).

When total microbial loads were low and dependent on GI location (as in P-13), StochQuant contamination filtering, LoD estimation, and inference of taxon abundances greatly reduced false positive discovery rates (FIGS. 15A-E). Although many of these taxa appeared to be present at moderately high (>1%) relative abundance, these taxa were present at low absolute abundances, and thus were stochastically detected with considerable measurement noise. Overall, by accounting for the measurement uncertainty of each detected and non-detected taxon and LoD of each sequenced sample, StochQuant overcomes the challenges of interpreting amplicon sequencing data from low-to-moderate abundance taxa and in low-to-moderate microbial load samples.

### Example 8 StochQuant Improves Interpretation of Amplicon Sequencing Results in these Samples with Low-to-Moderate Abundance Microbes

The results shown in this preceding examples emphasize the key role that stochastic sampling effects play in microbiome analysis of samples with low-to-moderate microbial biomass. By developing and validating this proof-of-concept example of StochQuant, it was shown that stochastic inference of absolute number of molecules is both necessary and sufficient to substantially improve interpretation of amplicon sequencing results in these samples with low-to-moderate abundance microbes. In particular, it was demonstrated that the StochQuant method uses a molecular count of a target molecule and a molecular count of a reference molecule obtained via a testing measurement and an absolute anchoring value of a reference measurement to obtain a probability distribution of the number of molecules of a target in an environment. This probability distribution of the number of molecules in a target environment can be used to obtain a measure of confidence, and this measure of confidence can be used to make a determination.

StochQuant is a combined experimental and computational approach that improves the quality of microbiome analysis of low-to-moderate biomass taxa, which are difficult to analyze with standard methods (FIGS. 3A-E). By relying on absolute quantification, the StochQuant forward measurement model mathematically explains how read-count data (a molecular count of the target molecule) is generated/yielded from small numbers of target molecules (see e.g. FIGS. 4A-C), including the possible range of read counts generated from a single molecule. It also informs experimental design because it describes the conditions under which sequencing of low-to-moderate abundance microbes intrinsically results in reliable or unreliable detection and quantification (FIGS. 5E-F). StochQuant simulations of sequencing accurately predict the detectability and measurement noise of target molecules (e.g., taxa) across a wide range of absolute and relative abundances.

This proof-of-concept example of StochQuant uses (i) absolute quantification (digital PCR) to obtain an absolute anchoring value of a reference molecule (total 16S rRNA molecules), (ii) other known experimental parameters (e.g., quantitative measurable amount of sample separated from an environment), and (iii) molecular counts of a target and of a reference molecule to generate probability distributions of taxon abundance (absolute or relative) from a single sequencing read-count measurement and other quantitative parameters (FIG. 7). StochQuant eliminates the need for special treatment of zero-count reads because it uses them, together with other quantitative experimental information, to generate probability distributions. Those StochQuant probability distributions are in turn used to estimate taxon abundances and measurement uncertainties.

The probability distributions of abundance are also used to perform comparative analyses, which are used to make determinations. Comparing probability distributions of absolute taxon abundances in a sample to those in NTCs is used to identify and computational remove sequencing artifacts and contaminants. Furthermore, these distributions are used to perform differential abundance analyses and to reduce false discovery rate without the need to “correct” data (such as downsampling or inferring noise from other measurements [96, 97]) (FIGS. 9, and 11). Sampling from these distributions can also be used to improve data visualization by presenting the “clouds”of probable values rather than single values (FIGS. 9A-H, and 11A-H).

StochQuant has several limitations for analysis of 16S rRNA gene sequencing data. The version of StochQuant described here assumes that the sequencing technology is performing near its theoretical limits, does not include additional sources of measurement noise such as inefficiencies of experimental steps (such as PCR and nucleic acid extraction), volume transfer error or user-error. While with these assumptions, the proof-of-concept example of StochQuant still properly described the data presented in this manuscript, future versions/examples of StochQuant may need to incorporate these additional physical parameters. These additional physical parameters can be incorporated into the workflow model. Importantly, StochQuant is not magic—it does not remove stochastic measurement noise intrinsically present in analysis of low and moderate abundance taxa. In some cases, there may be so few molecules loaded or reads sampled that few (if any) meaningful conclusions can be drawn about a taxon from a given dataset, exemplified by the PCA and differential abundance analysis of P-13 (FIGS. 13A-D, FIGS. 15A-E). Accordingly, in some cases, the uncertainty of number of molecules in an environment can be so high that a determination cannot be made with a certain degree of confidence.

However, StochQuant identifies when measurements are performed in this regime, identifies whether the measurement is limited by loading of molecules or sampling of reads, and can be used to redesign experiments to improve the measurements (FIG. 5F\). Furthermore, in contrast to many existing approaches, StochQuant cannot be run on any existing amplicon dataset because StochQuant requires additional quantitative information such as an absolute anchoring value of the reference molecule. The current implementation of the proof-of-concept example of StochQuant uses a forward measurement model in combination with “brute-force”(computationally inefficient) bootstrapping algorithms to iteratively compute the inverse probability of the forward measurement model to infer taxon abundance. In the future, to process much larger datasets, more efficient Bayesian inference strategies (based upon the StochQuant forward measurement model) may need to be developed.

It is anticipated that StochQuant will be used to carefully design and rigorously interpret quantitative measurements of taxa across a wide range of environmental and biomedical microbiome studies. Microbes associated with a range of human tissues [46, 55], including mucosal biopsies [38, 56], cancerous tumors [71], vaginal [64] and respiratory samples [48, 50, 51] are of particular interest. and respiratory samples [48, 50, 51] are of particular interest. Even in high-load samples, such as saliva and stool, StochQuant will be useful to analyze key microbes present at low abundance, such as pathogens. It is expected StochQuant-derived taxon probability distributions to be usable in other downstream analysis methods.

It is also expected that the StochQuant approach described here can be expanded—in combination with appropriate absolute quantification methods—to amplicon sequencing with other gene targets (e.g. fungal) and adapted to other types of sequencing such as shotgun sequencing, RNA-sequencing, and single-cell RNA-sequencing to handle stochastic effects arising from sampling small numbers of molecules and reads for each target. In the context of this examples, the terms “expanded” and “adapted” to show that although the StochQuant method workflow can remain the same, the steps of a detection procedure which provide a measurement workflow representation for the StochQuant method workflow can change dependent on the detection procedure.

### Example 9: Forward Measurement Model to Generate Simulated Read Counts

This is a proof-of-concept example of part of building a StochQuant workflow, as discussed in Example 2 and FIG. 1.

In this example, the target molecule of interest, environment of interest, and a testing measurement that yields a molecular count of the target molecule were identified
a. Target molecule: 16S rRNA gene sequence of a particular microbe of interest (e.g., Bacillus).
b. Environment: A solution of isolated nucleic acids. referred also as the dilutions MD1, MD2, MD3, and MD4.
c. Testing measurement: 16S rRNA gene sequencing (amplicon sequencing)
d. Molecular count of the target: a read count of number of 16S reads from the target microbe of interest yielded from the testing measurement (amplicon sequencing)
   and, the manipulations of the molecules of interest that comprise the testing measurement workflow were identified.

Then, a reference molecule and method to perform the absolute anchoring measurement of the reference molecule were selected.
a. Reference molecule: 16S rRNA gene sequences of all microbes
b. Molecular count of the reference: a read count of the total number of 16S reads yielded from the testing measurement (amplicon sequencing). referred to this as the “read depth”. because this is a common term in the field to refer to total number of sequenced 16S reads in a sample.
c. Absolute anchoring measurement of the reference: total number of 16S rRNA gene copies in a solution of nucleic acids. This solution is the environment from which (i) a sample is taken to perform the absolute anchoring measurement (which is digital PCR measurement of total number of 16S rRNA gene copies) and (ii) another sample is taken to perform amplicon sequencing (testing measurement).

Then, the measurement workflow representation was built according to the procedure described below

Build Measurement Workflow Representation—Identify Measurement Workflow Representation Segments and Perform Segmental Calibration for each Segment

First, the Measurement Workflow Representation Segments were identified by identifying the manipulations or series of manipulations of the testing measurement workflow that (i) can impact the molecular count of the target/reference molecule obtained via the testing measurement, (ii) can be measured via a segmental calibration (discussed below) that can yield a representation of the Segment that can yield output numbers of target/reference molecules that approximate the output numbers of target/reference molecules of the manipulation(s) of the testing measurement, and (iii) for which the Segment Representation can be parameterized by the number of input target/reference molecules and/or the physical parameter of the manipulation(s) of the testing measurement that can impact the molecular count of the target/reference.

Of the manipulations of the testing measurement workflow, two Segments were identified such that the measurement workflow representation consisting of these two Segments yielded an accurate representation of the measurement workflow, as determined by the subsequent assessment of the accuracy of the measurement Workflow.

Segment 1: The Loading of Target/Reference Molecules into the Library Preparation Reaction.

The loading of the target/reference molecules into the library preparation reaction was identified as a manipulation that can impact the molecular count of the target/reference, and in particular a manipulation that is the separation of a measurable amount of sample from an environment.

In particular, this manipulation consists of using a pipette (which contains a component that enables the measurement of liquid volumes) to separate a measurable amount of sample (in the context of the Example 2 this is about 2-5 microliters) from an environment (solution of isolated nucleic acids). Accordingly, the measurement provided by the pipette provides the physical parameter of the amount of sample separated from an environment.

In the implementation of the mathematical representation of the manipulation (described below), the number of molecules in an environment is described as number of molecules per microliter (referred to as an absolute abundance) and the measurable amount of sample separated from an environment is described as the “loading volume” because this is the volume of sample that is loaded into the library preparation reaction.

For a detailed description of the “separation of a measurable amount of sample from an environment”, the segmentation calibration, the mathematical representation and physical parameters of the manipulation, please see Example 29. Separation of sample from environment.

Thus, to mathematically represent or model the manipulation of the reference molecule, the mathematical representation is a Poisson distribution and the physical parameters are the “loading volume” and the absolute anchoring value of the reference molecule in the environment.

To mathematically represent or model the manipulation of the target molecule, the mathematical representation is a Poisson distribution and the physical parameters are the “loading volume” and an inputted value of the “absolute abundance” of the target molecule in the environment.

It can be understood that in the context of performing the Assessment of the Accuracy of the Measurement Workflow Representation, the number of target molecules in the environment is known and thus the known number of target molecules in the environment is the physical parameter and can be inputted as the “absolute abundance” in this example.

In the context of using the Measurement Workflow Representation in an Inference Method, the number of target molecules in an environment is unknown (the user desires to determine the probability distribution of the target molecule in the environment). Thus, the inference procedure provides the physical parameter of the number of target molecules in an environment for this Segment such that the output number of target molecules from this Segment leads to the molecular count of the target yielded by the measurement workflow.

### Segment 2: The Library Preparation, Flow Cell Binding, and Sequencing of the Target/Reference Molecules

The manipulations of the library preparation (described above in the identification of manipulations section), the flow cell binding, and sequencing of the target/reference molecules were identified as the series of manipulations that comprise Segment 2.

These manipulations were grouped together because collectively, the manipulations can (i) impact the molecular count of the target/reference, (ii) a segmentation calibration has been performed for a similar series of manipulations, and (iii) the Segment can be mathematically represented and can be parameterized by the number of input target/reference molecules and the physical parameters of the manipulations.

In particular, the mathematical representation takes the following physical parameters as inputs:
the number of target molecules loaded into the library preparation reaction (which is obtained from the output of Segment 1),
the number of reference molecules loaded into the library preparation reaction (which is obtained from the output of Segment 1, which is dependent on the absolute anchoring value),
   and the molecular count of the reference molecule yielded by the measurement workflow.

In this proof-of-concept example of building a StochQuant Workflow, the Measurement Workflow Representation was implemented as a forward measurement model (described in this example), which generates simulated read count data through the function simulate_readcounts ( ) The function takes the physical parameters of the measurement workflow (absolute abundance; number of target molecules in an environment per unit volume of the environment, total bacterial load; absolute anchoring value of the reference molecule, template input volume; measurable amount of sample separated from environment, and read depth; molecular count of the reference molecule yielded by the measurement workflow) as inputs, and as an output, generates an array (of user desired length) of simulated read counts (molecular count of the target yielded by the measurement representation workflow). Accordingly, the function takes a number of target molecules in an environment, an absolute anchoring value of the number of reference molecules in an environment, a quantitatively measurable amount of sample separated from an environment, and a molecular count of the reference molecule obtained via a testing measurement, and as an output, the function yields an array (of user defined length) of probable molecule counts of the target molecule that would be obtained via the measurement workflow.

It can be understood that the following procedures of the function implement Segment 1:

The function first simulates the stochastic loading of molecules into the library preparation reaction. To do so, the function uses the product of the absolute abundance and template loading volume as the rate parameter Marget.

$${λ}_{target} = ( absolute abundance ) \times ( loading volume )$$

A discrete number of target molecules (Loaded Target Molecules) is simulated by sampling from a Poisson distribution with the rate parameter set to l_{target}.

Loaded Target Molecules˜Pois(λ_{target})

Next, the stochastic loading of non-target molecules is simulated using the average concentration of nontarget molecules by subtracting the target absolute abundance from the total bacterial Load.

In this example, it was chosen to model the loading of the reference molecules as the loading of target molecules and the loading of “non-target molecules” that comprise the reference molecule because the target molecule comprises part of the plurality of molecules that comprise the reference molecule. It can be understood that Nontarget in this example refers to reference molecules that are not the target. Thus target+nontarget refers to the total of the reference. Accordingly since in this example the reference marker is total 16S, then total 16S=non-target 16S+target 16S.

$$Nontarget Concentration = Total Bacterial Load - Absolute Abundance$$

The stochastic loading of nontarget molecules is simulated by sampling an integer from a Poisson distribution, with the rate parameter set to the product of the concentration of non-target molecules and the template loading volume.

$${λ}_{nontarget} = ( Nontarget Concentration ) \times ( Loading Volume )$$

A discrete number of loaded nontarget molecules is generated by sampling from a Poisson distribution with the rate parameter set to l_{nontarget}.

Loaded Nontarget Molecules˜Pois(λ_{Nontarget})

It can be understood that the following procedures implement Segment 2 of the measurement workflow representation.

Next, the stochastic sampling of reads on the sequencing flow cell is simulated. To do so, an integer is sampled from a Poisson distribution, with the rate parameter Mtarget_reads set to the average number of sampled reads. Mtarget_reads is computed as follows. First, target relative abundance is calculated by dividing the number of target molecules by the total number of molecules loaded.

$$Target Relative Abundance = \frac{Loaded Target Molecules}{( Loaded Target Molecules + LoadedNontargetMolecules )}$$

Next, the average number of sampled reads is calculated by multiplying the target relative abundance by the read depth.

$${λ}_{target \_ reads} = ( Target Relative Abundance ) \times ( Read Depth )$$

A discrete number of target reads is generated by sampling from a Poisson distribution with the rate parameter set to target_reads.

Target Read Count˜Pois(λ_{target_reads})

### Example B010 Computational Simulations of the Defined Community Sequencing Experiment as Part of the Validation of an Example Model of the 16S rRNA Gene Sequencing Molecular Detection Workflow

It can be understood that this is an example of how to use the Testing Measurement Representation as part of an assessment of the Accuracy of the Measurement Workflow Representation.

Read counts for each of the top 5 defined-community taxa were simulated for each sequencing replicate under each dilution condition. In total, in the assessment of the accuracy of the measurement representation workflow, 450 sequencing measurements were generated the measurement workflow, and the physical parameters of the measurement workflow that yielded these measurements were used in the Measurement Workflow Representation measurements to simulate the sequencing experiment (90 environments*5 taxa). Each measurement was simulated using the simulate_readcounts function described in Generation of Simulated Read Counts. Total loads for each environment were estimated based on the methods described in Quantification of Total Bacterial Load. Absolute abundance estimates were obtained by multiplying the mean relative abundance of each taxon in the MD1 dilution by the total load estimate for each environment. Each recorded read depth from each environment was used for the read depth (physical parameter). Simulated relative abundances were calculated for each simulated measurement yielded from the Measurement Workflow Representation by dividing the simulated read count by the observed read depth.

### Simulations of Detection Frequency

To simulate the frequency of detection for each taxon in each dilution, the sequencing experiment of the defined community was simulated many (n=100,000) times, following the procedure described above. A taxon was considered detected if the simulated read count was greater than zero.

$$f ( {SimReads}_{iteration , dilution , taxon , replicate} ) = \{ \begin{matrix}1 & if SimReads > 0 \\ 0 & otherwise\end{matrix}$$

For each simulation (iteration), within each dilution (MD1, MD2, MD3, or MD4) and within each taxon, the frequency of detection was calculated by dividing the number of times the taxon was detected by the number of sequencing replicates for that dilution.

$$Frequency of {Detection}_{iteration , dilution , taxon} = \frac{{\sum }^{replicates} f ( {SimReads}_{iteration , dilution , taxon , replicate} )}{Number of {Replicates}_{dilution}}$$

Calculations were performed with the Pandas and Numpy libraries.

To obtain a level of confidence of 95%, a confidence interval of simulated values from the measurement workflow representation, was provided by using the Numpy quantiles function, to identify the values with 0.025 and 0.9725 set to the lower and upper quantiles, respectively. According, the measurement workflow representation yielded a distribution of the number of times a target would be detected in the sequencing experiment among a collection of replicate detections of a target in a sample of an environment, and a confidence interval with a confidence level of 95% was provided by the distribution.

### Simulations of Coefficient of Variation

The sequencing experiment of the defined community was simulated many (n=100,000) times, following the procedure described above. Next, % CV was calculated for each genus in each dilution (MD1, MD2, MD3, MD4) for each simulated experiment. % CV was calculated as follows:

$$C {V}_{iteration , dilution , taxon} ( \% ) = \frac{Standard Deviation ( Rel . Abund {.}_{iteration , dilution , taxon} )}{Mean ( Rel . Abund {.}_{iteration , dilution , taxon} )} \times 100$$

Calculations were performed in Python with the Pandas and Numpy libraries.

### Example 10: Generation of Probability Distributions of Total Bacterial Load from ddPCR Data

It can be understood that this is an example of obtaining an absolute anchoring value that is a distribution of number of reference molecule in an environment based on performing an absolute anchoring measurement in a sample of the environment.

In this example, the software implementation of StochQuant generates probability distributions of total bacterial load from droplet-digital PCR data through the function estimate_total_load ( ) The function takes the total load measurements from droplet-digital PCR (generated by the BioRad QuantaSoft software) and experimental handling parameters as inputs, and as an output, generates a probability distribution describing the concentration of molecules in a sample. The method contains additional hyperparameters that may be set by the user, including the resolution of the distribution, and the number of iterations the model runs to build the distribution.

First, the function computes the expected target molecule concentration in the sample by multiplying the observed concentration by the digital PCR fold-dilution and digital PCR reaction volume, and by dividing by the digital template volume. Next, the expected total number of molecules in the sample is computed by multiplying the expected target molecule concentration by the elution volume.

Next, an array of discrete number of molecules, sampled along a user-defined interval (in log 10 space), is generated for one order of magnitude above and below the expected number of target molecules. It is possible, particularly for low total load samples, that many of the values in the array may not correspond to integer values (e.g. 1.3 molecules). The function therefore converts all values in the array to integers, and only retains unique integer values. These integer values are then divided by the elution volume to obtain an array of concentrations.

To improve computational performance, the method does not consider all possible concentrations. Instead, the method begins building the probability distribution of target abundance at the expected value of the target molecule concentration in the sample. The method then progresses away from the expected value (in ascending and descending order), and periodically checks the sum of probabilities of the prior 10 concentrations. If the function repeatedly gets a series of zero-sum probabilities, the function does not continue computing probabilities for more target concentrations.

For each target concentration, the function performs the following operation many times (set by a user defined number). In each iteration, the function first accounts for any upstream dilutions prior to digital PCR by dividing the concentration by the digital PCR dilution (a user-specified experimental parameter). The function then uses a Numpy random number generator to randomly select an integer from a Poisson distribution with the rate parameter set to the loading concentration multiplied by the digital reaction template volume (e.g., volume of the original sample used for quantification). The function then estimates the concentration of target molecules per droplet by multiplying by the reaction volume per droplet and by dividing the simulated copies loaded by the digital reaction volume. For estimates based on the BioRad QX200 digital droplet generator, a volume of 8.49e-4 microliters was assumed.

Next, the number of positive droplets is simulated. To do this step, first a Numpy random number generator was used to instantiate a vector of length n, where n is the total number of droplets generated for the sample, and randomly sampled from a Poisson distribution with the rate parameter set to the concentration per droplet, to fill this array. The array of integers was then converted to a Boolean array, where nonzero values were converted to a value of one. Then the Boolean values of this array were summed to get the number of simulated positive droplets. If the number of simulated positive droplets is within the margin of error (2 (observed positive droplets) 1/2), then the iteration is considered a match, and the number of “matched” iterations for the tested concentration is increased by a value of one. This procedure is usually repeated approximately 10,000 times per tested concentration. The method then stores the final number of iterations in which a match was found, and then moves on to the next concentration to repeat this procedure.

Once the function has performed the above procedure on all tested concentrations for a given measurement, the method then normalizes the matched iterations value such that all values sum to 1. This normalization procedure results in probabilities for each tested concentration, and collectively, enables a probability distribution of target concentration to be constructed.

### Example 11: Generation of Probability Distributions of Taxon (Target) Abundance

This is an example of incorporating a measurement workflow representation and the physical parameters into an inference method to yield a probability distribution of target abundance in an environment.

To generate probability distributions of taxon absolute and relative abundance, a python function was written that takes the measurement workflow representation and the physical parameters (a read count measurement from amplicon sequencing (a molecular count of the target molecule obtained via the amplicon sequencing testing measurement), a total bacterial load measurement (an absolute anchoring value of the reference molecule), and experimental handling parameters (a measurable amount of sample separated from an environment and a molecular count of the reference molecule obtained via the amplicon sequencing testing measurement)) as inputs, and as an output generates a probability distribution of target abundance in an environment in the form of a 2D array containing either (a) concentrations of molecules (taxon absolute abundance) or (b) relative abundances of molecules (taxon relative abundance), and a probability distribution over the abundances. The function described is an inference method that incorporates the measurement workflow representation and the physical parameters to yield a probability distribution of target abundance in an environment.

The inference method function contains additional hyperparameters that may be set by the user, including the resolution of the distribution, and the number of iterations the function runs to build the distribution. At a minimum, the function requires the physical parameters (a read count, a total load estimate, experimental handling parameters (e.g., volume transfers), and a read depth). The method can work either on a single estimate of total bacterial load, or on a probabilistic array of total loads (see Example 10). Below, the implementation is described with an array of total loads.

### Generation of Probability Distributions of Taxon Absolute Abundance

To improve computational performance, the function does not consider all possible concentrations. Instead, the function begins building the probability distribution of target abundance at the expected value of the target molecule concentration in the sample. The method then progresses away from the expected value in the positive and negative direction, and periodically checks the sum of probabilities of the past few concentrations. If the method repeatedly gets a series of zero-sum probabilities, the method does not continue computing probabilities for more target concentrations.

First the method computes the expected number of target copies in the sample. To do so, the expected target copies is calculated by multiplying the relative read abundance by the expected value of the total bacterial load estimate and the elution volume. It can be understood that the expected number of target copies in the sample is obtained via a deterministic model of the molecular detection workflow.

$$Expected Target Copies = Expected Total Load \times \frac{Read Count}{Read Depth} \times Elution Volume$$

In this example, this equation can be re-written as:

Target_{Env}=Number of target molecules in an environment

Ref_{Anchor}=Absolute anchoring value of the reference

Target_{MolecCount} =Molecular count of the target molecule obtained via the testing measurement

Ref_{MolecCount} =Molecular count of the reference molecule obtained via the testing measurement

Envₛₐₘₚₗₑ=measurable amount of sample separated from the environment

$${Target}_{Env} = {Ref}_{Anchor} \times \frac{{Target}_{MolecCount}}{{Ref}_{MolecCount}} \times {Env}_{sample}$$

This equation was obtained by re-arranging the deterministic model:

$$\frac{{Target}_{env}}{{Env}_{sample}} \times \frac{{Ref}_{Moleccount}}{{Ref}_{Anchor}} = {Target}_{MolecCount}$$

The expected value of the total bacterial load (a deterministic approximation of the absolute anchoring value of the reference molecule) is calculated as follows:

First, an array of total bacterial loads of length (n=1000) for a sample is generated by using the np.random.choice package with the array of total bacterial loads and its associated array of corresponding total load probabilities as follows:

$$Total Loads = np . random . choice ( Possible Total Loads , size = n , p = Total Load Probabiltiies )$$

Note, in this example, “Possible Total Loads” and “Total Load Probabilities” collectively refer to the distribution of probable numbers of the reference molecule in an environment, expressed as a 2D array (discussed above in Methods).

Next, the expected value of the Total Loads array is calculated as follows:

$$Expected Total Load = 10 \frac{\sum {log}_{10} ( Total {Load}_{i} )}{n}$$

For each taxon absolute abundance, the following operation is performed many times (set by a user defined number). In each iteration, the function generates a simulated read count following the procedure described in the above section (Generation of simulated read counts).

If the simulated read count is within the margin of error, then the iteration is considered a match, and the number of “matched” iterations for the tested concentration is increased by a value of one. Accordingly, if the probable molecular count of the target molecule yielded by the StochQuant model of the molecular detection workflow is approximately equal (within a margin of error) to the observed molecular count of the target molecule.

In this example, for a given simulated read count, the allowed margin of error is 2\sqrt{ObsReads} where ObsReads is the observed read count from the sequencing data. In this example, each tested target abundance is indexed by j, and each simulated read count is indexed by i.

$$f ( {SimReads}_{j , i} ) = \{ \begin{matrix}1 if {SimReads}_{j , i} - \sqrt{ObsReads} \leq \\ {SimReads}_{j , i} \leq SimR {eads}_{j , i} + \sqrt{ObsReads} \\ 0 otherwise\end{matrix}$$

The number of matches for a given target abundance can be given by:

$${Matches}_{j} = \sum f ( {SimReads}_{j , i} )$$

Then, the number of matched iterations is normalized such that all values sum to 1. This normalization procedure results in probabilities for each tested abundance, and collectively, this enables a probability distribution of target abundance to be constructed.

An example calculation is as follows:

$${P}_{j} = \frac{{Matches}_{j}}{\sum {Matches}_{j}}$$

Where Pⱼ is the probability of target abundance j generating the observed read count.

When each probability distribution of target abundance in an environment is generated, in this example, the inference method performs a series of quality assurance steps. For example, the method checks the number of abundances for which a nonzero probability was assigned and checks the total number of iterations across all tested abundances that were used to build the distribution. If either of the values are below user-defined thresholds, the method will adjust the hyperparameters and attempt to rebuild the probability distributions of abundance.

One potential reason for poor-quality distributions is if the observed read count corresponds to less than one discrete molecule in the sample. This can occur for several reasons (such as the read count being from a sequencing artifact such as taxonomic misclassification or barcode hopping). If the probability distribution does not pass the quality assurance steps and the expected value of the number of molecules in the sample is less than one, the method will attempt to build a probability distribution of abundance by setting the observed read count to zero.

Another potential reason that a distribution may fail the quality-assurance check is if the number of abundances with nonzero probabilities is very low. One potential cause for this is that the resolution of abundances is too low. If this is the case, the method will incrementally increase the resolution by 2×, attempt to generate a probability distribution of abundance, and then check the quality of the distribution. This procedure may be repeated until a user-defined cutoff. In this manuscript, a cutoff of 16× was used.

If the above procedure did not work, the method will then simultaneously increase the resolution and the total number of iterations per tested abundance. If all of the above methods did not work, the measurement was flagged, and a warning was returned.

Generation of probability distributions of taxon relative abundance

This is another example of incorporating a measurement workflow representation and physical parameters into an inference method. The difference between this example and the previous example, is that the previous example yielded a distribution of target absolute abundance (target copies per unit volume) and here, this example yields a distribution of target relative abundance (target copies per reference copies in the environment).

To generate a probability distribution of target relative abundance, a procedure was followed similar to the one outlined above, with a few differences. The main difference is in how the array of abundances is generated to ensure that the relative abundances tested correspond to discrete molecules.

First, the expected value of target relative abundance is calculated by dividing the target read count by the read depth of the sample.

Next, an array of relative abundances (for which a probability distribution will be generated) is generated. To create the array of relative abundances, first, an array of discrete molecules that spans from zero to the total number of possible molecules (total bacterial load multiplied by the elution volume) is created. This array contains evenly spaced values at a user specified resolution in either linear or log space.

In example, base 10 logarithms are used. It is possible, particularly for low total load samples, that many of the values in the array may not correspond to integer values (e.g. 1.3 molecules). The method therefore converts all values in the array to integers, and only retains unique integer values. These integer values are then divided by the elution volume to obtain an array of concentrations. To obtain the array of relative abundances, each concentration is divided by the total bacterial load to obtain a relative abundance value. If an array of total loads is supplied, the maximum total load is used.

Next, probability distributions of relative abundance are built following a procedure similar to the procedure described for absolute abundances. However, one key difference is that here, a relative abundance is supplied. Therefore, a key first step is to convert the relative abundance to an absolute abundance. If an array of total loads is supplied, for each total load, an absolute abundance is calculated by multiplying the relative abundance by the total load. This calculated absolute abundance is then used to determine the probability that the tested relative abundance led to the observed read count.

### Example 12 Computationally Sampling Target Abundances from Probability Distributions of Taxon Abundance

This is an example of converting a probability distribution of target abundance from one form into a probability distribution of target abundance in another form. Both are probability distributions of target abundance.

To computationally sample discrete abundance values from a probability distribution of target abundance, the random.choice function from Numpy was used. To perform this operation, an array was supplied of tested target abundances, and an array of probabilities for the target abundances, and a user-defined number of bootstrap replicates to sample from the distribution. For most analyses, used a size value of 1,000 was used.

### Example 13: Contamination Filtering

In order to improve contamination filtering a python script was written to perform a contamination filtering approach using the model generated absolute abundance data. Contamination filtering was performed at the Phylum, Class, Order, Family, and Genus taxonomic levels. At each taxonomic level, the StochQuant absolute abundance estimates for each taxon in each sample were used. For each taxon in each NTC, the StochQuant absolute abundance estimates were used to compute the upper 99^{th} percentile absolute abundance. Accordingly, the probability distributions was used of number of target molecules in an NTC environment to obtain a value from the upper bound of confidence of the number of target molecules in an NTC environment. For each taxon in each biological environment, the StochQuant absolute abundance estimates were used to compute the lower 1^{st} percentile absolute abundance.

Accordingly, the probability distributions were used of number of target molecular in an environment from a biological specimen of interest to obtain a value from the lower bound of confidence of number of target molecules in the environment. Each NTC was then compared to each biological environment and determined whether 1^{st} percentile of the taxon in the biological sample was greater than the 99^{th} percentile absolute abundance of the taxon in the NTC. This procedure was repeated at each taxonomic level. If a taxon (at each taxonomic level) was found to be in higher absolute abundance than in all of the NTCs (using the approach described above), the taxon was determined to be present in the biological environment with an abundance (number of molecules in the environment) higher than the abundance in the NTC environment. This determination was used to infer that a taxon could be of biological relevance (at an abundance that could confidently be determined to be greater than an abundance of background contamination). Thus, these determinations were used to select which targets were to be retained in downstream analyses.

### Example B14: Statistical Analyses with StochQuant abundance estimates Differential Abundance Analyses

The StochQuant software contains a python function that as an input, takes a Pandas DataFrame that consists of the bootstrapped replicates from each taxonomic measurement of each sample, metadata variables of interest (e.g., control, treated), statistical test, and user-defined level of significance, and as an output, for each taxon, produces an array of statistical test values. For example, let's consider a simple case of twenty taxa that are in five samples that come from a control condition and five samples that come from a treated condition, and the Kruskal-Wallis statistical test can be performed for each taxon in these samples, and have specified that our significance level is 0.05. With traditional methods, a test statistic and P-value can be obtained for each taxon. However, it is possible that the observed result was heavily biased by stochastic events, and that in fact, if the same samples were to be re-sequenced, a different result would be observed.

To overcome this limitation, our function performs the statistical test on each bootstrapped replicate. In this example, for each taxon, 6 arrays of taxon abundance (one array each of the 3 measurements from control and 3 measurements from treated) ere prepared. The function selects one value from each array, performs the statistical test, and stores the test statistic value and P-value. The function iteratively repeats this procedure to obtain an array of test statistics and an array of P-values.

The function can take the array of statistical test values, and compute the frequency of differential abundance analyses, which can be interpreted to be the probability that the taxon of interest is differentially abundant between the groups of samples being analyzed. To compute this probability, the array of P-values is converted into an array of Booleans, where the P-value is converted to a zero if the P-value is greater than the significance level, and the P-value is converted to 1 if the P-value is less than the significance level. Then this array of Booleans is summed to determine the number of iterations for which a significant P-value was recorded. Finally, this value is normalized by dividing the total number of iterations.

The above example described the Kruskal Wallis Test, however, this procedure can be applied to other statistical tests and analyses, following similar principles, where analyses are repeatedly performed on values that are sampled from the arrays of abundances.

### Example 15: Statistical Analyses with StochQuant Abundance Estimates Principal Component Analysis (PCA)

PCA on the StochQuant estimates of taxon relative abundances was performed as follows. First, PCA was performed on standard estimates of taxon relative abundance using the Sklearn Decomposition package to obtain a loading matrix. Then, the loadings matrix was multiplied by the transpose of the pseudo center-log transformed StochQuant estimates matrix to project the StochQuant estimates onto the standard principal components.

### Example 16 Sampling from an Environment to Perform the Absolute Anchoring Measurement and Sampling from an Environment to Perform the Measurement Workflow

In this example the Environment is sampled twice. One sampling activity (for Sample 1) is performed to separate a sample from the environment to perform an absolute anchoring measurement to provide an absolute anchoring value of the reference molecule in an environment. Another sampling activity (Sample 2) is used to obtain a sample from the environment to perform the testing measurement (which involves a manipulation of the target and reference molecules) to yield the molecular count of the target molecule and the molecular count of the reference molecule via the testing measurement.

### Example 17 “Spike In” of Reference into the Environment

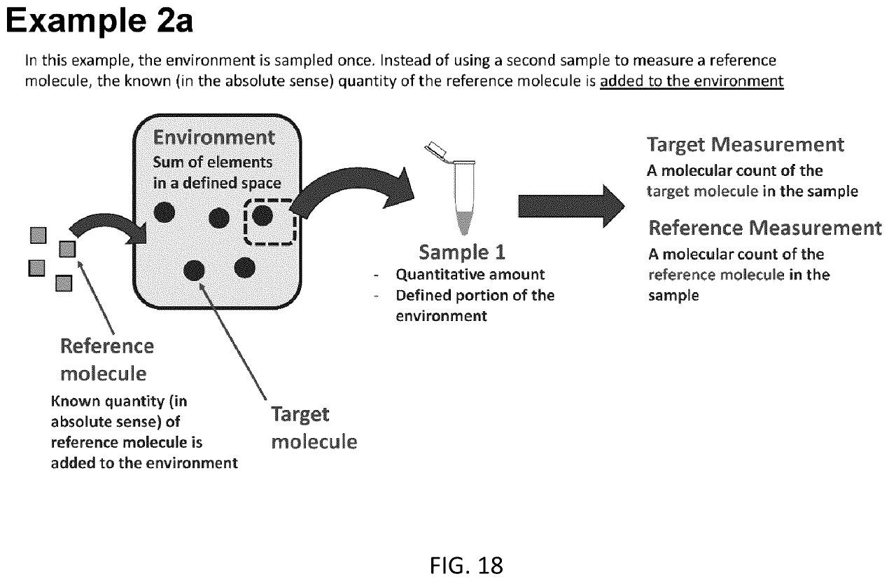

In this example, the reference molecule of a known quantity (in the absolute sense) is present in the environment (FIG. 18). The known quantity of the reference molecule in the environment is used as the absolute anchoring value of the reference molecule. The environment is sampled once, and the testing measurement is performed such that the target and reference molecules are manipulated to yield a molecular count of the target and a molecular count of the reference via the testing measurement. This example is in contrast to Example 16. Instead of using a second sample to measure a reference molecule (as in Example 16), the known (in the absolute sense) quantity of the reference molecule is added to the environment or known to be present in the environment at a known quantity.

### Example 18 “Spike In” of Reference into a Sample of the Environment

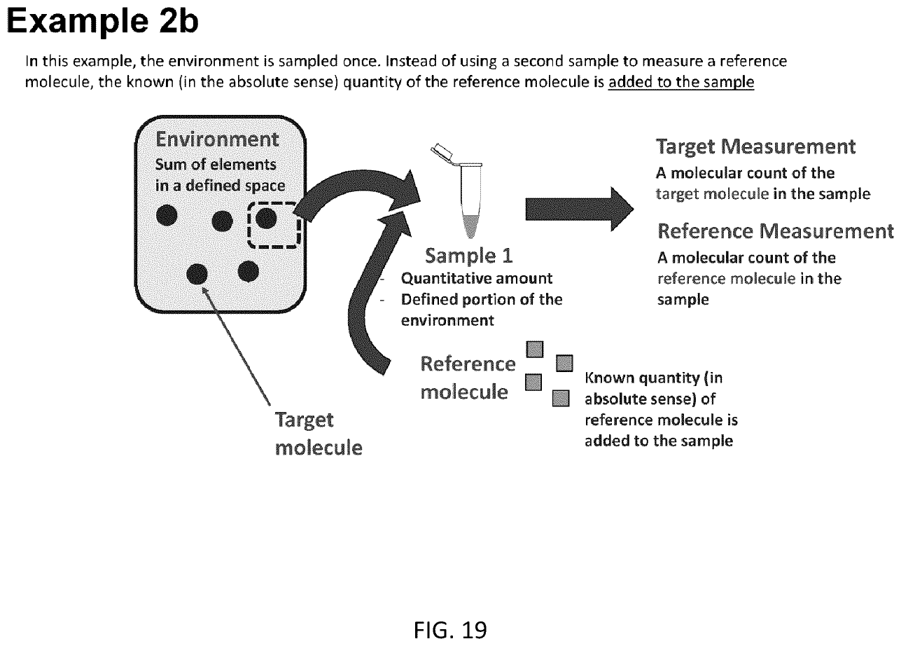

In this example, a quantitative amount of the environment is sampled from the environment once (the environment is sampled once) (FIG. 19). Then, a reference molecule, the known (in the absolute sense) quantity of the reference molecule is added to the sample. Then, the target/reference molecules of the sample are manipulated in the testing measurement workflow to yield a molecular count of the target and a molecular count of the reference.

### Example 19 Sub-Sampling of a Sample

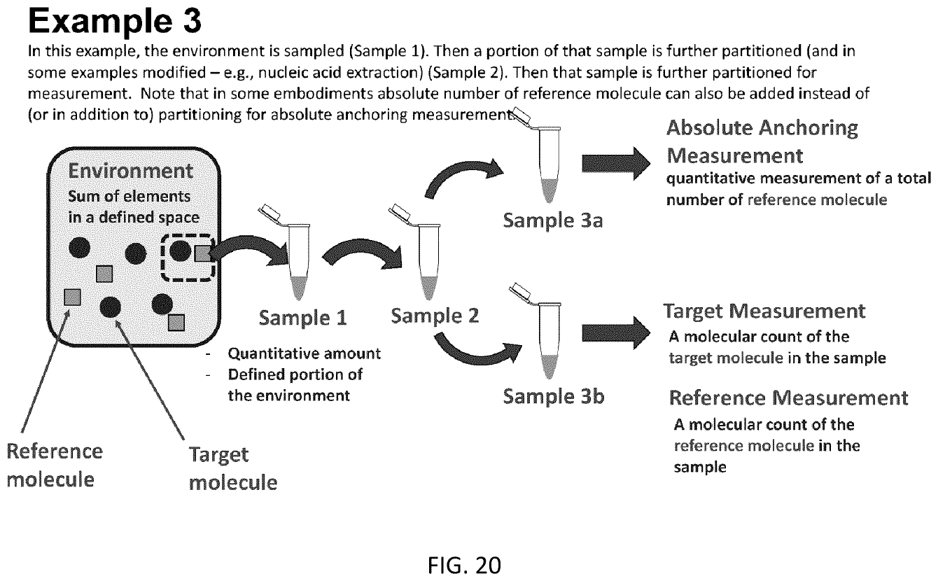

In this example (FIG. 20), a sample (Sample 1) of an environment is obtained by separating a quantitative measurable amount of sample from an environment. Then a sub-sample (Sample 2) is obtained by separating a quantitative amount of sample from Sample 1. Then, a sub-sample of Sample 2 (Sample 3a) is obtained by separating a quantitative amount of Sample 2. Then, an absolute anchoring measurement is performed in Sample 3a to yield an absolute anchoring value of the reference molecule. Another sub-sample of Sample 2 (Sample 3b) is obtained by separating a quantitative amount of Sample 2. Then the target/reference molecules of the sample are manipulated to perform a testing measurement to yield a molecular count of the target and a molecular count of the reference.

### Example 20 the Environment is a Sample of Another Environment

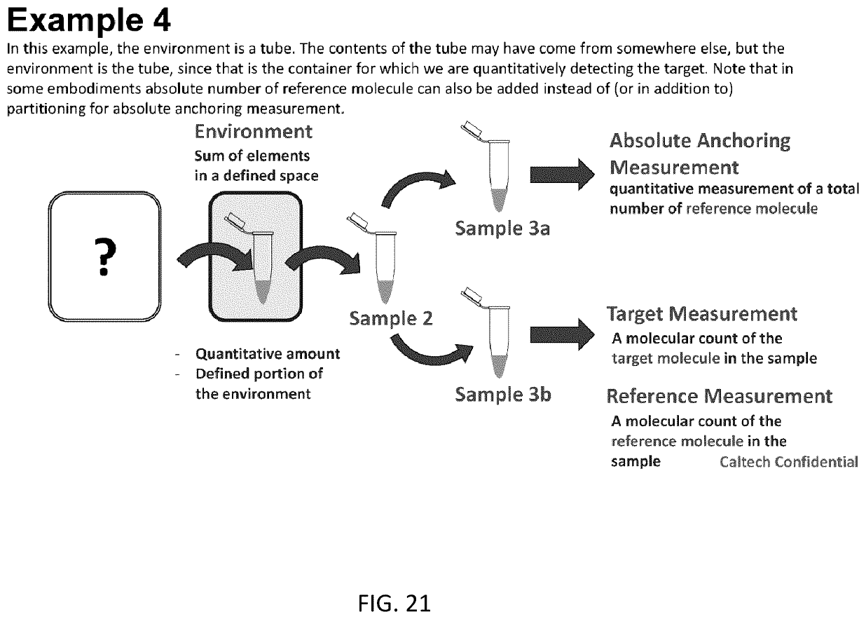

In this example (FIG. 21), the environment is a solution of nucleic acids in a tube. The contents of the tube may have come from somewhere else, but the environment is the tube, since that is the container for which the user is quantitatively detecting the target. Then, the same manipulations are performed as in Example 19. This example shows that the environment is dictated by the quantitative detection of the target in the Measurement Workflow.

### Example 21: General Procedure for Absolute Anchoring Measurement

This example describes the general procedure for making an absolute anchoring measurement to build a StochQuant Workflow

### Example 22: Absolute Anchoring Measurement of the 16S rRNA Gene Reference Molecule Via qPCR with a Standard Curve to Provide the Absolute Anchoring Value of the Reference Molecule

This example describes the general procedure for making an absolute anchoring measurement to build a StochQuant Workflow

In particular, this example discusses how to perform an absolute anchoring measurement to obtain an absolute anchoring value of a reference molecule with a qPCR measurement of the reference molecule.

In some embodiments, an absolute anchoring measurement is performed by using a qPCR with a standard curve measurement technique, which can be used to provide an absolute anchoring value of the reference molecule.

For example, qPCR with the “universal” 16S primers used for sequencing in Example 3 with a standard curve can be used to obtain an absolute anchoring value of the 16S rRNA gene reference molecule. A standard curve enables one to determine, for a given Cq (yielded by a qPCR measurement of a target molecule), the number of target molecules that would yield the observed

Cq [98]. This was accomplished by measuring serial dilutions of known numbers of reference molecules with qPCR, and then performing a linear regression between the known number of molecules (x-axis) and the observed Cq values (y axis).

A standard curve can also be obtained by measuring different numbers of a reference molecule with digital PCR (x-axis) and qPCR (y-axis). Note, it is preferable to take a log transformation (e.g., log 2 or log 10) of the number of reference molecule prior to performing the linear regression. The equation yielded by the linear regression takes a Cq value as an input, and as an output, yields number of reference molecules in a sample. In some embodiments, the number of reference molecules in a sample yielded by the linear regression is the absolute anchoring value.

In some embodiments, the number of reference molecules in a sample can be used to yield an absolute anchoring value of the reference in the environment. For example, if the standard curve yields the number of reference molecules in a sample, but an absolute anchoring value of number of reference molecules in an environment is needed as a physical parameter of the measurement workflow representation, then a model of separating a sample from an environment (see Example 2, 3) can be used to yield an absolute anchoring value of a reference molecule in an environment.

The absolute anchoring value of the reference can be used in a StochQuant model of a molecular detection workflow, as described in Example 2, 3, 6, 9.

### Example 23: RT-qPCR for the Absolute Anchoring Value

In some embodiments, RT-qPCR with a standard curve can be used to obtain an absolute anchoring value of a reference molecule. In this example, the reference molecule is an RNA molecule, such as the mRNA transcript of the human MYH9 gene. A standard curve can be obtained following the same procedure described in [qPCR for the absolute anchoring value], except in this example, a reverse-transcription is performed before qPCR.

In this example, the reference molecule is the mRNA transcript of the human MYH9 gene, and a standard curve was obtained from previous experimentation. The standard curve was used to obtain an absolute anchoring value of the reference molecule in a sample.

For example, serial dilutions (1×, 10×, 100×, 1000×, 10⁴×, 10⁵×, and 10⁶×) of a solution of RNA from a human (the solution containing the MYH9 mRNA transcript) were used. A reverse transcription manipulation was performed on the solutions followed by (a) digital PCR to obtain an absolute measurement of the MYH9 target or (b) qPCR to obtain a Cq measurement of the target. Then, linear regression was performed between the absolute concentration of the target obtained via RT-digital PCR (y-axis) and the RTqPCR Cq value (x-axis) with the y-axis log 10 transformed. The linear regression provided an equation, which was re-arranged for reference copies/μL as a function of input Cq:

$$\frac{{Reference}_{Copies}}{{environment}_{vol}} = {10}^{\frac{( Cq - 34.96 )}{( - 3.272 )}}$$

### Example 24 Performing Digital PCR Measurement to Provide an Absolute Anchoring Value for the Measurement Workflow

This is an example of performing a digital PCR measurement to provide an absolute anchoring value for a Measurement Workflow Representation.

Digital PCR is a technique that enables absolute quantification of the number of target molecules. Several digital PCR systems and instruments exist such as the Bio-Rad QX200 droplet digital PCR system, the ThermoFisher QuantStudio 3D digital PCR system, the Stilla Naica System, the RainDance RainDrop Digital PCR system, and the Combinati Absolute Q digital PCR system.

This example discusses performing digital PCR with the BioRad QX200 droplet digital PCR system. However, it can be understood that this example can apply to performing other absolute anchoring measurements that share similar features.

To perform the absolute anchoring measurement, a sample from an environment is taken (see Example 16) In this case, a pipette is used to measure and separate 2.5 μL of sample from an environment (a solution of nucleic acids). The sample is used in a digital PCR reaction that contains primers that can anneal to the reference molecule and the other necessary reagents to perform the QX200 droplet digital PCR system workflow. Thus, the QX200 droplet digital PCR system workflow can provide a measurement of the target molecule in a sample of the environment.

In this example, the absolute anchoring measurement provided by the QX200 droplet digital PCR system workflow is calculated by the QX200 droplet digital PCR system software (QuantaSoft Software) to yield the measurement in the form of reference molecules per microliter of the sample. The software can also yield the measurement in the form of the number of positive and negative droplets, and the user can calculate the concentration based on the formulas provided in the QuantaSoft Software Instruction Manual.

In this example, the user can use the quantitative measurable amount of sample separated from the environment (volume in microliters) and the absolute anchoring measurement provided by the QX200 droplet digital PCR measurement (reference copies per microliter) to yield the absolute anchoring value of the number of reference molecules in the environment.

This can be provided by the following mathematical operation:

$${Reference}_{environmentCopies} = \frac{{Reference}_{sampleCopies}}{μ L} \times {vol}_{sample} ( μ L )$$

### Where:

Reference_{environmentCopies} is the absolute anchoring value of reference molecules in the environment
Reference_{sampleCopies}/μL is the absolute anchoring measurement
And volₛₐₘₚₗₑ (μL) is the measured amount of sample separated from the environment.

A similar example is described in Example 3, except the absolute anchoring value is a probability distribution of numbers of reference molecules in the environment based on the absolute anchoring measurement provided by the QX200 droplet digital PCR measurement.

### Example 25: Spike-in Example 1

This is an example of including a “spike in” of the reference molecule in the environment to provide the absolute anchoring value of the reference in the environment (see Example 17).

It can also be understood that a reference molecule for the “spike in” should be chosen such that the environment (prior to the inclusion of the spike-in) is expected to contain zero molecules of the spike-in. Accordingly, after the inclusion of the spike-in, the number of reference molecules in the environment is known.

It can be understood that the “spike in” that provides the absolute anchoring value of the reference in the environment can also be used for additional purposes such as quality control, and/or detection of technical variability.

In this example:
The measurement workflow I the amplicon sequencing measurement workflow from Example 3 is the measurement workflow,
the target molecule is the 16S rRNA gene of a taxon of interest,
the environment is a solution of nucleic acids (e.g., MD1, MD2, MD3, MD4),
Molecular count of the target: a read count of number of 16S reads from the target microbe of interest yielded from the testing measurement (amplicon sequencing)

In this example, the genomic DNA from several organisms was added to form an environment (the defined community from the Example 3).

In this example, the 16S rRNA gene of Listeria, one of the organisms that was added to form the environment is the “spike in” and thus the reference molecule is the 16S rRNA gene of Listeria. In this example, the 16S rRNA gene of Listeria was chosen because the number of 16S rRNA gene molecules of Listeria added to the environment was a known amount and the 16S rRNA gene molecules of Listeria (the reference molecule) can be detected by the measurement workflow.
a. Absolute anchoring value: The number of reference molecules “spike-in” into an environment. In this case, it is the number of Listeria 16S rRNA gene molecules in the environment.
b. Molecular count of the target: a read count of number of 16S reads from the target microbe of interest yielded from the testing measurement (amplicon sequencing)
c. Molecular count of the reference: a read count of the number of 16S reads from the reference molecule (Listeria 16S rRNA gene molecules) yielded from the measurement workflow (amplicon sequencing)

In this example, the same measurement workflow representation from Example 3 was used.

In this example, the absolute anchoring value of the reference molecule in provided by the “spike in” was used as the physical parameter in the measurement workflow representation.

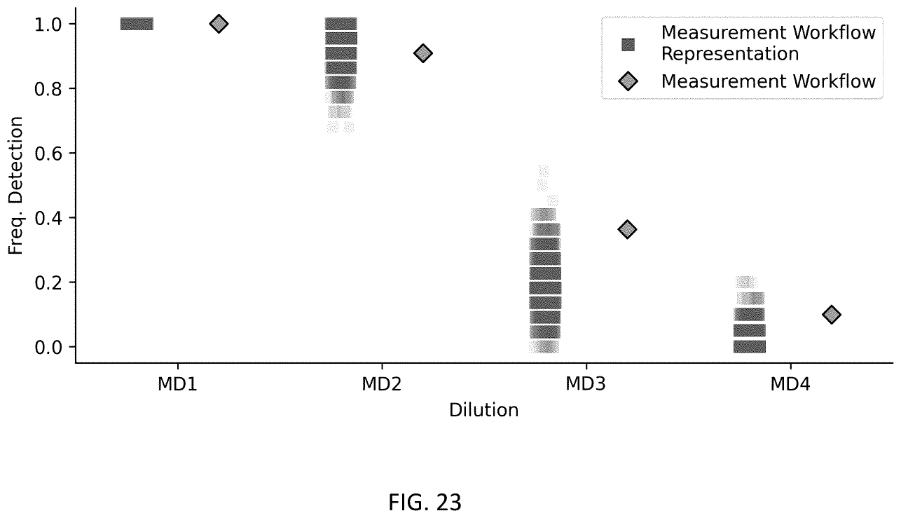

In this example, a similar assessment of accuracy of the measurement workflow representation was used as Example 5, and similar levels of performance/accuracy of the measurement workflow representation were observed (FIG. 23).

### Example 26 Spike-in Example 2

This is an example of including a “spike-in” of the reference molecule in a sample of the environment to provide the absolute anchoring value of the reference in the sample (see Example 18). It can be understood that the reference molecule is included in a sample (of the environment) that includes the target molecule as part of the testing measurement workflow. For example, in an amplicon sequencing measurement workflow, a “spike-in” may be included in the library preparation reaction, which contains a sample of the environment.

This example includes providing the measurement workflow representation and the physical parameters with particular focus on the selection and usage of the absolute anchoring value as it relates to the “spike-in” of the reference molecule.

It can be understood that the “spike-in” that provides the absolute anchoring value of the reference in a sample of the environment can also be used for additional purposes such as quality control, and/or detection of technical variability.

In this example, the same measurement workflow, target molecule, molecular count of the target obtained via the testing measurement workflow, reference molecule, and molecular count of the reference molecular obtained via the testing measurement workflow are the same as those described in Example 25: Spike-in Example 21 with the exception that
the environment is a solution of nucleic acids that does not contain the reference molecule,
the absolute anchoring value is the number of reference molecules in a sample of the environment.

In this example, the measurement workflow representation contains the same two Segments that were described in Example 5. In this example, it is understood that the number of reference molecules in the environment is zero. Because the number of reference molecules in an environment is zero, a user can choose to not perform a mathematical representation of the reference molecule during the separation of a sample from an environment manipulation, because the manipulation always yields zero reference molecules.

These examples show that in some embodiments one can model the uncertainty of the number of spike-in molecules in the sample of the environment. One may choose to do this if the number of spike-in molecules is low-to-moderate.

### Example 27 Unique Molecular Identifiers (UMIs) Example

This is an example of using the measurement of unique molecular identifiers (UMIs) to provide an absolute abundance value of the number of reference molecules in a sample of an environment. This is also an example of a measurement workflow that yields an absolute anchoring value of the reference molecule.
Measurement Workflow: single cell RNA sequencing
Reference molecule: plurality of UMI-tagged molecules
Absolute Anchoring Value: the number of UMI-tagged molecules in a sample of the environment.

In this example, the measurement workflow can yield a measurement of the reference molecule.

In this example, the use of UMIs in the context of single-cell RNA sequencing is shown. However, UMIs can be used in other contexts as well. Examples: UMIs can be used in amplicon sequencing, shotgun metagenomic sequencing, RNA-sequencing.

In this example, UMIs are used in the context of the Single-cell RNA sequencing Example 38 and Example 39.

In this example, the absolute anchoring value (number of UMI-tagged molecules in a sample of the environment) is obtained as follows. First, a measurement representation of the testing measurement that yields a count of the number of unique detected UMIs was built. This is done by using Poisson statistics to calculate the probability of detecting a given UMI based on the total number of UMIs in the sample and the total number of reads (molecular count of the reference) sequenced. Then, probable numbers of detected UMIs yielded by the testing measurement are provided by the mathematical representation via a binomial distribution parameterized by the number of UMIs in the sample (UMI_loaded) and the probability of obtaining a nonzero read count for each UMI. The mathematical representation is below, which was implemented as a Python function using the Numpy.random.binomial function:

$${λ}_{UMI \_ Detected} = \frac{1}{( {UMI}_{loaded} )} \times Total Reads$$

$${p}_{nonzero} = 1 - {e}^{- {λ}_{UMIdetected}}$$

$$Detected UMIs ∼ Binom ( {UMI}_{loaded} , {p}_{nonzero} )$$

Then, an Inference Method, such as the one described in Examples 6, 11, and 35 was used for each sample of a subset of (n=1,033) cells (e.g., samples) described in further detail in Example 38.

It is understood that other approaches can be used to obtain an absolute anchoring value of the number of UMIs in a sample, such as the use of software packages such as Cell Rnanger, UMI-tools, STARsolo, Alevin, Kallisto with bustools.

### Example 28 Case-Zero Step Specific Example

The following is an example general procedure to guide a skilled user to establish a step of a StochQuant molecular detection workflow.

First, identify a step of the molecular detection procedure which provides a StochQuant method workflow. A step is identifiable if a manipulation of the target/reference molecule occurs such that the manipulation can yield a change in the number of target/reference molecule. If such a manipulation is identified, the number of target/reference molecules at the start of the step (prior to the manipulation) is referred to as the input number of target/reference molecules of the step, and the number of target/reference molecules at the end of the step (after the manipulation) is referred to as the output number of target/refence molecules of the step.

It can be understood that in some cases, a step can be arbitrarily large or small or multiple manipulations may be combined into a single step. To determine whether a manipulation or series of manipulations can be considered a step, in practice, the manipulation must be able to be represented by a model of the manipulation, such that the model can yield a distribution of probable number of output target/reference molecules after the manipulation as a function of the number of input molecules of the manipulation and measurable factors of the manipulation.

Accordingly, a probability distribution (and measurable factors to parameterize the distribution) that enable tracking of probable number of output molecules for a given number of input molecules can be used to establish a step of a StochQuant molecular detection workflow.

There are several approaches one can take to select a model of a manipulation of a target/reference molecule.

(Approach 1) If the outputs of a step can be directly measured, one can perform an experiment in which known numbers of input target/reference molecules undergo the manipulation described in the step, and the number of output target/reference molecules of the step are measured.
(Approach 2) If the outputs of a step cannot be directly measured, but a “proxy” experiment can be performed to understand the relationship between the input number of target molecules, the manipulation, and the output number of target molecules, then this experiment can be performed.
(Approach 3) If the manipulation of interest is already commonly understood in the literature, a probability distribution (and measurable factors to parameterize the distribution) may already be known and can be used. An example of this is separating a sample of liquid from an environment via a pipette.

### Example 29 Separation of sample from environment

This is an example of identification of a segment of a measurement workflow representation and physical parameters for a provided manipulation of the target/reference molecule when a measurable amount of sample is separated from an environment. Separating a sample from an environment can be understood to be a manipulation that acts on the target/reference molecule as provided as a part of a testing measurement workflow.

In particular, separating a sample from an environment can be understood to be (i) a manipulation of the target/reference molecule that can impact the molecular count of the target/reference molecule obtained via the testing measurement, (ii) a manipulation that can be measured via a segmental calibration, and (iii) the segment representation can be parameterized by the number of input target/reference molecules and/or physical parameter.

An example of separating a sample from an environment is using a pipette (which can measure volumes) to obtain a portion of a liquid environment (a measurable amount of sample from environment), which is described in Examples 3, 16-20 and elsewhere throughout the document.

The example discussed below refers to the following:
Environment: a solution of target/reference nucleic acids
Target molecule: a nucleic acid of interest
Reference molecule: another nucleic acid of interest
Measurable amount of sample separated from an environment: a volume measured by a pipette and recorded by a user.
Absolute anchoring value of the reference in the environment: a value provided based on an absolute anchoring measurement of the reference molecule.

It is understood that the mathematical representation and physical parameters of this example can be used for other examples in different environments, and/or with different target/reference molecules, and/or different measurable amounts of sample separated from environment that undergo the same or similar manipulation. The example discussed below in particular refers to the manipulation when the number of target/reference molecule can be low-to-moderate such that the manipulation can affect the molecular count.

### Description of the Manipulation

This manipulation as part of a measurement workflow is the separation of a sample from an environment (e.g., a solution of isolated nucleic acids such as dilution MD4 from Example 3.

The manipulation of separating a sample from an environment is a common manipulation for which extensive calibration segmentation data has previously been generated, and for which the mathematical representation and the physical parameters of the manipulation have been provided where it can be found in literature. Based upon previous segmentation calibration of this manipulation, it is understood that the manipulation can be mathematically represented by a Poisson distribution and physical parameters that characterize the average number of molecules yielded by the manipulation.

When a sample is separated from the environment, the number of target/reference molecules that are separated from the environment can be modeled by a Poisson distribution with the following physical parameters:
the number of target/reference molecules in the environment,
a quantitatively measurable amount of sample separated from an environment (described as an amount of sample per an amount of environment)

Together, these physical parameters can be used to provide the average number of target/reference molecules separated from an environment (λ_{OutputTarget} or λ_{OutputReference} by multiplying the number of input target and/or reference molecules by the ratio of the volume of the sample separated from the environment to the volume of the environment), which can be modeled by the Poisson distribution. Thus, the mathematical representation of the manipulation and physical parameters of the manipulation that yield a distribution of probable output target and/or reference molecules can be described by sampling from a Poisson distribution parameterized by λ_{OutputTarget} for the target molecule and/or λ_{OutputReference} for the reference molecule.

$${λ}_{OutputTarget} = ( input target molecules ) \times \frac{{volume}_{sample}}{{volume}_{environment}}$$

$$Output Target Molecules ∼ Pois ( {λ}_{OutputTarget} )$$

$$and / or$$

$${λ}_{OutputReference} = ( input reference molecules ) \times \frac{{volume}_{sample}}{{volume}_{environment}}$$

$$Output ReferenceMolecules ∼ Pois ( {λ}_{OutputReference} )$$

It is understood that when a sample is separated from the environment, the input reference molecules (described above) is provided by the absolute anchoring value. In the context of assessment of the accuracy of the measurement workflow representation, the input target molecules is provided based on the known number of target molecules in the environment. In the context of the inference method, the inference method provides the input target molecules in connection to determining the number of input target molecules that yield the molecular count of the target via the testing measurement workflow.

Here, volume was chosen because these are readily measurable during many detection method workflow with commonly used pipettes.

It can be understood that if a sub-sample is separated from a sample of the environment (see Example 19), then the physical parameter of the manipulations are:
the number of target/reference molecules in the sample
a quantitatively measurable amount of sub-sample separated from the sample of the environment (described as an amount of sub-sample per an amount of sample)

In this case (subsample of a sample), the input number of reference molecules is provided by the output number of reference molecules of a previous Segment in connection to the absolute anchoring value of the reference. In this case (subsample of a sample), the input number of target molecules is provided by the output number of target molecules of a previous Segment in connection to the number of input molecules in the environment and/or the molecular count of the target molecule yielded by the measurement workflow.

It can be understood that if a user does not acquire the previously generated calibration segmentation data, then the user can generate new calibration segmentation data to provide the mathematical representation and physical parameters of the manipulation.

### Description of how to Generate New Calibration Segmentation to Provide the Mathematical Representation and Physical Parameters of the Manipulation

One can perform an experiment to generate segmentation calibration data, such as the one described in ref. or [99]. For example, in in the “Materials and Methods” sub-section “Experiment C: low-target copy number experiments (Poisson experiments), a segmental calibration is described for which average initial target molecule numbers of 0.5, 1, 1.5, 2, 3.5, 4, 5, 7, 10, and 20 were measured via PCR in 10 batches each containing 30 samples in each batch, and the validity of a Poisson distribution was tested for each initial target molecule number for each batch was assessed by performing a statistical concordance test between the empirical distribution of values (the distribution of observed measurements) with the theoretical Poisson distribution.

It is commonly understood that the Poisson distribution is a discrete probability distribution that for a mean or average number of events in a specific interval yields a distribution of probable number of times an event will occur.

Thus, for manipulations that contain these key features, a Poisson distribution can be used by default to yield a distribution of probable number of molecules yielded by the manipulation.

A user can confirm that a Poisson distribution can be used to represent the manipulation by using the segmentation calibration and a “fitting” approach such as the approaches described in other passages of the present disclosure identifiable by a skilled person. If the Poisson distribution cannot be used, a user can test alternative mathematical descriptions as described in other passages of the present disclosure identifiable by a skilled person.

This example of the identification of a segment that includes a manipulation of Separation of sample from environment can be implemented in a measurement workflow representation, such as the Representation described in Example 3, and Example 5.

### Example 30 PCR Amplification

This is an example of identification of a Segment of a Measurement Workflow Representation and physical parameters for a provided manipulation of the target/reference molecule when the manipulation is a polymerase chain reaction (PCR) that amplifies the target/reference molecule.

Performing PCR amplification of a target/reference can be understood to be a manipulation that acts on the target/reference molecule as provided as a part of a testing measurement workflow.

It can be understood that this example can apply to other manipulations that contain key features of the manipulation in this example. Key features can be described as:
The manipulation “amplifies” the target/reference at some “rate” where the rate can describe the amount of target that is amplified per a unit of time or number of cycles.

### Description of the Manipulation

This manipulation of a testing measurement workflow is the PCR amplification of a target/reference molecule in an environment or a sample of an environment (e.g., a solution of nucleic acids such as the sample of nucleic acids in a library preparation reaction). It is commonly understood in the literature that each PCR cycle, primers that contain complementary sequences to a target/reference molecule anneal to the input target/reference molecule, a polymerase anneals to the primer-template complex, the polymerase create a new copy of the input target/reference molecule, and a “melting” temperature denatures the DNA into single stranded target/reference molecules. Each PCR cycle, each input target/reference molecule has some probability of yielding one output target/reference molecule dependent on what is commonly referred to as the “efficiency” of the PCR reaction. Collectively, in each PCR cycle, for a given number of input target/reference molecules, there is a distribution of probable output target/reference molecules that is dependent on the number of input molecules and the efficiency of the PCR reaction.

The manipulation of PCR amplification is a common manipulation for which extensive calibration segmentation has previously been generated, and for which the mathematical representation and the physical parameters of the manipulation have been provided. Based upon previous segmentation calibration of this manipulation, it is understood that each PCR cycle of the manipulation can be mathematically represented by a Binomial distribution and physical parameters that characterize the average number of “amplicons” yielded by each PCR cycle of the manipulation [101-103].

Thus, the mathematical representation of this manipulation can be described:

n_{TargetInputMolecules}=number of target input molecules

P_{TargetAmpEffic}=PCR efficiency of the target molecules

### And/or

n_{ReferenceInputMolecules}=number of reference input molecules

P_{ReferenceAmpEffic}=PCR efficiency of the reference molecules

The distribution of probable output target and/or reference molecules can be obtained by sampling from a Binomial distribution parameterized by the number of input target/reference molecules and the PCR efficiency of the target/reference molecules.

$$New Output Target Molecules ∼ Binom ( {n}_{targetInputMolecules} , {p}_{TargetAmpEffic} )$$

$$Output Target Molecules = ( {n}_{TargetInputMolecules} ) + ( New Output Target Molecules )$$

$$And / or$$

$$New Output Reference Molecules ∼ Binom ( {n}_{ReferenceInputMolecules} , {p}_{ReferenceAmpEffic} )$$

$$Output Target Molecules = ( {n}_{ReferenceInputMolecules} ) + ( New Output Reference Molecules )$$

In some embodiments, the PCR efficiency of a reference/target is either known or assumed from previous segmentation calibration data.

In this example, the additional segmentation calibration data was generated to obtain the PCR efficiency (physical parameter) of the 16S rRNA gene of Listeria (the target molecule).

In the following passages a procedure is reported on how this data was generated and how a physical parameter of an efficiency of 0.964 was generated.

In this example, a Python script was used to implement the model above to track the probable number of output molecules at the end of each PCR cycle to yield a distribution of probably number of output molecules at the end of the library preparation PCR.

In some embodiments, qPCR with a standard curve can be used to obtain a PCR efficiency value. The standard curve can be yielded as discussed in Example 22. Then, the slope of the curve can be used to obtain a PCR efficiency value. The efficiency value can be computed as follows [10⁴]:

Assuming a log 10 transformation of the number of reference molecules (prior to linear regression)

$$PCR efficiency = {10}^{\frac{- 1}{slope}} - 1$$

where slope is the slope obtained from the linear regression of the standard curve fitting.

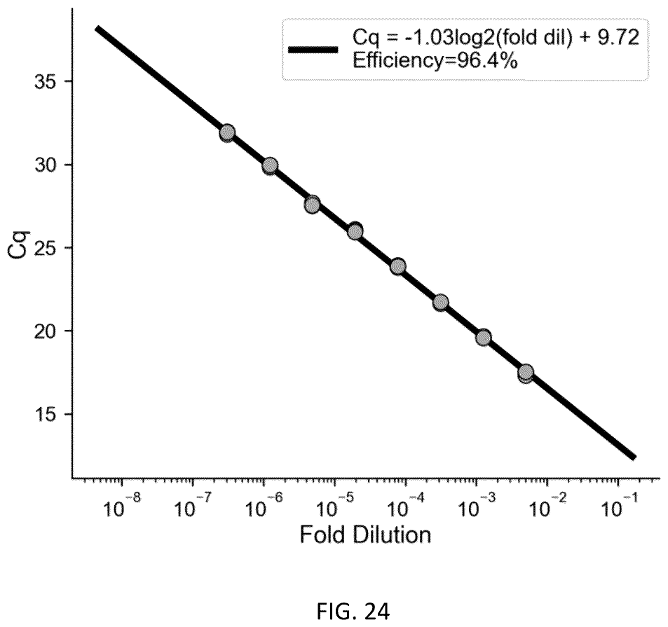

This procedure to obtain the PCR efficiency was performed for the Listeria gDNA target, in particular by using the reagents, primers and library preparation conditions described in Example 3. In this example, a large number (>10⁶ target molecules per microliter) of target Listeria 16S rRNA molecules was serially diluted approximately over several orders of magnitude, and (n=3) replicate qPCR measurements were obtained for each dilution. Then the data were fit to a linear curve (linear regression) to obtain a line of best fit based on the calibration data, and a PCR efficiency value obtained from the linear regression (FIG. 24).

In some cases, the PCR efficiency of a target/reference molecule may be approximated by measuring the PCR efficiency of another target/reference molecule. For example, for targets of similar characteristics (e.g., similar GC-content, length, and matches/mismatches to primer), a measured PCR efficiency for Target 1 may be used to approximate the PCR efficiency of Target 2.

Measurable qPCR efficiency for a target and/or reference can be used in step(s) of a StochQuant model of a molecular detection workflow to track the probable numbers of output target/reference molecules of a step yielded by a step with a given number of input target/reference molecules and a PCR efficiency for the target/reference molecule.

### Example 31—Fragmentation

This is an example of identification of a segment of a measurement workflow representation and physical parameters for a provided manipulation of the target/reference molecule when the Segment contains a fragmentation manipulation that fragments a molecule of interest into smaller molecules indicative of the molecule of interest.

For example, during library preparation of many sequencing workflows, molecules of interest are fragmented (and sometimes simultaneously tagmented) such that molecules are fragmented to a desired “fragment length” such that the target is broken into pieces, each piece being the desired fragment length. For example, a target molecule of length 1,000 bases fragmented to a fragment size of “250 bases” would yield on average 4 fragments of the target molecule (target size divided by fragment size). It can be understood that fragmentation is a stochastic process.

Thus, in a simple example, one can model fragmentation as a Poisson process parameterized by the average number of fragments yielded by the manipulation. In this simple “Poisson process” example, one can obtain the average number of fragments yielded by the manipulation from the physical parameters:

$${λ}_{OutputTargetFragments} = \frac{Target Size}{Fragment Size} \times {Target}_{InputMolecules}$$

$$and / or$$

$${λ}_{OutputReferenceFragments} = \frac{Reference Size}{Fragment Size} \times {Reference}_{InputMolecules}$$

Where Target Size and Fragment Size can be lengths (e.g., nucleotides or bases), molecular weights of the molecules, or other physical parameters related to the size of the target and/or the fragment, and where Target_inputMolecules and Reference_inputMolecules are the number of target/reference molecules in the environment prior to the manipulation and where λ_outputTargetFragments and λ_outputRferenceFragments are the average number of target/refence molecules after the manipulation.

Using a Poisson distribution, one can obtain a distribution of probable numbers of output molecules of target/reference from the manipulation by sampling from a Poisson distribution

Target_{OutputMolecules}˜Pois(λ_{outputTargetFragments})
and/or

Reference_{OutputMolecules}˜Pois(_{outputReferenceFragments})

the InputTargetMolecules/InputReferenceMolecules connected to the number of target/reference molecules in the environment and/or any manipulations upstream of this fragmentation manipulation and in connection to the downstream molecular count of the target/reference yielded by the testing measurement.

It is understood that in connection to testing measurements, such as shotgun metagenomic sequencing, where each individual sequenced fragment of a target/reference can yield a molecular count of the target/reference, that increasing the fragmentation of a target/reference can affect the molecular count of the target/reference. It can also be understood that the stochasticity of the fragmentation of the target/reference can affect the variability of the molecular count of the target/reference.

In another sub-example of the fragmentation of target/reference, a segmentation calibration can be performed to obtain data indicative of the distribution of the fragment lengths of the target/reference. For example, one can perform a shotgun metagenomic sequencing testing measurement (or repeated measurements) of target/reference of known genomic composition to obtain “paired end” reads for each molecular count, and then align the sequenced reads to the known genomic compositions of the target/reference. By doing so, one can obtain the length of the fragment based on the alignment positions of the forward and reverse reads, and one can compute this using common sequencing processing/analysis software such as Samtools. In this example, one can obtain a distribution of fragment lengths, and then perform a segmentation calibration to determine that a negative binomial distribution can approximate the distribution of read counts, and one can obtain the shape parameters (n and p) of the distribution indicative of the number and variability of the distribution of fragments yielded during the fragmentation step. In this sub-example using the negative binomial distribution, one can implement the distribution into the Segment by doing the following: One can obtain an array of probable fragment sizes by sampling from a negative binomial distribution, parameterized by the parameters yielded by the segmentation calibration. Then, one can calculate the probability of a fragment forming (per base of genomic content) by obtaining the total length of genomic content (e.g., the target length multiplied by the number of target molecules) and dividing by the per-base probability of fragmentation (1/fragment_length) with the fragment length being the value obtained from sampling from the negative binomial distribution.

To determine the best Segment representation of fragmentation for a particular workflow, one can assess the accuracy of the segment representation and/or the accuracy of the measurement workflow representation.

### Example 32 Molecular Detection of a Target and a Reference

This is an example of identification of a segment of a measurement workflow representation and physical parameters for a provided manipulation of the target/reference molecule such that the manipulation involves the key feature of:
the detection of the target molecule yielding a molecular count of the target,
the detection of the reference molecule yielding a molecular count of the reference,
provided a number of target/reference molecules in the sample (if the manipulation is occurring in the sample) or environment (if the manipulation is occurring in the environment),
the number of target molecules provided by (i) physical parameter; the known number of molecules in the environment as part of the Assessment of the Accuracy of a Measurement Workflow or (ii) by the Inference Method in relation to the molecular count of the target.

An example of a molecular detection of a target and a reference is discussed in Example 3 and Example 33 Flow-cell binding Example 1.

In particular, this is an example in which the molecular detection includes a sampling manipulation as discussed in Example 33 Flow-cell binding Example 1.

It can be understood that the molecular detection of the target and of the reference can be a stochastic process, particularly when the molecular detection includes a sampling manipulation. It can be understood that the mathematical relationship between (i) the number of reference molecules that are affected by the molecular detection manipulation and (ii) the molecular count of the reference molecule can be used to yield the average or expected molecular count of the target for a given number of molecules.

Accordingly, in the absence of other physical parameters that characterize a difference in the molecular detection of the target compared to the reference, the relationship of the number of target molecules to the number of reference molecules is proportional to the average or expected molecular count of the target to the molecular count of the reference.

$$\frac{Target Molecules}{Reference Molecules} ∝ \frac{{λ}_{TargetMolecCount}}{{Reference}_{MolecCount}}$$

This can be re-arranged to provide:

$${λ}_{TargetMolecCount} = \frac{Target Molecules}{Reference Molecules} \times {Reference}_{MolecCount}$$

and because this is a sampling manipulation (described in Example 33 Flow-cell binding Example 1) a Poisson distribution parameterized by the number of target molecules, the number of reference molecules, and the molecular count of the reference can yield a distribution of probable molecular counts of the target.

Targe_{tMoleccount}˜Pois(λ_{TargetColecCount})

In this example, Target Molecules and Reference Molecules refer to the number of target/reference molecules that are affected by the sampling manipulation. If this molecular detection manipulation occurs within the environment, the number of reference molecules is yielded by the absolute anchoring value.

### Example 33 Flow-cell binding Example 1

This is an example of identification of a Segment of a Measurement Workflow Representation and physical parameters for a provided manipulation of the target/reference molecule such that the manipulation involves the key features of:
A molecule of interest (target/reference molecule) binding to a flow cell that is used in part of a measurement workflow.

In particular, this example guides that the skilled user can identify a manipulation that can be represented at a Segment, the manipulation identified as a step in a measurement workflow that samples a portion of target/reference molecules to yield the molecular count of the target/reference. In this example, the sampling manipulation to yield a molecular count of the target/reference is referred to as “binding of a target/reference to a flow cell”.

In particular, the binding of a target/reference molecule to a flow cell can be understood to be a sampling manipulation in which the physical parameters of the manipulation of the target/reference molecule can impact the molecular count of the target/reference molecule obtained via the measurement workflow, (ii) a manipulation that can be measured via a segmental calibration, and (iii) the Segment Representation can be parameterized by the physical parameters of the manipulation.

An example of a sequencing flow cell is the Illumina MiSeq v3 flow cell, and an example of the target/reference molecule is a target/reference molecule that contains the Illumina Adapter Sequence such that the target/reference molecule can bind to the flow cell. It can be understood that the mathematical representation and physical parameters of this example can be used for other examples with similar features (sampling bias can be introduced because the measurement technology can sample a proportion of target/reference molecules) such as
nanopore Ion Chips such as the MinION flow cells, GridION flow cells, PromethION flow cells, Flongle flow cells, 2 PromethION flow cells,
PacBio SMRT cells,
Helicos Sequencing flow cells,
NanoString Technologies (nCounter) cartridge.

The example discussed below refers to the following:
Environment: a solution of target/reference nucleic acids,
Target molecule: a nucleic acid of interest,
Reference molecule: another nucleic acid of interest,
Absolute anchoring value of the reference in the environment: a value provided based on an absolute anchoring measurement of the reference molecule,
Measurable amount of sample separated from an environment: a volume measured by a pipette and recorded by a user,
   but it is understood that the mathematical representation and physical parameters of this example can be used for other examples in different environments, and/or with different target/reference molecules, and/or different measurable amounts of sample separated from an environment that undergo the same or similar manipulation.

The sampling manipulation of a target/reference molecule binding to a flow-cell is a common manipulation in measurement workflows, particularly next generation sequencing workflows, for which extensive calibration segmentation data has been previously generated, and for which the mathematical representation and the physical parameters of the manipulation have been provided where it can be found in literature. Based upon previous segmentation calibration of this manipulation, it is understood that the manipulation can be mathematically represented by a Poisson distribution and physical parameters that characterize the average number of molecules yielded by the manipulation (similarly to Example 29), and following the guidance provided by Example 32 Molecular Detection of a Target and a Reference

In other examples, the target and/or reference and/or sampling manipulation can include additional physical parameters that characterize the manipulation.

### Example 34 NanoPore Sampling

Nanopore sequencing is a measurement workflow that comprises one or more manipulations that can impact the molecular count of a molecule of interest detected by the measurement workflow. In particular, Nanopore sequencing comprises one or more sampling manipulations, “sampling processes”, or “sampling events” which can be described as stochastic processes that sample a portion of molecules in an environment.

Examples of sampling events in a Nanopore sequencing measurement workflow can include:
the separation of a measurable amount of sample from an environment (in the context of Example 29) in which the measurable amount of sample from an environment refers to volumes,
the target/reference molecule capture of the target/reference molecule by a nanopore on the flow cell (flow cell binding).

In some embodiments, sampling manipulation may be combined with other manipulations to form a Segment. An example may include combining the capture of the target/reference molecule by a nanopore to the translocation and subsequent sequencing of the target/reference molecule by the nanopore.

### Example 35 Shotgun Metagenomic Sequencing Example (User 1 Example)

This is an example of building a StochQuant Workflow for a shotgun metagenomic sequencing testing measurement that comprises:
Identify target molecule of interest, environment(s) of interest, testing measurement to yield a molecular count of the target molecule,
Identify the manipulations of the molecule(s) of interest in a testing measurement workflow,
Select at least one reference molecule and obtain a method to perform an absolute anchoring measurement,
Build Measurement Workflow Representation,
Select Inference Method,
Incorporate Measurement Workflow Representation into the Inference Method.

First, the target molecule of interest, environment of interest, and a testing measurement that yields a molecular count of the target molecule were identified.
Target molecule: a particular gene sequence of DNA
Environment: A solution of isolated nucleic acids. These are the same dilutions (MD1, MD2, MD3, MD4) as the proof-of-concept amplicon example.
Testing measurement: shotgun metagenomic sequencing
Molecular count of the target: a read count of the number of sequenced reads from a sample of an environment that align to the target molecule gene sequence.

Then, the manipulations of the molecules of interest that comprise the testing measurement workflow were identified.
A measurable amount of sample is separated from an environment and added into a library preparation reaction.
The target/reference molecules are fragmented and sequencing adapters are added to the ends of the fragments by transposase.
The tagmented and fragmented target/reference molecules are amplified via polymerase chain reaction (PCR).
A sub-sample is separated from the sample containing the tagmented/fragmented target/reference molecules.
The sub-sample is loaded onto a flow-cell.
The bound molecules of the flow cell are sequenced via a series of manipulations described by bridge amplification and sequencing-by-synthesis.

Then, a reference molecule and method to perform the absolute anchoring measurement of the reference molecule were selected.
Reference molecule: 16S rRNA gene sequences of all microbes
Molecular count of the reference: a read count of the number of sequenced reads from a sample of an environment that align to the reference molecule gene sequence. The reference alignment is a plurality of reference alignments of a region of the 16S rRNA gene. Because 16S with universal primers, these are read counts that align to any 16S sequence that we had in the database.
Absolute anchoring measurement of the reference: total number of 16S rRNA gene copies in a solution of nucleic acids. This solution is the environment from which (i) a sample is taken to perform the absolute anchoring measurement (which is digital PCR measurement of total number of 16S rRNA gene copies) and (ii) another sample is taken to perform amplicon sequencing (testing measurement).

Then, the measurement workflow representation was built as follows:

Identify Measurement Workflow Representation Segments and Perform Segmental Calibration for each Segment.

First, the measurement workflow representation segments were identified by identifying the manipulations or series of manipulations of the testing measurement workflow that (i) can impact the molecular count of the target/reference molecule obtained via the testing measurement, (ii) can be measured via a segmental calibration (discussed below) that can yield a representation of the segment that can yield output numbers of target/reference molecules that approximate the output numbers of target/reference molecules of the manipulation(s) of the testing measurement, and (iii) for which the Segment Representation can be parameterized by the number of input target/reference molecules and/or the physical parameter of the manipulation(s) of the testing measurement that can impact the molecular count of the target/reference.

Of the manipulations of the testing measurement workflow, three Segments were identified, described below:

Segment 1: The loading of target/reference molecules into the library preparation reaction. It can be understood that Segment 1 comprises a manipulation that contains the key features of Example 29 Separation of sample from environment, and the mathematical representation from Example 29 Separation of sample from environment can be used for this segment.

Segment 2: The fragmentation of the target/reference molecules in the library preparation reaction. It can be understood that Segment 2 comprises a manipulation that contains the key features of Example 31 Fragmentation of molecules, and the mathematical representation from Example 31 Fragmentation of molecules can be used for this Segment. Based on a segmentation calibration (see Example 31 discussion of negative binomial segmentation calibration), the procedure described for the negative binomial in Example 31 was used for Segment 2.

Segment 3: The remainder of the library preparation and sequencing of the target/reference molecules. In this example, this series of manipulations were grouped together to comprise Segment 3 because a segmentation calibration from a similar series of manipulations was used to provide the physical parameters of a similar series of manipulations that characterize the series of manipulations that impact the molecular count.

The series of Segment 1, Segment 2, and Segment 3 comprise the measurement workflow representation such that the output number of molecules yielded by Segment 1 is the input number of molecules into Segment 2, and so forth, and the output number of molecules of Segment 3 is the molecular count or distribution of molecular counts of the target molecule, given physical parameters of the testing measurement workflow.

The measurement workflow representation was implemented as a Python function with the Numpy and Numba libraries, and the probability distributions were represented using the numpy.random module with the corresponding probability distributions. For example, numpy.random.poisson ( ) was used for a Poisson distribution.

### Assess the Accuracy of the Measurement Workflow Representation

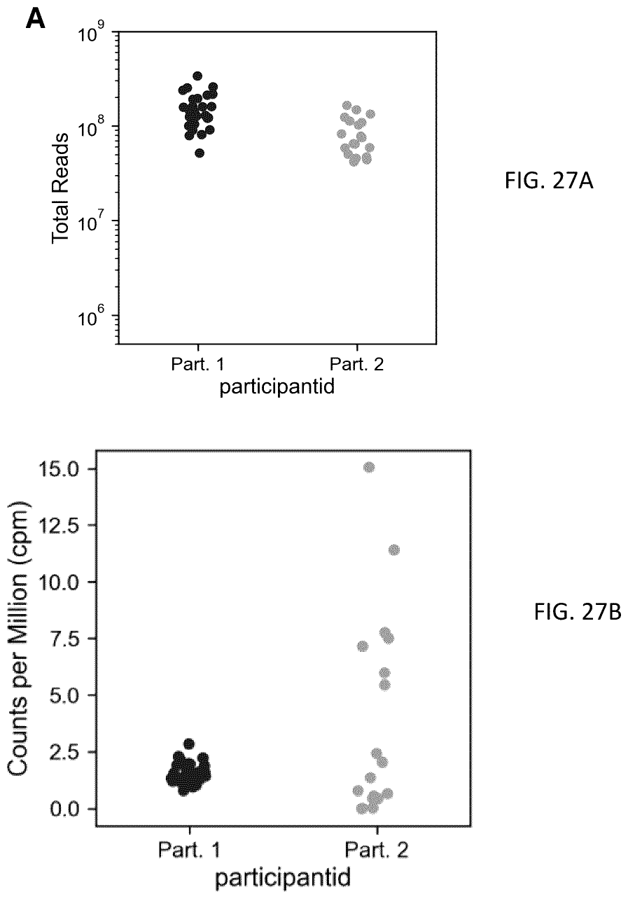

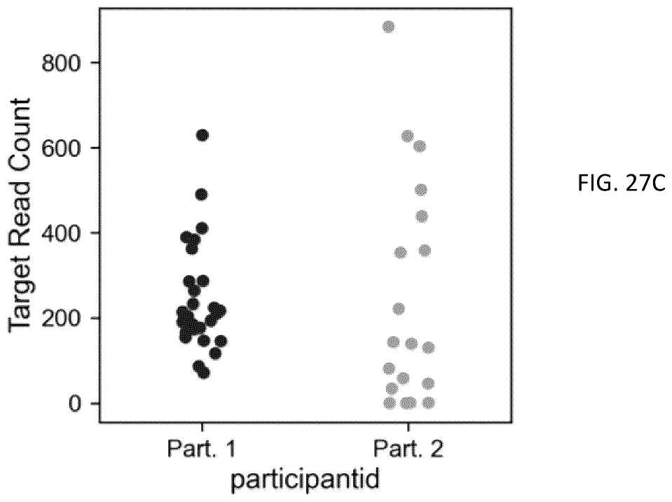

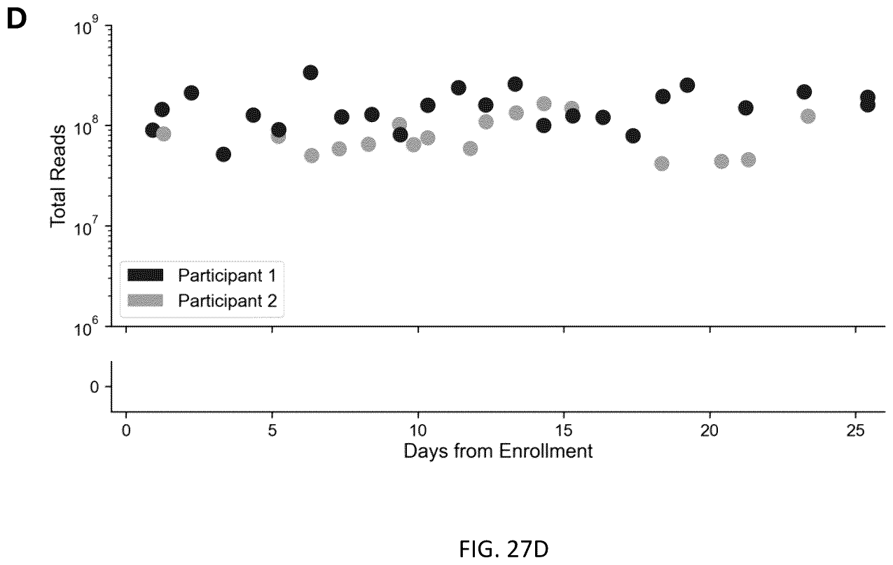

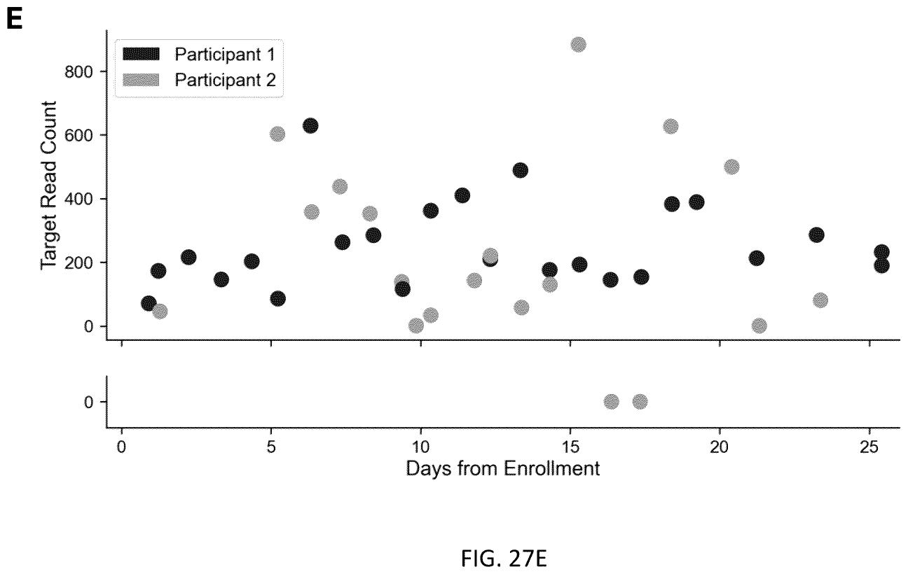

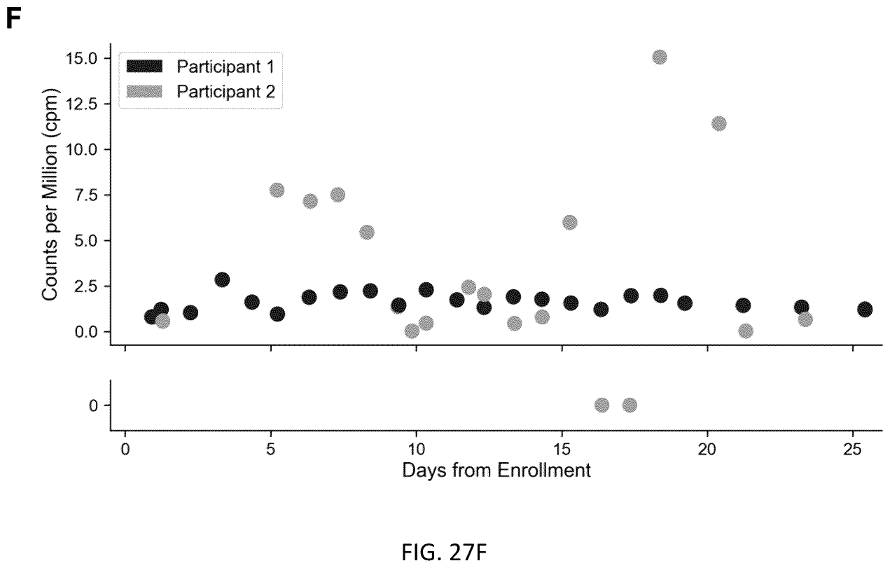

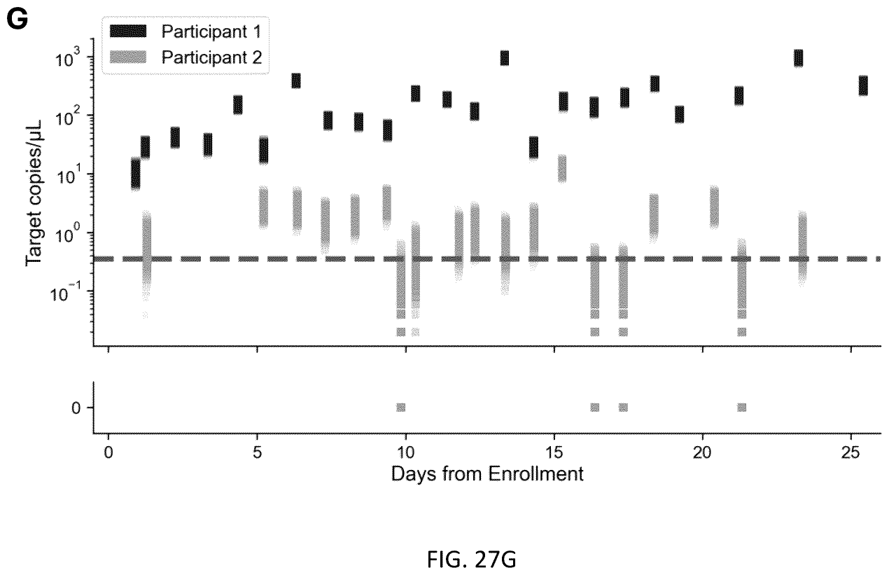

The Accuracy of the exemplary Measurement Workflow Representation was assessed (FIG. 27) using similar procedures to those described in the Assessment of Accuracy of the Measurement Workflow example described in Example 5.

Due to cost constraints, a limited number of replicate measurements of the measurement workflow, for a given number of target and reference molecules could be performed.

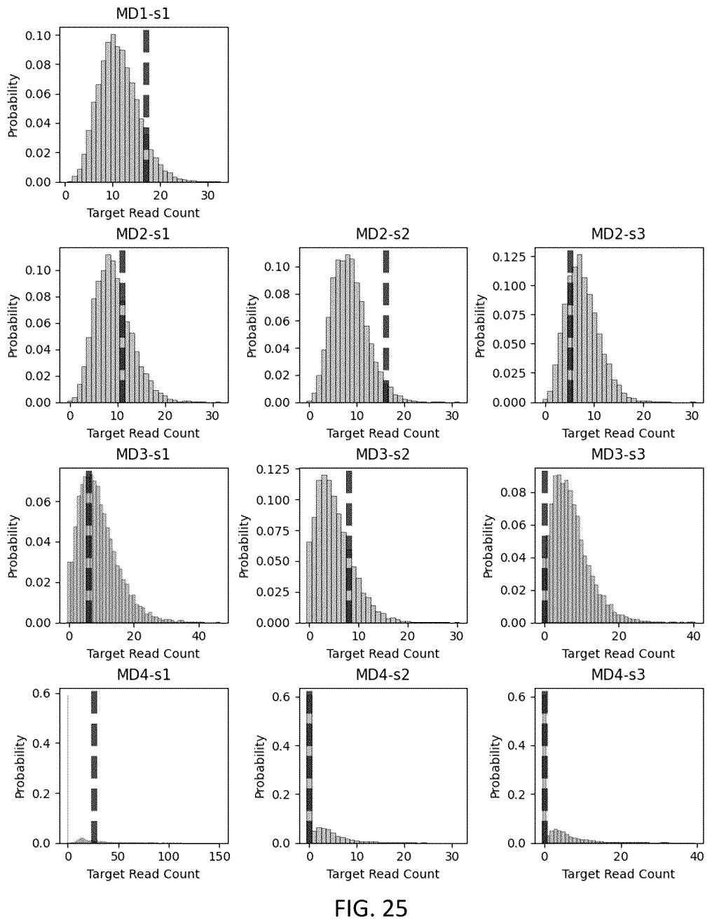

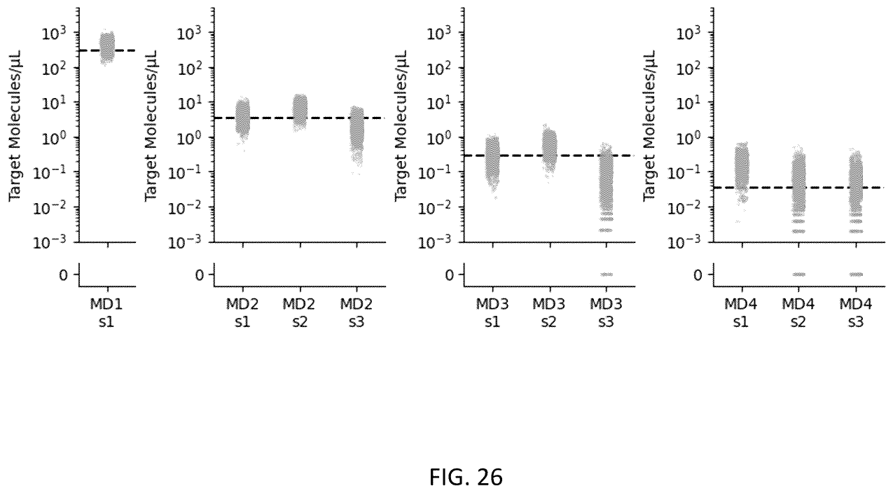

In this example of the assessment of the measurement representation accuracy, the Accuracy of the Measurement Representation for a particular gene target of Bacillus was evaluated (FIG. 25). In this example, the measurement representation was used to generate a probable distribution of (n=10,000) molecular counts of the gene target yielded by the shotgun sequencing measurement workflow using the physical parameters of the measurement workflow for one replicate of MD1 (MD1-s1), three measurement replicates of MD2 (MD2-s1, MD2-S2, MD3-s3), three measurement replicates of MD3 (MD3-s1, MD3-s2, MD3-s3), and three measurement replicates of MD4 (MD4-s1, MD4-s2, MD4-s3). The distribution of probable counts for each measurement was then compared against the observed measurement from the measurement workflow. For each measurement, it was confirmed that the observed molecular count yielded by the testing measurement was within the 1^{st} and 99^{th} percentiles of the probable counts yielded by the measurement representation, indicating the representation was accurate.

Incorporate the Measurement Representation and physical parameters into an Inference Method

An inference method similar to the method described in Example 7, and Example 11, was selected, and the Measurement Workflow Representation was incorporated into the Inference Method via a Python script. The probability distribution of target abundance was stored in the form of negative binomial shape parameters n and p. This was accomplished by sampling from the initial probability distribution of target abundance (See Example 12). In this case, the target abundance is the number of target molecules in an environment. Then, the mean (u) and variance (62) from the sampled number of target molecules in the environment were calculated. Then n and p (the shape parameters of the negative binomial distribution were calculated as follows:

$$p = \frac{μ}{{σ}^{2}}$$

$$n = \frac{{μ}^{2}}{( {σ}^{2} - μ )}$$

### Example 36-Shotgun Sequencing User 2

This is an example of using a measurement workflow representation and physical parameters that have been incorporated into an inference method for the generation of probability distributions of target abundance from a shotgun sequencing measurement workflow. In particular, this is an example of using the representation, inference method, and physical parameters from Example 35 for the gene and environments discussed in Example 35.

In this example, the inference method with the representation and physical parameters of the shotgun metagenomic sequencing testing measurement were provided for each environment, and the absolute anchoring value for each environment was also provided (see Example 35). The absolute anchoring value was obtained via previous measuring with digital PCR of a sample of each environment. The physical parameters were inputted into the Inference Method, which provided the shape parameters n and p for a negative binomial distribution of the number of target molecules in each environment (as discussed in Example 35).

Here, an example is shown of the probability distributions of target abundance (yielded by StochQuant) accurately containing the actual abundance value of the target in an environment at various abundances (e.g., concentrations) in MD1, MD2, MD3, MD4 including when the target is detected in (n=3/3) replicates in MD2, (n=2/3) replicates, in MD3 and only (n=1/3) replicate in MD4. In other words, the probability distributions correctly quantitatively detect the target within the 1^{st} to 99^{th} percentiles of the distribution.

To plot the distributions, the numpy.random.negative_binomial function was used to sample (n=10,000) values from each distribution indicative of the number of target molecules in the environment. Then the sampled values were dived by volume of the environment (one of the physical parameters) to provide the distribution in the form of an array of concentrations (target molecules per microliter).

To determine if the actual (“ground truth” or “known”) value of target abundance in an environment was contained within the 1^{st} and 99^{th} percentile of the probability distribution yielded by StochQuant, the Scipy.stats.nbinom.ppf function was used, which calculates the inverse of the cdf percentiles for the distribution, given the shape parameters n, p, and the percentile. Once the 1^{st} and 99^{th} percentile values were obtained from the distribution, a function checked whether the “ground truth” value was greater than the 1^{st} percentile and less than the 99^{th} percentile.

### Example 37 Bulk RNA Sequencing Example

This is an example of “Using a StochQuant Workflow” (see FIG. 1), in particular using a StochQuant Workflow that contains an Inference Method that has incorporated a Measurement Workflow Representation of bulk RNA sequencing, the physical parameters of the manipulations of the measurement workflow.

This example uses RTqPCR with a standard curve of the MYH9 gene (from Example 23) to obtain the absolute anchoring value of the reference molecule in the environment.

This is an example of a longitudinal (time series) example of quantitative detection of mRNA of the HPRT1 gene, which is expressed in almost all human tissues at relatively constant levels. It is considered a housekeeping gene. This example shows concentrations of the HPRT1 mRNA in the environment as the abundance value of the target. This example compares HPRT1 mRNA over time in an individual and between two individuals (Participant 1 and Participant 2).

This example shows that even though the testing measurements from environments from Participant 1 and Participant 2 had similar total reads (a measure of the number of reads that aligned to the human genome) (FIG. 27A) and similar normalized target counts (counts per million) (FIG. 27B), and similar molecular counts of the target (FIG. 27C), substantially more noise was observed among measurements of the environments of Participant 2 compared to Participant 1 (FIG. 27B-F). In particular, the normalized counts from Participant 2 seemed to show two time periods of their longitudinal profiling that indicated increases in HPRT1 expression, and in particular indicated increases in Participant 2 that were absent in Participant 1, and in particular that indicated higher expression levels in Participant 2 compared to Participant 1. Additionally, in between these periods of increase in Participant 2, some measurements in the longitudinal profile yielded non-detection of the target molecule HPRT1 in Participant 2 (FIG. 27E, 27F).

However, analysis with StochQuant probability distributions of target abundance revealed that in fact, the concentration of HPRT1 was (i) not substantially increasing in Participant 2, (ii) was in fact consistently lower in concentration in Participant 2 compared to Participant 1, and (iii) was present at levels that are near the Limit of Detection of the testing measurement (predicted by StochQuant measurement representation) indicating that the non-detection was likely due to stochasticity of the testing measurement (FIG. 27G).
Testing measurement: bulk RNA sequencing
Target molecule: mRNA of a particular human gene (in this example HPRT1)
Reference molecule: mRNA of the human MYH9 gene
Environment: A solution of isolated nucleic acids from a nasal swab of a human.
Absolute anchoring value: total number of MYH9 mRNA molecules (reference molecule) in an environment, obtained from a RT-qPCR measurement with a standard curve of the MYH9 mRNA in a sample of the environment (see Example 23)
Molecular count of the target: a read count of the number of sequenced reads from a sample of an environment that align to the target molecule gene sequence.
Molecular count of the reference: a read count of the number of sequenced reads from a sample of an environment that align to the reference molecule gene sequence.
   Brief description of the Inference Method: The inference method from Example 35 was used for this example.
   Brief description of the measurement workflow representation
   The measurement workflow representation contained three segments that were described elsewhere in this document:
   Segment 1 (Separation of a sample from an environment) as described in Example 29
   Segment 2 (Fragmentation of target/reference molecules). For fragmentation, the distributions of fragments were not measured, so the Poisson implementation of the fragmentation was used as described in Example 31.
   Segment 3 (Flow-Cell Binding) as described in Example 33.

### Example 38 Single-Cell RNA Sequencing Example

This is an example of Building a StochQuant workflow for a single-cell RNA sequencing (scRNA-seq) testing measurement that comprises:
identifying one or more target molecule of interest, one or more environments of interest, testing measurement to yield a molecular count of the target molecule,
identifying the manipulations of the one or more target molecule of interest in a testing measurement workflow,
selecting at least one reference molecule and obtain a method to perform an absolute anchoring measurement,
building a measurement workflow representation of the testing measurement,
selecting an inference method of the testing measurement,
incorporating the measurement workflow representation into the inference method

This is an example that uses previously obtained data provided by the work described in Ref. [105].

First, the target molecule of interest, environment of interest, and a testing measurement that yields a molecular count of the target molecule were identified.
Target molecule: RNA of a particular gene
Environment: A solution of isolated RNA molecules (the ERCC Spike-In Mix)
Testing measurement: inDrop single-cell RNA sequencing
Molecular count of the target: a read count of the number of sequenced reads from a sample of an environment that align to the target molecule gene sequence.

Then, the manipulations of the molecules of interest that comprise the testing measurement workflow were provided by the protocol of the testing measurement workflow [10⁵].
The target ERCC RNA Spike-in Mix is diluted 1,000 fold.
A mixture of cells and the diluted ERCC Spike-in Mix are encapsulated in microfluidic droplets such that each droplet contains one cell and a sample of the eRCC RNA Spike-In Mix.
Within each droplet, mRNA is reverse transcribed into cDNA, and each molecule is tagged with (1) a unique cell-barcode (all RNA molecules from one cell get the same cell-barcode), and (2) each molecule is tagged with a unique molecular identifier (each RNA molecule within a cell gets its own unique molecular identifier sequence).
Then, the droplets are broken, and the bulk mixture undergoes Exonluclease I treatment to digest single-stranded primers.
Then solid phase reversible immobilization purification to purify the cDNA.
Then second strand synthesis to synthesize a second strand of cDNA to generate double stranded cDNA.
Then SPRI purification to purify the double stranded cDNA. Then T7 in vitro transcription linear amplification.
Then SPRI purification of the amplified RNA. Then RNA fragmentation. Then SPRI purification of the fragmented RNA.
Then ligation of sequencing adapters.
Then Reverse Transcription of the RNA.
Then cDNA amplification via PCR
Then sequencing of the amplified cDNA

A reference molecule and method to perform the absolute anchoring measurement of the reference molecule were selected.
Reference molecule: number of unique molecule identifiers (UMIs)
Molecular count of the reference: a read count of the number of sequenced reads from a sample of an environment that align to the reference molecule gene sequence.
Absolute anchoring value of the reference: the total number of unique molecular identifiers (UMIs) in a droplet. This was obtained as discussed in Example 27.

Then, the measurement workflow representation was built as follows:

Identify Measurement Workflow Representation Segments and Perform Segmental Calibration for each Segment.

First, the measurement workflow representation segments were identified by identifying the manipulations or series of manipulations of the testing measurement workflow that (i) can impact the molecular count of the target/reference molecule obtained via the testing measurement, (ii) can be measured via a segmental calibration (discussed below) that can yield a representation of the Segment that can yield output numbers of target/reference molecules that approximate the output numbers of target/reference molecules of the one or more manipulations of the testing measurement, and (iii) for which the segment representation can be parameterized by the number of input target/reference molecules and/or the physical parameter of the one or more manipulations of the testing measurement that can impact the molecular count of the target/reference.

Of the manipulations of the testing measurement workflow, three Segments were identified, described below:

Segment 1: This is a sampling of an environment step and thus was modeled as discussed in example 29. Here the measured capture efficiency as part of the previous data generated in the Klein published work is used as part of the segmentation calibration to determine the physical parameters of the Segment. The following manipulations were grouped together into a segment as part of the previously obtained data for the segmentation representation.
a. The target ERCC RNA Spike-in Mix is diluted 1,000 fold.
b. A mixture of cells and the diluted ERCC Spike-in Mix are encapsulated in microfluidic droplets such that each droplet contains one cell and a sample of the eRCC RNA Spike-In Mix.
c. Within each droplet, mRNA is reverse transcribed into cDNA, and each molecule is tagged with (1) a unique cell-barcode (all RNA molecules from one cell get the same cell-barcode), and (2) each molecule is tagged with a unique molecular identifier (each RNA molecule within a cell gets its own unique molecular identifier sequence).
d. Then, the droplets are broken, and the bulk mixture undergoes Exonluclease I treatment to digest single-stranded primers.
e. Then solid phase reversible immobilization purification to purify the cDNA.
f. Then second strand synthesis to synthesize a second strand of cDNA to generate double stranded cDNA.
g. Then SPRI purification to purify the double stranded cDNA. Then T7 in vitro transcription linear amplification.
h. Then SPRI purification of the amplified RNA.

Segment 2: Fragmentation. This was modeled via a Poisson model of fragmentation, as discussed in Example 31.
a. Then RNA fragmentation.
b. Then SPRI purification of the fragmented RNA.

Segment 3: Sequencing (e.g., flow cell binding of target/reference to the flow cell) This was modeled as described in Example 33.
a. Then ligation of sequencing adapters.
b. Then Reverse Transcription of the RNA.
c. Then cDNA amplification via PCR
d. Then sequencing of the amplified cDNA

The following experiments show that StochQuant single-cell RNA sequencing (scRNA-seq) combines a measurement of a number of target molecules performed by a sequencing measurement and a measurement of a number of reference molecules performed by a sequencing measurement with an absolute anchoring measurement of the number of reference molecules in a sample which can be selected as the environment or obtained as a first step in a StochQuant scRNA-seq workflow. Then these sequencing measurements and absolute anchoring measurement are used to generate probability distributions of the absolute abundance of the target molecule in the environment, such as the number of RNA molecules of a of gene in a cell, thus allowing a determination of the absolute abundance of the RNA of a gene in a cell.

Here, it is demonstrated in a scRNA-seq experiment that stochastic modeling of the environment can improve the reliability of a scRNAseq pipeline that analyzes numbers of molecules, in particular when the number of small and stochasticity is usually higher. In this example, an ERCC RNA Spike-In Mix (Invitrogen Cat 4456740) with RNA target molecules of known abundance, sequence, and size (e.g., length) was diluted and spiked into the solution required to form droplets for inDrop scRNA-seq [105]. In this example it was shown that for a subset of ERCC RNA targets, a StochQuant model can accurately track the number of molecules through a scRNA-seq molecular detection workflow. It was also shown that the StochQuant method can be used for the detection and quantitative detection of these subset example ERCC target molecules in an ERCC Spike-In Mix (the environment) from the key features of the StochQuant detection approach (see below).

### Description of the Workflow for Context

In this molecular detection workflow, cells (and in this case, a dilution of the ERCC RNA Spike-In Mix) are encapsulated in microfluidic droplets such that each droplet contains one cell and a sample of the eRCC RNA Spike-In Mix. Within each droplet, mRNA is reverse transcribed into cDNA, and each molecule is tagged with (1) a unique cell-barcode (all RNA molecules from one cell get the same cell-barcode), and (2) each molecule is tagged with a unique molecular identifier (each RNA molecule within a cell gets its own unique molecular identifier sequence). Then, the droplets are broken, and the bulk mixture undergoes Exonluclease I treatment to digest single-stranded primers. Then solid phase reversible immobilization purification to purify the cDNA. Then second strand synthesis to synthesize a second strand of cDNA to generate double stranded cDNA. Then SPRI purification to purify the double stranded cDNA. Then T7 in vitro transcription linear amplification. Then SPRI purification of the amplified RNA. Then RNA fragmentation. Then SPRI purification of the fragmented RNA. Then ligation of sequencing adapters. Then reverse transcription (RT). Then cDNA amplification via PCR.

Through measurements obtained via the previous development and validation of inDrop scRNA-seq by others, a “capture efficiency” of the ERCC Spike-In Mix was obtained. This capture efficiency describes the probability that a target ERCC RNA molecule will be encapsulated in a microfluidic droplet, and the steps outlined above will yield a successfully amplified cDNA product via PCR. It can be understood, therefore, that instead of modeling each individual step, the collection of steps can be modeled as one stochastic step.

First, the key features of the StochQuant molecular detection approach were selected.
Testing measurement: inDrop single-cell RNA sequencing
Target molecule: RNA of a particular gene
Reference molecule: number of unique molecule identifiers (UMIs)
Environment: A solution of isolated RNA molecules (the ERCC Spike-In Mix)
Absolute anchoring value: the total number of unique molecular identifiers (UMIs) in a droplet.
Molecular count of the target: a read count of the number of sequenced reads from a sample of an environment that align to the target molecule gene sequence.
Molecular count of the reference: a read count of the number of sequenced reads from a sample of an environment that align to the reference molecule gene sequence.

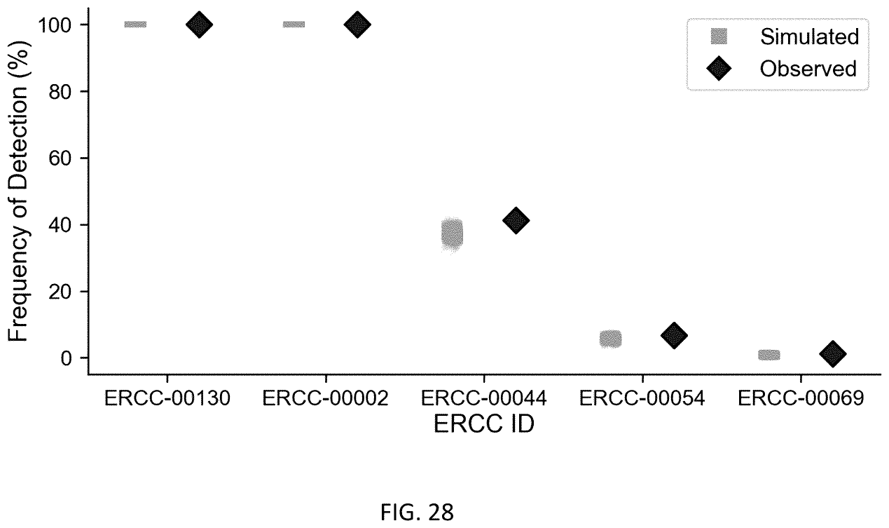

The mathematical representation of the workflow in this example (the three Segments of this workflow chained together) was implemented as a Python function, and an Assessment of Accuracy via detectability of the targets among the subset (n=1030) cells for four ERCC targets are shown in FIG. 28. Of the four shown ERCC targets, the measurement representation yielded detectability of each target that were within 3% of the observed detectability via the measurement workflow.

### Example 39 Single-Cell RNA Sequencing Example 1B (User 1 Example)

This example is the same as single-cell RNA sequencing Example 38, except alternative key features of the StochQuant molecular detection approach were selected. The selection of alternative key features resulted in a different Segment 3 of the measurement workflow representation (compared to Example 38) described in further detail below.

This is an example of using a molecular count of the number of unique UMI+target/reference molecule conjugates that are detected via the testing measurement. For example, if 5 reads of a specific UMI+target are detected, in Example 38, this would yield a molecular count of 5, but in this example would yield a molecular count of 1 (because the previous example 38 counts the number of reads of the gene that are detected and this example counts the number of unique UMIs associated with the gene that are detected).
Testing measurement: inDrop single-cell RNA sequencing
Target molecule: RNA of a particular gene
Reference molecule: number of unique molecule identifiers (UMIs)
Environment: A solution of isolated RNA molecules (the ERCC Spike-In Mix)
Absolute anchoring value: the total number of unique molecular identifiers (UMIs) in a droplet.
Molecular count of the target: a molecular count of the number of unique molecular identifiers from a sample of an environment that contain a sequence that aligns to the target molecule gene sequence.
Molecular count of the reference: a molecular count of the number of unique molecular identifiers from a sample of an environment that contain a sequence that aligns to the reference molecule gene sequence.

As such, to reflect the manipulation that yields the molecular count of unique UMIs that are detected via the testing measurement, Segment 3 (from the previous example) was modified such that the modeling was performed as follows:

Segment 3 still describes the manipulation of a sampling event that results in the building of the target to the sequencing flow cell. However, in this example, the molecular count is the number of UMIs, not the number of reads. Thus, to mathematically represent UMIs instead of reads, the following modeling can be used.

The number of UMIs in the sample is still provided by the absolute anchoring value of Example 38 in connection with the physical parameters of Segment 3.

Similarly to Example 33, the average number of target reads is determined by the number of target UMIs in the sample, the number of reference UMIs in the sample, and the number of reference reads obtained via the testing measurement.

$${λ}_{TargetReads} = \frac{Loaded Target UMIs}{Loaded Reference UMIs} \times Reference Reads$$

Here, the average number of target reads (MargetReads) can be used with Poisson statistics to determine the probability of yielding a non-zero read count of the target+UMI conjugate following similar procedures described in Example 27.

Here, the probability of a non-zero readcount for a given target molecule is provided by:

$${p}_{nonzero} = 1 - {e}^{- ( {λ}_{targetReads} )}$$

And similarly to Example 27, the number of unique target UMIs is given by the binomial distribution parameterized by the number of loaded target UMIs (the number of target+UMI molecules in the sample) and the probability of each UMI being detected as given by the Poisson statistics based on the physical parameters of the manipulation)

Detected Target UMIs˜Binom(Loaded Target UMIs,P_{nonzero})

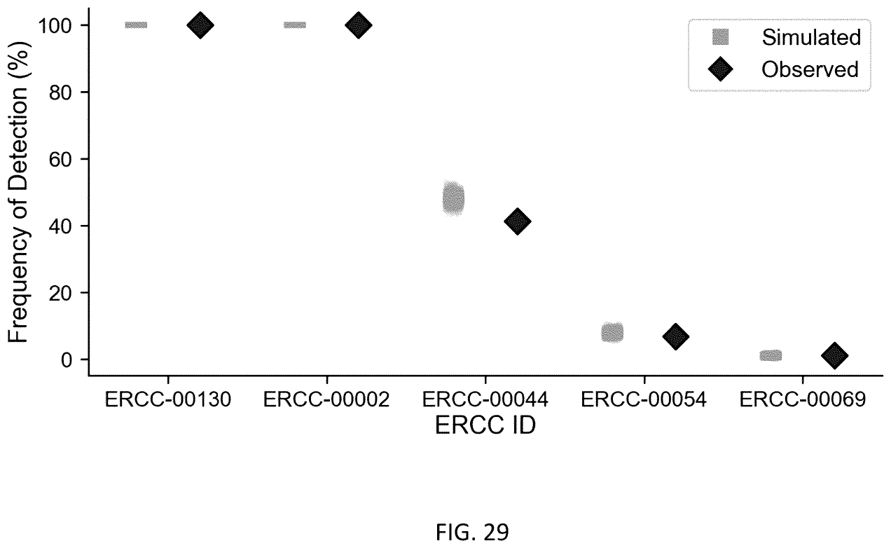

The mathematical representation of the workflow in this example (the three segments of this workflow chained together) was implemented as a Python function, and an assessment of accuracy via detectability of the targets among the same (n=10300) cells for the same four ERCC targets from Example 38 are shown in FIG. 29. Similarly to Example 38, of the four shown ERCC targets, the measurement representation yielded detectability of each target that were within 3% of the observed detectability via the measurement workflow.

### Example 40 Determination Examples

The following are examples in which a StochQuant probability distribution of number or abundance of target molecules in an environment is used to yield a level of confidence about the detection or quantitative detection of target(s) in environment(s), which is used to make a determination. The user can be directed to use (i) a number of target molecules, (ii) a value of the ratio of the number of target molecules in relation to the number of another target molecules, (iii) a value of the ratio of the number of target molecules in relation to the number of reference molecules, or (iv) another value indicative of the abundance of target(s) molecules in an environment. The user can be directed based on the value that is useful to make the determination for their particular system.
Examples include: Using a number of target molecules in an environment for the detection of a gene marker of Neisseria gonorrhoeae
Using a relative abundance of a target molecule in an environment (the ratio or % of the target compared to the reference molecule) for the quantitative detection of a gene marker for the diagnosis of bacterial vaginosis.
Using a ratio of a number of Target A molecules to number of Target B molecules in an environment for copy number variation analysis to identify genomic regions that have been duplicated or deleted.

In the following Examples 41 to 47, it is assumed that the user is Using the StochQuant Workflow (as described in the “Using the StochQuant Workflow” section of FIG. 1), and that the user has already obtained the necessary probability distributions for the determination example.

### Example 41 Detection of a Target

This is an example of how to use a StochQuant probability distribution of abundance of target molecules in an environment to detect or quantitatively detect a target in the environment; the detection provided by:
a probability distribution of target abundance yielded from using the StochQuant Workflow,
a confidence interval of target abundances in the environment possibly selected by the user and/or
a confidence level threshold possibly selected by the user.

In some embodiments, the confidence interval and/or confidence level threshold are provided to the user by another user. For example, the developer of a detection workflow (user A) can provide (i) a confidence interval of greater than 1,000 target molecules in an environment, and (ii) a confidence level threshold of 95%, and the provided confidence interval and confidence level threshold may be incorporated into a software, such that when user B or another software provide the probability distribution of target abundance in an environment, a determination is made based on the pre-provided confidence interval and confidence level threshold.

In this example, the confidence interval is “greater than 1,000 target molecules in an environment”. Accordingly, any target abundance value greater than 1,000 target molecules in an environment is included in the interval. Examples of confidence intervals can include:
less than 500 target molecules in an environment; accordingly, any target abundance value less than 500 target molecules in an environment,
between 300 and 600 target molecules in an environment; accordingly, any target abundance value greater than 300 but less than 600 target molecules in an environment,
greater than 3 Target A molecules per 1 Target B molecule in an environment.

In this example, the provided confidence level threshold is used to make the determination of “detection” or “non-detection” of the target molecule in the environment. Examples of a confidence level threshold can include:
a value between 0 and 1 such that values closer to zero indicate low levels of confidence and values closer to 1 indicate higher levels of confidence, such that the confidence is indicative of the confidence of detection; for example, a confidence level threshold of 0.95 can be indicative of a minimum threshold of 95% probability that the target abundance is within the confidence interval,
a value between 0 and 1 such that the confidence is indicative of the confidence of “non-detection; for example, a confidence level of 0.05 can be indicative of a minimum threshold of 5% probability of non-detection,
a value between 0 and 100 such that the confidence is expressed as a percent such that a confidence level threshold of 95% is indicative of a minimum threshold of 95% probability of detection.

Then, obtain a level of confidence (Confidence Level) that the number of target molecules in an environment is within the confidence interval. This can be accomplished in several way. Non limiting examples can include:

### (Approach A)

First, obtain probable numbers of target molecules in an environment from sampling from a distribution of target abundance in an environment. For further details of an example of how to do this, please see “Computationally sampling target abundances from probability distributions of taxon abundance” from the Example 3. Preferably, obtain at least 1000 probable numbers of target molecules in an environment. Then, obtain the frequency of the probable abundances of target molecules that are within the Confidence Interval. To do so, one (or a software package such as Numpy) can count the number of probable target molecules that are within the confidence interval and divide this number by the number of probable abundances sampled from the distribution (in this example, the number of probable abundances sampled from the distribution is 1000). This Confidence Level value is indicative of the confidence or probability that the abundance of target molecules in an environment within the Confidence Interval, given a probability distribution of target abundance from the StochQuant model of the molecular detection workflow.

### (Approach B)

In some examples, the distribution of probable numbers of target molecules in an environment is approximated by a known probability distribution, such as a negative binomial distribution. In such examples (assuming one has already obtained the shape parameters of the negative binomial distribution), one can obtain the probability that the number of target molecules in an environment is greater than the molecular detection by the following computation:

$$P ( Num Target Molecules > Detection Threshold ) = 1 - CDF ( Detection Threshold )$$

where CDF is the cumulative distribution function of the distribution of probable number of target molecules in an environment. In this example of a negative binomial distribution, the CDF can be obtained by using common software packages such as Scipy Stats Nbinom module.

Then, compare the Confidence Level obtained to the Confidence Level Threshold. If the Confidence Level obtained is greater than the confidence-level threshold, then the target is detected. If the level of confidence obtained is less than the confidence-level threshold, then the target is not detected.

In other examples, it is possible to have more than two outcomes (e.g., instead of just detected vs non-detected, the outcomes of the detection can be detected, indeterminant, and non-detected). An indeterminant determination can be used to change the action in response to the molecular detection, including re-running the molecular detection on the environment of interest, modifying the molecular detection workflow (e.g., increasing the amount of sample separated from an environment as referenced in FIG. 3A), or selecting another molecular detection workflow. In this example with more than two outcomes, multiple confidence-level threshold are established. For example, a confidence level less than 0.05 yields non-detection, a confidence level between 0.05 and 0.95 yields indeterminant, and a confidence level greater than 0.95 yields detection.

### Example 42 Quantitative Detection of a Target

This is an example of quantitative detection of a target based upon the probability distribution of the abundance of a target molecule in an environment.

Related examples can include: the quantitative detection of a target based upon the probability distribution of (i) the number of target molecules in the environment, (ii) the number of target molecules in an environment in relationship to another target, (iii) the number of target molecules in an environment in relationship to a reference molecule (e.g., a relative abundance).

Examples of confidence intervals used for the quantitative detection of a target can include:
3,000 to 4,000 target molecules in an environment
20-30% relative abundance (e.g., 20 target molecules per 100 reference molecules to 30 target molecules per 100 reference molecules),
2 target: 1 reference molecule to 3target: 1 refence molecule,
100 target copies per microliter in the environment.

A confidence level can be obtained by following a procedure outlined in Example 41. In this case, the confidence level can be used to determine the quantitative detection of the target, accordingly that the target is confidently detected within the confidence interval provided by the user.

### Example 43—Precision

This is an example of using a StochQuant probability distribution of target abundance in an environment to determine if a measurement workflow yielded a measure of target abundance in an environment that is within a (user-selected) minimum required precision of the measurement for a given confidence level threshold.

To make this determination, a user provides
a probability distribution of target abundance in an environment, e.g. derived via StochQuant,
a minimum required precision of the measurement,
and a confidence level threshold.

Examples of minimum required precision can include:
2× precision (2× being a multiplicative factor); meaning that there is a confidence interval of target abundances in an environment that are less than or equal to 2× greater than or 2×less than the target abundance value,
a range of 300; meaning that there is a confidence interval of target abundance in an environment such that the maximum value of the interval minus the minimum value of the interval is 300,
20% of the target abundance value; meaning that there is a confidence interval of target abundance in an environment that has a lower bound of the confidence interval at the target abundance value minus 20% of the target abundance value, and an upper bound of the confidence interval at the target abundance value plus 20% of the target abundance value.

Then, to determine if a measurement workflow yielded a measure of target abundance in an environment that is within a (user-selected) minimum required precision of the measurement for a given confidence level threshold, for a set of target abundance values, a user can calculate a confidence interval based on the target abundance value and the minimum precision, and then calculate a confidence level based on the confidence interval and the probability distribution of target abundance as described in Example 41.

Examples of calculating the confidence interval based on the target abundance value and the minimum precision can include:
Calculating the confidence interval for a target abundance value of 1000 and a minimum precision of 2×; in this example, the confidence interval can be calculated as follows:

$${CI}_{min} = ( target abundance ) \times \frac{1}{Precision} = ( 1000 ) \times \frac{1}{2} = 500$$

$${CI}_{max} = ( target abundance ) \times Precision = ( 1000 ) \times 2 = 2000$$

yielding a confidence interval of 500 to 2000 target molecules in an environment. Examples of sets of target abundance values can include:
a set of target abundance values that start at zero and incrementally increase in a linear or a logarithmic scale to a maximum target abundance value that one can expect to detect or quantitatively detect the target in an environment. For example, one can create that starts at zero, then one, then incrementally increases in logarithmic scale in increments of 0.01 logs until the target abundance value reaches 1010 target copies in an environment.
a set of target abundance values that are randomly selected from a random-uniform distribution from a minimum value to a maximum value,
a set can include a single target abundance value that is selected by the user,
a set can include a single target abundance value that is determined based on the probability distribution of target abundance; for example, the single target abundance value can be selected based on the “peak” of the probability distribution (also referred to as the point of the probability distribution with the highest probability or highest probability density) or of another value of the probability distribution such as the mean or median.

If a target abundance value from the set of target abundance values can yield a confidence level greater than the confidence level threshold for a given user-selected precision, then the measurement is determined to have yielded a level of precision required by the user.

### Example 44 Quantitative Detection of More than One Target in an Environment

This is an example of quantitative detection of more than one target in an environment. In particular, this example is an example of performing a quantitative detection of more than one target in an environment for which the following are provided by the user:
a probability distribution of Target A in an environment,
a confidence interval of Target A abundance values in the environment,
a confidence level threshold of Target A,
a probability distribution of Target B in an environment,
a confidence interval of Target B abundance values in the environment,
a confidence level threshold of Target B,

In this example, a confidence level is obtained each for Target A and Target B following the exemplary procedures described in Example 41.

If the confidence level of Target A is greater than the confidence level threshold of Target A, and the confidence level of Target B is greater than the confidence level threshold of Target B, then the targets are quantitatively detected.

### Example 45 Quantitative Detection of More than One Target in an Environment

This is an example of quantitative detection of more than one target in an environment. In particular, this example is an example of performing a quantitative detection of more than one target in an environment for which the following are provided by the user:
a probability distribution of Target A in an environment,
a confidence interval of Target A abundance values in the environment,
a probability distribution of Target B in an environment,
a confidence interval of Target B abundance values in the environment,
a confidence level threshold for Target A and B

In this example, a confidence level is obtained each for Target A and Target B following the exemplary procedures described in Example 41. Then the confidence level that Target A and Target B are both present within their confidence intervals can be calculated by multiplying the confidence level of Target A by the confidence level of Target B. If this confidence level for Target A and B is greater than the confidence level threshold for Target A and B, then the targets are quantitatively detected.

### Example 46 Quantitative Detection of a Target in Two or More Environments

In some examples, detection or quantitative detection is set based on the quantitative detection of a target in two or more environments.

For example, in the Contamination Filtering Example within the proof-of-concept Amplicon Example 13,
a probability distribution of a target abundance in an NTC (no-template control) environment is used to obtain the 99^{th} percentile value of the probability distribution of the target absolute abundance in the NTC environment,
and a probability distribution of the same target abundance in another environment (e.g., MD4) is used to obtain the 1^{st} percentile of the probability distribution of the target absolute abundance in the MD4 environment.

The target was determined to be detected in the MD4 environment if the target abundance value of the 1^{st} percentile of the probability distribution (of the target absolute abundance) in the MD4 environment was greater than the target abundance value of the 99^{th} percentile of the probability distribution (of the target absolute abundance) in the MD1 environment.

Detection of a target in the MD4 dilution environment is determined based on the quantitative detection of the target in the NTC environment and the MD4 environment.

In another example, the quantitative detection in multiple environments such as multiple clinical specimens collected from a human, the multiple clinical specimens being
collected from different locations/sites/organs of the human,
different specimen-types,
collected at different points in time.

In this example, for each environment, (i) a confidence level threshold, (ii) a probability distribution of target abundance in the environment, and (iii) a confidence interval are provided. A confidence level is obtained for the target in each environment following the exemplary procedures described in Example 41.

In this example, the target is detected or quantitatively detected if the confidence level is greater than the confidence level threshold of the environment in at least one, more than one, or all of the environments of interest.

### Example 47 Quantitative Detection of a Target in Two or More Groups of Environments

This example would cross-reference the differential abundance analyses. Can also cross reference the bulk RNA-seq longitudinal analysis example.

### Example 48 Inference with a Neural Network

In this example, a neural network is trained to take as inputs the physical parameters of a testing measurement workflow and as an output yield a probability distribution of target abundances. In this example, a neural network improves the computational speed of the StochQuant Workflow.

In this example, the Measurement Workflow Representation is provided in the form of a Python function that takes as inputs the following physical parameters:
the number of target molecules in an environment,
an absolute anchoring measurement of the number of reference molecules in an environment,
a measurable amount of sample separated from an environment (measured in microliters)
a measurable amount of an environment (measured in microliters),
a molecular count of the reference molecule yielded from the testing measurement,
   and as an output, the Measurement Workflow Representation provides a numpy array of probable molecular counts of the target molecule.

In this example, the Accuracy of the Measurement Workflow Representation has already been assessed, and the Measurement Workflow Representation and physical parameters have already been incorporated into an Inference Method that yields a probability distribution of target abundance in the form of shape parameters (n, p) of a negative binomial distribution as discussed in Example 35.

### Generation of Data to Train the Neural Network

Training data was generated as follows:

First, 50,000 random sets of physical parameters were generated by using a Numpy random number generator. The range of values of the physical parameters varied based upon the range of physical parameters for which the user desired to perform the StochQuant Workflow, guided by the range of physical parameter values expected to be encountered in the course of performing the testing measurement. For example:
the absolute anchoring measurement of the reference molecule (physical parameter) values ranged from 10,000 to 109 molecules,
the molecular count of the reference molecule (physical parameter) values ranged from 1,000 to 200,000 molecular counts,
the measurable amount of a sample separated from an environment (physical parameter) value was set to 2.5 microliters because the testing measurement in this example always separates 2.5 microliters of sample from an environment.
the measurable amount of an environment (physical parameter) value was set to 100 microliters because the testing measurement in this example always is always measuring the number of target molecules in an environment of 100 microliters.
the number of target molecules in an environment (physical parameter) values were set by randomly generating a relative abundance value (between 0.001 and 1) and then multiplying that value by the randomly generated value of the number of reference molecule in an environment,
and the molecular count of the target molecule (physical parameter) values were set by using the Measurement Workflow Representation to calculate the expected molecular count of the target molecule, given the other physical parameters.

Then, for each set of random parameter values, the Inference Procedure that incorporated the Measurement Workflow Representation and the physical parameters was used to yield a probability distribution in the form of the two negative binomial shape parameters (n and p).

The sets of random parameter values and the corresponding negative binomial shape parameters were saved as a dataset in the form of a CSV file.

### Training the Neural Network

The numpy, pandas, tensorflow, and sklearn Python libraries were used to train the Neural Network in a Python Jupyter Notebook.

A subset of the training data (the first 25,000 random sets of parameters) was used for training the neural network and a subset of the training data (the final 25,000 random sets of parameters) was used for validation of the trained neural network.

First, training data was split into NN input parameters (the physical parameters that the neural network will use to yield a given probability distribution of target abundance in an environment) and NN output parameters (the negative binomial shape parameters n and p that the neural network will yield for a given set of input parameters). For computational simplicity, the neural network was not trained on the amount of sample separated from an environment and was not trained on the measurable amount of the environment because these two parameters did not vary across any of the sets of physical parameters.

A log1p transformation was applied to the NN input parameters, then NN input and NN output parameters were scaled using the sklearn MinMaxScaler and sklearn fit_transform function.

Then a neural network architecture was formed using the tensorflow keras Sequential function. The network used 5 Dense layers, “relu” activation functions in each layer, and the following series of neurons per layer: 128, 64, 32, 16, 2.

The neural network was compiled with a mean squared error loss function and the “adam”optimizer.

The neural network was fit using the scaled training data with 1000 epochs, a batch size of 256, validation split of 0.3, with early stopping based on monitoring “val loss” with a patience of 60 and “the restore_best_weights” set to TRUE.

### Evaluation of the Neural Network Training

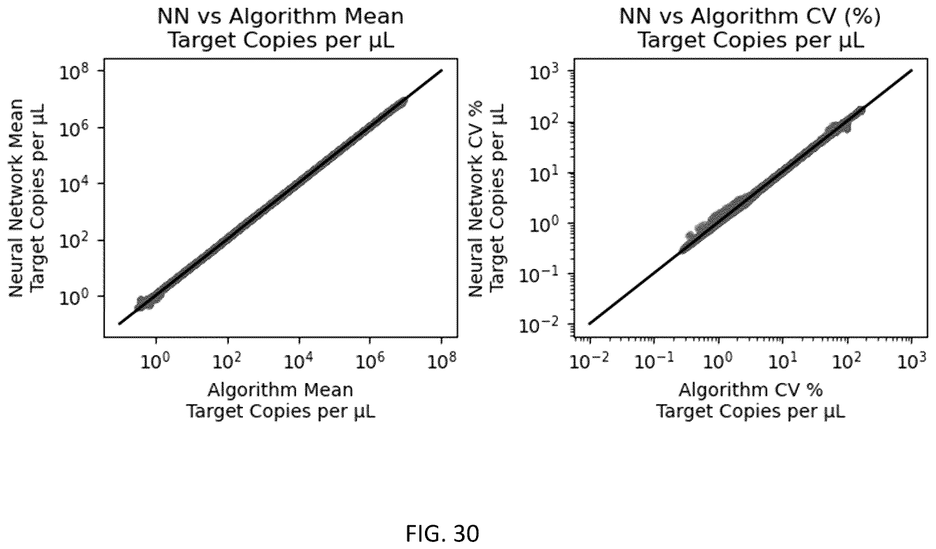

The neural network was evaluated by using the 50,000 parameter sets that were used to generate the initial probability distributions by the initial inference procedure as inputs into the neural network. The neural network then provided (for each of the parameter sets) the shape parameters (n and p) to parameterize a negative binomial distribution of the number of target molecules in an environment. Then, to assess the distributions provided by the neural network, the mean and variance of these distributions were compared to the mean and variances of the distributions provided by the initial inference procedure (FIG. 30).

### The Improvement

The use of the neural network to provide probability distributions of target abundance is a demonstration of the improvement in computational performance. In this example, on the same machine (a personal laptop), the original inference of the 50,000 distributions took approximately 2 hours total. On the same computer, the inference of the 50,000 distributions with the neural network too less than one second.

In summary, described herein are described of a stochastic quantitative approach (StochQuant) that uses molecular counts obtained from a testing measurement, an absolute anchoring measurement of a reference molecule, and possibly additional physical parameters such as quantitatively measurable amounts of a sample, to identify a probability distribution, a confidence interval and/or a confidence level in outcome of a testing measurement of a target molecule, thus improving reliability and accuracy of quantitative detection of the target molecule performed by the testing measurement . . .

The examples set forth above as well as in Appendix A and Appendix B of U.S. Provisional Application No. 63/579,291 incorporated by reference in its entirety are provided to give those of ordinary skill in the art a disclosure and description of how to make and use embodiments of the materials, compositions, systems and methods of the disclosure, and are not intended to limit the scope of what the inventors regard as their disclosure. Those skilled in the art will recognize how to adapt the features of the exemplified methods and systems based on the specific target molecule, reference molecule, anchoring measurements and samples as well as related quantitatively measured amount according to various embodiments and scope of the claims.

All patents and publications mentioned in the instant specification inclusive of Appendix A and Appendix B of U.S. Provisional Application No. 63/579,291 incorporated by reference in its entirety are indicative of the levels of skill of those skilled in the art to which the disclosure pertains.

The entire disclosure of each document cited (including webpages patents, patent applications, journal articles, abstracts, laboratory manuals, books, or other disclosures) in the instant disclosure inclusive of the Appendix A and Appendix B of U.S. Provisional Application No. 63/579,291 incorporated by reference in its entirety is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence. All references are taken as they were at filing date of the present disclosure.

The terms and expressions which have been employed in the instant disclosure inclusive of the Appendix A and Appendix B of U.S. Provisional Application No. 63/579,291 are used as terms of description and not of limitation, and there is no intention in the use of such terms and expressions of excluding any equivalents of the features shown and described or portions thereof, but it is recognized that various modifications are possible within the scope of the materials, compositions, systems and methods of the disclosure claimed. Thus, it should be understood that although the materials, compositions, systems and methods of the disclosure have been specifically described by embodiments, exemplary embodiments and optional features, modification and variation of the concepts herein described can be resorted to by those skilled in the art, and that such modifications and variations are considered to be within the scope of this disclosure in the instant disclosure inclusive of the Appendix A and Appendix B.

It is also to be understood that the terminology used herein is for the purpose of describing particular embodiments only and is not intended to be limiting. As used in this specification inclusive of Appendix A and Appendix B of U.S. Provisional Application No. 63/579,291 and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used in the instant disclosure, all individual members of the group and all combinations and possible subcombinations of the group are intended to be individually included in the disclosure. Every combination of components or materials described or exemplified herein can be used to practice the materials, compositions, systems and methods of the disclosure, unless otherwise stated. One of ordinary skill in the art will appreciate that methods, device elements, and materials other than those specifically exemplified can be employed in the practice of the materials, compositions, systems and methods of the disclosure without resort to undue experimentation. All art-known functional equivalents, of any such methods, device elements, and materials are intended to be included in the instant disclosure inclusive of Appendix A and Appendix B of U.S. Provisional Application No. 63/579,291

Whenever a range is given in the specification inclusive of the Appendix A and Appendix B of U.S. Provisional Application No. 63/579,291, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations, which is not specifically disclosed herein.

“Optional” or “optionally” in the instant disclosure inclusive of Appendix A and Appendix B of U.S. Provisional Application No. 63/579,291 means that the subsequently described circumstance may or may not occur, so that the description includes instances where the circumstance occurs and instances where it does not according to the guidance provided in the present disclosure. For example, the phrase “optionally substituted” means that a non-hydrogen substituent may or may not be present on a given atom, and, thus, the description includes structures wherein a non-hydrogen substituent is present and structures wherein a non-hydrogen substituent is not present. It will be appreciated that the phrase “optionally substituted” is used interchangeably with the phrase “substituted or unsubstituted.” Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned can be identified in view of the desired features of the compound in view of the present disclosure, and in view of the features that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

A number of embodiments of materials, compositions, systems and methods of the disclosure have been described. The specific embodiments provided herein are examples of useful embodiments of the materials, compositions, systems and methods of the disclosure and it will be apparent to one skilled in the art that the materials, compositions, systems and methods of the disclosure can be carried out using a large number of variations of the devices, device components, methods steps set forth in the present in the instant disclosure inclusive of the Appendix A and Appendix B of U.S. Provisional Application No. 63/579,291 incorporated by reference in its entirety. As will be obvious to one of skill in the art, methods and devices useful for the present methods can include a large number of optional composition and processing elements and steps.

In particular, it will be understood that various modifications may be made without departing from the spirit and scope of the present in the instant disclosure inclusive of the Appendix A and Appendix B. of U.S. Provisional Application No. 63/579,291 incorporated by reference in its entirety Accordingly, other embodiments are within the scope of the following claims.

### REFERENCES

ADDIN EN.REFLIST 1. Duffy, K., S. Arangundy-Franklin, and P. Holliger, Modified nucleic acids: replication, evolution, and next-generation therapeutics. BMC Biol, 2020. 18 (1): p. 112.
2. Wiki Aptamer. 2023; Available from: https://en.wikipedia.org/wiki/Aptamer (accessed at the filing date of the present disclosure).
3. Zhou, G., et al., Aptamers: A promising chemical antibody for cancer therapy. Oncotarget, 2016. 7 (12): p. 13446-63.
4. Raj, A., et al., Imaging individual mRNA molecules using multiple singly labeled probes. Nat Methods, 2008. 5 (10): p. 877-9.
5. Choi, H. M. T., et al., Third-generation in situ hybridization chain reaction: multiplexed, quantitative, sensitive, versatile, robust. Development, 2018. 145 (12).
6. Ismagilov, R. F., J. T. Barlow, and S. R. Bogatyrev, Absolute quantification of nucleic acids and related methods and systems, USPTO, Editor. 2021: United States.
7. Soltermann, F., et al., Quantifying Protein-Protein Interactions by Molecular Counting with Mass Photometry. Angew Chem Int Ed Engl, 2020. 59 (27): p. 10774-10779.
8. Wu, C., P. M. Garden, and D. R. Walt, Ultrasensitive Detection of Attomolar Protein Concentrations by Dropcast Single Molecule Assays. J Am Chem Soc, 2020. 142 (28): p. 12314-12323.
9. Sze, J. Y. Y., et al., Single molecule multiplexed nanopore protein screening in human serum using aptamer modified DNA carriers. Nat Commun, 2017. 8 (1): p. 1552.
10. Kantak, M., P. Batra, and P. Shende, Integration of DNA barcoding and nanotechnology in drug delivery. Int J Biol Macromol, 2023. 230: p. 123262.
11. Liszczak, G. and T. W. Muir, Nucleic Acid-Barcoding Technologies: Converting DNA Sequencing into a Broad-Spectrum Molecular Counter. Angew Chem Int Ed Engl, 2019. 58 (13): p. 4144-4162.
12. Pampel, J. Housekeeping Genes. 2024; Available from: https://www.genomics-online.com/resources/16/5049/housekeeping-genes/(accessed at the filing date of the present disclosure).
13. ResearchGate. Q&A: What are the commonly used housekeeping/constitutive genes in bacteia (sic) for gene expression studies? 2018; Available from: https://www.researchgate.net/post/What_are_the_commonly_used_housekeeping_constit utive_genes_in_bacteia_for_gene_expression_studies #: ˜: text=generally % 2C %20so %2D called % 20%22house,B %2C %20mdoG %2C %20arcA).%20DOI: %2010.1186/s12864-015-1224-y (accessed at the filing date of the present disclosure).
14. Llanos, A., J. M. François, and J.-L. Parrou, Tracking the best reference genes for RT-qPCR data normalization in filamentous fungi. BMC Genomics, 2015. 16 (1): p. 71.
15. Quail, M. A., H. Swerdlow, and D. J. Turner, Improved protocols for the illumina genome analyzer sequencing system. Curr Protoc Hum Genet, 2009. Chapter 18: p. Unit 18 2.
16. Quan, P. L., M. Sauzade, and E. Brouzes, dPCR: A Technology Review. Sensors (Basel), 2018. 18 (4).
17. Collier L, B. A., Sussman M Mahy B., Topley and Wilson's Microbiology and Microbial Infections. Virology. Vol. 1 (9^{th} ed.) 1998: Collier LA (eds.).
18. Caspar DL, K. A., Physical principles in the construction of regular viruses. 1962: Cold Spring Harbor Symposia on Quantitative Biology.
19. Crick FH, W. J., Structure of small viruses. Nature, March 1956. 177 (4506): : p. 473-75.
20. Wikipedia. Viral Envelope. Available from: https://en.wikipedia.org/wiki/Viral_envelope (accessed at the filing date of the present disclosure).
21. Fajardo, G. and H. Hornicke, Problems in estimating the extent of coprophagy in the rat.Br J Nutr, 1989. 62 (3): p. 551-61.
22. Scispot. Exploring the Human Microbiome: Unveiling the Top 20 Companies Leading Microbiome Research in the US. 2023; Available from: https://www.scispot.com/blog/top-20-microbiome-companies-in-the-us (accessed at the filing date of the present disclosure).
23. Illumina. Wastewater Surveillance. 2024; Available from: https://www.illumina.com/areas-of-interest/microbiology/public-health-surveillance/wastewater-surveillance.html (accessed at the filing date of the present disclosure).
24. Klein, E. A., T. M. Beer, and M. Seiden, The Promise of Multicancer Early Detection. Comment on Pons-Belda et al. Can Circulating Tumor DNA Support a Successful Screening Test for Early Cancer Detection? The Grail Paradigm. Diagnostics 2021, 11, 2171. Diagnostics (Basel), 2022. 12 (5).
25. Natera. Altera Comprehensive Genomic Profiling. 2024; Available from: https://www.natera.com/oncology/signatera-advanced-cancer-detection/clinicians/altera/(accessed at the filing date of the present disclosure).
26. Dekker, S. E., et al., Using Measurable Residual Disease to Optimize Management of AML, ALL, and Chronic Myeloid Leukemia. Am Soc Clin Oncol Educ Book, 2023. 43 (43): p. e390010.
27. O'Sullivan, H. M., A. Feber, and S. Popat, Minimal Residual Disease Monitoring in Radically Treated Non-Small Cell Lung Cancer: Challenges and Future Directions. Onco Targets Ther, 2023. 16: p. 249-259.
28. Teixeira, A., et al., Current and Emerging Techniques for Diagnosis and MRD Detection in AML: A Comprehensive Narrative Review. Cancers (Basel), 2023. 15 (5).
29. Yu, Z., et al., The evolution of minimal residual disease: key insights based on a bibliometric visualization analysis from 2002 to 2022. Front Oncol, 2023. 13: p. 1186198.
30. Fu, F., et al., Application of exome sequencing for prenatal diagnosis of fetal structural anomalies: clinical experience and lessons learned from a cohort of 1618 fetuses. Genome Medicine, 2022. 14 (1): p. 123.
31. Dar, P., et al., Cell-free DNA screening for prenatal detection of 22q11.2 deletion syndrome. Am J Obstet Gynecol, 2022. 227 (1): p. 79.e1-79.e11.
32. Dar, P., et al., Cell-free DNA screening for trisomies 21, 18, and 13 in pregnancies at low and high risk for aneuploidy with genetic confirmation. Am J Obstet Gynecol, 2022. 227 (2): p. 259.e1-259.e14.
33. Chiu, C. Y. and S. A. Miller, Clinical metagenomics. Nat Rev Genet, 2019. 20 (6): p. 341-355.
34. Kalantar, K. L., et al., Integrated host-microbe plasma metagenomics for sepsis diagnosis in a prospective cohort of critically ill adults. Nat Microbiol, 2022. 7 (11): p. 1805-1816.
35. Casalini, G., A. Giacomelli, and S. Antinori, The WHO fungal priority pathogens list: a crucial reappraisal to review the prioritisation. The Lancet Microbe, 2024. 5 (7): p. 717-724.
36. Fisher, M. C. and D. W. Denning, The WHO fungal priority pathogens list as a game-changer. Nature Reviews Microbiology, 2023. 21 (4): p. 211-212.
37. WHO, WHO fungal priority pathogens list to guide research, development and public health action. 2022. p. 48; accessed on the date of this disclosure.
38. Barlow, J. T., S. R. Bogatyrev, and R. F. Ismagilov, A quantitative sequencing framework for absolute abundance measurements of mucosal and lumenal microbial communities. Nature Communications, 2020. 11 (1): p. 2590.
39. Wu-Woods, N. J., et al., Microbial-enrichment method enables high-throughput metagenomic characterization from host-rich samples. Nat Methods, 2023. 20 (11): p. 1672-1682.
40. Barlow, J. T., S. R. Bogatyrev, and R. F. Ismagilov, A quantitative sequencing framework for absolute abundance measurements of mucosal and lumenal microbial communities. Nat Commun, 2020. 11 (1): p. 2590.
41. Bolyen, E., et al., Reproducible, interactive, scalable and extensible microbiome data science using QIIME 2. Nat Biotechnol, 2019. 37 (8): p. 852-857.
42. Callahan, B. J., et al., DADA2: High-resolution sample inference from Illumina amplicon data. Nat Methods, 2016. 13 (7): p. 581-3.
43. Bokulich, N. A., et al., Optimizing taxonomic classification of marker-gene amplicon sequences with QIIME 's q2-feature-classifier plugin. Microbiome, 2018. 6 (1): p. 90.
44. Quast, C., et al., The SILVA ribosomal RNA gene database project: improved data processing and web-based tools. Nucleic Acids Res, 2013. 41 (Database issue): p. D590-6.
45. Galeano Nino, J. L., et al., Effect of the intratumoral microbiota on spatial and cellular heterogeneity in cancer. Nature, 2022. 611 (7937): p. 810-817.
46. Earley, Z. M., et al., GATA4 controls regionalization of tissue immunity and commensal—driven immunopathology. Immunity, 2023. 56 (1): p. 43-57 e10.
47. Donald, K. and B. B. Finlay, Early-life interactions between the microbiota and immune system: impact on immune system development and atopic disease. Nature Reviews Immunology, 2023. 23 (11): p. 735-748.
48. Claassen-Weitz, S., et al., Optimizing 16S rRNA gene profile analysis from low biomass nasopharyngeal and induced sputum specimens. BMC Microbiol, 2020. 20 (1): p. 113.
49. Wong, S. H. and J. Yu, Gut microbiota in colorectal cancer: mechanisms of action and clinical applications. Nat Rev Gastroenterol Hepatol, 2019. 16 (11): p. 690-704.
50. Man, W. H., W. A. de Steenhuijsen Piters, and D. Bogaert, The microbiota of the respiratory tract: gatekeeper to respiratory health. Nat Rev Microbiol, 2017. 15 (5): p. 259-270.
51. Natalini, J. G., S. Singh, and L. N. Segal, The dynamic lung microbiome in health and disease. Nat Rev Microbiol, 2023. 21 (4): p. 222-235.
52. Kastl, A. J., Jr., et al., The Structure and Function of the Human Small Intestinal Microbiota: Current Understanding and Future Directions. Cell Mol Gastroenterol Hepatol, 2020. 9 (1): p. 33-45.
53. Libertucci, J. and V. B. Young, The role of the microbiota in infectious diseases. Nat Microbiol, 2019. 4 (1): p. 35-45.
54. Muller, P. A., et al., Microbiota modulate sympathetic neurons via a gut-brain circuit. Nature, 2020. 583 (7816): p. 441-446.
55. Barlow, J. T., et al., Quantitative sequencing clarifies the role of disruptor taxa, oral microbiota, and strict anaerobes in the human small-intestine microbiome. Microbiome, 2021. 9 (1): p. 214.
56. Ringel, Y., et al., High throughput sequencing reveals distinct microbial populations within the mucosal and luminal niches in healthy individuals. Gut Microbes, 2015. 6 (3): p. 173-81.
57. Debroas, D., C. Hochart, and P. E. Galand, Seasonal microbial dynamics in the ocean inferred from assembled and unassembled data: a view on the unknown biosphere. ISME Commun, 2022. 2 (1): p. 87.
58. Kurm, V., et al., Low abundant soil bacteria can be metabolically versatile and fast growing. Ecology, 2017. 98 (2): p. 555-564.
59. Bickel, S. and D. Or, The chosen few—variations in common and rare soil bacteria across biomes. The ISME Journal, 2021. 15 (11): p. 3315-3325.
60. Blauwkamp, T. A., et al., Analytical and clinical validation of a microbial cell-free DNA sequencing test for infectious disease. Nat Microbiol, 2019. 4 (4): p. 663-674.
61. Poore, G. D., et al., Microbiome analyses of blood and tissues suggest cancer diagnostic approach. Nature, 2020. 579 (7800): p. 567-574.
62. Schlaberg, R., Microbiome Diagnostics. Clin Chem, 2020. 66 (1): p. 68-76.
63. France, M., et al., Towards a deeper understanding of the vaginal microbiota. Nat Microbiol, 2022. 7 (3): p. 367-378.
64. Muzny, C. A., et al., State of the Art for Diagnosis of Bacterial Vaginosis. J Clin Microbiol, 2023. 61 (8): p. e0083722.
65. Aagaard, K., et al., The placenta harbors a unique microbiome. Sci Transl Med, 2014. 6 (237): p. 237ra65.
66. de Goffau, M. C., et al., Human placenta has no microbiome but can contain potential pathogens. Nature, 2019. 572 (7769): p. 329-334.
67. Rackaityte, E., et al., Viable bacterial colonization is highly limited in the human intestine in utero. Nat Med, 2020. 26 (4): p. 599-607.
68. Notarbartolo, V., et al., Composition of Human Breast Milk Microbiota and Its Role in Children's Health. Pediatr Gastroenterol Hepatol Nutr, 2022. 25 (3): p. 194-210.
69. Lopez Leyva, L., N. J. B. Brereton, and K. G. Koski, Emerging frontiers in human milk microbiome research and suggested primers for 16S rRNA gene analysis. Comput Struct Biotechnol J, 2021. 19: p. 121-133.
70. Al Alam, D., et al., Human Fetal Lungs Harbor a Microbiome Signature. Am J Respir Crit Care Med, 2020. 201 (8): p. 1002-1006.
71. York, A., Tumour-specific microbiomes. Nat Rev Microbiol, 2020. 18 (8): p. 413.
72. Riquelme, E., et al., Tumor Microbiome Diversity and Composition Influence Pancreatic Cancer Outcomes. Cell, 2019. 178 (4): p. 795-806 e12.
73. Glassing, A., et al., Inherent bacterial DNA contamination of extraction and sequencing reagents may affect interpretation of microbiota in low bacterial biomass samples. Gut Pathog, 2016. 8 (1): p. 24.
74. de Steenhuijsen Piters, W. A. A. and D. Bogaert, Bacterial DNA in Fetal Lung Samples May Be Explained by Sample Contamination. Am J Respir Crit Care Med, 2020. 201 (10): p. 1310-1311.
75. Lauder, A. P., et al., Comparison of placenta samples with contamination controls does not provide evidence for a distinct placenta microbiota. Microbiome, 2016. 4 (1): p. 29.
76. Kennedy, K. M., et al., Questioning the fetal microbiome illustrates pitfalls of low-biomass microbial studies. Nature, 2023. 613 (7945): p. 639-649.
77. Tan, C. C. S., et al., No evidence for a common blood microbiome based on a population study of 9,770 healthy humans. Nat Microbiol, 2023. 8 (5): p. 973-985.
78. Karstens, L., et al., Controlling for Contaminants in Low-Biomass 16i S rRNA Gene Sequencing Experiments. mSystems, 2019. 4 (4).
79. Salter, S. J., et al., Reagent and laboratory contamination can critically impact sequence-based microbiome analyses. BMC Biol, 2014. 12 (1): p. 87.
80. Gihawi, A., et al., Major data analysis errors invalidate cancer microbiome findings. bioRxiv, 2023.
81. Eisenhofer, R., et al., Contamination in Low Microbial Biomass Microbiome Studies: Issues and Recommendations. Trends Microbiol, 2019. 27 (2): p. 105-117.
82. Davis, N. M., et al., Simple statistical identification and removal of contaminant sequences in marker-gene and metagenomics data. Microbiome, 2018. 6 (1): p. 226.
83. Bolyen, E., et al., Reproducible, interactive, scalable and extensible microbiome data science using QIIME 2. Nat Biotechnol, 2019. 37 (8): p. 852-857.
84. Reitmeier, S., et al., Handling of spurious sequences affects the outcome of high-throughput 16S rRNA gene amplicon profiling. ISME Commun, 2021. 1 (1): p. 31.
85. Gloor, G. B., et al., Microbiome Datasets Are Compositional: And This Is Not Optional. Front Microbiol, 2017. 8: p. 2224.
86. Fernandes, A. D., et al., ANOVA-like differential expression (ALDEx) analysis for mixed population RNA-Seq. PLOS One, 2013. 8 (7): p. e67019.
87. Yang, L. and J. Chen, A comprehensive evaluation of microbial differential abundance analysis methods: current status and potential solutions. Microbiome, 2022. 10 (1): p. 130.
88. Cappellato, M., G. Baruzzo, and B. Di Camillo, Investigating differential abundance methods in microbiome data: A benchmark study. PLOS Comput Biol, 2022. 18 (9): p. e1010467.
89. Hawinkel, S., et al., A broken promise: microbiome differential abundance methods do not control the false discovery rate. Brief Bioinform, 2019. 20 (1): p. 210-221.
90. Kaul, A., et al., Analysis of Microbiome Data in the Presence of Excess Zeros. Front Microbiol, 2017. 8: p. 2114.
91. Weiss, S., et al., Normalization and microbial differential abundance strategies depend upon data characteristics. Microbiome, 2017. 5 (1): p. 27.
92. Nearing, J. T., et al., Microbiome differential abundance methods produce different results across 38 datasets. Nat Commun, 2022. 13 (1): p. 342.
93. Sinha, R., et al., Assessment of variation in microbial community amplicon sequencing by the Microbiome Quality Control (MBQC) project consortium. Nat Biotechnol, 2017. 35 (11): p. 1077-1086.
94. Bokulich, N. A., et al., Optimizing taxonomic classification of marker-gene amplicon sequences with QIIME 2's q2-feature-classifier plugin. Microbiome, 2018. 6 (1): p. 90.
95. Silverman, J. D., et al., Naught all zeros in sequence count data are the same. Comput Struct Biotechnol J, 2020. 18: p. 2789-2798.
96. Love, M. I., W. Huber, and S. Anders, Moderated estimation of fold change and dispersion for RNA-seq data with DESeq2. Genome Biol, 2014. 15 (12): p. 550.
97. Fernandes, A. D., et al., Unifying the analysis of high-throughput sequencing datasets: characterizing RNA-seq, 16S rRNA gene sequencing and selective growth experiments by compositional data analysis. Microbiome, 2014. 2 (1): p. 15.
98. Taylor, S. C., et al., The Ultimate qPCR Experiment: Producing Publication Quality, Reproducible Data the First Time. Trends Biotechnol, 2019. 37 (7): p. 761-774.
99. Wang, Z. and J. Spadoro, Determination of Target Copy Number of Quantitative Standards Used in PCR-Based Diagnostic Assays, in Gene Quantification, F. Ferré, Editor. 1998, Birkhäuser Boston: Boston, MA. p. 31-43.

100. Rossmanith, P. and M. Wagner, A novel poisson distribution-based approach for testing boundaries of real-time PCR assays for food pathogen quantification. J Food Prot, 2011. 74 (9): p. 1404-12.

101. Stolovitzky, G. and G. Cecchi, Efficiency of DNA replication in the polymerase chain reaction. Proc Natl Acad Sci USA, 1996. 93 (23): p. 12947-52.

102. Kebschull, J. M. and A. M. Zador, Sources of PCR-induced distortions in high-throughput sequencing data sets. Nucleic Acids Res, 2015. 43 (21): p. e143.

103. Asogawa, M., Framework for qPCR modeling and analysis of low copy number sample. Forensic Science International: Genetics Supplement Series, 2022. 8: p. 344-346.

104. Svec, D., et al., How good is a PCR efficiency estimate: Recommendations for precise and robust qPCR efficiency assessments. Biomol Detect Quantif, 2015. 3: p. 9-16.

105. Klein, A. M., et al., Droplet barcoding for single-cell transcriptomics applied to embryonic stem cells. Cell, 2015. 161 (5): p. 1187-1201.

1. A method for detection of an abundance of a target molecule in a physical environment, the method comprising:
i) providing a testing measurement comprising a measuring workflow for a molecular count of a target molecule in presence of a molecular count of a reference molecule,
ii) dividing the measuring workflow into one or more measuring segments arranged in a measuring workflow order, each of the one or more measuring segments comprising one or more physical manipulations impacting the molecular count of the target molecule and/or the molecular count of the reference molecule;
iii) calibrating the one or more measuring segments by building corresponding one or more stochastic representations of each of the one or more measuring segments into a computer-based system, the one or more stochastic representations taking as inputs physical parameters of the measuring workflow;
iv) chaining the corresponding one or more stochastic representations together into a model of the measuring workflow by connecting outputs of measuring segments into inputs of other measuring segments in the measuring workflow order, such that the model takes as model inputs the physical parameters input to the model including at least a target molecule molecular count, a reference molecule molecular count, and an absolute anchoring value of the reference molecule; and
v) configuring the computer-based system to provide a probability distribution of an abundance of the target molecule based on the model of the measuring workflow once provided with the model inputs.

2. The method of claim 1, wherein at least one of the one or more physical manipulations comprises sampling the physical environment or a sample or a subsample thereof from a previous measuring segment.

3. The method of claim 1, wherein the one or more physical manipulations comprise one or more of: separation of a sample from the physical environment, flow cell binding, amplification manipulations, isolation of the target molecule, reverse transcription, and target enrichment.

4. The method of claim 1, wherein one or more measuring segments comprise at least a separation of a sample from the physical environment and a measurement.

5. The method claim 1, wherein the target molecule is related to at least one of: prenatal testing, cancer testing, an infectious testing such as testing for a sexually transmitted infection and/or bacterial vaginosis.

6. The method claim 1, wherein the reference molecule is one of: a synthetic nucleic acid that contains a unique sequence detectably different from sequences of the target molecule and other molecules in the environment; a synthetic nucleic acid having physical properties affected by physical manipulations having effect on the target molecule and the reference molecule; a plurality of 16S rRNA gene molecules; and a molecule known or expected to be in the physical environment and to be detectable with the testing measurement.

7. The method claim 1, wherein the reference molecule is one of: a gene marker of a commensal organism known or expected be in the environment and to be detectable with the testing measurement or a non-mutated human sequence be in the environment and to be detectable with the testing measurement.

8. The method claim 1, wherein the absolute anchoring value is determined by one or more of: a spike-in of the reference molecule into the environment, a digital PCR measurement, or a qPCR with a standard curve.

9. The method of claim 1, wherein the measuring workflow includes one or more of: amplicon sequencing; multiplex amplicon sequencing; shotgun metagenomic sequencing; bulk RNA sequencing; and single cell RNA sequencing.

10. The method claim 1, wherein the physical environment comprises one or more of: a sample obtained from a human, plant, fungi, bacteria colony, or animal; material derived from a sample obtained from a human, plant, fungi, bacteria colony, or animal; food; a tagged or encoded library of molecules; wastewater; and a pooled sample of any of the human, plant, fungi, bacteria colony and animal samples, any material derived therefrom, any tagged or encoded library of molecules, and/or wastewater sample.

11. The method of claim 1, wherein at least one of the one or more measuring segments comprise one or more of: separation of a sample from the environment, flow cell binding, amplification, isolation of the target molecule, reverse transcription, sequencing and target enrichment.

12. The method of claim 1, wherein at least one of the one or more stochastic representations of the one or more measuring segments comprises calculating a distribution of data for output for said at least one stochastic representation.

13. The method of claim 12, wherein the distribution is one of: a Poisson distribution, a binomial distribution, a gamma-Poisson distribution, or a negative binomial distribution.

14. The method of claim 1, further configuring the computer-based system to provide a confidence level of an abundance of the target molecule based on the model of the measuring workflow when further provided with a confidence interval and/or a threshold abundance.

15. The method of claim 14, wherein the computer-based system provides the confidence level by determining a total amount of probability above the threshold abundance value within the probability distribution.

16. The method of claim 1, further comprising configuring the computer-based system to provide a confidence level of an abundance of the target molecule by calculating a total amount of probability within a confidence interval within the probability distribution.

17. The method of claim 16, wherein the confidence interval is a pre-set value.

18. The method of claim 1, further comprising configuring the computer-based system to provide a confidence interval of an abundance of the target molecule matching a given confidence level by calculating a total amount of probability matching the given confidence level within the confidence interval within the probability distribution.

19. The method of claim 18, wherein the given confidence level is input by the user of the computer-based system.

20.-174. (canceled)

175. The method of claim 1, further comprising performing the measuring workflow on the target molecule and the reference molecule according to the one or more measuring segments, to obtain a measured target molecule count and a measured reference molecule count serving as the physical parameters input to the model.

176. The method of claim 1, wherein the target molecule comprises a plurality of target molecules.

177. The method of claim 1, wherein the target molecule comprises a polynucleotide, the testing measurement is performed through detection of a sequence of the polynucleotide and the molecular count of the target molecule is in form of read counts.

178. The method of claim 1, wherein the measuring workflow of the testing measurement comprises nucleic acid sequencing, and the molecular count of a detected nucleic acid is in form of read counts.

179. The method of claim 1, wherein the measuring workflow includes one or more of next generation sequencing, sequencing by synthesis, and nanopore sequencing.

180. The method of claim 9, wherein the measuring workflow comprises amplicon sequencing and the amplicon sequencing includes one or more of: 16S rRNA gene sequencing, ITS gene sequencing, 18S rRNA gene sequencing, COI gene sequencing, ITS2 gene sequencing, RBP1 gene sequencing, RBP2 gene sequencing, V (D) J region sequencing, mitochondrial gene sequencing, functional gene sequencing, cancer biomarker gene sequencing, synthetic barcode sequencing.

181. The method of claim 1, wherein the reference molecule is selected from: Glyceraldehyde-3-phosphate dehydrogenase (GAPDH), Phosphoglycerate kinase 1 (PGK1), Peptidylpropyl isomerase A (PPIA), ribosomal protein L13a (RPL13A), ribosomal protein large P0 (RPLP0), Beta-2-microglobulin (B2M), YWHAZ, SDHA, TFRC, GUSB, HMBS, HPRT1, TBP; bacterial housekeeping genes and fungal housekeeping genes.

182. The method of claim 1, wherein the reference molecule is a plurality of types of molecules.

183. The method of claim 1, wherein the reference molecule comprises a mRNA of a gene.

184. The method of claim 1, wherein the absolute anchoring value of the reference molecule is obtained by performing in the environment an absolute anchoring measurement of the reference molecule.

185. The method of claim 1, wherein the absolute anchoring value of the reference molecule is a value obtained by a previous measurement.

186. The method of claim 1, wherein the reference molecule is added to the environment and the absolute anchoring value of the reference molecule is a known absolute count or distribution of absolute counts of the reference molecule added to the environment, sample, and/or subsample thereof.

187. The method of claim 1, wherein the molecular count of the target molecule and the molecular count of the reference molecule are obtained in a same sample or in subsamples of a same sample.

188. The method of claim 2, wherein the sample is a plurality of samples and the absolute anchoring measurement, the molecular count of the target molecule, and the probability distribution are obtained in one or more same or different samples of the plurality of samples.
